(19) United States
(12) Patent Application Publication
(10) Pub. No.: US 2011/0183978 A1
(43) Pub. Date: Jul. 28, 2011

(54) Phenylpyri(mi)dinylazoles

(75) Inventors: Alexander Sudau, Leichlingen (DE); Mazen Es-Sayed, Lyon (FR); Christoph Andreas Braun, Dusseldorf (DE); Ruth Meissner, Leverkusen (DE); Catherine Sirven, Langenfeld (DE); Jürgen Benting, Leichlingen (DE); Peter Dahmen, Neuss (DE); Daniela Portz, Vettweiss (DE); Ulrike Wachendorff-Neumann, Neuwied (DE); Philippe Desbordes, Lyon (FR); Samir Bennabi, Caluire (FR); Christophe Catherin, Le Perreon (FR); Anne-Sophie Rebstock, Lyon (FR); Marie-Claire Grosjean-Cournoyer, Curis au Mont d'Or (FR); Hiroyuki Hadano, Tochigi (JP); Thomas Knobloch, Chatillon (FR); Philippe Rinolfi, Chatillon d'Azergues (FR)

(73) Assignee: Bayer CropScience AG, Monheim (DE)

(21) Appl. No.: 12/902,985

(22) Filed: Oct. 12, 2010

Related U.S. Application Data
(60) Provisional application No. 61/250,149, filed on Oct. 9, 2009.

(30) Foreign Application Priority Data
Oct. 9, 2009 (EP) 09172677.8

(51) Int. Cl. A01N 43/84; C07D 401/04; A01N 43/40; C07D 401/14; C07D 413/14; A01N 43/54; A01P 3/00

(52) U.S. Cl. 514/236.5; 546/275.4; 514/341; 544/124; 546/256; 514/333; 544/333; 514/256

(57) ABSTRACT

Phenylpyri(mi)dinylazoles of the formula [I-a] and [I-b],

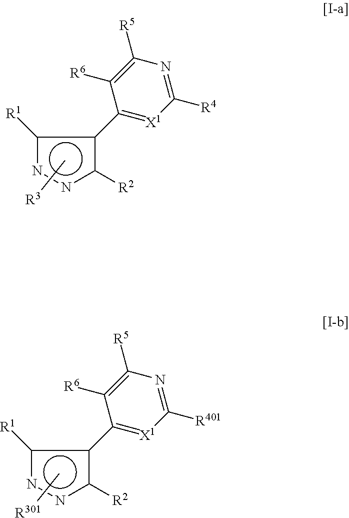

wherein the symbols have the meanings stated in the description, and agrochemically active salts thereof and the use thereof for the control of undesired microorganisms in the protection of plants and materials and for the reduction of mycotoxins in plants and plant parts and methods for the production of compounds of the formula [I-a] and [I-b].

The present invention relates to novel phenylpyri(mi)dinylazoles, several processes for the production thereof and the use thereof for the control of undesired microorganisms in the protection of plants and materials and for the reduction of mycotoxins in plants and plant parts. The present invention further relates to a process for the control of phytopathogenic fungi and for the reduction of mycotoxins in plants and plant parts in plant protection and to pesticides containing phenylpyri(mi)dinylazoles.

It is already known that certain arylpyrazoles possess fungicidal properties (e.g. see WO 03/049542, WO 01/030154 and Pharmazie 1999, 54(2), 106-11). The effectiveness of the substances described there is good, but in many cases leaves something to be desired.

In WO 98/052937, certain heteroaryl-substituted pyrazoles are described which can be used medicinally, here for the inhibition of the production of inflammatory cytokines and for the treatment of human p38 kinase-mediated diseases. Similar compounds are also described in EP-A-1 553 096, WO 04/029043, WO 98/052940, WO 00/031063, WO 95/031451, WO 02/057265 and WO 00/039116. However, an effect on fungal pathogens is not described.

In WO 07/105058 certain heteroaryl-substituted pyrazoles are described which can be used as modulators or inhibitors of the human Raf enzyme. However, the action on fungal pathogens is not described.

Since the ecological and economic requirements for modern pesticides are steadily increasing, for example as regards activity spectrum, toxicity, selectivity, application dose, residue formation and ease of production, and in addition for example problems with resistances can arise, there is the constant task of developing novel pesticides, in particular fungicides, which at least in some fields have advantages compared to the known ones.

Surprisingly it has now been found that the present phenylpyri(mi)dinylazoles solve the said problems at least in some regards and are suitable as pesticides, in particular as fungicides.

The subject of the invention are compounds of the formula [I-a],

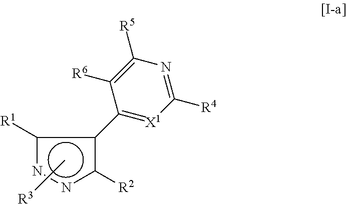

wherein the symbols have the following meanings:
X¹ stands for C—H or N,
R¹ stands for phenyl, naphthalenyl, quinolin-5-yl, quinolin-8-yl, isoquinolin-5-yl, isoquinolin-8-yl, 1-benzothiophen-4-yl, 1-benzothiophen-7-yl, 1-benzofuran-4-yl, 1-benzofuran-7-yl, 1,3-benzodioxol-4-yl or 1,3-benzodioxol-5-yl, each optionally singly or multiply, identically or differently substituted with R⁷,
R² stands for cyano, nitro, halogen, C₁-C₆ alkyl, C₁-C₆ alkoxy, C₁-C₆ haloalkyl, C₁-C₆ haloalkoxy, C₁-C₆ alkylthio, C₃-C₆ cycloalkyl, C₃-C₆ halocycloalkyl, C₂-C₉ heterocyclyl or hydrogen,
R³ stands for C₁-C₆ alkyl, C₂-C₆ alkenyl, C₂-C₆ alkynyl, C₃-C₆ cycloalkyl, C₃-C₆ cycloalkyl-C₁-C₆ alkyl, C₃-C₆ cycloalkyl-oxy, C₁-C₆ alkoxy, C₁-C₆ alkoxy-C₁-C₆ alkyl, acyloxy-C₁-C₆ alkyl, heteroaryl-C₁-C₆ alkyl (preferably C₂-C₉ heteroaryl-C₁-C₆ alkyl), aryl-C₁-C₆ alkyl (preferably C₆-C₁₄ aryl-C₁-C₆ alkyl), C₁-C₆ alkylthio-C₁-C₆ alkyl, C₃-C₆ cycloalkyl-C(O)—C₁-C₄ alkyl, C₂-C₉ heterocyclyl-C(O)—C₁-C₄ alkyl, C₁-C₄ alkyl-C(O)—C₃-C₆ cycloalkyl, C₁-C₄ alkyl-C(O)heterocyclyl (preferably C₁-C₄ alkyl-C(O)—C₂-C₉ heterocyclyl), C₁-C₄ alkyl-C(O)O—C₁-C₆ alkyl, acyloxy-C₃-C₆ cycloalkyl, acyloxy-heterocyclyl, heterocyclyl-C₁-C₆ alkyl (preferably C₂-C₉ heterocyclyl-C₁-C₆ alkyl), heterocyclyl (preferably C₂-C₉ heterocyclyl), C₂-C₉ oxoheterocycyl or heteroaryl (preferably C₂-C₉ heteroaryl), each optionally singly or multiply, identically or differently substituted with halogen, cyano, hydroxy, C₁-C₆ alkyl, C₁-C₆ alkoxy, haloalkoxy (preferably C₁-C₆ haloalkoxy), phenyl or phenoxy,
R⁴ stands for hydrogen, halogen, cyano, —C(O)OR¹², —SR¹², —NR¹²R¹³, —C(O)NR¹²R¹³ or —NR¹²R¹⁴, —N═C═NR²², —N═C(H)OR²², —N═C(OR²²)R²³, —N═C(SR²²)R²³, —C(═NR²²)NR²²R²³, —SO(═NR²²)R²³ or —SO₂R²⁰,
   or for C₁-C₆ alkyl, C₃-C₈ cycloalkyl, C₂-C₆ alkenyl, C₆-C₁₄ aryl, C₂-C₉ heterocyclyl or C₂-C₉ heteroaryl, each optionally singly or multiply, identically or differently substituted with R¹¹,
   wherein R⁴ preferably stands for hydrogen or —NHR¹³, wherein R¹³ stands for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with fluorine, chlorine, bromine, —OH, cyano, C₁-C₆ alkyl, —O—C(O)R¹¹, —O—P(O)(OR¹¹)₂, —O—B(OR¹¹)₂ or —O—(C₁-C₄ alkyl),
R⁵ and R⁶ mutually independently stand for hydrogen, fluorine, chlorine, bromine, cyano, nitro, —OH or —SH,
   or for C₁-C₆ alkyl, C₃-C₆ cycloalkyl, C₂-C₆ alkenyl, C₂-C₆ alkynyl, C₆-C₁₄ aryl, —O—(C₁-C₄ alkyl), —O—(C₆-C₁₄ aryl), —S—(C₁-C₄ alkyl), —S(O)—(C₁-C₆ alkyl) or —C(O)—(C₁-C₆ alkyl), each optionally singly or multiply, identically or differently substituted with R¹¹,
   or else together with the carbon atom to which they are bound form a ring (preferably a saturated, unsaturated or partially unsaturated single ring) with 3, preferably 5 to 8 ring atoms, wherein the ring can contain 1 to 4 hetero atoms from the range oxygen, sulphur or —NR¹⁹, optionally singly or multiply, identically or differently substituted with halogen, oxygen, cyano or C₁-C₄ alkyl,
R⁷ mutually independently stands for one or more of the following groups: fluorine, chlorine, bromine, cyano, nitro, —OH or —SH,
   or for C₁-C₆ alkyl, C₃-C₆ cycloalkyl, C₂-C₆ alkenyl, C₂-C₆ alkynyl, tri(C₁-C₄ alkyl)silyl, C₆-C₁₄ aryl, —O—(C₁-C₄ alkyl), —O—(C₆-C₁₄ aryl), —S—(C₁-C₄ alkyl), —S(O)—(C₁-C₆ alkyl), or —S(O)₂—(C₁-C₆ alkyl), each optionally singly or multiply, identically or differently substituted with fluorine, chlorine, bromine, —OH, cyano, C₁-C₆ alkyl or —O—(C₁-C₄ alkyl)
R¹¹ stands for —OH, fluorine, chlorine, bromine, cyano, —NH—C(O)R²⁰, —NR²⁰R²¹, —C(O)R²⁰, —C(O)OR²⁰, —C(O)NR²⁰R²¹ or —SO₂R²⁰,
   or for C₁-C₆ alkyl, C₂-C₆ alkenyl, C₂-C₆ alkynyl, C₁-C₁₁ heteroalkyl, C₃-C₈ cycloalkyl, —O—(C₁-C₄ alkyl), —S—(C₁-C₄ alkyl), —O—(C₃-C₈ cycloalkyl), —S—(C₃-C₈ cycloalkyl), C₆-C₁₄ aryl, —O—(C₆-C₁₄ aryl), —S—(C₆-C₁₄ aryl), C₂-C₉ heterocyclyl or C₂-C₉ heteroaryl, each optionally singly or multiply, identically or differently substituted with fluorine, chlorine, bromine, —OH, carbonyl, cyano, C₁-C₆ alkyl or —O—(C₁-C₄ alkyl),
R¹² and R¹³ mutually independently stand for one or more of the following groups: H, —C(S)R¹⁵, —C(O)R¹⁵, —SO₂R¹⁵, —C(O)OR¹⁵, —OR¹⁵ or —C(O)NR¹⁵R¹⁶
   or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with fluorine, chlorine, bromine, —OH, cyano, C₁-C₆ alkyl, —O—C(O)R¹¹, —O—P(O)(OR¹¹)₂, —O—B(OR¹¹)₂ or —O—(C₁-C₄ alkyl),
R¹⁴ stands for —CH₂—NR²²R²³, piperidin-1-ylmethyl or morpholin-4-ylmethyl
   or for C₁-C₆ alkyl or —O—(C₁-C₄ alkyl), each optionally singly or multiply, identically or
R¹⁵ and R¹⁶ mutually independently stand for hydrogen or —OH
   or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
   or (when R¹² and/or R¹³ stands for —C(O)NR¹⁵R¹⁶) together with the nitrogen atom to which they are bound form a 3 to 7-membered ring, which can contain a further hetero atom from the range N or O not (directly) adjacent to the nitrogen,
R¹⁷ and R¹⁸ mutually independently stand for one or more of the following groups: H, —C(O)OR¹¹
   or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with fluorine, chlorine, bromine, —OH, cyano, C₁-C₆ alkyl, and —O—(C₁-C₄ alkyl),
R¹⁹ stands for H, C₁-C₆ alkyl, C₃-C₆ cycloalkyl, C₂-C₆ alkenyl, C₂-C₆ alkynyl, —C(S)R¹⁵, —C(O)R¹⁵, —SO₂R¹⁵ or —C(O)OR¹⁵,
R²⁰ and R²¹ mutually independently stand for C₁-C₆ alkyl, C₃-C₆ cycloalkyl, C₂-C₆ alkenyl or C₂-C₆ alkynyl, each optionally singly or multiply, identically or differently substituted with fluorine, chlorine, bromine, —OH or cyano, or hydrogen, and
R²² and R²³ mutually independently stand for C₁-C₆ alkyl, C₃-C₆ cycloalkyl, C₂-C₆ alkenyl, C₂-C₆ alkynyl or hydrogen,

and agrochemically active salts thereof.

Combinations wherein the symbols of the formula [I-a] have the following meanings:

Compounds wherein
X¹ stands for N, and
R¹ stands for an optionally substituted phenyl,
R³ stands for butyl or propyn-2-yl,
R⁴ stands for NHR₁₂ and
R¹² stands for optionally substituted C₆-C₁₄ aryl,
and
compounds wherein
X¹ stands for N
R², R⁴, R⁵, R⁶ stand for H,
R³ stands for methyl, ethyl, allyl, 2-methoxyethyl or benzyl, when R¹ stands for 4-chlorophenyl, or
R³ stands for methyl, when R¹ stands for phenyl, 4-methoxyphenyl or 4-fluorophenyl

are excepted from the residue definitions or explanations expounded generally or expounded in preferred ranges above.

Finally, it has been found that the phenylpyri(mi)dinylazoles of the formula [I-a] according to the invention possess very good microbicidal properties and can be used for the control of undesired microorganisms in the protection of plants and materials and for the reduction of mycotoxins in plants and plant parts.

The phenylpyri(mi)dinylazoles according to the invention are generally defined by the formula [I-a]. Preferred residue definitions of the formulae named above and below are stated below. These definitions apply equally for the final products of the formula [I-a] and for all intermediates.

Preferred compounds of the formula [I-a] of the present invention are those wherein one or more of the symbols have one of the following meanings:
X¹ stands for C—H or N,
R¹ stands for phenyl, naphthalenyl, quinolin-5-yl, quinolin-8-yl, isoquinolin-5-yl, isoquinolin-8-yl, 1,3-benzodioxol-4-yl or 1,3-benzodioxol-5-yl, each optionally singly or multiply, identically or differently substituted with R⁷,
R² stands for cyano, nitro, halogen, C₁-C₆ alkyl, C₁-C₆ alkoxy, C₁-C₆ haloalkyl, C₁-C₆ haloalkoxy, C₁-C₆ alkylthio, C₃-C₆ cycloalkyl, C₃-C₆ halocycloalkyl, C₂-C₉ heterocyclyl or hydrogen,
R³ stands for C₁-C₆ alkyl, C₂-C₆ alkenyl, C₂-C₆ alkynyl, C₃-C₆ cycloalkyl, C₃-C₆ cycloalkyl-C₁-C₆ alkyl, C₆-C₁₄ aryl-C₁-C₆ alkyl, C₁-C₆ alkoxy-C₁-C₆ alkyl, C₁-C₆ alkoxy, C₂-C₉ heterocyclyl-C₁-C₆ alkyl and C₂-C₉ heteroaryl, each optionally singly or multiply, identically or differently substituted with halogen, cyano, hydroxy or haloalkoxy or for pyridin-2-ylmethyl, pyridine-3-ylmethyl, pyridin-4-ylmethyl, 2-chloro-1,3-thiazol-5-yl)methyl, 5-(trifluoromethyl)-1,3,4-thiadiazol-2-yl, 2-(methylsulphanyl)-ethyl, 2-cyclohexyl-2-oxoethyl, 2-cyclopentyl-2-oxoethyl, 1-acetylpiperidin-4-yl, tetra-hydrofuran-3-yl, 2-oxotetrahydrofuran-3-yl, 2-acetoxyethyl, 2-tert-butoxy-2-oxoethyl, 1-methoxy-3-methyl-1-oxobutan-2-yl, 1-methoxy-1-oxopropan-2-yl, 2-[2-(2-methoxyethoxy)ethoxy]ethyl, 2-(2-methoxyethoxy)ethyl, biphenyl-4-ylmethyl, biphenyl-3-ylmethyl, biphenyl-2-ylmethyl, 3-phenoxybenzyl, 4-fluoro-3-phenoxy-benzyl, cyclopentyloxy, 2-(1,5-dimethyl-1H-pyrazol-3-yl)-2-oxoethyl or 3-ethoxy-3-oxopropyl,
R⁴ stands for hydrogen, —NR¹²R¹³ or —C(O)NR¹²R¹³,
   wherein R⁴ preferably stands for hydrogen or NHR¹³, wherein R¹³ then stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with fluorine, chlorine, bromine, —OH or cyano,
R⁵ and R⁶ mutually independently stand for hydrogen, fluorine, chlorine, bromine, cyano, nitro, —OH or —SH,
   or for C₁-C₆ alkyl, C₃-C₆ cycloalkyl, C₂-C₆ alkenyl, C₂-C₆ alkynyl, —O—(C₁-C₄ alkyl), —S—(C₁-C₄ alkyl) or —S(O)—(C₁-C₆ alkyl), each optionally singly or multiply, identically or differently substituted with R¹¹,
   or else together with the carbon atom to which they are bound form a saturated, unsaturated or partially unsaturated single ring with 3 to 8 ring atoms, wherein the single ring can contain hetero atoms from the range oxygen, sulphur or —N—R¹⁹, optionally singly or multiply, identically or differently substituted with halogen, oxygen, cyano or C₁-C₄ alkyl,
R⁷ mutually independently stands for one or more of the following groups: fluorine, chlorine, bromine, cyano, nitro, methyl, ethyl, isopropyl, —CF₃, —CHF₂, —C₂F₅, —CCl₃, —OCH₃, —OC₂H₅, —O—CH(CH₃)₂, —OCF₃, —OCHF₂, —OC₂F₅, —SCH₃, —SO₂CH₃ or —SCF₃,
R¹¹ stands for —OH, fluorine, chlorine, bromine, cyano, —NH—C(O)R²⁰, —C(O)R²⁰, —C(O)OR²⁰, —C(O)NR²⁰R²¹ or —SO₂R²⁰
   or for C₁-C₆ alkyl, C₂-C₆ alkenyl, C₂-C₆ alkynyl, C₁-C₁₁ heteroalkyl, C₃-C₈ cycloalkyl, —O—(C₁-C₄ alkyl), —S—(C₁-C₄ alkyl), —O—(C₃-C₈ cycloalkyl), —S—(C₃-C₈ cycloalkyl), C₂-C₉ heterocyclyl or C₂-C₉ heteroaryl, each optionally singly or multiply, identically or differently substituted with fluorine, chlorine, bromine, —OH, cyano, C₁-C₆ alkyl or O—(C₁-C₄ alkyl),
R¹² and R¹³ mutually independently stand for one or more of the following groups: H, —C(S)R¹⁵, —C(O)R¹⁵, —SO₂R¹⁵, —C(O)OR¹⁵, —OR¹⁵ or —C(O)NR¹⁵R¹⁶
   or for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with fluorine, chlorine, bromine, —OH or cyano,
R¹⁵ and R¹⁶ mutually independently stand for H or —OH
   or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
   or, when R¹² and/or R¹³ stands for —C(O)NR¹⁵R¹⁶, together with the nitrogen atom to which they are bound form a 3 to 7-membered ring, which can contain a further hetero atom from the range N or O not directly adjacent to the nitrogen,
R¹⁹ stands for H, C₁-C₆ alkyl, C₃-C₆ cycloalkyl, C₂-C₆ alkenyl, C₂-C₆ alkynyl, —C(S)R¹⁵, —C(O)R¹⁵, —SO₂R¹⁵ or —C(O)OR¹⁵,
R²⁰ and R²¹ mutually independently stand for C₁-C₆ alkyl, C₃-C₆ cycloalkyl, C₂-C₆ alkenyl or C₂-C₆ alkynyl, each optionally singly or multiply, identically or differently substituted with fluorine, chlorine, bromine, —OH or cyano or for hydrogen, and
R²² and R²³ mutually independently stand for C₁-C₆ alkyl, C₃-C₆ cycloalkyl, C₂-C₆ alkenyl, C₂-C₆ alkynyl or hydrogen,

and agrochemically active salts thereof.

In a first embodiment of the present invention, compounds of the formula [I-a] wherein one or more of the symbols have one of the following meanings:
X¹ stands for C—H,
R¹ stands for phenyl or naphthalenyl, each optionally singly or multiply, identically or differently substituted with R⁷,
R² stands for halogen, C₁-C₆ alkyl, C₃-C₆ cycloalkyl or hydrogen,
R³ stands for propan-2-yl, isobutyl, butan-2-yl, 2-methylpropyl, 2,2-dimethylpropyl, 3-methylbut-2-en-1-yl, but-2-en-1-yl, but-3-en-2-yl, propadienyl, 4-methylpent-3-en-2-yl, prop-2-yn-1-yl, but-2-yn-1-yl, but-3-yn-2-yl, 2-methylbut-3-yn-2-yl, 2-methylbut-3-yn-2-yl, cyanomethyl, 2-cyanoethyl, 1-cyanopropan-2-yl, cyclopropyl, cyclobutyl, cyclopentyl, cyclopentyloxy, cyclohexyl, (2,2-dichlorocyclopropyl)-methyl, cyclopropylmethyl, 1-cyclopropylethyl, trichloromethyl, trifluoromethyl, 2-fluoroethyl, 2,2-difluoroethyl, 2,2,2-trifluoroethyl, 2,2,2-trichloroethyl, 2-chloroethyl, 2-bromoethyl, 2-fluoropropyl, 3-fluoropropyl, 2-chloropropyl, 3-chloropropyl, 1,1,1-trifluoropropan-2-yl, 1,1-difluoropropan-2-yl, 1,1,1-trifluoro-2-methylpropan-2-yl, 1,3-difluoropropan-2-yl, 3,3,3-trifluoro-2-hydroxypropyl, pyridin-2-ylmethyl, pyridine-3-ylmethyl, pyridin-4-ylmethyl, 2-chloro-1,3-thiazol-5-yl)methyl, benzyl, 2-fluorobenzyl, 3-fluorobenzyl, 4-fluorobenzyl, 1-(2-chlorophenyl)ethyl, 2,3-difluorobenzyl, 2-chloro-6-fluorobenzyl, 1-(3-chlorophenyl)ethyl, 1-(4-chloro-phenyl)ethyl, 2-cyanobenzyl, 3-cyanobenzyl, 4-cyanobenzyl, 4-(difluoro-methoxy)benzyl, biphenyl-3-ylmethyl, biphenyl-4-ylmethyl, biphenyl-2-ylmethyl, 3-phenoxybenzyl, 4-fluoro-3-phenoxybenzyl, 2-(3-chlorophenyl)ethyl, 2-(2-chloro-phenyl)ethyl, 1-naphthylmethyl, methoxymethyl, 2-methoxyethyl, 2-methoxypropyl, 2-(methylsulphanyl)ethyl, 2-(trifluoromethoxy)ethyl, 1-methoxypropan-2-yl, 2-[2-(2-methoxyethoxy)ethoxy]ethyl, 2-(2-methoxyethoxy)ethyl, 1,3-dimethoxypropan-2-yl, 2-(cyclopropyloxy)ethyl, propoxy, (2-methylprop-2-en-1-yl)oxy, (3-methyl-butanoyl)oxy, 1-cyanoethoxy, 2-chloroethoxy, but-2-yn-1-yloxy, cyanomethoxy, prop-2-yn-1-yloxy, 2-cyclohexyl-2-oxoethyl, 2-cyclopentyl-2-oxoethyl, tetrahydro-furan-2-ylmethyl, (3-methyloxetan-3-yl)methyl, 1H-imidazol-2-ylmethyl, 2-(1,5-dimethyl-1H-pyrazol-3-yl)-2-oxoethyl, 1-acetylpiperidin-4-yl, tetrahydrofuran-3-yl, 2-oxotetrahydrofuran-3-yl, 5-(trifluoromethyl)pyridin-2-yl, 5-(trifluoromethyl)-1,3,4-thiadiazol-2-yl, 6-(trifluoromethyl)pyrimidin-4-yl, 2-hydroxypropyl, 2-hydroxy-2-methylpropyl, 2,3-dihydroxypropyl, 2-acetoxyethyl, cyano, 2-tert-butoxy-2-oxoethyl, 1-methoxy-3-methyl-1-oxobutan-2-yl, 1-methoxy-1-oxopropan-2-yl or 3-ethoxy-3-oxopropyl, propan-2-yloxy, methyl, ethyl, 2-ethoxyethyl, or 2-chloroethyl,
R⁴ stands for hydrogen, —C(O)NR¹²R¹³ or —NR¹²R¹³,
   wherein R⁴ preferably stands for hydrogen,
R⁵ and R⁶ mutually independently stand for hydrogen, fluorine, chlorine, bromine, cyano, nitro,
   or for C₁-C₆ alkyl, C₃-C₆ cycloalkyl, C₂-C₆ alkenyl, C₂-C₆ alkynyl, —O—(C₁-C₄ alkyl), —S—(C₁-C₄ alkyl) or —S(O)—(C₁-C₆ alkyl), each optionally singly or multiply, identically or differently substituted with R¹¹,
   or together with the carbon atoms to which they are bound form a single ring wherein they together stand for —(CH═CH—CH═CH)—, —(CH═CH—N(R¹⁹), —(CH═CH—CH═N)—, —(NH—CH═N)— or —(CH₂—C(O)—N(R¹⁹)—, optionally singly or multiply identically or differently substituted with R¹¹,
R⁷ mutually independently stands for one or more of the following groups: fluorine, chlorine, cyano, nitro, methyl, ethyl, isopropyl, —CF₃, —CHF₂, —C₂F₅ or —CCl₃,
R¹¹ stands for —OH, fluorine, chlorine, bromine, cyano, —NHC(O)R²⁰, —C(O)R²⁰, —C(O)OR²⁰, —C(O)NR²⁰R²¹ or —SO₂R²⁰
   or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with fluorine, chlorine, bromine, —OH, cyano, C₁-C₆ alkyl or —O—(C₁-C₄ alkyl),
R¹² and R¹³ mutually independently stand for one or more of the following groups: H, —C(S)R¹⁵, —C(O)R¹⁵, —SO₂R¹⁵, —C(O)OR¹⁵, —OR¹⁵ or —C(O)NR¹⁵R¹⁶
   or for C₁-C₆ alkyl, C₃-C₆ cycloalkyl, C₂-C₆ alkenyl, C₂-C₆ alkynyl, C₂-C₉ hetero-cyclyl or C₇-C₉ heteroaryl, each optionally singly or multiply, identically or differently substituted with fluorine, chlorine, bromine, —OH or cyano,
R¹⁵ and R¹⁶ mutually independently stand for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with R¹¹,
   or for hydrogen
   or together with the nitrogen atom to which they are bound form a 3 to 7-membered ring, which can contain a further hetero atom from the range N or O not adjacent to the nitrogen,
R¹⁹ stands for H, C₂-C₆ alkynyl, C(O)R¹⁵, SO₂R¹⁵ or C(O)OR¹⁵, and
R²⁰ and R²¹ mutually independently stand for C₁-C₆ alkyl, C₃-C₆ cycloalkyl, C₂-C₆ alkenyl or C₂-C₆ alkynyl, each optionally singly or multiply, identically or differently substituted with fluorine, chlorine, bromine, —OH or cyano,
   or for hydrogen,

and agrochemically active salts thereof, are particularly preferred.

In this first embodiment of the present invention, compounds of the formula [I-a] wherein one or more of the symbols have one of the following meanings:
X¹ stands for C—H
R¹ stands for phenyl, optionally singly or multiply identically or differently substituted with R⁷,
R² stands for methyl, ethyl, isopropyl, cyclopropyl or hydrogen,
R³ stands for propan-2-yl, isobutyl, butan-2-yl, 2-methylpropyl, 2,2-dimethylpropyl, 3-methylbut-2-en-1-yl, but-2-en-1-yl, but-3-en-2-yl, propadienyl, prop-2-yn-1-yl, but-2-yn-1-yl, but-3-yn-2-yl, 2-methylbut-3-yn-2-yl, 2-methylbut-3-yn-2-yl, cyano-methyl, 2-cyanoethyl, 1-cyanopropan-2-yl, cyclopropyl, cyclobutyl, cyclopentyl, cyclohexyl, (2,2-dichlorocyclopropyl)methyl, cyclopropylmethyl, 1-cyclopropylethyl, trichloromethyl, trifluoromethyl, 2-fluoroethyl, 2,2-difluoroethyl, 2,2,2-trifluoroethyl, 2,2,2-trichloroethyl, 2-chloroethyl, 2-bromoethyl, 2-fluoropropyl, 3-fluoropropyl, 2-chloropropyl, 3-chloropropyl, 1,3-difluoropropan-2-yl, 2-fluorobenzyl, 3-fluoro-benzyl, 4-fluorobenzyl, 2,3-difluorobenzyl, 2-chloro-6-fluorobenzyl, 1-(2-chloro-phenyl)ethyl, 1-(3-chlorophenyl)ethyl, 1-(4-chlorophenyl)ethyl, 3-cyanobenzyl, 4-cyanobenzyl, 4-(difluoromethoxy)benzyl, 2-cyanobenzyl, 2-(3-chlorophenyl)ethyl, 2-(2-chlorophenyl)ethyl, 1-naphthylmethyl, (pyridine-3-ylmethyl, 2-chloro-1,3-thiazol-5-yl)methyl, methoxymethyl, 2-methoxyethyl, 2-(methylsulphanyl)ethyl, 2-(trifluoromethoxy)ethyl, 1-methoxypropan-2-yl, 2-[2-(2-methoxyethoxy)ethoxy]-ethyl, 2-(2-methoxyethoxy)ethyl, tetrahydrofuran-2-ylmethyl, (3-methyloxetan-3-yl)methyl, 1H-imidazol-2-ylmethyl, tetrahydrofuran-3-yl, 2-oxotetrahydrofuran-3-yl, 2-tert-butoxy-2-oxoethyl, 1-methoxy-3-methyl-1-oxobutan-2-yl, 1-methoxy-1-oxopropan-2-yl or 3-ethoxy-3-oxopropyl, propan-2-yloxy, 2-ethoxyethyl, methyl, ethyl, 1-propyl or 2-chloroethyl, in a preferred modification R³ is as defined above but does not include methyl or ethyl.
R⁴ stands for hydrogen, —NR¹²R¹³ or —NHR¹³,
   wherein R⁴ preferably stands for hydrogen,
R⁵ and R⁶ mutually independently stand for hydrogen, fluorine or cyano
   or together with the carbon atoms to which they are bound form a ring wherein they together stand for —(CH═CH—CH═CH)—, —(CH═CH—N(R¹⁹), —(CH═CH—CH═N)—, —(NH—CH═N)— or —(CH₂—C(O)—N(R¹⁹)—, optionally singly or multiply identically or differently substituted with R¹¹,
R⁷ mutually independently stands for one or more of the following groups: fluorine, chlorine, cyano or methyl,
R¹¹ stands for —OH, fluorine, chlorine, bromine, cyano, —NH—C(O)R²⁰, —C(O)R²⁰, —C(O)OR²⁰, —C(O)NR²⁰R²¹ or —SO₂R²⁰
   or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with fluorine, chlorine, bromine, OH, cyano, C₁-C₆ alkyl or —O—(C₁-C₄ alkyl),
R¹² stands for —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₂-C₆ alkenyl, C₂-C₆ alkynyl, C₂-C₉ heterocyclyl or C₂-C₉ heteroaryl or hydrogen,
R¹⁵ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with halogen, —OH, cyano or C₁-C₄ alkyl,
   or for hydrogen
R¹⁶ stands for hydrogen, methyl, ethyl or propyl,
R¹⁹ stands for H, C₂-C₆ alkynyl, —C(O)R¹⁵, —SO₂R²⁵ or —C(O)OR¹⁵, and
R²⁰ and R²¹ mutually independently stand for methyl, ethyl, propyl, isopropyl, cyclopropyl or cyclobutyl, each optionally singly or multiply, identically or differently substituted with fluorine, chlorine, bromine, OH or cyano,
   or for hydrogen,

and agrochemically active salts thereof, are quite particularly preferred,

In this first embodiment of the present invention, compounds of the formula [I-a] wherein one or more of the symbols have one of the following meanings:
X¹ stands for C—H
R¹ stands for phenyl, 3-methylphenyl, 2-fluorophenyl, 3-fluorophenyl, 4-fluorophenyl, 3-trifluoromethylphenyl, 4-chlorophenyl, 2,5-difluorophenyl, 2,4-difluorophenyl, 2,6-difluorophenyl, 3-methyl-4-fluorophenyl, 3-cyano-4-fluorophenyl or 2,4,6-trifluorophenyl,
R² stands for methyl, ethyl, isopropyl, cyclopropyl, or hydrogen,
R³ stands for propan-2-yl, isobutyl, butan-2-yl, 2-methylpropyl, 2,2-dimethylpropyl, prop-2-yn-1-yl, but-3-yn-2-yl, cyclopropyl, cyclobutyl, cyclopentyl, (2,2-dichloro-cyclopropyl)methyl, cyclopropylmethyl, 1-cyclopropylethyl, trifluoromethyl, trichloromethyl, 2-fluoroethyl, 2,2-difluoroethyl, 2,2,2-trifluoroethyl, 2,2,2-trichloro-ethyl, 2-chloroethyl, 2-fluoropropyl, 3-fluoropropyl, 1,3-difluoropropan-2-yl, 2-fluorobenzyl, 2,3-difluorobenzyl, 2-chloro-6-fluorobenzyl, 1-(2-chlorophenyl)ethyl, 1-(3-chlorophenyl)ethyl, 2-(trifluoromethoxy)ethyl, or 1-methoxypropan-2-yl, 2,2-difluoroethyl, 2-cyanoethyl, cyanomethyl, 1-cyanopropan-2-yl, 1-propyl, 2-ethoxyethyl, 2-chloroethyl or 2-methoxyethyl,
R⁴ stands for hydrogen
R⁵ and R⁶ mutually independently stand for hydrogen, fluorine or cyano
   or together with the carbon atoms to which they are bound form a ring wherein they together stand for —(CH═CH—CH═CH)—, —(CH═CH—N(R¹⁹)—, —(CH═CH—CH═N)—, —(NH—CH═N)— or —(CH₂—C(O)—N(R¹⁹)—, optionally singly or multiply identically or differently substituted with R¹¹,
R¹¹ stands for —OH, fluorine, chlorine, cyano, C₁-C₆ alkyl, C₂-C₆ alkenyl or C₂-C₆ alkynyl or C₃-C₆ cycloalkyl,
   or for hydrogen, and
R¹⁹ stands for H, acetyl, ethoxycarbonyl, methoxycarbonyl, prop-2-yn-1-yl or but-2-yn-1-yl,

and agrochemically active salts thereof are especially preferred.

In this first embodiment of the present invention, compounds of the formula [I-a], wherein one or more of the symbols have one of the following meanings:
X¹ stands for C—H,
R⁴ stands for H, and
R³ stands for propan-2-yl, isobutyl, butan-2-yl, 2-methylpropyl, prop-2-yn-1-yl, cyclo-propyl, cyclobutyl, cyclopentyl, (2,2-dichlorocyclopropyl)methyl, cyclopropylethyl, 1-cyclopropylethyl, 2,2,2-trifluoroethyl, 2-fluorobenzyl, 2-(trifluoromethoxy)ethyl, 1-methoxypropan-2-yl, 2,2-difluoroethyl, 2-cyanoethyl, cyanomethyl, 1-cyano-propan-2-yl, 1-propyl, 2-ethoxyethyl, 2-chloroethyl or 2-methoxyethyl,

wherein the other substituents have one or more of the aforesaid meanings,

and the agrochemically active salts thereof are further especially preferred.

Further especially preferred are compounds of the first embodiment of the invention of the formula [I-a], wherein
R³ stands for propan-2-yl, isobutyl, butan-2-yl, 2-methylpropyl, prop-2-yn-1-yl, cyclopropyl, cyclobutyl, cyclopentyl, (2,2-dichlorocyclopropyl)methyl, cyclopropyl-methyl, 2,2,2-trifluoroethyl, 2-fluorobenzyl, 2-(trifluoromethoxy)ethyl or 1-methoxy-propan-2-yl,

wherein the other substituents have one or more of the aforesaid meanings,

and the agrochemically active salts thereof.

In a second embodiment of the present inventions compounds of the formula [I-a] wherein one or more of the symbols have one of the following meanings:
X¹ stands for N,
R¹ stands for phenyl or naphthalenyl, each optionally singly or multiply, identically or differently substituted with R⁷,
R² stands for halogen, C₁-C₆ alkyl, C₃-C₆ cycloalkyl or hydrogen,
R³ stands for C₁-C₆ alkyl, C₂-C₆ alkenyl, C₂-C₆ alkynyl, C₃-C₆ cycloalkyl, C₃-C₆ cycloalkyl-C₁-C₆ alkyl, C₆-C₁₄ aryl-C₁-C₆ alkyl, C₁-C₆ alkoxy-C₁-C₆ alkyl, C₁-C₆ alkoxy, C₂-C₉ heterocyclyl-C₁-C₆ alkyl and C₂-C₉ heteroaryl, each optionally singly or multiply, identically or differently substituted with halogen, cyano, hydroxy or haloalkoxy,
R⁴ stands for hydrogen, —C(O)NR¹²R¹³ or —NR¹²R¹³,
   wherein R⁴ preferably stands for —NHR¹³, and R¹³ stands for C₁-C₆ alkyl, C₃-C₆ cycloalkyl, C₂-C₆ alkenyl, C₂-C₆ alkynyl, C₂-C₉ heterocyclyl or C₂-C₉ heteroaryl, each optionally singly or multiply, identically or differently substituted with fluorine, chlorine, bromine, —OH or cyano,
R⁵ and R⁶ mutually independently stand for hydrogen, fluorine, chlorine, bromine, cyano or nitro, or for C₁-C₆ alkyl, C₃-C₆ cycloalkyl, C₂-C₆ alkenyl, C₂-C₆ alkynyl, —O—(C₁-C₄ alkyl), —S—(C₁-C₄ alkyl) or —S(O)—(C₁-C₆ alkyl), each optionally singly or multiply, identically or differently substituted with R¹¹,
   or together with the carbon atoms to which they are bound form a single ring wherein they together stand for —(CH═CH—CH═CH)—, —(CH═CH—N(R¹⁹), —(CH═CH—CH═N)—, —(NH—CH═N)— or —(CH₂—C(O)—N(R¹⁹)—, optionally singly or multiply identically or differently substituted with R¹¹,
R⁷ mutually independently stands for one or more of the following groups: fluorine, chlorine, cyano, nitro, methyl, ethyl, isopropyl, —CF₃, —CHF₂, —C₂F₅ or —CCl₃,
R¹¹ stands for —OH, fluorine, chlorine, bromine, cyano, —NHC(O)R²⁰, —C(O)R²⁰, —C(O)OR²⁰, —C(O)NR²⁰R²¹ or —SO₂R²⁰
   or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with fluorine, chlorine, bromine, —OH, cyano, C₁-C₆ alkyl or —O—(C₁-C₄ alkyl),
R¹² and R¹³ mutually independently stand for one or more of the following groups: H, —C(S)R¹⁵, —C(O)R¹⁵, —SO₂R¹⁵, —C(O)OR¹⁵, —OR¹⁵ or —C(O)NR¹⁵R¹⁶
   or for C₁-C₆ alkyl, C₃-C₆ cycloalkyl, C₂-C₆ alkenyl, C₂-C₆ alkynyl, C₂-C₉ heterocyclyl or C₂-C₉ heteroaryl, each optionally singly or multiply, identically or differently substituted with fluorine, chlorine, bromine, —OH or cyano,
R¹⁵ and R¹⁶ mutually independently stand for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with R¹¹,
   or for hydrogen
   or together with the nitrogen atom to which they are bound form a 3 to 7-membered ring, which can contain a further hetero atom from the range N or O not adjacent to the nitrogen,
R¹⁹ stands for H, C₂-C₆ alkynyl, —C(O)R¹⁵, or —C(O)OR¹⁵, and
R²⁰ and R²¹ mutually independently stand for C₁-C₆ alkyl, C₃-C₆ cycloalkyl, C₂-C₆ alkenyl or C₂-C₆ alkynyl, each optionally singly or multiply, identically or differently substituted with fluorine, chlorine, bromine, —OH or cyano,
   or for hydrogen,

and agrochemically active salts thereof, are particularly preferred.

In this second embodiment of the invention, compounds of the formula [I-a], wherein one or more of the symbols have one of the following meanings:
X¹ stands for N,
R¹ stands for phenyl, optionally singly or multiply identically or differently substituted with R⁷,
R² stands for methyl, ethyl, isopropyl, cyclopropyl or hydrogen,
R³ stands for methyl, ethyl, 1-propyl, propan-2-yl, isobutyl, butan-2-yl, 2-methylpropyl, 2,2-dimethylpropyl, 3-methylbut-2-en-1-yl, but-2-en-1-yl, but-3-en-2-yl, propadienyl, prop-2-yn-1-yl, but-2-yn-1-yl, but-3-yn-2-yl, 2-methylbut-3-yn-2-yl, 2-methylbut-3-yn-2-yl, cyanomethyl, 2-cyanoethyl, 1-cyanopropan-2-yl, cyclopropyl, cyclobutyl, cyclopentyl, cyclohexyl, (2,2-dichlorocyclopropyl)methyl, cyclopropylmethyl, 1-cyclopropylethyl, trichloromethyl, trifluoromethyl, 2-fluoroethyl, 2,2-difluoroethyl, 2,2,2-trifluoroethyl, 2,2,2-trichloroethyl, 2-chloroethyl, 2-bromoethyl, 2-fluoropropyl, 3-fluoropropyl, 2-chloropropyl, 3-chloropropyl, 1,3-difluoropropan-2-yl, 2-fluoro-benzyl, 3-fluorobenzyl, 4-fluorobenzyl, 2,3-difluorobenzyl, 2-chloro-6-fluorobenzyl, 1-(2-chlorophenyl)ethyl, 1-(3-chlorophenyl)ethyl, 1-(4-chlorophenyl)ethyl, 3-cyano-benzyl, 4-cyanobenzyl, 4-(difluoromethoxy)benzyl, 2-cyanobenzyl, 2-(3-chloro-phenyl)ethyl, 2-(2-chlorophenyl)ethyl, 1-naphthylmethyl, (pyridine-3-ylmethyl, 2-chloro-1,3-thiazol-5-yl)methyl, methoxymethyl, 2-methoxyethyl, 2-(methyl-sulphanyl)ethyl, 2-(trifluoromethoxy)ethyl, 1-methoxypropan-2-yl, 2-[2-(2-methoxy-ethoxy)ethoxy]ethyl, 2-(2-methoxyethoxy)ethyl, tetrahydrofuran-2-ylmethyl, (3-methyloxetan-3-yl)methyl, 1H-imidazol-2-ylmethyl, tetrahydrofuran-3-yl, 2-oxo-tetrahydrofuran-3-yl, 2-tert-butoxy-2-oxoethyl, 1-methoxy-3-methyl-1-oxobutan-2-yl, 1-methoxy-1-oxopropan-2-yl or 3-ethoxy-3-oxopropyl, propan-2-yloxy, 2-ethoxy-ethyl, or 2-chloroethyl,
R⁴ stands for hydrogen, —NR¹²R¹³ or —NHR¹³,
   wherein R⁴ preferably stands for —NHR¹³, and R¹³ for C₁-C₆ alkyl, C₃-C₆ cycloalkyl, C₂-C₆ alkenyl, C₂-C₆ alkynyl, C₂-C₉ heterocyclyl or C₂-C₉ heteroaryl, each optionally singly or multiply, identically or differently substituted with fluorine, chlorine, bromine, OH or cyano,
R⁵ and R⁶ mutually independently stand for hydrogen, fluorine or cyano
   or together with the carbon atom to which they are bound form a ring wherein they together stand for —(CH═CH—CH═CH)—, —(CH═CH—N(R¹⁹), —(CH═CH—CH═N)—, —(NH—CH═N)— or —(CH₂—C(O)—N(R¹⁹)—, optionally singly or multiply identically or differently substituted with R¹¹,
R⁷ mutually independently stands for one or more of the following groups: fluorine, chlorine, cyano or methyl,
R¹¹ stands for —OH, fluorine, chlorine, bromine, cyano, —NH—C(O)R²⁰, —C(O)R²⁰, —C(O)OR²⁰, —C(O)NR²⁰R²¹ or —SO₂R²⁰
   or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with fluorine, chlorine, bromine, OH, cyano, C₁-C₆ alkyl or —O—(C₁-C₄ alkyl),
R¹² stands for —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₂-C₆ alkenyl, C₂-C₆ alkynyl C₂-C₉ heterocyclyl or C₂-C₉ heteroaryl, or hydrogen,
R¹⁵ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, optionally singly or multiply, identically or differently substituted with halogen, OH, cyano or C₁-C₄ alkyl,
   or for hydrogen,
R¹⁶ stands for hydrogen, methyl, ethyl or propyl,
R¹⁹ stands for H, C₂-C₆ alkynyl, —C(O)R¹⁵, —SO₂R¹⁵ or —C(O)OR¹⁵, and
R²⁰ and R²¹ mutually independently stand for methyl, ethyl, propyl, isopropyl, cyclopropyl or cyclobutyl, each optionally singly or multiply, identically or differently substituted with fluorine, chlorine, bromine, —OH or cyano,
   or for hydrogen,

and agrochemically active salts thereof, are quite particularly preferred.

In this second embodiment of the invention compounds of the formula [I-a], wherein one or more of the symbols have one of the following meanings:
X¹ stands for N,
R¹ stands for phenyl, 4-fluorophenyl,
R² stands for methyl, ethyl, isopropyl, cyclopropyl, or hydrogen,
R³ stands for methyl, ethyl, 1-propyl, propan-2-yl, isobutyl, butan-2-yl, 2-methylpropyl, 2,2-dimethylpropyl, prop-2-yn-1-yl, but-3-yn-2-yl, cyclopropyl, cyclobutyl, cyclo-pentyl, (2,2-dichlorocyclopropyl)methyl, cyclopropylmethyl, 1-cyclopropylethyl, trifluoromethyl, trichloromethyl, 2-fluoroethyl, 2,2-difluoroethyl, 2,2,2-trifluoroethyl, 2,2,2-trichloroethyl, 2-chloroethyl, 2-fluoropropyl, 3-fluoropropyl, 1,3-difluoro-propan-2-yl, 2-fluorobenzyl, 2,3-difluorobenzyl, 2-chloro-6-fluorobenzyl, 1-(2-chlorophenyl)ethyl, 1-(3-chlorophenyl)ethyl, 2-(trifluoromethoxy)ethyl, or 1-methoxypropan-2-yl, 2,2-difluoroethyl, 2-cyanoethyl, cyanomethyl, 1-cyano-propan-2-yl, 1-propyl, 2-ethoxyethyl, 2-chloroethyl or 2-methoxyethyl,
R⁴ stands for hydrogen , —NHR¹² or for —NHR¹³, preferably for —NHR¹³
R⁵ and R⁶ mutually independently stand for hydrogen, fluorine or cyano
   or together with the carbon atoms to which they are bound form a ring wherein they together stand for —(CH═CH—CH═CH)—, —(CH═CH—N(R¹⁹)—, —(CH═CH—CH═N)— or —(CH₂—C(O)—N(R¹⁹)—, optionally singly or multiply identically or differently substituted with R¹¹,
R¹¹ stands for —OH, fluorine, chlorine, cyano, C₁-C₆ alkyl, C₂-C₆ alkenyl or C₂-C₆ alkynyl or C₃-C₆ cycloalkyl,
R¹² stands for —C(S)R¹⁵, —SO₂R¹⁵, —C(O)OR¹⁵ or —C(O)R¹⁵,
R¹³ stands for allyl, benzyl, cyclobutyl, cyclopent-3-en-1-yl, cyclopentyl, cyclopropyl, (1-cyclopropylethyl), (1-cyclopropyl ethyl), (cyclopropylmethyl), (cyclopropyl-methyl), (dicyclopropylmethyl), (2,2-difluoroethyl), (2,2-dimethoxyethyl), [2-(dimethylamino)-2-oxoethyl], [(2,2-dimethylcyclopropyl)methyl], (2-ethoxy-ethyl), ethyl, (3-fluorobenzyl), (4-fluorobenzyl), (2-fluorobenzyl), [1-(2-fluoro-phenyl)ethyl], (2-hydroxy-2-methylpropyl), (2-hydroxyethyl), (2-hydroxypropyl), (2-hydroxypropyl), isopropyl, (2-methoxyethyl), (1-methoxypropan-2-yl), (1-methoxypropan-2-yl), methyl, [2-(morpholin-4-yl)ethyl], oxetan-3-yl, (1-phenyl-ethyl), prop-2-yn-1-yl, propyl, [1-(pyridin-2-yl)ethyl], (pyrimidin-2-ylmethyl), sec-butyl, tetrahydro-2H-pyran-3-yl, tetrahydro-2H-pyran-4-yl or tetrahydro-2H-pyran-4-yl,
R¹⁵ stands for methyl, ethyl, n-propyl, isopropyl, sec-butyl, isobutyl, cyclopropyl, cyclobutyl, cyclopentyl, cyclohexyl, 2-methoxyethyl, (2-methoxyethoxy)methyl, cyclopentenyl, cyclohexenyl, oxetanyl, tetrahydrofuran-2-yl, ethynyl, prop-1-yn-1-yl, prop-1-en-1-yl, aminomethyl, aminoethyl, aminopropyl, aminobutyl, aminoisopropyl, aminocyclopropyl, aminocyclobutyl, aminocyclopentyl, dimethylamino, ethyl-(methyl)amino, pyrrolidinyl, diethylamino, 2-pyridyl, 3-pyridyl, 4-pyridyl, ethoxycarbonyl, benzyl or phenyl, each optionally singly or multiply, identically or differently substituted with halogen, —OH, cyano or C₁-C₄ alkyl,
   or for hydrogen, and
R¹⁹ stands for H, acetyl, ethoxycarbonyl, methoxycarbonyl, prop-2-yn-1-yl or but-2-yn-1-yl,

and agrochemically active salts thereof, are especially preferred.

Further especially preferred are compounds of the second embodiment of the invention of the formula [I-a], wherein
R⁴ stands for —NHR¹³ or for H,

wherein the other substituents have one or more of the aforesaid meanings,

and the agrochemically active salts thereof.

Quite particularly preferred compounds of the formula [I-a] of the first and second embodiment of the present invention above are those wherein one or more of the symbols have one of the following meanings:
R¹ quite especially preferably stands for 4-fluorophenyl, 3-chlorophenyl, 2,6-difluoro-phenyl, or 3-methylphenyl and in particular for 4-fluorophenyl,
R² quite especially preferably stands for cyclopropyl, ethyl, methyl or hydrogen and in particular for hydrogen,
R⁵ and R⁶ quite especially preferably both stand for hydrogen.

The residue definitions or explanations expounded generally or expounded in preferred ranges above can however also be mutually combined, i.e. between the relevant ranges and preferred ranges. They apply for the final products and for the precursors and intermediates correspondingly. In addition, some individual definitions may not apply.

Those compounds of the formula [I-a], in which all residues each have the aforesaid preferred meanings are preferred.

Those compounds of the formula [I-a], in which all residues each have the aforesaid particularly preferred meanings are particularly preferred.

Those compounds of the formula [I-a], in which all residues each have the aforesaid quite particularly preferred meanings are quite particularly preferred.

Those compounds of the formula [I-a], in which all residues each have the aforesaid especially preferred meanings are especially preferred.

In addition, novel phenylpyri(mi)dinylazoles of the formula [I-b] have been found,

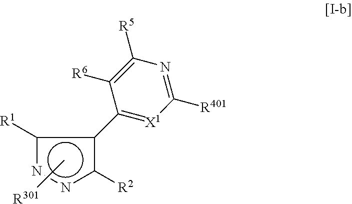

wherein the symbols have the following meanings:
X¹ stands for C—H or N,
R¹ stands for phenyl, naphthalenyl, quinolin-5-yl, quinolin-8-yl, isoquinolin-5-yl, isoquinolin-8-yl, 1-benzothiophen-4-yl, 1-benzothiophen-7-yl, 1-benzofuran-4-yl, 1-benzofuran-7-yl, 1,3-benzodioxol-4-yl or 1,3-benzodioxol-5-yl, each optionally singly or multiply, identically or differently substituted with R⁷,
R² stands for cyano, nitro, halogen, C₁-C₆ alkyl, C₁-C₆ alkoxy, C₁-C₆ haloalkyl, C₁-C₆ haloalkoxy, C₁-C₆ alkylthio, C₃-C₆ cycloalkyl, C₃-C₆ halocycloalkyl, C₂-C₉ heterocyclyl or hydrogen,
R³⁰¹ stands for —C(O)N(R⁹R¹⁰), —C(O)R⁹, —C(O)OR⁹, —S(O)₂R⁹ or for C₁-C₆ alkyl, C₂-C₆ alkenyl, C₃-C₆ allenyl, C₂-C₆ alkynyl, C₃-C₈ cycloalkyl, C₁-C₆ alkoxy, C₂-C₉ heterocyclyl, C₂-C₉ oxoheterocyclyl, or heteroaryl, (preferably C₂-C₉ heteroaryl), each optionally singly or multiply, identically or differently substituted with R⁸,
R⁴⁰¹ stands for —NR¹²R¹³, —C(O)NR¹²R¹³ or for —N(R¹²)₂
R⁵ and R⁶ mutually independently stand for hydrogen, fluorine, chlorine, bromine, cyano, nitro, —OH or —SH,
   or for C₁-C₆ alkyl, C₃-C₆ cycloalkyl, C₂-C₆ alkenyl, C₂-C₆ alkynyl, C₆-C₁₄ aryl, —O—(C₁-C₄ alkyl), —O—(C₆-C₁₄ aryl), —S—(C₁-C₄ alkyl), —S(O)—(C₁-C₆ alkyl) or —C(O)—(C₁-C₆ alkyl), each optionally singly or multiply, identically or differently substituted with R¹¹,
   or else together with the carbon atom to which they are bound form a ring (preferably saturated, unsaturated or partially unsaturated single ring) with 3, preferably 5 to 8 ring atoms, wherein the ring can contain 1 to 4 hetero atoms from the range oxygen, sulphur or —N—R¹⁹, optionally singly or multiply, identically or differently substituted with halogen, oxygen, cyano or C₁-C₄ alkyl,
R⁷ mutually independently stands for one or more of the following groups: fluorine, chlorine, bromine, cyano, nitro, —OH or —SH,
   or for C₁-C₆ alkyl, C₃-C₆ cycloalkyl, C₂-C₆ alkenyl, C₂-C₆ alkynyl, tri(C₁-C₄ alkyl)-silyl, C₆-C₁₄ aryl, —O—(C₁-C₄ alkyl), —O—(C₆-C₁₄ aryl), —S—(C₁-C₄ alkyl), —S(O)—(C₁-C₆ alkyl) or —S(O)₂—(C₁-C₆ alkyl), optionally singly or multiply identically or differently substituted with fluorine, chlorine, bromine, OH, cyano, C₁-C₆ alkyl or —O—(C₁-C₄ alkyl),
R⁸ stands for —OH, halogen (preferably fluorine, chlorine or bromine), —NO₂, cyano, —NR⁹R¹⁰, —C(O)N(R⁹R¹⁰), —C(O)R⁹, —C(O)OR⁹, —O—C(O)R⁹ or —(CH₂)ₙC(O)R⁹, wherein n=a whole number between 1 and 6,
   or for C₁-C₆ alkyl, C₂-C₈ alkenyl, C₂-C₈ alkynyl, C₃-C₈ cycloalkyl, C₁-C₆ haloalkyl, C₆-C₁₄ aryl, C₂-C₉ heterocyclyl, C₂-C₉ heteroaryl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with R¹¹,
R⁹ and R¹⁰ mutually independently stand for C₁-C₆ alkyl, C₂-C₈ alkenyl, C₂-C₈ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
   or for hydrogen,
R¹¹ stands for —OH, fluorine, chlorine, bromine, cyano, —NH—C(O)R²⁰, —NR²⁰R²¹, —C(O)R²⁰, —C(O)OR²⁰, —C(O)NR²⁰R²¹ or —SO₂R²⁰
   or for C₁-C₆ alkyl, C₂-C₆ alkenyl, C₂-C₆ alkynyl, C₁-C₁₁ heteroalkyl, C₃-C₈ cycloalkyl, —O—(C₁-C₄ alkyl), —S—(C₁-C₄ alkyl), —O—(C₃-C₈ cycloalkyl), —S—(C₃-C₈ cycloalkyl), C₆-C₁₄ aryl, —O—(C₆-C₁₄ aryl), —S—(C₆-C₁₄ aryl), C₂-C₉ heterocyclyl or C₂-C₉ heteroaryl, optionally singly or multiply identically or differently substituted with fluorine, chlorine, bromine, OH, carbonyl, cyano, C₁-C₆ alkyl or —O—(C₁-C₄ alkyl),
R¹² stands for —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with fluorine, chlorine, bromine, —OH, cyano, C₁-C₆ alkyl, —O—C(O)—C₁-C₄ alkyl, —O—P(O)(O—C₁-C₄ alkyl)₂, —O—B(O—C₁-C₄ alkyl)₂or —O—(C₁-C₄ alkyl),
   or for hydrogen,
R¹⁵ and R¹⁶ mutually independently stand for hydrogen or —OH,
   or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
   or (when R¹² stands for —C(O)NR¹⁵R¹⁶) together with the nitrogen atom to which they are bound form a 3 to 7-membered ring, which can contain a further hetero atom from the range N or O not directly adjacent to the nitrogen,
R¹⁹ stands for H, C₁-C₆ alkyl, C₃-C₆ cycloalkyl, C₂-C₆ alkenyl, C₂-C₆ alkynyl, —C(S)R¹⁵, —C(O)R¹⁵, —SO₂R¹⁵ or —C(O)OR¹⁵, and
R²⁰ and R²¹ mutually independently stand for C₁-C₆ alkyl, C₃-C₆ cycloalkyl, C₂-C₆ alkenyl, C₂-C₆ alkynyl or hydrogen, each optionally singly or multiply, identically or differently substituted with fluorine, chlorine, bromine, —OH or cyano,

and agrochemically active salts thereof.

Combinations wherein the symbols of the formula [I-b] have the following meanings:

a) Compounds wherein,
R³⁰¹ stands for optionally substituted [1,2,4[triazolo[4,3-b]pyridazin-6-yl, 7,8-dihydro-[1,2,4[triazolo[4,3-b]pyridazin 6-yl, 6-oxo-1,6-dihydropyridazin-3-yl, 6-oxo-1,4,5,6-tetrahydropyridazin-3-yl or 6-chloropyridazin-3-yl and
R⁵, R⁶ stand for H, and

b) Compounds: 4-{1-[2-(dimethylamino)ethyl]-3-(4-fluorophenyl)-1H-pyrazol-4-yl}-N,N-dimethyl-pyridin-2-amine and 1-(4-{4-[1-ethyl-3-(4-nitrophenyl)-1H-pyrazol-4-yl]-1H-pyrrolo[2,3-b]pyridine-6-yl}phenyl)-N,N-dimethylmethanamine

are excepted from the residue definitions or explanations expounded generally or expounded in preferred ranges above.

Finally it has been found that the phenylpyri(mi)dinylazoles according to the invention of the formula [I-b] possess very good microbicidal properties and can be used material protection and for the reduction of mycotoxins in plants and plant parts.

The phenylpyri(mi)dinylazoles according to the invention are defined generally by the formula [I-b]. Preferred residue definitions for the formulae named above and below are stated below. These definitions apply equally for the final products of the formula [I-b] and for all intermediates.

Compounds of the formula [I-b], wherein one or more of the symbols have one of the following meanings:
X¹ stands for C—H or N,
R¹ stands for phenyl, naphthalenyl, quinolin-5-yl, quinolin-8-yl, isoquinolin-5-yl, isoquinolin-8-yl, 1,3-benzodioxol-4-yl or 1,3-benzodioxol-5-yl, each optionally singly or multiply, identically or differently substituted with R⁷,
R² stands for cyano, halogen, C₁-C₆ alkyl, C₁-C₆ alkoxy, C₁-C₆ haloalkyl, C₁-C₆ haloalkoxy, C₁-C₆ alkylthio, C₃-C₆ cycloalkyl, C₃-C₆ halocycloalkyl, C₂-C₉ heterocyclyl or hydrogen,
R³⁰¹ stands for —C(O)R⁹, —C(O)OR⁹ or —S(O)₂R⁹
   or for C₁-C₆ alkyl, C₂-C₆ alkenyl, C₃₋₆ allenyl, C₂₋₆ alkynyl, C₃-C₈ cycloalkyl, C₁-C₆ alkoxy, C₂-C₉ heterocyclyl, C₂-C₉ oxoheterocyclyl, or heteroaryl, optionally singly or multiply identically or differently substituted with R⁸,
R⁴⁰¹ stands for —NR¹²R¹³ or —C(O)NR¹²R¹³,
R⁵ and R⁶ mutually independently stand for hydrogen, fluorine, chlorine, bromine, cyano, nitro, —OH or —SH,
   or for C₁-C₆ alkyl, C₃-C₆ cycloalkyl, C₂-C₆ alkenyl, C₂-C₆ alkynyl, —O—(C₁-C₄ alkyl), —S—(C₁-C₄ alkyl) or —S(O)—(C₁-C₆ alkyl), each optionally singly or multiply identically or differently substituted with R¹¹,
   or else together with the carbon atom to which they are bound form a ring (preferably a saturated, unsaturated or partially unsaturated single ring) with 3, preferably 5 to 8 ring atoms, wherein the ring can contain 1 to 4 hetero atoms from the range oxygen, sulphur or —NR¹⁹, optionally singly or multiply, identically or differently substituted with halogen, oxygen, cyano or C₁-C₄ alkyl,
   R² stands for one or more of the following groups: fluorine, chlorine, bromine, cyano, nitro, methyl, ethyl, isopropyl, —CF₃, —CHF₂, —C₂F₅, —CCl₃, —OMe, —OEt, —O-iPr, —OCF₃, —OCHF₂, —OC₂F₅, —SMe or —SCF₃,
R⁸ stands for —OH, fluorine, chlorine, cyano, —NR⁹R¹⁰, —C(O)N(R⁹R¹⁰), —C(O)R⁹, —C(O)OR⁹, —O—C(O)R⁹, —(CH₂)ₙC(O)R⁹, wherein n=a whole number between 1 and 6, or for C₁-C₆ alkyl, C₂-C₈ alkenyl, C₂-C₈ alkynyl, C₃-C₈ cycloalkyl, C₁-C₆ haloalkyl, C₆-C₁₄ aryl, C₂-C₉ heterocyclyl, C₂-C₉ heteroaryl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with R¹¹,
R⁹ and R¹⁰ mutually independently stand for C₁-C₆ alkyl, C₂-C₈ Alkenyl, C₂-C₈ alkynyl, C₃-C₈ cycloalkyl, C₂-C₉ heterocyclyl, or C₂-C₉ heteroaryl, each optionally singly or multiply, identically or differently substituted with R¹¹,
   or for hydrogen,
R¹¹ stands for one or more of the following groups: —OH, fluorine, chlorine, bromine, cyano, —NH—C(O)R²⁰, —C(O)R²⁰, —C(O)OR²⁰, —C(O)NR²⁰R²¹ or —SO₂R²⁰
   or for C₁-C₆ alkyl, C₂-C₆ alkenyl, C₂-C₆ alkynyl, C₁-C₁₁ heteroalkyl, C₃-C₈ cycloalkyl, —O—(C₁-C₄ alkyl), —S—(C₁-C₄ alkyl), —O—(C₃-C₈ cycloalkyl), —S—(C₃-C₈ cycloalkyl), C₂-C₉ heterocyclyl or C₂-C₉ heteroaryl, each optionally singly or multiply, identically or differently substituted with fluorine, chlorine, bromine, —OH, cyano, C₁-C₆ alkyl or —O—(C₁-C₄ alkyl),
R¹² stands for —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with fluorine, chlorine, bromine, —OH or cyano, or for hydrogen
R¹⁵ and R¹⁶ mutually independently stand for hydrogen or —OH
   or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
   or together with the nitrogen atom to which they are bound foam a 3 to 7-membered ring, which can contain a further hetero atom from the range N or O not (directly) adjacent to the nitrogen,
R¹⁹ stands for H, C₁-C₆ alkyl, C₃-C₆ cycloalkyl, C₂-C₆ alkenyl, C₂-C₆ alkynyl, —C(S)R¹⁵, —C(O)R¹⁵, —SO₂R¹⁵ or —C(O)OR¹⁵, and
R²⁰ and R²¹ mutually independently stand for C₁-C₆ alkyl, C₃-C₆ cycloalkyl, C₂-C₆ alkenyl or C₂-C₆ alkynyl, each optionally singly or multiply, identically or differently substituted with fluorine, chlorine, bromine, —OH or cyano or for hydrogen,

and agrochemically active salts thereof, are preferred.

Compounds of the fo nula [I-b], wherein one or more of the symbols have one of the following meanings:
X¹ stands for C—H or N,
R¹ stands for phenyl or naphthalenyl, each optionally singly or multiply, identically or differently substituted with R⁷,
R² stands for halogen, C₁-C₆ alkyl, C₃-C₆ cycloalkyl or hydrogen,
R³⁰¹ for C₁-C₆ alkyl, C₂-C₆ alkenyl, C₃-₆ allenyl, C₂-₆ alkynyl, C₃-C₈ cycloalkyl, C₁-C₆ alkoxy, C₂-C₉ heterocyclyl, C₂-C₉ oxoheterocyclyl, or heteroaryl, each optionally singly or multiply, identically or differently substituted with R⁸,
R⁴⁰¹ stands for —NR¹²R¹³,
R⁵ and R⁶ mutually independently stand for hydrogen, fluorine, chlorine, bromine, cyano, nitro,
   or for C₁-C₆ alkyl, C₃-C₆ cycloalkyl, C₂-C₆ alkenyl, C₂-C₆ alkynyl, —O—(C₁-C₄ alkyl), S—(C₁-C₄ alkyl) or —S(O)—(C₁-C₆ alkyl), each optionally singly or multiply, identically or differently substituted with R¹¹,
   or together with the carbon atom to which they are bound form a ring with 5 to 8 ring atoms, wherein the ring can contain 1 to 4 further hetero atoms from the range oxygen, sulphur or —N—R¹⁹, optionally singly or multiply, identically or differently substituted with halogen, oxygen, cyano or C₁-C₄ alkyl,
R⁷ stands for fluorine, chlorine, cyano, nitro, methyl, ethyl, isopropyl, —CF₃, —CHF₂, C₂F₅ or CCl₃,
R⁸ stands for —OH, fluorine, chlorine, cyano, —C(O)R⁹, —C(O)OR⁹, —O—C(O)R⁹, —(CH₂)ₙC(O)R⁹ , wherein n=a whole number between 1 and 6, or for C₁-C₆ alkyl, C₂-C₈ alkenyl, C₂-C₈ alkynyl, C₃-C₈ cycloalkyl, C₁-C₆ haloalkyl, C₂-C₉ heterocyclyl, C₂-C₉ heteroaryl, —O—(C₁-C₄ alkyl), —S—(C₁-C₄ alkyl), —O—(C₃-C₈ cycloalkyl), —S—(C₃-C₈ cycloalkyl), each optionally singly or multiply, identically or differently substituted with R¹¹,
R⁹ and R¹⁰ mutually independently stand for C₁-C₆ alkyl, C₂-C₈ alkenyl, C₂-C₈ alkynyl or C₃-C₈ cycloalkyl, each optionally singly or multiply, identically or differently substituted with R¹¹,
   or for hydrogen,
R¹¹ stands for —OH, fluorine, chlorine, bromine, cyano, —NH—C(O)R²⁰, —C(O)R²⁰, —C(O)OR²⁰, —C(O)NR²⁰R²¹ or —SO₂R²⁰
   or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or, —S—(C₃-C₈ cycloalkyl), optionally singly or multiply identically or differently substituted with fluorine, chlorine, bromine, —OH, cyano, C₁-C₆ alkyl or —O—(C₁-C₄ alkyl),
R¹² stands for —C(S)R¹⁵, —C(O)R¹⁵, —SO₂R¹⁵, —C(O)OR¹⁵ or —C(O)NR¹⁵R¹⁶,
R¹³ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with fluorine or chlorine,
   or for hydrogen,
R¹⁵ and R¹⁶ mutually independently stand for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
   or for hydrogen
   or together with the nitrogen atom to which they are bound form a 3 to 7-membered ring, which can contain a further hetero atom from the range N or O not (directly) adjacent to the nitrogen,
R¹⁹ stands for H, C₂-C₆ alkynyl, —C(O)R¹⁵, —SO₂R¹⁵ or —C(O)OR¹⁵, and
R²⁰ and R²¹ mutually independently stand for C₁-C₆ alkyl, C₃-C₆ cycloalkyl, C₂-C₆ alkenyl or C₂-C₆ alkynyl, each optionally singly or multiply, identically or differently substituted with fluorine, chlorine, bromine, —OH or cyano
   or for hydrogen,

and agrochemically active salts thereof, are particularly preferred.

Compounds of the formula [I-b], wherein one or more of the symbols have one of the following meanings:
X¹ stands for C—H or N,
R¹ stands for phenyl, optionally singly or multiply identically or differently substituted with R⁷,
R² stands for methyl, ethyl, isopropyl, cyclopropyl, or hydrogen,
R³⁰¹ stands for C₁-C₆ alkyl, C₂-C₆ alkenyl, C₃-C₆ allenyl, C₂-C₆ alkynyl, C₃-C₈ cycloalkyl or C₁-C₆ alkoxy, each optionally singly or multiply, identically or differently substituted with R⁸,
R⁴⁰¹ stands for —NR¹²R¹³,
R⁵ and R⁶ mutually independently stand for hydrogen, fluorine or cyano
   or together with the carbon atoms to which they are bound form a ring wherein they together stand for —(CH═CH—CH═CH)—, —(CH═CH—N(R¹⁹)—, —(CH═CH—CH═N)— or —(CH₂—C(O)—N(R¹⁹)—, optionally singly or multiply identically or differently substituted with R¹¹,
R⁷ stands for fluorine, chlorine, cyano or methyl,
R⁸ stands for fluorine, chlorine or cyano or for C₁-C₆ alkyl, C₂-C₈ alkenyl, C₂-C₈ alkynyl, C₃-C₈ cycloalkyl, C₆-C₁₄ aryl, heterocyclyl, heteroaryl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with R¹¹,
R¹¹ stands for one or more of the following groups: —OH, fluorine, chlorine, bromine, cyano, —NH—C(O)R²⁰, —C(O)R²⁰, C(O)OR²⁰, —C(O)NR²⁰R²¹ or —SO₂R²⁰
   or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or S—(C₃-C₈ cycloalkyl), each optionally singly or multiply, identically or differently substituted with fluorine, chlorine, bromine, OH, cyano, C₁-C₆ alkyl or O—(C₁-C₄ alkyl),
R¹² stands for: —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₂-C₆ alkenyl, C₂-C₆ alkynyl or hydrogen,
R¹⁵ stands for C₁-C₆ alkyl, C₃-C₆ cycloalkyl, C₁-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with halogen, —OH, cyano or C₁-C₄ alkyl,
   or for hydrogen,
R¹⁶ stands for hydrogen, methyl, ethyl or propyl,
R¹⁹ stands for H, C₂-C₆ alkynyl, —C(O)R¹⁵, —SO₂R¹⁵ or —C(O)OR¹⁵, and
R²⁰ and R²¹ mutually independently stand for each methyl, ethyl, propyl, isopropyl, cyclopropyl, or cyclobutyl, optionally singly or multiply, identically or differently substituted with fluorine, chlorine, bromine, —OH or cyano,
   or for hydrogen,

and agrochemically active salts thereof, are quite particularly preferred.

In a further embodiment of the present invention, in particular compounds of the formula [I-b], wherein one or more of the symbols have one of the following meanings:
X¹ stands for N,
R¹ stands for phenyl, 3-methylphenyl, 2-fluorophenyl, 3-fluorophenyl, 4-fluorophenyl, 3-trifluoromethylphenyl, 4-chlorophenyl, 2,5-difluorophenyl, 2,4-difluorophenyl, 2,6-difluorophenyl, 3-methyl-4-fluorophenyl, 3-cyano-4-fluorophenyl or 2,4,6-tri-fluorophenyl,
R² stands for methyl, ethyl, isopropyl, cyclopropyl, or hydrogen,
R³⁰¹ stands for methyl, ethyl, 1-propyl, propan-2-yl, isobutyl, butan-2-yl, 2-methylpropyl, 2,2-dimethylpropyl, 2-(morpholin-4-yl)ethyl, 2-cyanoethyl, cyanomethyl, 2-cyano-2-methylpropyl, 3-methylbut-2-en-1-yl, but-2-en-1-yl, but-3-en-2-yl, propadienyl, prop-2-en-1-yl, prop-2-yn-1-yl, but-2-yn-1-yl, but-3-yn-2-yl, 2-methylbut-3-yn-2-yl, 2-methylbut-3-yn-2-yl, cyclopropyl, cyclobutyl, cyclopentyl, cyclohexyl, (2,2-dichlorocyclopropyl)methyl, cyclopropylmethyl, 1-cyclopropylethyl, trichloromethyl, trifluoromethyl, 2-fluoroethyl, 2,2-difluoroethyl, 2,2,2-trifluoroethyl, 2,2,2-trichloro-ethyl, 2-chloroethyl, 2-bromoethyl, 2-fluoropropyl, 3-fluoropropyl, 2-chloropropyl, 3-chloropropyl, 1,3-difluoropropan-2-yl, 1,1,1-trifluoropropan-2-yl, 1,1,1-trifluoro-2-methylpropan-2-yl, 3,3,3-trifluoro-2-hydroxypropyl, 2-fluorobenzyl, 3-fluorobenzyl, 4-fluorobenzyl, 2,3-difluorobenzyl, 2-chloro-6-fluorobenzyl, 1-(2-chlorophenyl)ethyl, 1-(3-chlorophenyl)ethyl, 1-(4-chlorophenyl)ethyl, 3-cyanobenzyl, 4-cyanobenzyl, 4-(difluoromethoxy)benzyl, 2-cyanobenzyl, 2-(3-chlorophenyl)ethyl, 2-(2-chloro-phenyl)ethyl, 1-naphthylmethyl, (pyridine-3-ylmethyl, 2-chloro-1,3-thiazol-5-yl)-methyl, methoxymethyl, 2-methoxyethyl, 2-methoxypropyl, 2-(methylsulphanyl)-ethyl, 2-(trifluoromethoxy)ethyl, 1-methoxypropan-2-yl, 2-[2-(2-methoxyethoxy)-ethoxy]ethyl, 2-(2-methoxyethoxy)ethyl, 1,3-dimethoxypropan-2-yl, 2-(cyclopropyl-oxy)ethyl, tetrahydrofuran-2-ylmethyl, (3-methyloxetan-3-yl)methyl, 1H-imidazol-2-ylmethyl, tetrahydrofuran-3-yl, 2-oxotetrahydrofuran-3-yl, 2-tert-butoxy-2-oxoethyl, 1-methoxy-3-methyl-1-oxobutan-2-yl, 1-methoxy-1-oxopropan-2-yl or 3-ethoxy-3-oxopropyl, 1-cyanopropan-2-yl, 1-propyl, propan-2-yloxy or 2-ethoxyethyl,
R⁴⁰¹ stands for —NHR¹²,
R⁵ and R⁶ mutually independently stand for hydrogen, fluorine or cyano
   or together with the carbon atoms to which they are bound form a ring wherein they together stand for —(CH═CH—CH═CH)—, —(CH═CH—N(R¹⁹)—, —(CH═CH—CH═N)— or —(CH₂—C(O)—N(R¹⁹)—, optionally singly or multiply identically or differently substituted with R¹¹,
R¹¹ stands for one or more of the following groups: —OH, fluorine, chlorine, cyano, methyl, ethyl or cyclopropyl,
R¹² stands for —C(S)R¹⁵, —SO₂R¹⁵, —C(O)OR¹⁵ or —C(O)R¹⁵,
R¹³ stands for methyl, ethyl, n-propyl, isopropyl, sec-butyl, isobutyl, cyclopropyl, cyclobutyl, cyclopentyl, cyclohexyl, 2-methoxyethyl, (2-methoxyethoxy)methyl, cyclopentenyl, cyclohexenyl, oxetanyl, tetrahydrofuran-2-yl, ethinyl, prop-1-in-1-yl, prop-1-en-1-yl, aminomethyl, aminoethyl, aminopropyl, aminobutyl, aminoisopropyl, aminocyclopropyl, aminocyclobutyl, aminocyclopentyl, dimethylamino, ethyl-(methyl)amino, pyrrolidinyl, diethylamino, 2-pyridyl, 3-pyridyl, 4-pyridyl, ethoxy-carbonyl, benzyl, phenyl, 2-thienyl or 3-thienyl, each optionally singly or multiply, identically or differently substituted with halogen, —OH, cyano or C₁-C₄ alkyl,
   or for hydrogen, and
R¹⁹ stands for H, acetyl, ethoxycarbonyl, methoxycarbonyl, prop-2-yn-1-yl or but-2-yn-1-yl,

and agrochemically active salts thereof, are also preferred.

In a further embodiment of the present invention in particular compounds of the formula [I-b], wherein one or more of the symbols have one of the following meanings:
X¹ stands for C—H,
R¹ stands for 4-fluorophenyl, 3-chlorophenyl, 2,6-difluorophenyl or 3-methylphenyl
R² stands for cyclopropyl, methyl, H or difluoromethoxy, and
R⁴⁰¹ stands for acetylamino, n-propionylamino, isobutyrylamino, (cyclopropylcarbonyl)-amino, (methoxyacetyl)amino, 2-methoxypropanoyl, (2-methylbutanoyl)amino, but-2-enoylamino, prop-2-ynoylamino, 3-(dimethylamino)prop-2-enoyl]amino, 3,3,3-tri-fluoropropanoyl)amino, 3,3-difluoropropanoyl)amino, (cyclopropylacetyl)amino, lactoylamino, (cyclobutylcarbonyl)amino, (cyclopentylacetyl)amino, 2-methylcyclo-propyl)carbonyl]amino, (3-methylbutanoyl)amino, (phenylacetyl)amino, benzoyl-amino, (3-thienylcarbonyl)amino, (2-thienylcarbonyl)amino (2-hydroxy-2-methyl-propanoyl)amino, [(2-methoxyethoxy)acetyl]amino or 2,3-dihydroxypropanoyl)-amino,

wherein the other substituents have one or more of the aforesaid meanings,

and the agrochemically active salts thereof, are preferred.

Further, compounds of the formula [I-b], wherein one or more of the symbols have one of the following meanings:
X¹ stands for C—H,
R¹ stands for 4-fluorophenyl, 3-chlorophenyl, 2,6-difluorophenyl or 3-methylphenyl,
R² stands for cyclopropyl, methyl, H or difluoromethoxy,
R³⁰¹ stands for methyl, ethyl, 1-propyl, propan-2-yl, isobutyl, butan-2-yl, 2-methylpropyl, 2,2-dimethylpropyl, 2-(morpholin-4-yl)ethyl, 2-cyanoethyl, cyanomethyl, 2-cyano-2-methylpropyl, 3-methylbut-2-en-1-yl, but-2-en-1-yl, but-3-en-2-yl, propadienyl, prop-2-en-1-yl, prop-2-yn-1-yl, but-2-yn-1-yl, but-3-yn-2-yl, 2-methylbut-3-yn-2-yl, 2-methylbut-3-yn-2-yl, cyclopropyl, cyclobutyl, cyclopentyl, cyclohexyl, (2,2-dichlorocyclopropyl)methyl, cyclopropylmethyl, 1-cyclopropylethyl, trichloromethyl, trifluoromethyl, 2-fluoroethyl, 2,2-difluoroethyl, 2,2,2-trifluoroethyl, 2,2,2-trichloro-ethyl, 2-chloroethyl, 2-bromoethyl, 2-fluoropropyl, 3-fluoropropyl, 2-chloropropyl, 3-chloropropyl, 1,3-difluoropropan-2-yl, 1,1,1-trifluoropropan-2-yl, 1,1,1-trifluoro-2-methylpropan-2-yl, 3,3,3-trifluoro-2-hydroxypropyl, 2-fluorobenzyl, 3-fluorobenzyl, 4-fluorobenzyl, 2,3-difluorobenzyl, 2-chloro-6-fluorobenzyl, 1-(2-chlorophenyl)ethyl, 1-(3-chlorophenyl)ethyl, 1-(4-chlorophenyl)ethyl, 3-cyanobenzyl, 4-cyanobenzyl, 4-(difluoromethoxy)benzyl, 2-cyanobenzyl, 2-(3-chlorophenyl)ethyl, 2-(2-chloro-phenyl)ethyl, 1-naphthylmethyl, (pyridine-3-ylmethyl, 2-chloro-1,3-thiazol-5-yl)-methyl, methoxymethyl, 2-methoxyethyl, 2-methoxypropyl, 2-(methylsulphanyl)-ethyl, 2-(trifluoromethoxy)ethyl, 1-methoxypropan-2-yl, 2-[2-(2-methoxyethoxy)-ethoxy]ethyl, 2-(2-methoxyethoxy)ethyl, 1,3-dimethoxypropan-2-yl, 2-(cyclopropyl-oxy)ethyl, tetrahydrofuran-2-ylmethyl, (3-methyloxetan-3-yl)methyl, 1H-imidazol-2-ylmethyl, tetrahydrofuran-3-yl, 2-oxotetrahydrofuran-3-yl, 2-tert-butoxy-2-oxoethyl, 1-methoxy-3-methyl-1-oxobutan-2-yl, 1-methoxy-1-oxopropan-2-yl, 3-ethoxy-3-oxopropyl, 1-cyanopropan-2-yl, propan-2-yloxy, 2-ethoxyethyl, 3-methoxypropyl, 2-(trifluoromethoxy)ethyl or 1,3-dioxolan-2-ylmethyl, and
R⁴⁰¹ stands for —NHR¹²,

wherein the other substituents have one or more of the aforesaid meanings,

and the agrochemically active salts thereof, are especially preferred.

Compounds of the formula [I-b], wherein
X¹ stands for C—H,

wherein the other substituents have one or more of the aforesaid meanings,

and the agrochemically active salts thereof, are further especially preferred.

Compounds of the formula [I-b], wherein
R¹ has the same meaning as the general residue definition of R¹ or that expounded in preferred ranges in the compounds of the formula [I-a],

wherein the other substituents have one or more of the aforesaid meanings, and the agrochemically active salts thereof, are further especially preferred.

Compounds of the formula [I-b], wherein
R² has the same meaning as the general residue definition of R² or that expounded in preferred ranges in the compounds of the formula [I-a],

wherein the other substituents have one or more of the aforesaid meanings, and the agrochemically active salts thereof, are further especially preferred.

Compounds of the formula [I-b], wherein
R³⁰¹ stands for methyl, ethyl, 1-propyl, propan-2-yl, isobutyl, butan-2-yl, 2-methylpropyl, prop-2-yn-1-yl, cyclopropyl, cyclobutyl, cyclopentyl, (2,2-dichlorocyclopropyl)-methyl, 2-cyanoethyl, 2-chloroethyl, cyclopropylmethyl, 2,2-difluoroethyl, 2,2,2-trifluoroethyl, 2-fluorobenzyl, 2-(trifluoromethoxy)ethyl, 1-methoxypropan-2-yl, 1-cyclopropylethyl, 1-cyanopropan-2-yl, propan-2-yloxy, 2-ethoxyethyl, 3-methoxy-propyl, 2-(trifluoromethoxy)ethyl or 1,3-dioxolan-2-ylmethyl,

wherein the other substituents have one or more of the aforesaid meanings,

and the agrochemically active salts thereof, are further especially preferred.

Compounds of the formula [I-b], wherein
R⁴⁰¹ stands for —NH—COR¹⁵,

wherein the other substituents have one or more of the aforesaid meanings,

and the agrochemically active salts thereof, are further especially preferred.

Compounds of the formula [I-b], wherein
R¹⁵ stands for C₁-C₆ alkyl or C₃-C₆ cycloalkyl,

wherein the other substituents have one or more of the aforesaid meanings, and the agrochemically active salts thereof, are further especially preferred.

Compounds of the formula [I-b], wherein
R¹ has the same meaning as the general residue definitions of R¹ or those expounded in preferred ranges in the compounds of the formula [I-a],
R² has the same meaning as the general residue definitions of R² or those expounded in preferred ranges in the compounds of the formula [I-a],
R⁵ and R⁶ have the same meaning as the general residue definitions of R⁵ and R⁶ or those expounded in preferred ranges in the compounds of the formula [I-a],
R⁷ has the same meaning as the general residue definitions of R⁷ or those expounded in preferred ranges in the compounds of the formula [I-a],
R¹⁹ has the same meaning as the general residue definitions of R¹⁹ or those expounded in preferred ranges in the compounds of the formula [I-a],
R²⁰ and R²¹ have the same meaning as the general residue definitions of R²⁰ and R²¹ or those expounded in preferred ranges in the compounds of the formula [I-a],

wherein the other substituents have one or more of the aforesaid meanings, and the agrochemically active salts thereof, are further especially preferred.

The aforesaid residue definitions can be mutually combined in any manner. In addition, some individual definitions may not apply.

The residue definitions or explanations expounded generally or expounded in preferred ranges above can however also be mutually combined, i.e. between the relevant ranges and preferred ranges. They apply for the final products and for the precursors and intermediates correspondingly. In addition, some individual definitions may not apply.

Those compounds of the formula [I-b], in which all residues each have the aforesaid preferred meanings are preferred.

Those compounds of the formula [I-b], in which all residues each have the aforesaid particularly preferred meanings are particularly preferred.

Those compounds of the formula [I-b], in which all residues each have the aforesaid quite particularly preferred meanings are quite particularly preferred.

Those compounds of the formula [I-b], in which all residues each have the aforesaid especially preferred meanings are especially preferred.

The compounds according to the invention of the formulae [I-a] and [I-b] can in some cases be present as mixtures of different possible isomeric forms, in particular of stereoisomers such as for example E and Z, threo and erythro, and optical isomers, but some times also tautomers. Both the E and also the Z isomers, and also the threo and erythro, and the optical isomers, any mixtures of these isomers and the possible tautomeric forms are claimed.

Optionally substituted groups can be singly or multiply substituted, wherein in the case of multiple substitutions the substituents can be the same or different.

Depending on the nature of the substituents defined above, the compounds of the formula (I) exhibit acidic or basic properties and can form salts with inorganic or organic acids or with bases or with metal ions, and in some cases also internal salts or adducts. If the compounds of the formula (I) bear amino, alkylamino or other groups inducing basic properties, then these compounds can be converted to salts with acids, or arise directly as the salt through the synthesis. If the compounds of the formula (I) bear hydroxy, carboxy or other groups inducing acidic properties, then these compounds can be converted to salts with bases. Suitable bases are for example hydroxides, carbonates and hydrogen carbonates of the alkali and alkaline earth metals, in particular those of sodium, potassium, magnesium and calcium, and also ammonia, primary, secondary and tertiary amines with C₁-C₄ alkyl groups, mono-, di- and trialkanolamine from C₁-C₄ alkanols, choline and chlorocholine.

Examples of inorganic acids are hydrohalic acids such as hydrogen fluoride, hydrogen chloride, hydrogen bromide and hydrogen iodide, sulphuric acid, phosphoric acid and nitric acid and acidic salts such as NaHSO₄ and KHSO₄. As organic acids, for example formic acid, carbonic acid and alkanoic acids such as acetic acid, trifluoroacetic acid, trichloroacetic acid and propionic acid and also glycolic acid, thiocyanic acid, lactic acid, succinic acid, citric acid, benzoic acid, cinnamic acid, oxalic acid, saturated or singly or doubly unsaturated C₆-C₂₀ fatty acids, saturated or singly or doubly unsaturated C₆-C₂₀ alkylenedicarboxylic acids, alkylsulphuric acid monoesters, alkylsulphonic acids (sulphonic acids with straight-chain or branched alkyl residues with 1 to 20 carbon atoms), arylsulphonic acids or aryldisulphonic acids (aromatic residues such as phenyl and naphthyl which bear one or two sulphonic acid groups), alkylphosphonic acids (phosphonic acids with straight-chain or branched alkyl residues with 1 to 20 carbon atoms), arylphosphonic acids or aryldiphosphonic acids (aromatic residues such as phenyl and naphthyl which bear one or two phosphonic acid residues), wherein the alkyl or aryl residues can bear further substituents, e.g. p-toluenesulphonic acid, salicylic acid, p-aminosalicylic acid, 2-phenoxybenzoic acid, 2-acetoxybenzoic acid etc.

Possible metal ions are in particular the ions of the elements of the second main group, in particular calcium and magnesium, the third and fourth main group, in particular aluminium, tin and lead, and the first to eighth transition group, in particular chromium, manganese, iron, cobalt, nickel, copper, zinc and others. The metal ions of the elements of the fourth period are particularly preferred. Here the metals can be present in the various valencies available to them.

The salts thus obtainable also exhibit fungicidal and mycotoxin-reducing properties.

In the definitions of the symbols stated in the above formulae, collective terms were used, which generally representatively stand for the following substituents:

Alkyl: saturated, straight-chain or branched hydrocarbon residues with 1 to 8 carbon atoms, e.g. (but not limited to) C₁-C₆ alkyl such as methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-di-methylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Preferably alkyl stands for saturated, straight-chain or branched hydrocarbon residues with 1 to 6 and preferably 1 to 4 carbon atoms.

Haloalkyl: straight-chain or branched alkyl groups with 1 to 8 (preferably 1 to 6 and still more preferably 1 to 4) carbon atoms (as aforesaid), wherein in these groups the hydrogen atoms can be partly or wholly replaced by

halogen atoms as aforesaid, e.g. (but not limited to) C₁-C₃ haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoro-prop-2-yl;

Cycloalkyl: monocyclic, saturated hydrocarbon groups with 3 to 8 (preferably 3 to 6) carbon ring members, e.g. (but not limited to) cyclopropyl, cyclopentyl and cyclohexyl;

Halocycloalkyl: monocyclic, saturated hydrocarbon groups with 3 to 8 (preferably 3 to 6) carbon ring members (as aforesaid), wherein in these groups the hydrogen atoms can be partly or wholly replaced by halogen atoms as aforesaid, e.g. (but not limited to) 2-fluorocyclopropyl, 2,2-difluorocyclopropyl, 3,3-difluorocyclobutyl, 2-fluorocyclopentyl and 3-fluorocyclopentyl;

Heterocyclyl: three to fifteen-membered preferably three to nine-membered saturated or partly unsaturated heterocycle, containing one to four hetero atoms from the group oxygen, nitrogen or sulphur: mono-, bi- or tricyclic heterocycles containing apart from carbon ring members one to three nitrogen atoms and/or one oxygen or sulphur atom or one or two oxygen and/or sulphur atoms; if the ring contains several oxygen atoms, then these are not situated directly adjacent; such as for example (but not limited to) oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazol-idinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydro-fur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-di-hydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydroopyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidineyl, 3-piperidineyl, 4-piperidineyl, 1,3-dioxan-5-yl, 2-tetrahydropyranyl, 4-tetrahydropyranyl, 2-tetrahydrothienyl, 3-hexahydropyrid-azinyl, 4-hexahydropyridazinyl, 2-hexahydropyrimidinyl, 4-hexahydropyrimidinyl, 5-hexahydro-pyrimidinyl, 2-piperazinyl, 1,3,5-hexahydro-triazin-2-yl and 1,2,4-hexahydrotriazin-3-yl;

Oxoheterocyclyl: three to fifteen-membered preferably three to nine-membered saturated or partly unsaturated heterocycle, (as aforesaid), wherein in these groups the hydrogen atoms of one or more CH₂ groups can be replaced by one or more carbonyl groups, e.g. (but not limited to) 2-oxooxetan-3-yl, 5-oxotetrahydrofuran-3-yl, 2-oxotetrahydrofuran-3-yl, 2,5-dioxotetrahydrofuran-3-yl, 5-oxo-2,5-dihydrofuran-3-yl, 2-oxo-2,5-dihydrofuran-3-yl, 5-oxopyrrolidin-3-yl, 2-oxopyrrolidin-3-yl, 5-oxo-pyrrolidin-2-yl), 3-oxopyrrolidin-2-yl and 4-oxo-3,4-dihydro-2H-pyran-5-yl;

Alkenyl: unsaturated, straight-chain or branched hydrocarbon residues with 2 to 8 (preferably 2 to 6) carbon atoms and a double bond in any position, e.g. (but not limited to) C2-C6 alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1,-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

Allcynyl: straight-chain or branched hydrocarbon groups with 2 to 8 (preferably 2 to 6) carbon atoms and a triple bond in any position, e.g. (but not limited to) C₂-C₆ alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

Aryl: 6 to 14-membered, completely unsaturated carbocyclic ring system, e.g. (but not limited to) phenyl, 1-naphthyl, 2-naphthyl, 2-anthryl and 1-anthryl;

Heteroaryl: 5 or 6-membered, completely unsaturated monocyclic ring system, containing one to four hetero atoms from the group oxygen, nitrogen or sulphur, if the ring contains several oxygen atoms, then these are not situated directly adjacent;

Alkoxy: a straight-chain or branched alkoxy residue, preferably C₁-C₆ alkoxy residue and particularly preferably a C₁-C₃ alkoxy residue, such as for example (but not limited to) methoxy, ethoxy, n-propoxy, 1-methylethoxy, n-butoxy, 1-methylpropoxy, 2-methylpropoxy or 1,1-dimethylethoxy, in particular for methoxy or ethoxy;

Alkylthio: stands for straight-chain or branched alkylthio e.g. (but not limited to) methylthio, ethylthio, n- and i-propylthio, n-, i-, sec.- and tert-butylthio, n-pentylthio and isomers thereof such as 1-, 2- and 3-methyl-butylthio. The alkylthio groups can be substituted with 1 to 3 halogen atoms (preferably chlorine and/or fluorine), e.g. (but not limited to) are di- and trifluoromethylthio and difluorochloromethylthio.

Haloalkoxy: stands for a straight-chain or branched alkoxy residue wherein one or more hydrogen atoms have been replaced by fluorine, chlorine or bromine, e.g. (but not limited to) —OCF₃ or —OCHF₂. A one- to threefold substitution with fluorine or chlorine is preferred.

Acyloxy: stands for a straight-chain, branched, cyclic, saturated or unsaturated acyloxy residue bound via the oxygen atom, e.g. (but not limited to) acetyloxy, propionyloxy and isobutyryloxy.

Heteroalkyl: saturated or unsaturated, straight-chain or branched hydrocarbon residues with 2 to 10 (preferably 2 to 8) carbon atoms and at least one hetero atom, wherein two hetero atoms must not be directly adjacent.

Combinations which contradict the laws of nature and which those skilled in the art would therefore have excluded on the basis of their specialist knowledge are not included. For example, ring structures with three or more adjacent O atoms are excluded.

Explanation of the Processes and Intermediates

The phenylpyri(mi)dinylazoles according to the invention of the formulae [I-a] and [I-b] can be produced in different ways. For the purposes of the process description, the compounds of the formulae [I-a] and [I-b] are taken together under the formula [I], since the process according to the invention can be applied to both formulae. Below, the possible processes are firstly shown schematically. Unless otherwise stated, the residues stated have the meanings stated above.

The phenylpyri(mi)dinylazoles according to the invention of the formula [I] can be produced by process A according to the following scheme.

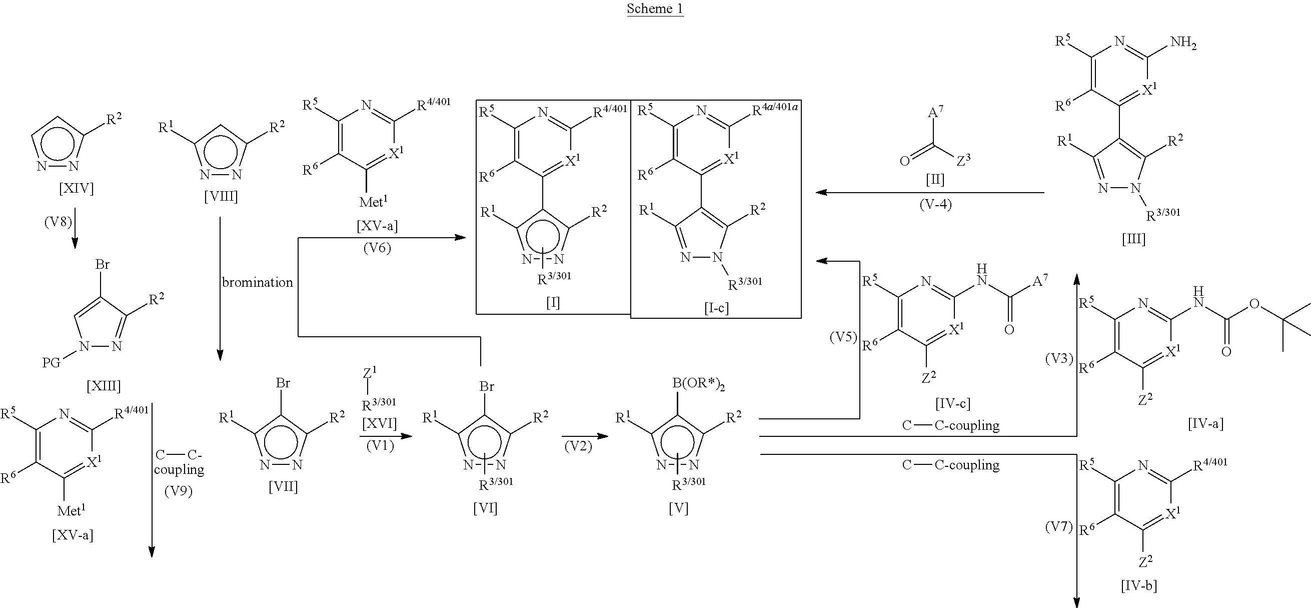

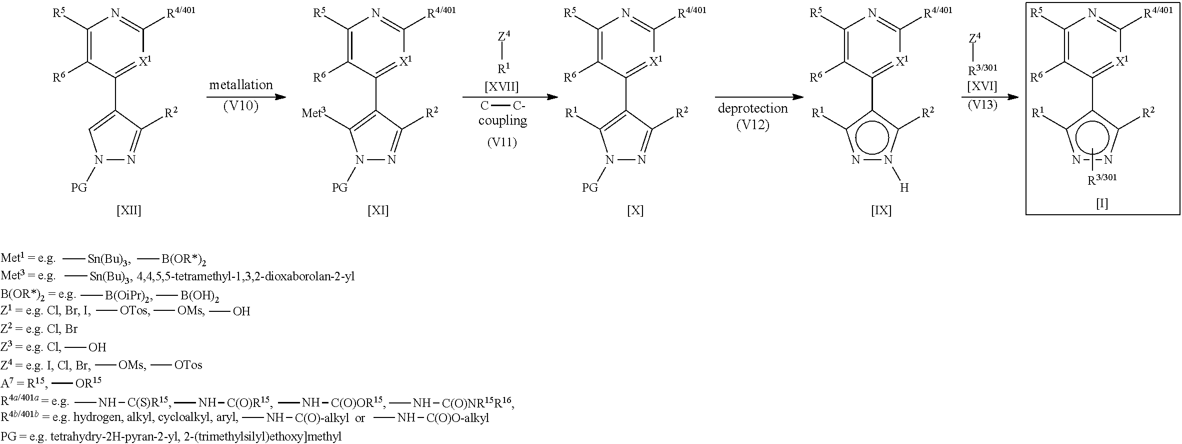

In addition, the phenylpyri(mi)dinylazoles according to the invention of the formula [I-d] can also be produced by process B (Scheme 2)

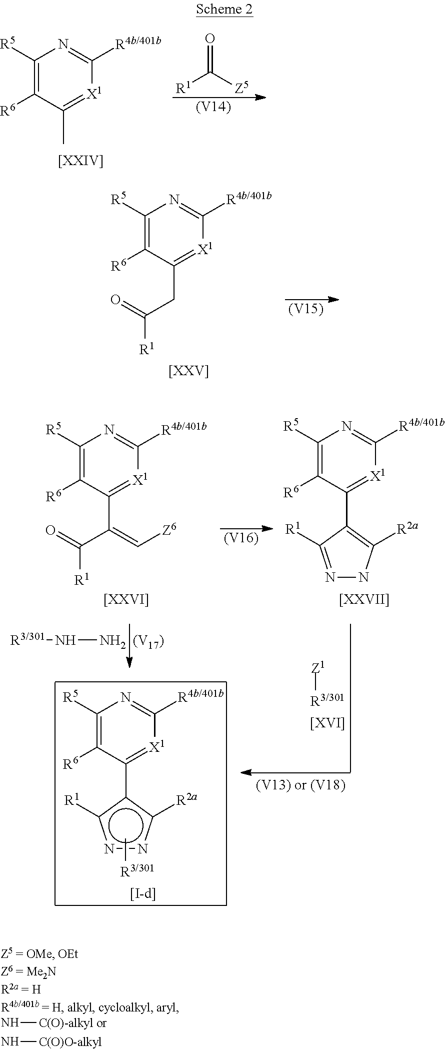

Alternatively the arylpyrazoles according to the invention of the formula [I-e] and intermediates of the formula [IX-b] can also be produced by process C (Scheme 3).

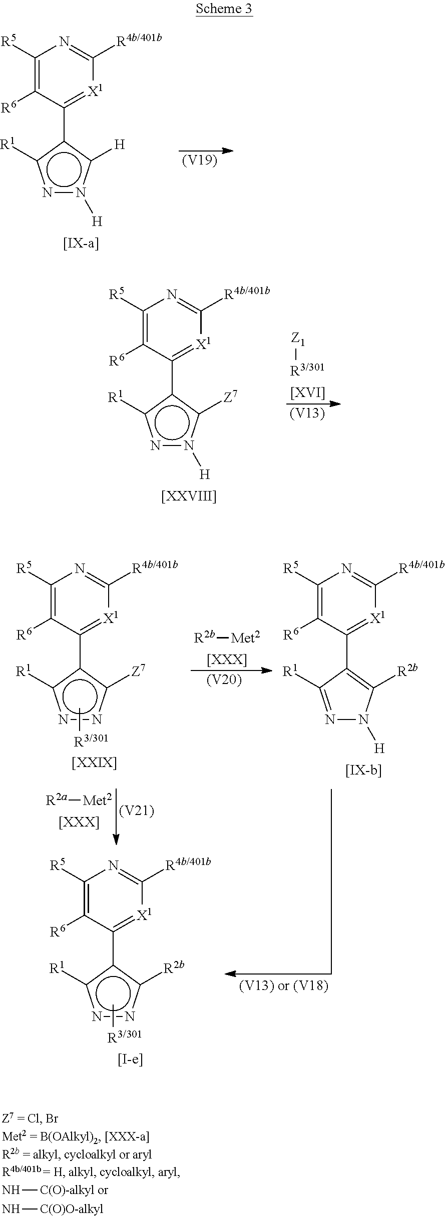

Alternatively the intermediates of the formula [VI-a] can also be produced by process D (Scheme 4).

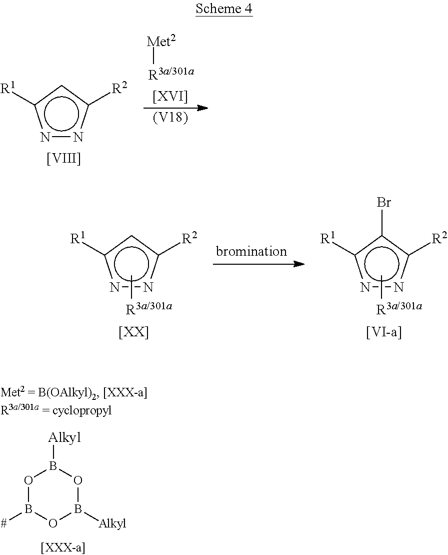

Alternatively the intermediates of the formula [VI-b] and intermediates of the formula [VI-c] can also be produced by process E (Scheme 5).

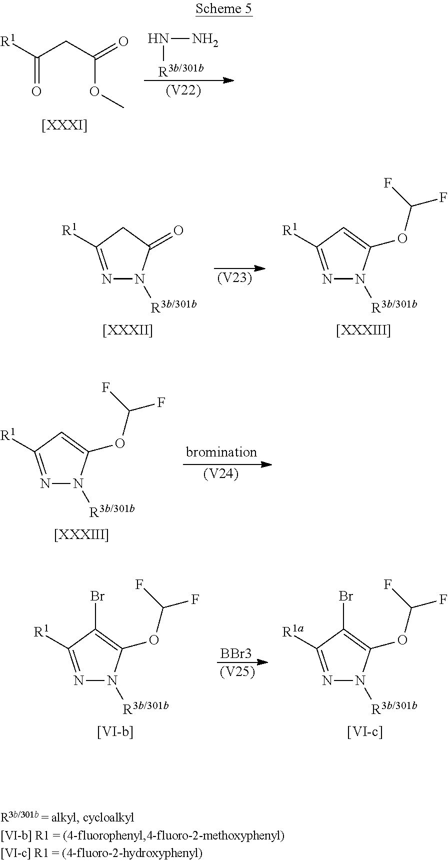

Alternatively the intermediates of the formula [III] can also be produced by process F (Scheme 6).

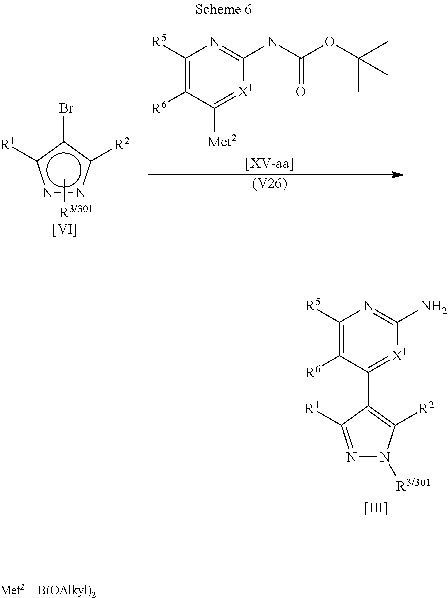

Intermediates of the type [XV-a] can be produced by process G (Scheme 7).

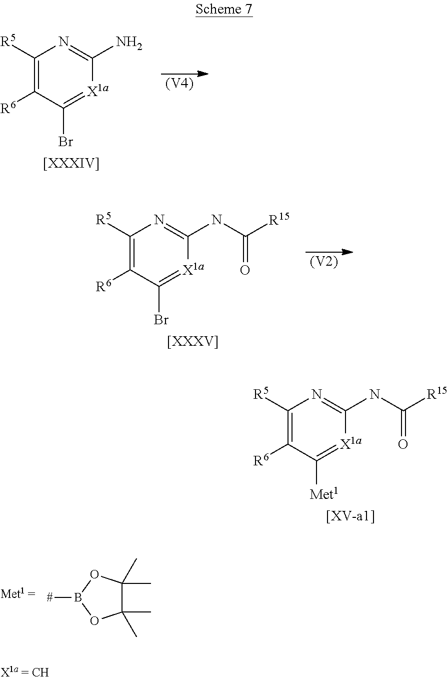

In addition, the phenylpyri(mi)dinylazoles according to the invention of the formula [I-f] and [I-g] can also be produced by process H (Scheme 8)

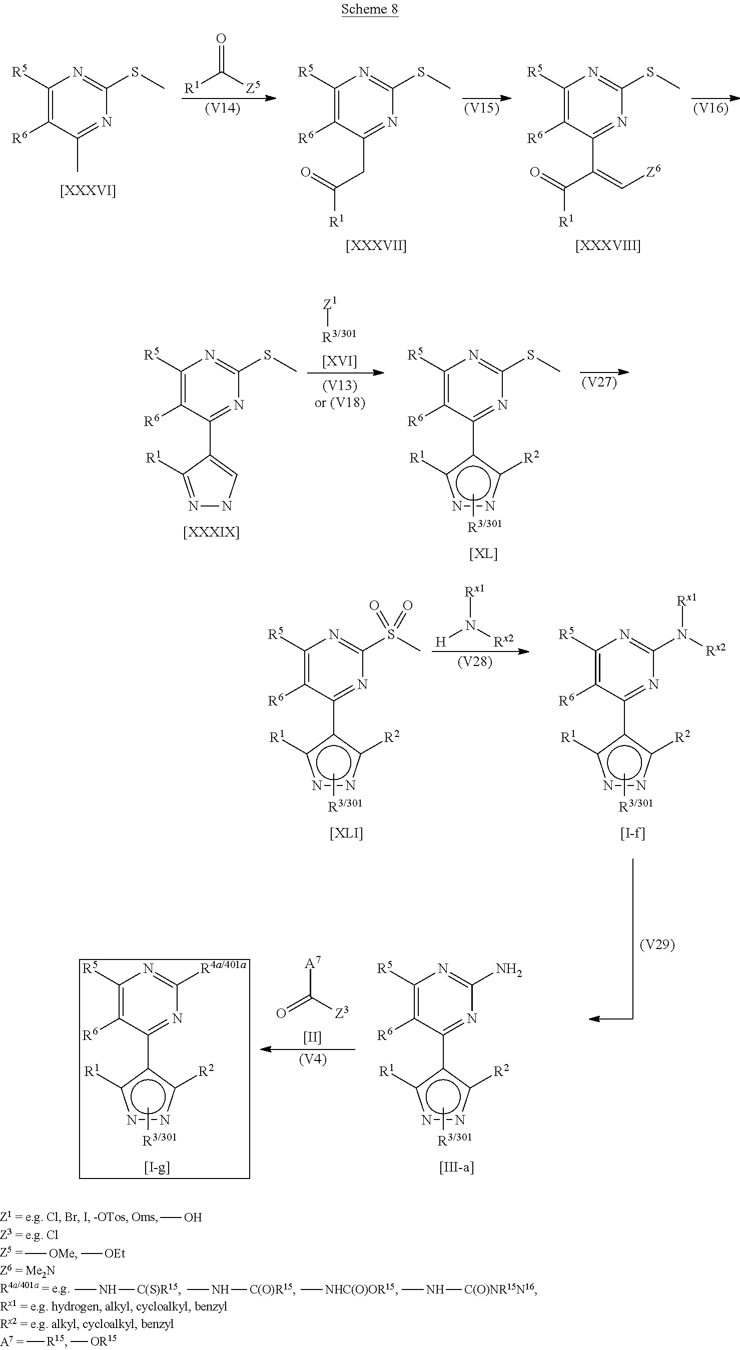

In addition, the phenylpyri(mi)dinylazoles according to the invention of the formula [I-h] can be produced by process I (Scheme 9)

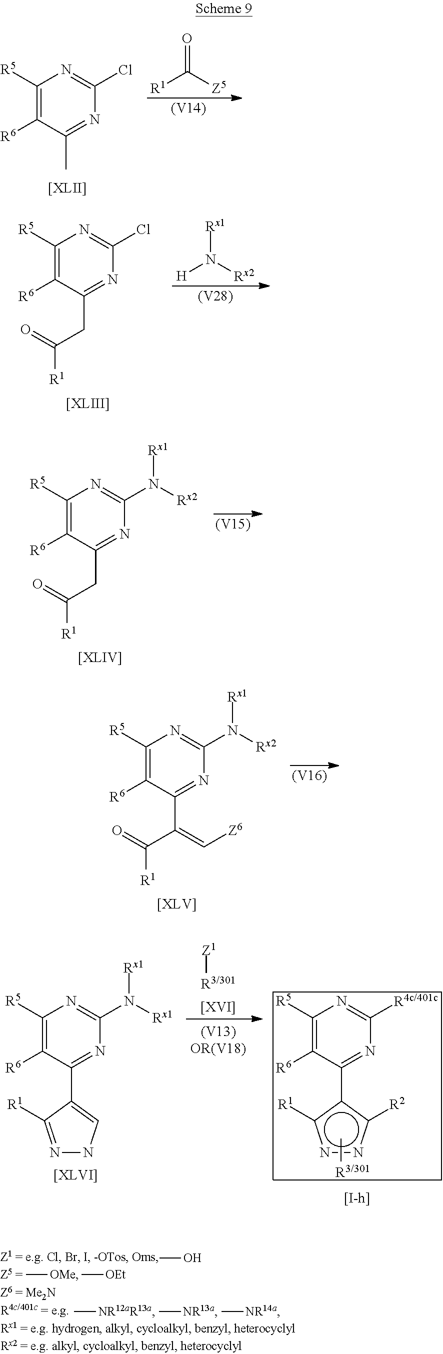

Compounds of the formula [III]

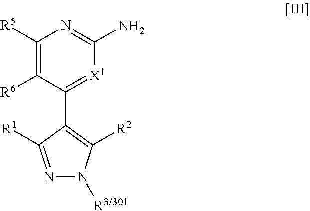

wherein the symbols R¹, R², R^{3/301}, R⁵, R⁶ and X¹ have the aforesaid general, preferred, particularly preferred, quite particularly preferred, most preferred or especially preferred meanings, and salts thereof, are novel.

For example the compounds of the type [III] listed in the following table are novel:

| | | | | | LogP | |
|---|---|---|---|---|---|---|
| | | | | | (pH | [M + H]⁺ |
| No. | Name | R¹ | R² | R^{3/301} | 2.3)¹ | Peak² |
| [III-1] | 4-[3-(4-fluorophenyl)-1- | 4- | H | isopropyl | 1.22 | 297.13 |
| | isopropyl-1H-pyrazol-4- | fluorophenyl | | | | |
| | yl]pyridin-2-amine | | | | | |
| [III-2] | 4-[3-(4-fluorophenyl)-1- | 4- | H | 2-methoxyethyl | 0.98 | 313.15 |
| | (2-methoxyethyl)-1H- | fluorophenyl | | | | |
| | pyrazol-4-yl]pyridin-2- | | | | | |
| | amine | | | | | |
| [III-3] | 4-[1-ethyl-3-(4- | 4- | H | ethyl | 0.97 | 283.20 |
| | fluorophenyl)-1H-pyrazol- | fluorophenyl | | | | |
| | 4-yl]pyridin-2-amine | | | | | |
| [III-4] | 4-[1-(2,2-difluoroethyl)-3- | 4- | H | 2,2- | 1.03 | 319.48 |
| | (4-fluorophenyl)-1H- | fluorophenyl | | difluoroethyl | | |
| | pyrazol-4-yl]pyridin-2- | | | | | |
| | amine | | | | | |
| [III-5] | 4-[3-(4-fluorophenyl)-1- | 4- | H | methyl | 0.71 | 269.2 |
| | methyl-1H-pyrazol-4- | fluorophenyl | | | | |
| | yl]pyridin-2-amine | | | | | |

wherein
X¹ stands for ═C—H and
R⁵, R⁶ for H.

The compounds 4-[3-(4-fluorophenyl)-5-methyl-1H-pyrazol-4-yl]pyridin-2-amine, 4-[3-(4-chloro-phenyl)-5-methyl-1H-pyrazol-4-yl]pyridin-2-amine, 4-[3-(4-methoxyphenyl)-5-methyl-1H-pyrazol-4-yl]pyridin-2-amine, 4-[3-(4-fluorophenyl)-1H-pyrazol-4-yl]pyrimidin-2-amine, 4-(5-methyl-3-phenyl-1H-pyrazol-4-yl)pyrimidin-2-amine and [4-(2-aminopyrimidin-4-yl)-3-(3-chloro-5-hydroxyphenyl)-1H-pyrazol-1-yl]acetonitrile are excepted.

Compounds of the formula [V]

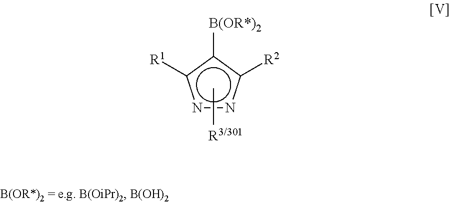

wherein the symbols R², R^{3/301} have the aforesaid general, preferred, particularly preferred, quite particularly preferred or especially preferred meanings, and

R¹ has the aforesaid preferred, particularly preferred, quite particularly preferred, most preferred or especially preferred meanings, and salts thereof, are also novel.

For example the compounds of the type [V] listed in the following table are novel:

| | | | | | LogP | |
|---|---|---|---|---|---|---|
| | | | | | (pH | [M + H]⁺ |
| No. | Name | R¹ | R² | R^{3/301} | 2.3)¹ | Peak² |
| [V-1] | 3-(4-fluorophenyl)-1- | 4- | H | isopropyl | 4.46 | 331.2 |
| | isopropyl-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-2] | 3-(4-fluorophenyl)-1-(2- | 4- | H | 2-methoxyethyl | 3.58 | 347.2 |
| | methoxyethyl)-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-3] | 3-(4-fluorophenyl)-1- | 4- | H | isobutyl | 4.89 | 345.2 |
| | isobutyl-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-4] | 1-(2,2-difluoroethyl)-3-(4- | 4- | H | 2,2-difluoroethyl | 3.84 | 353.2 |
| | fluorophenyl)-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-5] | 3-(4-fluorophenyl)-1- | 4- | H | isopropoxy | 4.81 | 347.2 |
| | isopropoxy-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-6] | 1-(cyclopentyloxy)-3-(4- | 4- | H | cyclopentyloxy | 5.51 | 373.2 |
| | fluorophenyl)-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-7] | 1-cyclopropyl-3-(4- | 4- | H | cyclopropyl | 3.47 | 329.2 |
| | fluorophenyl)-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-8] | 1-(cyclopropylmethyl)-3- | 4- | H | cyclopropylmethyl | 4.42 | 343.2 |
| | (4-fluorophenyl)-4- | fluorophenyl | | | | |
| | (4,4,5,5-tetramethyl-1,3,2- | | | | | |
| | dioxaborolan-2-yl)-1H- | | | | | |
| | pyrazole | | | | | |
| [V-9] | 3-(4-fluorophenyl)-1- | 4- | H | methyl | 3.47 | 303.2 |
| | methyl-4-(4,4,5,5- | fluorophenyl | | | | |
| | tetramethyl-1,3,2- | | | | | |
| | dioxaborolan-2-yl)-1H- | | | | | |
| | pyrazole | | | | | |
| [V-10] | 1-(2-chloroethyl)-3-(4- | 4- | H | 2-chloroethyl | 4.06 | 351.1 |
| | fluorophenyl)-4-(4,4,5,5- | fluorophenyl | | | | |
| | tetramethyl-1,3,2- | | | | | |
| | dioxaborolan-2-yl)-1H- | | | | | |
| | pyrazole | | | | | |

The compound 1-methyl-3-phenyl-4-(4,4,5,5-tetramethyl-1,3,2-dioxaborolan-2-yl)-1H-pyrazole is excepted.

Compounds of the formula [VI]

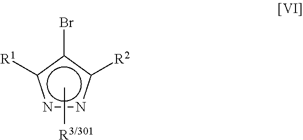

wherein R¹ has the aforesaid particularly preferred, quite particularly preferred, most preferred or especially preferred meanings, and

R² and R^{3/301} have the aforesaid preferred, particularly preferred, quite particularly preferred, most preferred or especially preferred meanings,

and salts thereof, are novel.

For example the compounds of the type [VI] listed in the following table are novel:

| | | | | | LogP | |
|---|---|---|---|---|---|---|
| | | | | | (pH | [M + H]⁺ |
| No. | Name | R¹ | R² | R^{3/301} | 2.3)¹ | Peak² |
| [VI-2] | 4-bromo-1-ethyl-3-(4- | 4-fluoro- | H | C2H5 | 3.32 | 269.0 |
| | fluorophenyl)-1H-pyrazole | phenyl | | | | |
| [VI-4] | 4-bromo-3-(4-fluorophenyl)- | 4-fluoro- | H | isobutyl | 4.34 | 297.0 |
| | 1-isobutyl-1H-pyrazole | phenyl | | | | |
| [VI-6] | 4-bromo-3-(4-fluorophenyl)- | 4-fluoro- | H | 2-methoxyethyl | 3.08 | 299.0 |
| | 1-(2-methoxyethyl)-1H- | phenyl | | | | |
| | pyrazole | | | | | |
| [VI-9] | 4-bromo-3-(4-fluorophenyl)- | 4-fluoro- | H | 1-(2-fluorophenyl)ethyl | 4.77 | 363.0 |
| | 1-[1-(2-fluorophenyl)ethyl]- | phenyl | | | | |
| | 1H-pyrazole | | | | | |
| [VI-10] | 4-bromo-1-[(2,2-dichloro- | 4-fluoro- | H | (2,2-dichlorocyclopropyl)- | 4.43 | 364.9 |
| | cyclopropyl)methyl]-3-(4- | phenyl | | methyl | | |
| | fluorophenyl)-1H-pyrazole | | | | | |
| [VI-11] | 5-(4-bromo-1-isobutyl-1H- | 3-cyano-4- | H | isobutyl | 4.15 | 324.1 |
| | pyrazol-3-yl)-2- | fluorophenyl | | | | |
| | fluorobenzonitrile | | | | | |
| [VI-12] | 3-{[4-bromo-3-(4-fluoro- | 4-fluoro- | H | 3-cyanobenzyl | 3.93 | 358.0 |
| | phenyl)-1H-pyrazol-1- | phenyl | | | | |
| | yl]methyl}benzonitrile | | | | | |
| [VI-13] | 4-bromo-1-(2-fluorobenzyl)- | 4-fluoro- | H | 2-fluorobenzyl | 4.39 | 349.0 |
| | 3-(4-fluorophenyl)-1H- | phenyl | | | | |
| | pyrazole | | | | | |
| [VI-14] | 4-bromo-3-(4-fluorophenyl)- | 4-fluoro- | H | 1-propyl | 3.89 | 283.0 |
| | 1-propyl-1H-pyrazole | phenyl | | | | |
| [VI-15] | 4-bromo-3-(4-fluorophenyl)- | 4-fluoro- | H | 2-(methylsulphanyl)ethyl | 3.63 | 315.0 |
| | 1-[2-(methylsulphanyl)- | phenyl | | | | |
| | ethyl]-1H-pyrazole | | | | | |
| [VI-16] | methyl-2-[4-bromo-3-(4- | 4-fluoro- | H | 1-methoxy-3-methyl-1- | 4.27 | 357.1 |
| | fluorophenyl)-1H-pyrazol-1- | phenyl | | oxobutan-2-yl | | |
| | yl]-3-methylbutanoate | | | | | |
| [VI-17] | 4-bromo-1-(1,3-dioxolan-2- | 4-fluoro- | H | 1,3-dioxolan-2-ylmethyl | 3.01 | 329.0 |
| | ylmethyl)-3-(4-fluoro- | phenyl | | | | |
| | phenyl)-1H-pyrazole | | | | | |
| [VI-18] | 4-bromo-1-(cyclopropyl- | 4-fluoro- | H | cyclopropylmethyl | 3.90 | 297.0 |
| | methyl)-3-(4-fluorophenyl)- | phenyl | | | | |
| | 1H-pyrazole | | | | | |
| [VI-19] | 4-bromo-1-sec-butyl-3-(4- | 4-fluoro- | H | sec-butyl | 4.39 | 299.0 |
| | fluorophenyl)-1H-pyrazole | phenyl | | | | |
| [VI-20] | 4-bromo-1-(2-ethoxyethyl)- | 4-fluoro- | H | 2-ethoxyethyl | 3.51 | 313.0 |
| | 3-(4-fluorophenyl)-1H- | phenyl | | | | |
| | pyrazole | | | | | |
| [VI-21] | 3-[4-bromo-3-(4-fluoro- | 4-fluoro- | H | 1-cyanopropan-2-yl | 3.08 | 310.1 |
| | phenyl)-1H-pyrazol-1- | phenyl | | | | |
| | yl]butanonitrile | | | | | |
| [VI-22] | 4-bromo-1-(1-cyclopropyl- | 4-fluoro- | H | 1-cyclopropylethyl | 4.43 | 309.0 |
| | ethyl)-3-(4-fluorophenyl)- | phenyl | | | | |
| | 1H-pyrazole | | | | | |
| [VI-23] | 3-[4-bromo-3-(4-fluoro- | 4-fluoro- | H | 2-cyanoethyl | 2.69 | 296.0 |
| | phenyl)-1H-pyrazol-1- | phenyl | | | | |
| | yl]propanonitrile | | | | | |
| [VI-24] | 4-bromo-3-(4-fluorophenyl)- | 4-fluoro- | CF₃ | isopropyl | 5.51 | 353.1 |
| | 1-isopropyl-5-(trifluoro- | phenyl | | | | |
| | methyl)-1H-pyrazole | | | | | |
| [VI-25] | 4-bromo-3-(4-fluorophenyl)- | 4-fluoro- | H | isopropoxy | 4.11 | 301.1 |
| | 1-isopropoxy-1H-pyrazole | phenyl | | | | |
| [VI-26] | 4-bromo-1-cyclopropyl-3-(4- | 4-fluoro- | H | cyclopropyl | 3.59 | 281.0 |
| | fluorophenyl)-1H-pyrazole | phenyl | | | | |
| [VI-27] | tert-butyl-4-[4-bromo-3-(4- | 4-fluoro- | H | 1-(tert-butoxycarbonyl)- | 4.77 | 368 |
| | fluorophenyl)-1H-pyrazol-1- | phenyl | | piperidin-4-yl | | [M+- |
| | yl]piperidin-1-carboxylate | | | | | C4H9]+ |
| [VI-b-1] | 4-bromo-5-(difluoro- | 4-fluoro- | CHF₂ | methyl | 3.52 | 321.1 |
| | methoxy)-3-(4-fluoro- | phenyl | | | | |
| | phenyl)-1-methyl-1H- | | | | | |
| | pyrazole | | | | | |
| [VI-b-2] | 4-bromo-5-(difluoro- | 4-fluoro- | CHF₂ | isopropyl | 4.61 | 351.0 |
| | methoxy)-3-(4-fluoro- | phenyl | | | | |
| | phenyl)-1-isopropyl-1H- | | | | | |
| | pyrazole | | | | | |
| [VI-b-3] | 4-bromo-5- | 4-fluoro- | CHF₂ | isobutyl | 4.91 | 365.0 |
| | (difluoromethoxy)-3-(4- | phenyl | | | | |
| | fluorophenyl)-1-isobutyl-1H- | | | | | |
| | pyrazole | | | | | |
| [VI-c-1] | 2-[4-bromo-5-(difluoro- | 4-fluoro- | CHF₂ | methyl | 3.48 | 336.9 |
| | methoxy)-1-methyl-1H- | phenyl | | | | |
| | pyrazol-3-yl]-5-fluorophenol | | | | | |

Compounds in which R^{3/301} = H, CH₃ or C(CH₃)₃ are excepted.

Compounds of the formula [X]

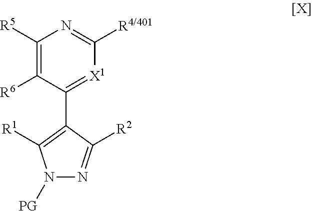

wherein R², R^{4/401}, R⁵, R⁶ and X¹ have the aforesaid general, preferred, particularly preferred, quite particularly preferred, most preferred or especially preferred meanings are novel.

For example, compounds:

wherein
R², R^{4/401}, R⁵, R⁶ stand for H,
X¹ for ═C—H and
PG for tetrahydro-2H-pyran-2-yl are novel

| | | | LogP | [M + H]⁺ |
|---|---|---|---|---|
| No. | Name | R¹ | (pH 2.3)¹ | Peak² |
| [X-1] | 4-[5-(4-fluorophenyl)-1- (tetrahydro-2H-pyran-2-yl)-1H- pyrazol-4-yl]pyridine | 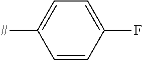 | 1.23 | 324.18 |
| [X-2] | 4-[5-(4-methoxy-1-naphthyl)-1- (tetrahydro-2H-pyran-2-yl)-1H- pyrazol-4-yl]pyridine | 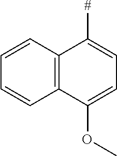 | 1.61 | 386.04 |
| [X-3] | 4-[5-(4-fluoro-1-naphthyl)-1- (tetrahydro-2H-pyran-2-yl)-1H- pyrazol-4-yl]pyridine | 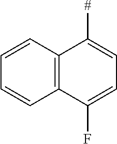 | 1.72 | 374.01 |
| [X-4] | 4-[5-(2,2-difluoro-1,3- benzodioxol-4-yl)-1-(tetrahydro- 2H-pyran-2-yl)-1H-pyrazol-4- yl]pyridine | 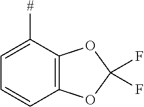 | 1.64 | 386.10 |
| [X-5] | 4-[5-(4-fluoro-2-methylphenyl)-1- (tetrahydro-2H-pyran-2-yl)-1H- pyrazol-4-yl]pyrid | 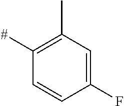 | 1.40 | 338.05 |
| [X-6] | 4-[5-(3-chloro-4-fluorophenyl)-1- (tetrahydro-2H-pyran-2-yl)-1H- pyrazol-4-yl]pyridine | 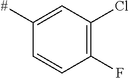 | 1.57 | 357.95 |
| [X-7] | 4-[5-(4-fluoro-3-methylphenyl)-1- (tetrahydro-2H-pyran-2-yl)-1H- pyrazol-4-yl]pyrid | 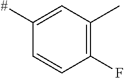 | 1.47 | 338.05 |
| [X-8] | 4-[5-(2,4-difluorophenyl)-1- (tetrahydro-2H-pyran-2-yl)-1H- pyrazol-4-yl]pyridine | 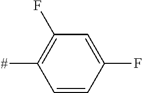 | 1.35 | 342.04 |
| [X-9] | 4-[5-(4-tert-butylphenyl)-1- (tetrahydro-2H-pyran-2-yl)-1H- pyrazol-4-yl]pyridine | 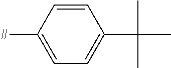 | 2.02 | 362.17 |
| [X-10] | 4-[5-(4-phenoxyphenyl)-1- (tetrahydro-2H-pyran-2-yl)-1H- pyrazol-4-yl]pyridine | 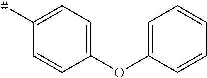 | 1.88 | 398.13 |
| [X-11] | 3-[4-(pyridin-4-yl)-1-(tetrahydro- 2H-pyran-2-yl)-1H-pyrazol-5- yl]benzonitrile | 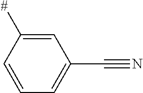 | 1.23 | 331.06 |
| [X-12] | 2-fluoro-5-[4-(pyridin-4-yl)-1- (tetrahydro-2H-pyran-2-yl)-1H- pyrazol-5-yl]benzonitrile | 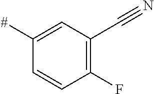 | 1.45 | 349.01 |
| [X-13] | N-{4-[4-pyridin-4-yl)-1- (tetrahydro-2H-pyran-2-yl)-1H- pyrazol-5-yl]phenyl}acetamide | 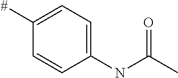 | 0.97 | 363.11 |
| [X-14] | 4-[5-(4-fluoro-2-methoxyphenyl)- 1-(tetrahydro-2H-pyran-2-yl)-1H- pyrazol-4-yl]pyridine | 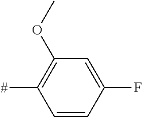 | 1.26 | 354.05 |
| [X-15] | 4-{4-[4-(pyridin-4-yl)-1- (tetrahydro-2H-pyran-2-yl)-1H- pyrazol-5-yl]phenyl}morpholine | 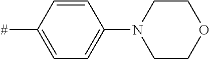 | 1.26 | 391.14 |
| [X-16] | 4-[5-(3-phenoxyphenyl)-1- (tetrahydro-2H-pyran-2-yl)-1H- pyrazol-4-yl]pyridine | 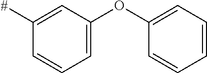 | 1.88 | 398.13 |
| [X-17] | 4-{5-[4-(methylsulphonyl)phenyl]- 1-(tetrahydro-2H-pyran-2-yl)-1H- pyrazol-4-yl}pyridine | 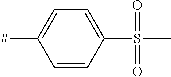 | 0.96 | 384.02 |
| [X-18] | 4-[5-(4-chlorophenyl)-1- (tetrahydro-2H-pyran-2-yl)-1H- pyrazol-4-yl]pyridine | 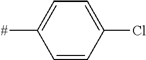 | 1.52 | 340.15 |
| [X-19] | 4-{1-(tetrahydro-2H-pyran-2-yl)-5- [4-(trifluoromethoxy)phenyl]-1H- pyrazol-4-yl}pyridine | 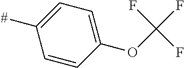 | 1.78 | 390.07 |
| [X-20] | 4-[5-(2,3-dichlorophenyl)-1- (tetrahydro-2H-pyran-2-yl)-1H- pyrazol-4-yl]pyridine | 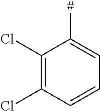 | 1.66 | 374.07 |
| [X-21] | N,N-dimethyl-3-[4-(pyridin-4-yl)- 1-(tetrahydro-2H-pyran-2-yl)-1H- pyrazol-5-yl]aniline | 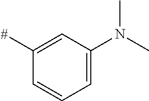 | 1.33 | 349.20 |
| [X-22] | 4-{5-[2-(benzyloxy)phenyl]-1- (tetrahydro-2H-pyran-2-yl)-1H- pyrazol-4-yl}pyridine | 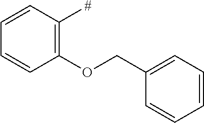 | 1.64 | 412.17 |
| [X-23] | 4-[4-(pyridin-4-yl)-1-(tetrahydro- 2H-pyran-2-yl)-1H-pyrazol-5- yl]benzenesulphonamide | 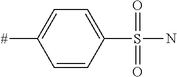 | 0.92 | 385.03 |
| [X-24] | 8-[4-(pyridin-4-yl)-1-(tetrahydro- 2H-pyran-2-yl)-1H-pyrazol-5- yl]quinoline | 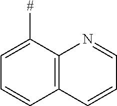 | 1.09 | 357.13 |

¹In the determination of the logP values, the methods described below were used.
²The mass stated is the peak of the isotope pattern of the [M + H]⁺ ion with the highest intensity; if the [M − H]⁻ ion was detected, the mass value is marked with a 2.

Compounds of the formula [XI]

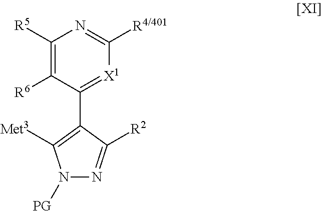

wherein the symbols R², R^{4/401}, R⁵, R⁶ and X¹ have the aforesaid general, preferred, particularly preferred, quite particularly preferred, most preferred, or especially preferred meanings, PG stands for a protective group, such as for example tetrahydro-2H-pyran-2-yl or 2-(trimethylsilyl)ethoxy]methyl, Met³ stands for a substituted metal atom, such as for example tributylstannyl or 4,4,5,5-tetramethyl-1,3,2-dioxaborolan-2-yl, and salts thereof, are novel

for example [XI-1]:

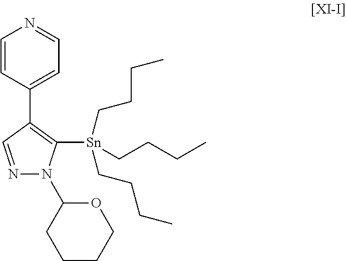

The compound 1-({4-[1-(2,2-difluoroethyl)-3-(trimethylstannyl)-1H-pyrazol-4-yl]pyrimidin-2-yl}-amino)propan-2-ol is excepted.

The production of the compounds with the general formula [I] by process A can be effected as follows:

A compound with the general formula [XIV] is brominated and then provided with a protective group, in order to obtain a compound with the formula [XIII] This compound can be reacted with a substrate of the formula [XV-a] in a C—C coupling reaction, whereby a compound with the formula [XII] is formed. This compound can be converted to a compound of the formula [XI] by reaction with a strong base and subsequent reaction with a boron or tin compound. This compound is converted to compounds of the formula [X] in a C—C coupling reaction with substrates of the general formula [XVII]. Next this compound is deprotected, whereby a compound of the general formula [IX] is obtained. The pyrazole of the formula [IX] obtained is now reacted with substrates of the type [XVI], whereby the arylpyrazoles according to the invention of the formula [I] are obtained (Scheme 1).

Alternatively, another process route can also be selected. A compound with the general formula [VIII] is brominated and a compound of the formula [VII] is obtained. This is converted to a compound of the type [VI] by reaction with substrates of the type [XVI], whereby mixtures of pyrazole regioisomers can be formed. These can be separated into the individual regioisomers by common processes e.g. chromatographic processes. The compounds of the general formula [VI] can be reacted with substrates of the formula [XV-a] in a C—C coupling, whereby compounds of the formula [I] are obtained (Scheme 1).

Alternatively the pyrazole compounds of the general formula [VI] can be converted into compounds of the type [V] by reaction with a boronic acid ester. These can be converted into compounds of the formula [I-c] by reaction with a substrate of the formula [IV-c] in a C—C coupling reaction (Scheme 1).

Alternatively, compounds of the type [V] can be converted into compounds of the formula [III] by reaction with a substrate of the formula [IV-a] in a C—C coupling reaction. These compounds are likewise converted into the compounds of the type [I-c] by reaction with substrates of the formula [II].

Furthermore, compounds of the type [V] can be converted into the arylpyrazoles according to the invention of the formula [I] by reaction with a substrate of the formula [IV-b] in a C—C coupling reaction (Scheme 1).

The production of the compounds with the general formula [I-d] wherein R^{4b/401b} stands for hydrogen, alkyl, cycloalkyl, aryl, —NH—C(O)-alkyl or —NH—C(O)O-alkyl, can be effected as follows by process B:

Compounds of the general formula [XXIV] are either commercially available or can be prepared by known literature methods.

Compounds of the general formula [XXIV] are reacted with a carboxylic acid ester, nitrile, dialkylamide or N,O-dialkylamide of the general formula R¹—COZ⁵, whereby compounds of the general formula [XXV] are obtained. These compounds [XXV] are converted into compounds of the general formula [XXVI] by reaction with DMF dialkyl acetal. From compounds of the general formula [XXVI], compounds of the formula [XXVII] are then obtained by reaction with hydrazine or hydrazine hydrate. The pyrazoles of the formula [XXVII] obtained are now reacted with substrates of the type [XVI], whereby the arylpyrazoles according to the invention of the formula [I-d] are obtained.

In the case where R^{3/301}=cyclopropyl, a compound of the formula [I-d] can also be obtained by C—C coupling reaction of a substrate of the formula [XXVII] with a cyclopropylboronic acid.

Compounds of the general formula [I-d] can also be obtained by direct reaction of a hydrazine derivative with substrates of the formula [XXVI].

The production of the compounds with the general formula [I-e] can be effected as follows by process C:

Compounds of the general formula [IX-a] are either commercially available or can be prepared by process A. The compounds of the formula [IX-a] are converted into compounds of the formula [XXVIII] by a halogenation reaction. The pyrazoles of the formula [XXVIII] obtained are now reacted with substrates of the type [XVI], whereby compounds of the formula [XXIX] are obtained. These compounds can be converted into intermediates of the formula [IX-b] by C—C coupling reaction with a boronic acid derivative of the formula [XXX] and subsequent deprotection reaction by removal of the R^{3/301} residue (e.g. in the case of the p-methoxybenzyl residue). These can be functionalized on the nitrogen atom of the pyrazole by the methods described in processes A and B, whereby the pyrazoles according to the invention of the formula [I-e] are obtained.

Alternatively the intermediates of the formula [XXIX] can also be converted directly into the pyrazoles according to the invention of the formula [I-e] by C—C coupling reaction with a boronic acid derivative of the formula [XXX].

The production of the intermediates with the general formula [VI-a] can be effected as follows by process D:

Pyrazole compounds of the general formula [VIII] can be converted into compounds of the type [XX] by reaction with a boronic acid ester. These compounds are converted into intermediates of the general formula [VI-a] by bromination.

The production of the intermediates with the general formula [VI-b] and [VI-c] can be effected as follows by process E:

According to known literature methods (WO1996/015115, U.S. Pat. No. 5,928,999) pyrazolinones [XXXII] are produced starting from the corresponding β-keto esters [XXXI] by reaction with hydrazines. These pyrazolones are converted into compounds of the type [XXXIII] by difluoromethylation according to known literature methods (Org. Lett. 2006, 8, 17, 3805-3808). The compounds of the formula [XXXIII] are next converted into compounds of the formula [VI-b] by a halogenation reaction. Compounds of the type [VI-b] wherein R¹ stands for 4-fluoro-2-methoxyphenyl can be converted into compounds of the type [VI-c] wherein R^{1a} stands for 4-fluoro-2-hydroxyphenyl by reaction with BBr₃.

The production of the intermediates with the general formula [III] can alternatively also be effected as follows by process F:

In a C—C coupling reaction intermediates of the type [VI] are reacted with substrates of the general formula [XV-aa], wherein Met^{z} stands for a boronic acid ester. In the course of the reaction, the free amine is formed by removal of the amino protecting group, whereby the intermediates of the general formula [III] are obtained.

The production of the intermediates with the general formula [XV-a] can be effected as follows by process G:

Compounds of the general formula [XXXIV] are converted into compounds of the formula [XXXV] by an acylation reaction. Next these compounds are converted into boronic acid esters of the formula [XV-a1] in a coupling reaction.

The production of the compounds with the general formula [I-f] and [I-g] can be effected as follows by process H:

Compounds of the general formula [XXXVI] are reacted according to known literature methods (J. Med. Chem. 1999, 42, 12, 2180-2190) with a carboxylic acid ester, nitrile, dialkylamide or —N,O-dialkylamide of the general formula R¹—COZ⁵, whereby compounds of the general formula [XXXVII] are obtained. Here the compounds of the general formula R¹—COZ⁵ must not contain any groups with acidic protons, such as for example NH or OH groups. These compounds [XXXVII] are converted into compounds of the general formula [XXXVIII] by reaction with DMF dialkyl acetal. From compounds of the general formula [XXXVIII], compounds of the formula [XXIX] are then obtained by reaction with hydrazine or hydrazine hydrate. The pyrazoles of the formula [XXIX] obtained are now reacted with substrates of the type [XVI], whereby compounds of the general formula [XL] are obtained. These are converted into compounds of the formula [XLI] by reaction with oxidizing agents, e.g. m-chloroperbenzoic acid. The arylpyrazoles according to the invention of the formula [I-f] are obtained from these by a substitution reaction in the presence of primary or secondary amines. If necessary, these compounds can be converted into compounds of the general formula [III-a] by removal of the amine substituents (e.g. in the case of benzylamines by a hydrogenation reaction). These compounds [III-a] are converted into the arylpyrazoles according to the invention of the formula [I-g] by reaction with substrates of the formula [II].

The production of the compounds with the general formula [I-h] be effected as follows by process I:

Compounds of the general formula [XLII] are reacted according to known literature methods (Tetrahedron Lett. 2009, 50, 21, 2552-2554) with a carboxylic acid ester of the general formula R¹—COZ⁵, whereby compounds of the general formula [XLIII] are obtained. Here the compounds of the general formula R¹—COZ⁵ must not contain any groups with acidic protons, such as for example NH or OH groups. Compounds of the formula [XLIV] are obtained from these by a substitution reaction in the presence of primary or secondary amines. These compounds [XLIV] are converted into compounds of the general formula [XLV] by reaction with DMF dialkyl acetal. Compounds of the formula [XLVI] are then obtained from compounds of the general formula [XLV] by reaction with hydrazine or hydrazine hydrate. The pyrazoles of the formula [XLVI] obtained are now reacted with substrates of the type [XVI], whereby the compounds according to the invention of the general formula [I-h] are obtained.

Step (V1)

One possibility for the synthesis of compounds of the formula [VI] is shown in Scheme 1.

Compounds of the formula [VI], wherein R^{3/301} does not stand for hydrogen, can be synthesized analogously to procedures described in the literature (Bioorg. Med. Chem. Lett. 2000, 10, 1351-1356 or J. Am. Chem. Soc. 2007, 129, 26, 8064-8065), by reaction of compounds of the type [VII] with a substrate of the general formula [XVI] (wherein Z¹ represents a leaving group, such as for example Cl, Br, I, —OTos, —OMs or the like), if necessary in the presence of a solvent and an acid scavenger/base.

Compounds of the formula [VI], wherein R^{3/301} does not stand for hydrogen, can moreover be synthesized analogously to procedures described in the literature (Mitsunobu, O. Synthesis 1981, 1-28), e.g. by reaction of compounds of the type [VII] with a substrate of the general formula [XVI] (wherein Z¹ stands for —OH) in the presence of a phosphane (e.g. triphenylphosphane) and an azodicarboxylate (e.g. diethyl azodicarboxylate) and a solvent (e.g. THF).

The bromine-substituted pyrazoles of the formula [VII] are either commercially available or can be produced by literature methods. One method for the production of suitable bromopyrazoles is for example the bromination of corresponding pyrazoles [VIII] (e.g. described in EP-A 1382 603) by reaction with N-bromosuccinimide in acetic acid.

Compounds of the type [VIII], such as for example 3-(4-fluorophenyl)-1H-pyrazole, 3-(4-chloro-phenyl)-1H-pyrazole or 3-(3-chlorophenyl)-1H-pyrazole are commercially available or can be produced e.g. by known literature methods (Tetrahedron, 2003, 59, 555-560) from commercial acetophenones by reaction with dimethylformamide dimethyl acetal and hydrazine.

The compounds of the formula [XVI] required for the reaction are commercially available or can be produced by literature methods (R. C. Larock, Comprehensive Organic Transformations, 2nd Edition, 1999, Wiley-VCH, p. 690 ff. and p. 1929 ff. and literature cited therein)

One method for the production of suitable compounds of the formula [XVI] (wherein R^{3/301} in the case of an alkylation reaction e.g. stands for a substituted or unsubstituted alkyl or cycloalkyl residue), is for example the reaction of alcohols with methanesulphonyl chloride and triethylamine (Org. Lett. 2008, 10, 4425-4428) or by Appel reaction with triphenylphosphine and CCl₄ (e.g. described in Tetrahedron 2008, 64, 7247-7251).

The production of suitable compounds of the formula [XVI] (wherein in R^{3/301} in the case of an acylation reaction a carbonyl group is directly bound to Z¹), is effected by known literature methods (e.g. Jerry March, Advanced Organic Chemistry, 4^{th} Edition, John Wiley & Sons, p. 437 ff. and the literature cited therein).

From the chemical structure of the substrates of the general formula [XVI], certain preferred combinations in the selection of a suitable solvent and a suitable base can be found.

In the case of an alkylation reaction with substrates of the formula [XVI] (wherein R^{3/301} in the case of an alkylation reaction e.g. stands for a substituted or unsubstituted alkyl or cycloalkyl residue) all usual solvents inert under the reaction conditions, such as for example cyclic and acyclic ethers (e.g. diethyl ether, tetrahydrofuran, dioxan), aromatic hydrocarbons (e.g. benzene, toluene, xylene), halogenated hydrocarbons (e.g. dichloromethane, chloroform, carbon tetrachloride), halogenated aromatic hydrocarbons (e.g. chlorobenzene, dichlorobenzene), nitriles (e.g. acetonitrile), carboxylic acid esters (e.g. ethyl acetate), amides (e.g. N,N-dimethylformamide, N,N-dimethylacetamide), dimethyl sulphoxide or 1,3-dimethyl-2-imidazolinone, can be used or the reaction can be effected in mixtures of two or more of these solvents. The preferred solvents are dimethylformamide and acetonitrile.

In the case of an alkylation reaction with substrates of the formula [XVI] (wherein R^{3/301} in the case of an alkylation reaction e.g. stands for a substituted or unsubstituted alkyl or cycloalkyl residue) bases which can be used for this reaction are for example lithium hexamethyldisilazide (LiHMDS), potassium carbonate, caesium carbonate and sodium hydride. The preferred base is sodium hydride. As a rule at least 1 equivalent of base is used.

In the case of an acylation reaction with substrates of the formula [XVI] (wherein in R^{3/301} a carbonyl group is directly bound to Z¹) all usual solvents inert under the reaction conditions, such as for example cyclic and acyclic ethers (e.g. diethyl ether, tetrahydrofuran, dioxan), aromatic hydrocarbons (e.g. benzene, toluene, xylene), halogenated hydrocarbons (e.g. dichloromethane, chloroform, carbon tetrachloride), halogenated aromatic hydrocarbons (e.g. chlorobenzene, dichlorobenzene), nitriles (e.g. acetonitrile) and aromatic heterocyclic amine (pyridine) can be used or the reaction can be effected in mixtures of two or more of these solvents. The preferred solvents are tetrahydrofuran and dichloromethane.

In the case of an acylation reaction with substrates of the formula [XVI] (wherein in R^{3/301} a carbonyl group is directly bound to Z¹) e.g. one equivalent of an acid scavenger/a base (e.g. pyridine, diisopropylethylamine, triethylamine or commercially available polymeric acid scavengers) relative to the starting material of the general formula [VII] can be used. If the starting material is a salt, at least two equivalents of the acid scavenger are needed. If pyridine is used as the solvent, analogously to the literature described, the addition of a further base can in some cases be omitted (EP-A-1 000 062).

The reaction is normally effected at temperatures of 0° C.-100° C. and preferably at 20° C.-30° C., but it can also be effected at the reflux temperature of the reaction mixture. The reaction time varies depending on the scale of the reaction and the reaction temperature, but generally lies between a few minutes and 48 hours.

After completion of the reaction, the compounds [VI] are separated from the reaction mixture by one of the usual separation techniques. Depending on the nature of the substrate of the formula [XVI] used and the reaction conditions, the compounds of the formula [VI], wherein R^{3/301} does not stand for hydrogen, can be obtained as pure regioisomers or as a mixture of both possible regioisomers (wherein the group R^{3/301} can occupy both positions on the N atom of the pyrazole). In the event that mixtures of regioisomers are obtained, these can be purified by physical methods (such as for example crystallization or chromatography methods) or can optionally also be used in the next step without prior purification.

Step (V2)

One possibility for the synthesis of compounds of the formula [V] is shown in Scheme 1.

Compounds of the formula [V] can be produced by described methods e.g. via reaction of the bromopyrazoles [VI] with boronic acid esters such as for example bispinacolatodiboron (4,4,4′,4′,5,5,5′,5′-octamethyl-2,2′-bi-1,3,2-dioxaborolane) in the presence of a catalyst such as for example 1,1′-bis(diphenyl-phosphino)ferrocene-palladium(II) dichloride in the presence of a base and a suitable solvent (see U.S. Pat. No. 0,018,156 A, WO 07/024843 or EP-A-1 382 603).

As the solvent, all common solvents inert under the reaction conditions, such as

for example sulphoxides (e.g. dimethyl sulphoxide), cyclic ethers (e.g. dioxan) and amides (e.g. N,N-dimethylformamide) can be used and the reaction can be effected in mixtures of two or more of these solvents. The preferred solvents are dimethyl sulphoxide and dioxan.

The reaction will normally be effected at temperatures of 80° C.-120° C., and the preferred reaction temperature is about 85° C.-90° C. The reaction time varies depending on the scale of the reaction and the reaction temperature, but generally lies between one hour and 16 hours.

Other synthetic methods described in the literature can likewise be used for the production of the compounds of the formula [V]. For example compounds of the formula [V] can be produced by metallation of the bromopyrazoles [VI] with bases such as for example n-butyllithium and reaction with boronic acid esters such as for example trimethyl borate and subsequent reaction of the pyrazole-boronic acid obtained with pinacol (see e.g. J. het. Chem. 2004, 41, 931-940 or EP-A-1 382 603 and WO2007/16392).

Step (V3)

One possibility for the synthesis of compounds of the formula [III] is shown in Scheme 1.

Compounds of the formula [III] can be produced for example by coupling of the pyrazoleboronic acids [V] with heterocycles of the formula [IV-a] (wherein Z² represents a leaving group such as for example Cl or Br) in the presence of a catalyst, a base and a suitable solvent at suitable temperatures by known literature procedures (Top. Curr. Chem. 2002, 219, 11; Organomet. Chem. 1999, 28, 147 and literature cited therein).

Compounds of the formula [IV-a] (wherein X¹ stands for C—H) are commercially available or can be produced by literature methods (Scheme 10). One method for the production of suitable N-Boc-haloheterocycles [IV-a-1] is the reaction of suitable acids (e.g. 4-bromo-picolinic acid) [L] with diphenylphosphoryl azide and tert-butanol (Aust. J. Chem. 1982, 35, 2025-2034, J. Med. Chem. 1992, 35, 15, 2761-2768 or U.S. Pat. No. 5,112,837 A).

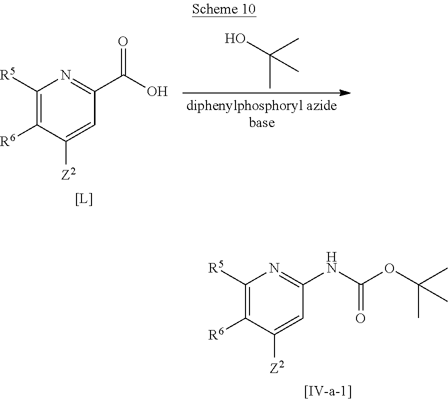

The carboxylic acids [L] are known or can be produced from commercially available precursors by procedures described in the literature (see e.g. EP-A-1 650 194), for example from the commercially available pyridine-2-carboxylic acid by reaction with thionyl chloride in dimethylformamide. Alternatively, compounds of the general formula [L] can also be produced by oxidation of commercially available 4-halo-2-methyl-pyridine derivatives by known literature procedures (Aust. J. Chem. 1982, 35, 2025-2034).

Compounds of the formula [IV-a] (wherein X¹ stands for N) are commercially available or can be produced by literature methods (Scheme 11). One method for the production of suitable N-Boc-haloheterocycles [IV-a-2] is the chlorination of the hydroxy compounds (e.g. (4-hydroxy-pyrimidin-2-yl)carbamate) with phosphorus oxychloride (Chem. Pharm. Bull. 2003, 51, 8, 975-977).

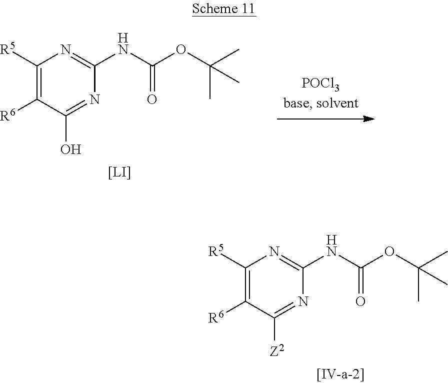

The hydroxy compounds [LI] are known or can be produced from commercially available precursors by procedures described in the literature (Chem. Pharm. Bull. 2003, 51, 8, 975-977).

As the solvent for the synthesis of compounds of the formula [III] all usual solvents inert under the reaction conditions, such as for example alcohols (e.g. methanol, ethanol, 1-propanol, 2-propanol, ethylene glycol, 1-butanol, 2-butanol, tert-butanol), cyclic and acyclic ethers (diethyl ether, dimethoxymethane, diethylene glycol dimethyl ether, tetrahydrofuran, dioxan, diisopropyl ether, tert-butyl methyl ether), aromatic hydrocarbons (e.g. benzene, toluene, xylene), hydrocarbons (e.g. hexane, iso-hexane, heptane, cyclohexane), ketones (e.g. acetone, ethyl methyl ketone, iso-butyl methyl ketone), nitriles (e.g. acetonitrile, propionitrile, butyronitrile) and amides (e.g. dimethyl-formamide, dimethylacetamide, N-methylpyrrolidone) and water can be used or the reaction can be effected in mixtures of two or more of these solvents. The preferred solvent is dioxan.

Bases which are preferably used in the process according to the invention are alkali and alkaline earth metal hydroxides, alkali and alkaline earth metal carbonates, alkali metal hydrogen carbonates, alkali and alkaline earth metal acetates, alkali and alkaline earth metal alcoholates, and primary, secondary and tertiary amines. Preferred bases are alkali metal carbonates such as for example caesium carbonate, sodium carbonate and potassium carbonate.

In the process according to the invention, the base is preferably used in a proportion of 100 to 1000 mol. %, based on the aromatic boronic acid. The preferred proportion is 600 to 800 mol. %.

As catalysts, for example palladium metal, palladium compounds and/or nickel compounds can be used. The catalysts can also be applied onto a solid carrier, such as activated charcoal or aluminium oxide. Palladium catalysts wherein the palladium is present in the oxidation state (0) or (II), such as tetrakis(triphenylphosphine)-palladium, bis(triphenylphosphine)palladium dichloride, bis(diphenyl-phosphino)ferrocenepalladium dichloride, palladium ketonates, palladium acetylacetonates (such as for example palladium bisacetylacetonate), nitrilepalladium halides (such as for example bis-(benzonitrile)palladium dichloride, bis(acetonitrile)-palladium dichloride), palladium halides (PdCl₂, Na₂PdCl₄, Na₂PdCl₆), allylpalladium halides, palladium biscarboxylates (such as for example palladium-II acetate) and tetrachloropalladic acid are preferred. Particularly preferred catalysts are tetrakis(triphenylphosphine)-palladium, bis(triphenylphosphine)-palladium dichloride and bis-(diphenylphosphino)ferrocenepalladium dichloride. The palladium compound can also be generated in situ, such as for example palladium(II) acetate from palladium(II) chloride and sodium acetate.

The quantity of catalyst, based on the heteroaromatics [IV-a] bearing the leaving group Z², is preferably 0.001 to 0.5 mol. % and particularly preferably 0.01 to 0.2 mol. %.

The catalyst can contain phosphorus-containing ligands or phosphorus-containing ligands can be added separately to the reaction mixture. Preferably suitable as phosphorus-containing ligands are tri-n-alkylphosphanes, triarylphosphanes, dialkylarylphosphanes, alkyldiarylphosphanes and/or heteroarylphosphanes, such as tripyridylphosphane and trifurylphosphane, wherein the three substituents on the phosphorus can be the same or different and wherein one or more substituents can link the phosphorus groups of several phosphanes, wherein one part of this linkage can also be a metal atom. Particularly preferable are phosphanes such as triphenylphosphane, tri-tert-butylphosphane and tricyclohexylphosphane.

The total concentration of phosphorus-containing ligands, based on the heteroaromatics [IV-a] bearing the leaving group Z² is preferably up to 1 mol. %, particularly preferably 0.01 to 0.5 mol. %.

To effect the process according to the invention, expediently the educts, the solvent, the base, the catalyst and if appropriate the ligand are thoroughly mixed and reacted preferably at a temperature of 0° C.-200° C., particularly preferably at 100-170° C. The reaction time varies depending on the scale of the reaction and the reaction temperature, but generally lies between a few minutes and 48 hours. Other than as a one-pot reaction, the reaction can also be run such that the various reactants are metered in a controlled way in the course of the reaction, different metering variants being possible.

The molar reactant ratio of the heteroaromatic [IV-a] to the organoboron compound [V] is preferably 0.9 to 1.5.

The processes according to the invention are generally performed under normal pressure. It is however also possible to operate under increased or reduced pressure. The reaction is generally performed with the use of a blanket gas such as for example argon or nitrogen. After completion of the reaction, the catalyst arising as a solid is removed by filtration, the crude product freed from the solvent or solvents and then purified by methods known to those skilled in the art and appropriate for the particular product, e.g. by recrystallization, distillation, sublimation, zone melting, melt crystallization or chromatography.

Step (V4)

One possibility for the synthesis of compounds of the formula [I-c] is shown in Scheme 1.

A compound with the general formula [I-c] can be synthesized, analogously to procedures described in the literature (see e.g. WO 04/052880 and e.g. T. W. Greene, P. G. M. Wuts, Protective Groups in Organic Synthesis, 1999, John Wiley & Sons, Inc.), by a coupling reaction of a compound with the corresponding general formula [III] with a substrate of the general formula [II] (with Z³ e.g. ═Cl, Br, F or —OH) if necessary in the presence of an acid scavenger/base wherein the definitions of the residues R¹, R², R^{3/301}, R^{4a/401a}, R⁵, R⁶ and X¹ in the above schemes correspond to the aforesaid definitions.

Acid halides [II] (Z³═Cl) or the corresponding carboxylic acids [II] (Z³═OH) are commercially available or preparable by processes described in the literature. In addition, a substrate with the general formula [II], with Z³═Cl, can be prepared from the corresponding acid (Z³═OH) by chlorination using known literature processes (R. C. Larock, Comprehensive Organic Transformations, 2nd Edition, 1999, Wiley-VCH, page 1929 ff. and literature cited therein).

As the solvent, all usual solvents inert under the reaction conditions, such as for example cyclic and acyclic ethers (e.g. diethyl ether, tetrahydrofuran, dioxan), aromatic hydro-carbons (e.g. benzene, toluene, xylene), halogenated hydrocarbons (e.g. dichloromethane, chloroform, carbon tetrachloride), halogenated aromatic hydrocarbons (e.g. chlorobenzene, dichlorobenzene) and nitriles (e.g. acetonitrile) can be used or the reaction can be effected in mixtures of two or more of these solvents. The preferred solvents are tetrahydrofuran and dichloromethane.

At least one equivalent of an acid scavenger/a base (e.g. Hünig base, triethylamine or commercially available polymeric acid scavengers) relative to the starting material of the general formula [III] is used. If the starting material is a salt, at least two equivalents of the acid scavenger are needed.

The reaction is normally effected at temperatures of 0° C.-100° C. and preferably at 20° C.-30° C., but it can also be effected at the reflux temperature of the reaction mixture. The reaction time varies depending on the scale of the reaction and the reaction temperature, but generally lies between a few minutes and 48 hours.

To effect the process (V4) according to the invention for the production of the compounds of the formula [I-c] in general 0.2 to 2 mol, preferably 0.5 to 0.9 mol, of amino derivative of the formula [III] is used per mol of the carboxylic acid halide of the formula [II]. The workup is effected by evaporation of the volatile components under vacuum and treatment of the crude material with ammoniacal methanol solution (7 molar).

After completion of the reaction, the compounds [I-c] are separated from the reaction mixture by one of the usual separation techniques. If necessary, the compounds are purified by recrystallization, distillation or chromatography.

Alternatively, a compound of the formula [I-c] can also by synthesized from the corresponding compound of the formula [III] with a substrate of the formula [II] with Z³═—OH in the presence of a coupling reagent analogously to procedures described in the literature (e.g. Tetrahedron 2005, 61, 10827-10852, and references cited therein).

Suitable coupling reagents are for example peptide coupling reagents (for example, N-(3-dimethyl-aminopropyl)-N′-ethyl-carbodiimide mixed with 4-dimethylamino-pyridine, N-(3-dimethylamino-propyl)-N′-ethyl-carbodiimide mixed with 1-hydroxy-benzotriazole, bromo-tripyrrolidino-phosphonium hexafluorophosphate, O-(7-azabenzotriazol-1-yl)-N,N,N′,N′-tetramethyluronium hexafluorophosphate, etc.).

If necessary, a base, such as for example triethylamine or Hünig base can be used in the reaction.

As the solvent, all usual solvents inert under the reaction conditions, such as for example alcohols (e.g. methanol, ethanol, propanol), cyclic and acyclic ethers (e.g. diethyl ether, tetrahydrofuran, dioxan), aromatic hydrocarbons (e.g. benzene, toluene, xylene), halogenated hydrocarbons (e.g. dichloromethane, chloroform, carbon tetrachloride), halogenated aromatic hydrocarbons (e.g. chlorobenzene, dichlorobenzene), nitriles (e.g. acetonitrile) and amides (e.g. N,N-dimethylformamide, N,N-dimethylacetamide) can be used or the reaction can be performed in mixtures of two or more of these solvents. The preferred solvent is dichloromethane.

The reaction is normally performed at temperatures of 0° C.-100° C. and preferably at 0° C.-30° C., but it can also be performed at the reflux temperature of the reaction mixture. The reaction time varies depending on the scale of the reaction and the reaction temperature, but generally lies between a few minutes and 48 hours.

After completion of the reaction, the compounds [I-c] are separated from the reaction mixture by one of the usual separation techniques. If necessary, the compounds are purified by recrystallization, distillation or chromatography.

Compounds of the general formula [I-c] in which R^{4a/401a} stands for —NR¹²R^{12′} (symmetrically or unsymmetrically bisacylated aminopyridines) can be produced directly by the aforesaid method from compounds of the general formula [I-c], in which R^{4a/401a} stands for —NHR¹² (monoacylated aminopyridines), by reaction with acid halides of the formula [II] (Z³═ e.g. Cl, F).

Step (V5)

A further possibility for the synthesis of compounds of the formula [I-c] is shown in Scheme 1.

Compounds of the formula [I-c] can be produced for example by coupling of the pyrazoleboronic acids [V] with heterocycles of the formula [IV-c] (wherein Z² is a leaving group, such as for example Cl or Br) in the presence of a catalyst, a base and a suitable solvent at suitable temperatures by known literature procedures (Top. Curr. Chem. 2002, 219, 11; Organomet. Chem. 1999, 28, 147 and literature cited therein).

Compounds of the formula [IV-c] (wherein X¹ stands for C—H) are commercially available or can be produced by literature methods (Scheme 12). One method for the production of suitable haloheterocycles [IV-c-1] is the reaction of aminoheterocycles of the formula [XX] with acid chlorides in the presence of a base and a solvent (Synth. Commun. 1997, 27, 5, 861-870). The selection of solvent, base and temperature can vary depending on the substrate [XX] used and comprises the possible variations described under step (V4) for reaction of the aminoheterocycles of the formula [III] with substrates of the formula [II] for production of compounds of the formula [I-c].

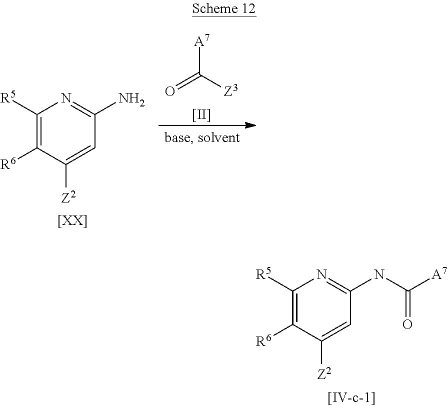

The aminoheterocycles [XX] (wherein X¹ stands for C—H) are known or can be produced by removal of the N-BOC protective group from compounds of the formula [IV-a] by procedures described in the literature (Aust. J. Chem. 1982, 35, 10, 2025-2034 and references contained therein).

The aminoheterocycles [XX] (wherein X¹ stands for N) are known or can be produced by halogenation of the hydroxy compounds (Z²═—OH) by procedures described in the literature (e.g. after J. Med. Chem. 2006, 49, 14, 4409-4424).

The selection of solvent, base, temperature, catalysts and added ligands if necessary can vary depending on the substrate [IV-c] used and comprises the possible variations described under step (V3) for the C—C coupling of compound of the formula [V].

After completion of the reaction, the catalyst arising as a solid is removed by filtration, the crude product freed from the solvent or solvents and then purified by methods known to those skilled in the art and appropriate for the particular product, e.g. by recrystallization, distillation, sublimation, zone melting, melt crystallization or chromatography.

Step (V6)

A further possibility for the synthesis of compounds of the formula [I] is shown in Scheme 1.

Compounds of the formula [I] can be produced for example by coupling of the halopyrazoles [VI] with metallated heterocycles of the formula [XV-a] (wherein Met¹ stands for a borate ester or boronic acid such as for example B(OiPr)₃, B(OH)₂) in the presence of a catalyst, a base, if necessary a ligand and a suitable solvent at suitable temperatures by known literature procedures (Top. Curr. Chem. 2002, 219, 11; Organomet. Chein. 1999, 28, 147 and literature cited therein, 2005, 7, 21, 4753-4756). (Scheme 13)

Compounds of the formula [I] can also be produced for example by coupling of the halopyrazoles [VI] with metallated heterocycles of the formula [XV-a] in the presence of a catalyst, if necessary an inorganic or organic halide salt, if necessary a ligand and a suitable solvent at suitable temperatures by known literature procedures (see Synthesis 1992, 803-815).

Compounds of the formula [XV-a1] (wherein X¹ stands for C-H) are commercially available or can be produced by literature procedures. One method for the production of suitable haloheterocycles [XV-a1.] is the reaction of haloheterocycles of the formula [XXI] with bispinacolatodiboron in the presence of a catalyst (such as for example Pd(OAc)₂ or PdCl₂(dppf)), if necessary a ligand (such as for example 1,3-bis(2,6-diisopropylphenyl)-4,5-dihydroimidazolium chloride), a base (such as for example potassium acetate or sodium acetate) and a solvent (such as for example tetrahydrofuran or dimethyl sulphoxide) by methods described in the literature (Bioorg. Med. Chem. Lett. 2006, 16, 5, 1277-1281 and WO 04/014913) (Scheme 13).

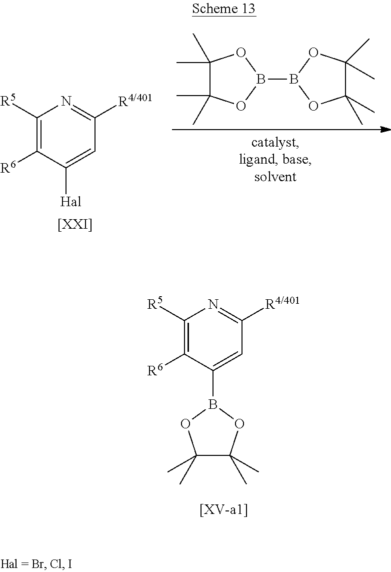

Alternatively, compounds of the formula [XV-a1] (wherein X¹ stands for C—H) can also be prepared by other known literature methods. One method for the production of suitable heterocycles [XV-a1] is the metallation of the halopyridine [XXI] with a base (such as for example n-butyllithium) in a solvent (such as for example diethyl ether or tetrahydrofuran) and subsequent reaction with a boronic acid ester (such as for example B(i-PrO)₃ or B(OMe)₃) and pinacol by known literature methods (Synthesis 2004, 4, 469-483 and literature described therein) (Scheme 14).

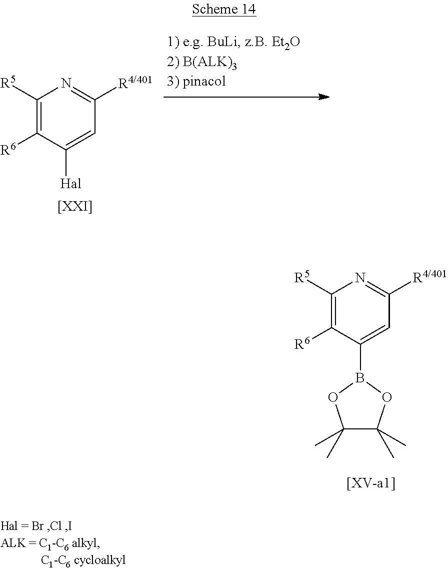

Compounds of the formula [XV-a2] (wherein X¹ stands for N) are commercially available or can be produced by literature procedures. One method for the production of suitable haloheterocycles [XV-a2] is the reaction of haloheterocycles of the formula [XXII] with hexaalkylditin compounds (such as for example 1,1,1,2,2,2-hexabutylditin) in the presence of a catalyst (such as for example bis(triphenylphosphine)palladium(II) acetate), if necessary a fluoride ion source (such as for example tetrabutylammonium fluoride) and a solvent (such as for example tetrahydrofuran or diethyl ether) by methods described in the literature (WO 03/095455 or WO 07/104538) (Scheme 15).

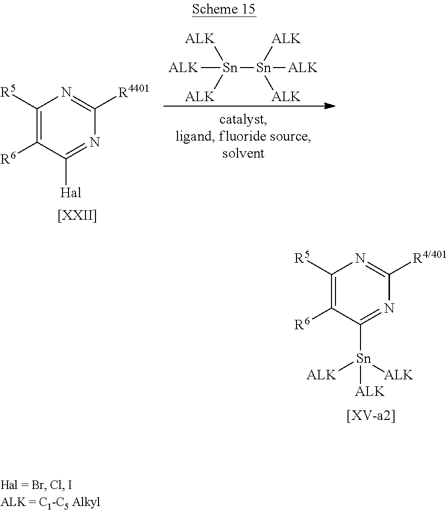

Alternatively, compounds of the formula [XV-a2] (wherein X¹ stands for N) can also be prepared by other known literature methods. One method for the production of suitable haloheterocycles [XV-a2] is the metallation of the halopyridine [XXII] using a metallation reagent (an alkyllithium compound such as for example n-butyllithium or a Grignard reagent such as for example isopropylmagnesium chloride) in a solvent (such as for example diethyl ether or tetrahydrofuran) and subsequent reaction with a trialkyltin halogen compound (such as for example Bu₃SnCl) by known literature methods (WO 08/008747 or Tetrahedron 1994, 275-284 and literature described therein) (Scheme 16).

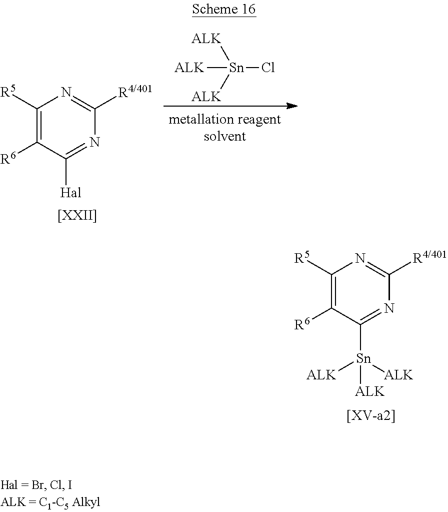

Compounds of the formula [XXI] and [XXII] are commercially available or can be prepared for example by acylation of corresponding amine (in the case R^{4/401}═—NH₂) by known literature methods (e.g. J. Org. Chem. 2004, 69, 543-548). Another method for the preparation of the compounds of the type [XXI] and [XXII] consists in the halogenation of the corresponding hydroxyheterocycles analogously to the halogenation methods stated for the synthesis of the compounds [XX] and [IV-b].

In the coupling of the halopyrazoles [VI] with metallated heterocycles of the formula [XV-a] (wherein Met stands for a borate ester or boronic acid such as for example B(OiPr)₃ or B(OH)₂), the selection of solvent, base, temperature, catalysts and added ligands if necessary can vary depending on the borate ester substrate used and comprises the possible variations described under step (V3) for the C-C coupling of compound of the formula [V] with substrates of the formula [IV-a].

In the coupling of the halopyrazoles [VI] with metallated heterocycles of the formula [XV-a] (wherein Met stands for an alkyltin bearing group such as for example Sn(Bu)₃), the selection of a catalyst, if necessary an inorganic or organic halide salt, if necessary a ligand and a suitable solvent at suitable temperatures can vary depending on the alkyltin substrate used.

As the solvent for the reaction of compounds of the formula [XV-a], all usual solvents inert under the reaction conditions, such as for example cyclic and acyclic ethers (diethyl ether, dimethoxymethane, diethylene glycol dimethyl ether, tetrahydrofuran, dioxan, diisopropyl ether, tert-butyl methyl ether), aromatic hydrocarbons (e.g. benzene, toluene, xylene), amides (e.g. dimethylformamide, dimethyl-acetamide, N-methylpyrrolidone) and sulphoxides (e.g. dimethyl sulphoxide) can be used or the reaction can be performed in mixtures of two or more of these solvents. The preferred solvent is dimethylformamide.

Halide salts for the reaction of compounds of the formula [XV-a] which are preferably used in the process according to the invention are for example copper halides (e.g. CuBr or Cul), caesium halides (CsF) and tetraalkylammonium halides (TBAF).

The halide salts are preferably used in the process according to the invention in a proportion of 1 to 400 mol. %, based on the organic tin compound. However, mixtures of the halide salts can also be used in proportions of 1-400 mol. %. The addition of a mixture of copper iodide and caesium fluoride in proportions of 1-200 mol. % is particularly preferable.

As catalysts for the reaction of compounds of the formula [XV-a] the same catalysts can be used as were described above for the production of the compounds of the formula [III], by reaction of the compounds of the formula [V] and [IV-a].

The quantity of catalyst, based on the heteroaromatics [XV-a] bearing the leaving group Met¹, is preferably 0.001 to 0.5 mol. % and particularly preferably 0.01 to 0.2 mol. %.

The catalyst can contain phosphorus-containing or arsenic-containing ligands or phosphorus-containing or arsenic-containing ligands can be added separately to the reaction mixture. As phosphorus-containing ligands, preferably tri-n-alkylphosphanes, triarylphosphanes, dialkylaryl-phosphanes, alkyldiarylphosphanes and/or heteroarylphosphanes, such as tripyridylphosphane and trifurylphosphane, wherein the three substituents on the phosphorus can be the same or different, can be chiral or achiral and wherein one or more substituents can link the phosphorus groups of several phosphanes, wherein one part of this linkage can also be a metal atom, are suitable. Particularly preferable are phosphanes such as triphenylphosphane, tri-tert-butylphosphane and tricyclohexyl-phosphane. As arsenic-containing ligands, for example tri-n-alkylarsanes and triarylarsanes, wherein the three substituents on the arsenic can be the same or different, are suitable.

The total concentration of ligands, based on the heteroaromatics [XV-a] bearing the leaving group Met¹, is preferably up to 1 mol. %, particularly preferably 0.01 to 0.5 mol. %.

To effect the process according to the invention, advantageously the educts, the solvent, the base, the halide salt, the catalyst and if necessary the ligand are thoroughly mixed and reacted preferably at a temperature of 0° C.-200° C., particularly preferably at 60-150° C. The reaction time varies depending on the scale of reaction and the reaction temperature, but generally lies between a few minutes and 48 hours. Other than as a one-pot reaction, the reaction can also be run such that the various reactants are metered in a controlled manner in the course of the reaction, whereby different metering variants are possible.

The processes according to the invention are in general performed under normal pressure. However it is also possible to operate under increased or reduced pressure. The reaction is in general performed using a blanket gas such as for example argon or nitrogen.

The molar reactant ratio of the halopyrazole [VI] to the organotin compound [XV-a2] is preferably 0.9 to 2.

After completion of the reaction, the catalyst arising as a solid is removed by filtration, the crude product freed from the solvent or solvents and then purified by methods known to those skilled in the art and appropriate for the particular product, e.g. by recrystallization, distillation, sublimation, zone melting, melt crystallization or chromatography.

Step (V7)

A further possibility for the synthesis of compounds of the formula [I] is shown in Scheme 1.

Compounds of the formula [I] can be produced for example by coupling of the pyrazoleboronic acids [V] with heterocycles of the formula [IV-b] (wherein Z² represents a leaving group such as for example Cl or Br) in the presence of a catalyst, a base and a suitable solvent at suitable temperatures by known literature procedures (Top. Curr. Chem. 2002, 219, 11; b-A. Suzuki, Organomet. Chem. 1999, 28, 147 and literature cited therein).

Compounds of the formula [IV-b1] (wherein X¹ stands for C—H) are commercially available or can be produced by literature procedures (Scheme 17). One method for the production of suitable haloheterocycles [IV-b1] is the reaction of the pyridine N-oxides with halogenating agents (e.g. PCl₃, POCl₃, SOCl₂ or methanesulphonyl chloride) (see Bioorg. Med. Chem. Lett. 2007, 17, 7, 1934-1937).

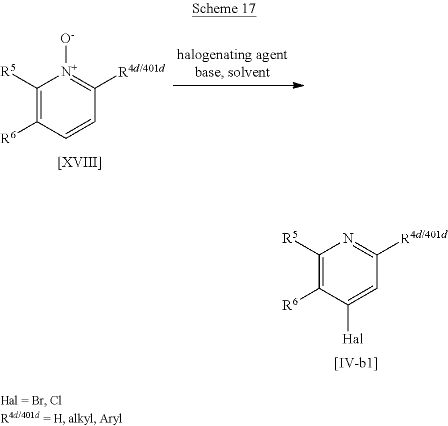

The pyridine N-oxides [XVIII] are known or can be produced by oxidation of the corresponding pyridines (e.g. with H₂O₂, H₂O₂+methyltrioxorhenium, m-chloroperoxybenzoic acid, dimethyl-dioxirane or H₂O₂+manganese tetrakis(2,6-dichlorophenyl)porphyrin) by procedures described in the literature (ARKIVOC 2001 (i) 242-268 and references contained therein).

A further method for the production of suitable haloheterocycles [IV-b1] is the reaction of the 4-hydroxypyridine compounds [XIX] with halogenating agents (e.g. PCl₃, POCl₃) by known literature procedures (Pol. J. Chem. 1981, 55, 4, 925-929) (Scheme 18).

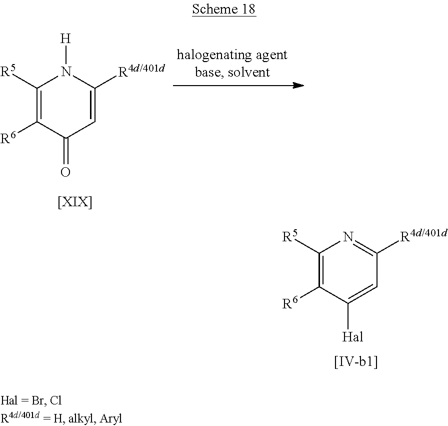

The hydroxypyridines [XIX] are known.

Compounds of the formula [IV-b2] (wherein X¹ stands for C-H) can be produced by literature procedures (Scheme 19). One method for the production of suitable haloheterocycles [IV-b2] is the reaction of aminoheterocycles of the formula [XX] with trifluoromethyl ketones in the presence of titanium-IV chloride, a base and a solvent (J. Am. Chem. Soc. 1996, 118, 7134-7138). The imine arising in the course of this reaction can be converted into the amine [IV-b2] by reduction by literature procedures (Tetrahedron 2009, 65, 9807-9813).

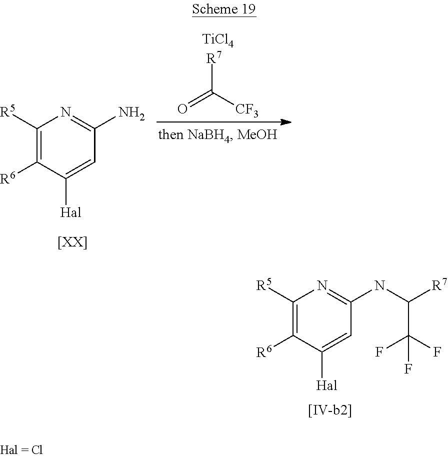

The aminoheterocycles [XX] (wherein X¹ stands for C—H) are known (US2006/189617).

The selection of solvent, base, temperature, catalysts and added ligands if necessary can vary depending on the substrate [IV-b] used and comprises the possible variations described under step (V3) for the C—C coupling of compound of the formula [V].

After completion of the reaction, the catalyst arising as a solid is removed by filtration, the crude product freed from the solvent or solvents and then purified by methods known to those skilled in the art and appropriate for the particular product, e.g. by recrystallization, distillation, sublimation, zone melting, melt crystallization or chromatography.

Step V8

One possibility for the synthesis of compounds of the formula [XIII] is shown in Scheme 1.

Compounds with the general formula [XIII] are known (R²═H) or can be synthesized analogously to procedures described in the literature (see e.g. Acta Chem. Scand., Series B: Organic Chemistry and Biochemistry 1982, 36, 2, 101-108 and EP-A-1 382 603). One possibility for the production of the compounds [XIII] is halogenation of the pyrazoles [XIV] with a halogenating agent in a suitable solvent to the pyrazole [XXIV] followed by conversion of the halopyrazole obtained into compounds of the formula [XIII] with a suitable protective group PG (e.g. 3,4-dihydro-2H-pyran) (Scheme 20).

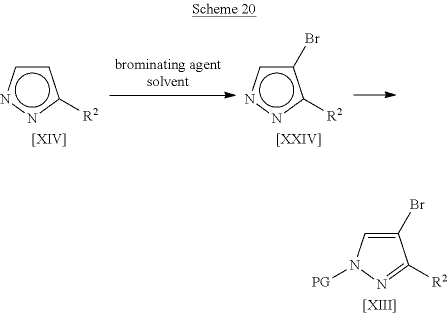

Pyrazoles of the formula [XIV] (R²═H, CH₃) are commercially available or preparable by processes described in the literature. Methods for the production of suitable pyrazoles [XIV] are for example the reaction of alkynes with TMS-diazomethane (Scheme 21) or the reaction of methyl ketones with dimethylformamide dimethyl acetal and hydrazine (Scheme 22) by described methods (U.S. Pat. No. 0,063,744 A).

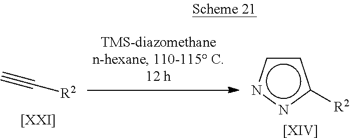

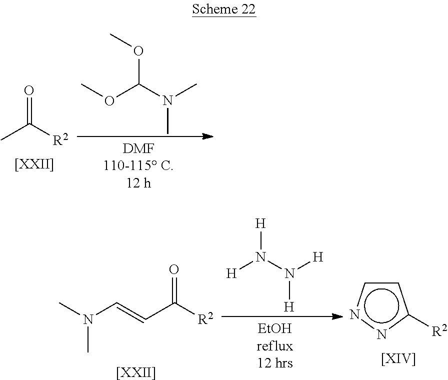

As the halogenating agent, for example N-bromosuccinimide and bromine can be used.

As the solvent for the halogenation reaction, all usual solvents inert under the reaction conditions, such as for example amides (e.g. dimethylformamide, dimethylacetamide, N-methylpyrrolidone), halogenated hydrocarbons (e.g. dichloromethane, chloroform, carbon tetrachloride), and acetic acid can be used or the reaction can be performed in mixtures of two or more of these solvents. The selection of the solvent can vary depending on the halogenation reagent used. The preferred solvents are acetic acid and dimethylfolinamide.

The halogenation reaction is normally performed at temperatures of 0° C.-100° C. and preferably at 20° C.-30° C. The reaction time varies depending on the scale of the reaction and the reaction temperature, but generally lies between a few minutes and 48 hours.

After completion of the halogenation reaction, the crude products are separated from the reaction mixture by one of the usual separation techniques. If necessary, the compounds are purified by

recrystallization, distillation or chromatography or can optionally also be used directly for further conversion without prior purification.

The bromopyrazoles [XXIV] obtained are protected on the nitrogen atom by heating in 3,4-dihydro-2H-pyran in the presence of a catalytic quantity of Lewis acid (e.g. p-toluenesulphonic acid). The products obtained can arise as regioisomers. If necessary, the compounds are purified by distillation or chromatography or can optionally also be used directly for further conversion without prior purification.

Step V9

One possibility for the synthesis of compounds of the formula [XII] is shown in Scheme 1.

Compounds of the formula [XII] can be produced for example by coupling of the halopyrazoles [XIII] with metallated heterocycles of the formula [XV-a] (wherein Met stands for a borate ester or boronic acid such as for example B(OiPr)₃ or B(OH)₂) in the presence of a catalyst, a base, if necessary a ligand and a suitable solvent at suitable temperatures by known literature procedures (Top. Curr. Chem. 2002, 219, 11; Organomet. Chem. 1999, 28, 147 and literature cited therein, Org. Lett. 2005, 7, 21, 4753-4756).

Compounds of the formula [XII] can be moreover produced for example by coupling of the halopyrazoles [XIII] with metallated heterocycles of the formula [XV-a], in the presence of a catalyst, if necessary an inorganic or organic halide salt, if necessary a ligand and a suitable solvent at suitable temperatures by known literature procedures (see Synthesis 1992, 803-815).

The production of the compounds of the type [XV-a] is described under step (V6) for the analogous reaction of the halopyrazoles [VI].

The selection of solvent, base or halide salt added if necessary, temperature, catalysts and ligands added if necessary can vary depending on the substrate [XV-a] used and comprises the possible variations described under step (V6) for the C—C coupling of compound of the formula [VI]. Here, in the reaction of compounds of the formula [XV-a], wherein Met¹ stands for an alkyltin-bearing group (such as for example Sn(Bu)₃), the addition of a base is usually omitted and instead of this a halide salt is added, as described under step (V6).

Step V10

One possibility for the synthesis of compounds of the formula [XI] is shown in Scheme 1.

One method for the production of the compounds of the formula [XI] is the metallation of the protected pyrazole [XII] with a base (such as for example n-butyllithium) in a solvent (such as for example diethyl ether or tetrahydrofuran) and subsequent reaction with a boronic acid ester (such as for example B(i-PrO)₃ or B(OMe)₃) and pinacol by known literature methods (see Tetrahedron Letters 2006, 47; 27; 2006; 4665-4669 and literature described therein) or with a trialkyltin halogen compound (such as for example Bu₃SnCl) analogously to known literature methods (WO 06/108591)

As the solvent, all usual solvents inert under the reaction conditions, such as for example cyclic and acyclic ethers (e.g. diethyl ether, tetrahydrofuran, dioxan) can be used or the reaction can be performed in mixtures of two or more of these solvents. The preferred solvent is tetrahydrofuran.

The reaction is noimally performed at temperatures of −80° C. to 0° C. and preferably at −78° C. to −20° C. In the course of the reaction, a change in the reaction temperature (e.g. after the metallation step) can be beneficial or necessary, in order to ensure the reaction with the second reaction partner (e.g. the alkyltin halide or the borate ester). The reaction time varies depending on the scale of the reaction and the reaction temperature, but generally lies between a few minutes and 48 hours.

The workup is usually effected by addition of a proton source (e.g. a saturated aqueous ammonium chloride solution) and subsequent phase separation. Next, the compounds [XI] are separated from the reaction mixture by one of the usual separation techniques.

Alternatively, however, the reaction mixture can also be concentrated without aqueous workup and the crude products [XI] distilled directly out of the reaction mixture.

If necessary, the compounds thus obtained are purified by recrystallization, distillation or chromatography.

Step V11

One possibility for the synthesis of compounds of the formula [X] is shown in Scheme 1.

Compounds of the formula [X] can be produced for example by coupling of the pyrazoles of the formula [XI] (wherein Met stands for a borate ester or boronic acid such as for example B(OiPr)₃ or B(OH)₂) with compounds of the formula [XVII] (wherein Z⁴ represents a leaving group such as for example Cl, Br, I, mesylate or triflate) in the presence of a catalyst, a base, if necessary a ligand and a suitable solvent at suitable temperatures by known literature procedures (Top. Curr. Chem. 2002, 219, 11; Organomet. Chem. 1999, 28, 147 and literature cited therein, Org. Lett. 2005, 7, 21, 4753-4756).

Compounds of the formula [X] can be moreover produced for example by coupling of the pyrazoles of the formula [XI] (wherein Mee stands for an alkyltin-bearing group such as for example —Sn(Bu)₃) with compounds of the formula [XVII] (wherein Z⁴ represents a leaving group such as for example Cl, Br, I, mesylate or triflate) in the presence of a catalyst, if necessary an inorganic or organic halide salt, if necessary a ligand and a suitable solvent at suitable temperatures by known literature procedures (see Synthesis 1992, 803-815).

Compounds of the formula [XVII] such as for example 4-bromo-1-fluorobenzene are known and commercially available.

In the coupling of the pyrazoles [XI] with compounds of the formula [XVII] the selection of solvent, base, temperature, catalysts and added ligand if necessary can vary depending on the pyrazole [XI] used and comprises the possible variations described under (V6).

In the coupling of the pyrazoles [XI] with compounds of the formula [XVII], the selection of a catalyst, if necessary an inorganic or organic halide salt, if necessary a ligand and a suitable solvent at suitable temperatures, can vary depending on the pyrazole [XI] used and comprises the possible variations described under step (V3).

The processes according to the invention are in general performed under normal pressure. However it is also possible to operate under increased or reduced pressure.

The reaction is in general performed using a blanket gas such as for example argon or nitrogen.

The molar reactant ratio of the pyrazole [XI] to the compound of the formula [XVII] is preferably 0.9 to 2.

After completion of the reaction, the catalyst arising as a solid is removed by filtration, the crude product freed from the solvent or solvents and then purified by methods known to those skilled in the art and appropriate for the particular product, e.g. by recrystallization, distillation, sublimation, zone melting, melt crystallization or chromatography.

Step (V12)

One possibility for the synthesis of compounds of the faimula [IX] is shown in Scheme 1.

A compound of the formula [X] is converted into a compound of the formula [IX] by suitable methods for the removal of protective groups, which are described in the literature (“Protective Groups in Organic Synthesis”; Third Edition; Theodora W. Greene, Peter G. M. Wuts; 1999, Wiley-VCH, p. 494-653, and literature cited there).

2-(Trimethylsilyl-ethoxy)methyl and tetrahydropyran-2-yl protective groups can for example be removed in an acidic medium (e.g. with methanolic HCl or trifluoroacetic acid) by known literature procedures (WO 03/099822 and J. Org. Chem. 2008, 73, 4309-4312 and literature contained therein). Benzylic protective groups can be removed hydrogenolytically with a hydrogen source (e.g. hydrogen, ammonium formate, formic acid or cyclohexene) in the presence of a catalyst (e.g. palladium on activated charcoal or palladium hydroxide on activated charcoal) by known literature procedures (EP-A-1 228 067).

As the solvent, all usual solvents inert under the reaction conditions, such as for example alcohols (e.g. methanol, ethanol, propanol), cyclic and acyclic ethers (e.g. diethyl ether, tetrahydrofuran, dioxan), aromatic hydrocarbons (e.g. benzene, toluene, xylene), halogenated hydrocarbons (e.g. dichloromethane, chloroform, carbon tetrachloride), halogenated aromatic hydrocarbons (e.g. chlorobenzene, dichlorobenzene), nitriles (e.g. acetonitrile), carboxylate ester (e.g. ethyl acetate), amides (e.g. N,N-dimethylformamide, N,N-dimethylacetamide), dimethyl sulphoxide, 1,3-dimethyl-2-imidazolinone, water and acetic acid can be used or the reaction can be performed in mixtures of two or more of these solvents.

The reaction is noi ially performed at temperatures of 0° C.-150° C. and preferably at room temperature, but it can also be performed at the reflux temperature of the reaction mixture. The reaction time varies depending on the scale of the reaction and the reaction temperature, but generally lies between half an hour and 72 hours.

After completion of the reaction, the compounds [IX] are separated from the reaction mixture by one of the usual separation techniques. If necessary, the compounds are purified by recrystallization, distillation or chromatography or if desired can also be used in the next step without prior purification. It is moreover possible to isolate the compound of the general formula [IX] as a salt, e.g. as a salt of hydrochloric acid or trifluoroacetic acid.

Step (V13)

A further possibility for the synthesis of compounds of the formula [I] is shown in Scheme 1.

Compounds of the formula [IX] can be converted into compounds of the formula [I] analogously to the methods described in step (V1) (Scheme 1), for which in the compound of the formula [IX] no functionality with reactive acidic H atoms should be contained in R^{4/401}.

The selection of solvent, base and temperature can vary depending on the substrate [IX] used and comprises the possible variations described under step (V1).

After completion of the reaction, the compounds [I] are separated from the reaction mixture by one of the usual separation techniques. Depending on the nature of the substrate of the formula [XVI] used and the reaction conditions, the compounds of the formula [I], wherein R³ does not stand for hydrogen, can be obtained as pure regioisomers or as a mixture of both possible regioisomers (wherein the group R^{3/301} can occupy both positions on the N atom of the pyrazole). In the event that mixtures of regioisomers are obtained, these can be purified by physical methods (such as for example crystallization or chromatography methods).

The synthesis of the pyrazoles [I-d] described in Scheme 2, and the synthesis of the pyrazoles [I-e] and [XXIX] described in Scheme 3 can be performed analogously, for which in the compounds of the formula [IX-b], [XXVII] and [XXVIII] no functionality with reactive acidic H atoms should be contained in R⁴.

Step (V14)

One possibility for the synthesis of compounds of the formula [XXV] is shown in Scheme 2.

By known literature methods (J. Med. Chem. 2007, 50, 2732-2736, WO 05/040155, WO 01/74811, U.S. Pat. No. 6,342,608 A), a carboxylic acid ester, nitrile, dialkylamide or —N,O-dialkylamide is reacted with an alkylpyridine or alkylpyrimidine of the formula [XXIV] in the presence of a strong base.

Bases which are preferably used in the process according to the invention are alkali metal alkoxides (such as for example KOtBu or NaOtBu), lithium amides (such as for example LDA or LiHMDS) or metal hydrides (such as for example KH or NaH).

As the solvent, all usual solvents inert under the reaction conditions, such as for example cyclic and acyclic ethers (e.g. diethyl ether, tetrahydrofuran, dioxan, dimethoxyethane), amides (e.g. N,N-dimethylformamide, N,N-dimethylacetamide), dimethyl sulphoxide or HMPT can be used or the reaction can be performed in mixtures of two or more of these solvents. The use of polar solvents such as N,N-dimethylformamide, dimethyl sulphoxide or HMPT is preferred.

The reaction is normally performed at temperatures of −78° C. up to the boiling point of the solvent, preferably in the range from −20° C. to 40° C. The reaction time varies depending on the scale of the reaction and the reaction temperature, but generally lies between half an hour and 72 hours.

After completion of the reaction, the compounds [XXV] are separated from the reaction mixture by one of the usual separation techniques. If necessary, the compounds are purified by recrystallization, distillation or chromatography or if desired can also be used in the next step without prior purification.

The alkylpyridines or alkylpyrimidines of the formula [XXIV] are commercially available or can be produced by known literature methods (e.g. WO 04/058776 or WO 04/035545).

The synthesis of the compounds of the formula [XXXVII] described in Scheme 8, and the synthesis of the compounds of the formula [XLIII] described in Scheme 9 can be performed analogously, for which in the compounds of the formula [XXXVI] and [XLII] no functionality with reactive acidic H atoms should be contained in R⁵ and R⁶.

Step (V15)

One possibility for the synthesis of compounds of the formula [XXVI] is shown in Scheme 2.

Compounds of the general formula [XXVI] are obtained by known literature methods (J. Med. Chem. 2007, 50, 2732-2736 and WO 05/040155, for R^{4b/401b}═NHC(O)Oalkyl e.g. EP-A-1 553 096) by reaction of a compound of the formula [XXV] with DMF dialkyl acetal. The reaction can be performed in the presence of a solvent, suitable solvents are alcohols (such as for example ethanol), esters (such as for example ethyl acetate), cyclic ethers (such as for example tetrahydrofuran) or amides (e.g. N,N-dimethylformamide or N-methylpyrrolidone). The reaction can be performed in the presence of a base (e.g. triethylamine).

The reaction is normally performed at temperatures of −78° C. up to the boiling point of the solvent.

The synthesis of the compounds of the formula [XXXVIII] described in Scheme 8, and the synthesis of the compounds of the formula [XLV] described in Scheme 9 can be performed analogously.

Step (V16)

One possibility for the synthesis of compounds of the fon [XXVII] is shown in Scheme 2.

Compounds of the general formula [XXVII] are obtained by reaction of compounds of the general formula [XXVI] with hydrazine or hydrazine hydrate by known literature methods (e.g. EP-A-1 553 096, EP-A-1 188 754). Here the group Z⁶ named in Scheme 2 stands for a leaving group such as for example NMe₂.

The reaction can be performed in the presence of a base such as for example triethylamine.

As the solvent, all usual solvents inert under the reaction conditions, such as for example cyclic and acyclic ethers (e.g. tetrahydrofuran, dioxan, dimethoxyethane) or alcohols (e.g. ethanol, methanol) can be used or the reaction can be performed in mixtures of two or more of these solvents. The use of polar solvents such as for example ethanol is preferred.

The reaction is normally performed at temperatures of 0° C. up to the boiling point of the solvent, preferably in the region of 25° C. The reaction time varies depending on the scale of the reaction and the reaction temperature, but generally lies between half an hour and 72 hours. The reaction can be performed in a microwave apparatus (e.g. CEM Explorer) at elevated temperature, whereby the reaction time required can be shortened.

The synthesis of the compounds of the formula [XXXIX] described in Scheme 8, and the synthesis of the compounds of the folinula [XLVI] described in Scheme 9 can be performed analogously.

Step (V17)

One possibility for the synthesis of compounds of the formula [I-d] is shown in Scheme 2.

Compounds of the general formula [I-d] are obtained by reaction of compounds of the general formula [XXVI] with alkylhydrazines of the formula R^{3/301}—NH—NH₂ by known literature methods (e.g. U.S. Pat. No. 6,335,336 A). Here the group Z⁶ named in Scheme 2 stands for a leaving group such as for example NMe₂.

The reaction can be performed in the presence of a base such as for example triethylamine.

As the solvent, all usual solvents inert under the reaction conditions, such as for example cyclic and acyclic ethers (e.g. tetrahydrofuran, dioxan, dimethoxyethane) or alcohols (e.g. ethanol, methanol) can be used or the reaction can be performed in mixtures of two or more of these solvents. The use of polar solvents such as for example ethanol is preferred.

The reaction is normally performed at temperatures of 0° C. up to the boiling point of the solvent, preferably in the region of 25° C. The reaction time varies depending on the scale of the reaction and the reaction temperature, but generally lies between half an hour and 72 hours. The reaction can be performed in a microwave apparatus (e.g. CEM Explorer) at elevated temperature, whereby the reaction time required can be shortened.

Step (V18)

One possibility for the synthesis of compounds of the formula [I-d] in which R^{3/301} stands for cyclopropyl, is the reaction of pyrazoles of the formula [XXVII] with a cyclopropylboronic acid by known literature procedures (J. Org. Chem. 2008, 73, 6441-6444 or WO 08/088692).

The reaction is performed in the presence of a base (such as for example triethylamine, pyridine, sodium carbonate, potassium phosphate or caesium carbonate) and a Cu(II) salt (such as for example Cu(OAc)₂ or CuCl₂).

In addition, the reaction can take place with addition of a suitable ligand (such as for example pyridine or 2,2-bipyridine, N,N,N′,N′-tetramethylethylenediamine or 1,10-phenanthridine).

As the solvent, all usual solvents inert under the reaction conditions, such as for example cyclic and acyclic ethers (e.g. tetrahydrofuran, dioxan, dimethoxyethane), halogenalkane (e.g. dichloroethane) or aromatic hydrocarbons (e.g. benzene, toluene) can be used or the reaction can be performed in mixtures of two or more of these solvents. The use of haloalkanes such as for example dichloroethane is preferred.

The reaction is normally performed at temperatures of 50° C. up to the boiling point of the solvent, preferably in the region of 70° C. The reaction time varies depending on the scale of the reaction and the reaction temperature, but generally lies between half an hour and 72 hours. The reaction can be performed in a microwave apparatus (e.g. CEM Explorer) at elevated temperature, whereby the reaction time required can be shortened.

Analogously to the synthesis of the pyrazoles [I-d] described in Scheme 2, the synthesis of the pyrazoles [I-e] described in Scheme 3 and the synthesis of the pyrazoles [XX] described in Scheme 4 can be effected with this process.

Step (V19)

One possibility for the synthesis of compounds of the formula [XXVIII] is shown in Scheme 3.

Compounds of the general formula [XXVIII] are obtained by halogenation of pyrazoles of the formula [XXVII] by known literature procedures (e.g. Bioorg. Med. Chem. Lett. 2008, 18, 509-512).

As halogenating agents, for example N-bromosuccinimide and bromine can be used.

As solvents for the halogenation reaction, all usual solvents inert under the reaction conditions, such as for example amides (e.g. dimethylformamide, dimethylacetamide, N-methylpyrrolidone), halogenated hydrocarbons (e.g. dichloromethane, chloroform, carbon tetrachloride), and acetic acid can be used or the reaction can be performed in mixtures of two or more of these solvents. The selection of the solvent can vary depending on the halogenation reagent used. The preferred solvents are acetic acid and dimethylformamide.

The halogenation reaction is normally performed at temperatures of 0° C. to 100° C. and preferably at 20° C. to 80° C. The reaction time varies depending on the scale of the reaction and the reaction temperature, but generally lies between a few minutes and 48 hours.

After completion of the halogenation reaction, the crude products are separated from the reaction mixture by one of the usual separation techniques. If necessary, the compounds are purified by

recrystallization, distillation or chromatography or can optionally also be used for further reaction without prior purification.

Step (V20)

One possibility for the synthesis of compounds of the formula [IX-b] is shown in Scheme 3.

Compounds of the formula [IX-b] in which R^{2a} stands for alkyl or cycloalkyl, can be produced by C—C coupling of pyrazoles of the formula [XXIX] with boronic acids or boric acid esters (e.g. trimethylboroxine or cyclopropylboronic acid esters) by known literature procedures (U.S. Pat. No. 0,018,132).

The reaction is perfoimed in the presence of a base (such as for example sodium hydroxide, potassium hydroxide, sodium hydrogen carbonate, sodium carbonate or caesium carbonate) and a palladium catalyst (such as for example dichloro[1.1′-ferrocenylbis(diphenylphosphane)]-palladium(II)*CH₂Cl₂).

As the solvent, all usual solvents inert under the reaction conditions, such as for example cyclic and acyclic ethers (e.g. tetrahydrofuran, dioxan, dimethoxyethane) can be used or the reaction can be performed in mixtures of two or more of these solvents.

The reaction is normally performed at temperatures of 50° C. up to the boiling point of the solvent, preferably in the region of 90° C. The reaction time varies depending on the scale of the reaction and the reaction temperature, but generally lies between half an hour and 72 hours. The reaction can be performed in a microwave apparatus (e.g. CEM Explorer) at elevated temperature, whereby the reaction time required can be shortened.

In the C—C coupling of the pyrazoles of the formula [XXIX] with compounds [XXX] (wherein Met² stands for a borate ester or boronic acid such as for example B(OiPr)₃ or B(OH)₂) the selection of a catalyst, a base, a ligands and a suitable solvent at suitable temperatures can vary depending on pyrazole [XXIX] used and likewise comprises the possible variations described under step (V3).

For the workup the reaction mixture is treated with water and extracted with ethyl acetate. The organic phase is separated and the solvent is removed under vacuum.

The crude product obtained is reacted with trifluoroacetic acid by known literature methods (e.g. “Protective Groups in Organic Synthesis”; Third Edition; Theodora W. Greene, Peter G. M. Wuts; 1999, Wiley-VCH, p. 639-640, and literature cited there) in order to remove the group R³ located on the pyrazole (e.g. in the case R³=p-methoxybenzyl) whereby the compounds of the formula [IX-b] are obtained.

After completion of the reaction, the compounds [IX-b] are separated from the reaction mixture by one of the usual separation techniques. If necessary, the compounds are purified by recrystallization, distillation or chromatography.

Step (V21)

One possibility for the synthesis of compounds of the formula [I-e] is shown in Scheme 3.

Compounds of the formula [I-e] in which R^{2a} stands for alkyl or cycloalkyl can be produced by C—C coupling of pyrazoles of the formula [XXIX] with boronic acids or boronic acid esters (e.g. trimethylboroxine or cyclopropylboronic acid ester) by known literature procedures (U.S. Pat. No. 0,018,132 A).

The conditions for the coupling correspond to the conditions stated under the above process (V20) without the removal of the group R^{3/301} by a deprotection reaction.

After completion of the reaction, the compounds [I-e] are separated from the reaction mixture by one of the usual separation techniques. If necessary, the compounds are purified by recrystallization, distillation or chromatography.

Step (V22)

One possibility for the synthesis of compounds of the formula [XXXII] is shown in Scheme 4.

Compounds of the general formula [XXXII] are obtained by reaction of compounds of the general formula [XXXI] with alkylhydrazines of the formula R^{3/301}—NH—NH₂ by known literature methods (e.g. U.S. Pat. No. 5,744,426).

The reaction can be performed in the presence of an acid such as for example acetic acid.

As the solvent, all usual solvents inert under the reaction conditions, such as for example cyclic and acyclic ethers (e.g. tetrahydrofuran, dioxan, dimethoxyethane), alcohols (e.g. ethanol, methanol) or esters (acetate esters) can be used or the reaction can be performed in mixtures of two or more of these solvents. The use of polar solvents such as for example ethanol is preferred.

The reaction is normally performed at temperatures of 0° C. up to the boiling point of the solvent, preferably under reflux. The reaction time varies depending on the scale of the reaction and the reaction temperature, but generally lies between half an hour and 72 hours. The reaction can be performed in a microwave apparatus (e.g. CEM Explorer) at elevated temperature, whereby the reaction time required can be shortened.

Step (V23)

One possibility for the synthesis of compounds of the formula [XXXIII] is shown in Scheme 4.

Compounds of the general formula [XXXIII] are obtained by reaction of compounds of the general formula [XXXII] with halodifluoromethane compounds (such as for example chlorodifluoro-methane or sodium chlorodifluoracetate) by known literature methods (e.g. U.S. Pat. No. 5,861,359, Org. Lett. 2006, 8, 17, 3805-3808).

The reaction is performed in the presence of a base such as for example potassium carbonate.

As the solvent, all usual solvents inert under the reaction conditions, such as for example amides (e.g. dimethylformamide, dimethylacetamide, N-methylpyrrolidone), cyclic and acyclic ethers (e.g. tetrahydrofuran, dioxan, dimethoxyethane) or nitriles (e.g. acetonitrile) can be used.

The reaction is normally performed at temperatures of 25° C. up to the boiling point of the solvent. The reaction time varies depending on the scale of the reaction and the reaction temperature, but generally lies between half an hour and 72 hours.

Step (V24)

One possibility for the synthesis of compounds of the formula [VI-b] is shown in Scheme 4.

Compounds of the general formula [VI-b] are obtained by halogenation of pyrazoles of the formula [XXXIII] by known literature procedures (e.g. U.S. Pat. No. 6,482,774).

As the halogenating agent, for example N-bromosuccinimide or bromine can be used.

As the solvent for the halogenation reaction, all usual solvents inert under the reaction conditions, such as for example amides (e.g. dimethylformamide, dimethylacetamide, N-methylpyrrolidone), halogenated hydrocarbons (e.g. dichloromethane, chloroform, carbon tetrachloride) or acetic acid can be used or the reaction can be performed in mixtures of two or more of these solvents. The selection of the solvent can vary depending on the halogenation reagent used. The preferred solvents are dichloromethane and tetrachloromethane.

The halogenation reaction is normally performed at temperatures of 0° C.-100° C. and preferably at 20° C.-80° C. The reaction time varies depending on the scale of the reaction and the reaction temperature, but generally lies between a few minutes and 48 hours.

After completion of the halogenation reaction, the crude products are separated from the reaction mixture by one of the usual separation techniques. If necessary, the compounds are purified by recrystallization, distillation or chromatography or can optionally also be used for further reaction without prior purification.

Step (V25)

One possibility for the synthesis of compounds of the formula [VI-c] is shown in Scheme 4.

Compounds of the general formula [VI-c] are obtained by ether cleavage of pyrazoles of the foimula [VI-b], wherein R² stands for 4-fluoro-2-methoxyphenyl, by known literature procedures (e.g. WO2007/105058).

The reaction is performed in the presence of a Lewis acid e.g. boron tribromide and a solvent inert under the reaction conditions (e.g. dichloromethane). The reaction is usually performed at temperatures of −20° C. +20° C., preferably at −5° C. to 0° C.

Step (V26)

One possibility for the synthesis of compounds of the formula [III] is shown in Scheme 6.

Compounds of the formula [III] can be produced for example by coupling of the halopyrazoles [VI] with metallated heterocycles of the formula [XV-a] (wherein Met stands for a borate ester or boronic acid such as for example B(OiPr)₃ or B(OH)₂) in the presence of a catalyst, a base, if necessary a ligand and a suitable solvent at suitable temperatures by known literature procedures (Top. Curr. Chem. 2002, 219, 11; Organomet. Chem. 1999, 28, 147 and literature cited therein, Org. Lett. 2005, 7, 21, 4753-4756).

The production of the compounds of the type [VI] is described under step (V6).

The selection of solvent, added base, temperature, catalysts and ligands added if necessary can vary depending on the substrate [VI] used and comprises the possible variations described under step (V6) for the C—C coupling of compound of the formula [VI]

Step (V27)

One possibility for the synthesis of compounds of the formula [XLI] is shown in Scheme 8.

Compounds of the general formula [XLI] are obtained by oxidation of thioalkylpyrimidines of the formula [XL] by known literature procedures (e.g. WO2009/16460 or WO2007/24843).

As oxidizing agents, for example e.g. m-chloroperbenzoic acid (m-CPBA) or Oxone (potassium peroxomonosulphate) can be used.

As the solvent for the oxidation reaction, all usual solvents inert under the reaction conditions, such as for example halogenated hydrocarbons (e.g. dichloromethane), ethers (e.g. tetrahydrofuran), alcohols (e.g. methanol) or water can be used or the reaction can be performed in mixtures of two or more of these solvents. The selection of the solvent can vary depending on the oxidizing reagent used. The preferred solvents are dichloromethane (m-CPBA) and water/THF mixtures (Oxone).

The oxidation reaction is normally performed at temperatures of 0° C. to 20° C. The reaction time varies depending on the scale of the reaction and the reaction temperature, but generally lies between a few hours and 48 hours.

After completion of the oxidation reaction, the crude products are separated from the reaction mixture by one of the usual separation techniques. If necessary, the compounds are purified by recrystallization, distillation or chromatography or can optionally also be used for further reaction without prior purification.

Step (V28)

One possibility for the synthesis of compounds of the formula [I-f] is shown in Scheme 8.

Compounds of the general formula [I-f] are obtained by reaction of compounds of the general formula [XLI] with primary or secondary amines by known literature methods (e.g. WO2007/105058 or U.S. Pat. No. 6,423,713).

The reaction is if necessary performed in the presence of a salt such as for example caesium fluoride.

As the solvent, all usual solvents inert under the reaction conditions can be used, such as for example amides (e.g. dimethylformamide, dimethylacetamide, N-methylpyrrolidone), cyclic and acyclic ethers (e.g. tetrahydrofuran, dioxan, dimethoxyethane), nitriles (e.g. acetonitrile), sulphoxides (e.g. dimethyl sulphoxide) or alcohols (e.g. ethanol, n-butanol). Alternatively, the reaction can be performed with no solvent, e.g. with the use of an excess of amine.

The reaction is normally performed at temperatures of 50° C. up to the boiling point of the solvent. The reaction time varies depending on the scale of the reaction and the reaction temperature, but generally lies between half an hour and 72 hours. The reaction can be performed in a microwave apparatus (e.g. CEM Explorer) at elevated temperature, whereby the reaction time required can be shortened.

Analogously to the synthesis of the pyrazoles [I-f] described in Scheme 8, the synthesis of the pyrazoles [XLIV] from the compounds of the type [XLIII] described in Scheme 9 can be effected with this process.

Step (V29)

One possibility for the synthesis of compounds of the formula [III-a] is shown in Scheme 8.

Compounds of the general formula [III-a] are obtained by dealkylation of compounds of the general formula [I-f] wherein

R^{x1} stands for H and

R^{x2} for benzyl, 4-methoxybenzyl or 3,4-dimethoxybenzyl,

by known literature methods (e.g. J. Med. Chem. 1999, 42, 12, 2180-2190 or Bioorg. Med. Chem. Lett. 2008, 18, 14, 4006-4010).

The reaction is usually performed in the presence of a strong acid e.g. sulphuric acid, hydrochloric acid or trifluoroacetic acid.

The reaction is normally performed at temperatures of 0° C. up to 120° C. The reaction time varies depending on the scale of the reaction and the reaction temperature, but generally lies between half an hour and 72 hours. The reaction can be performed in a microwave apparatus (e.g. CEM Explorer) at elevated temperature, whereby the reaction time required can be shortened.

A further subject of the invention relates to the nonmedicinal use of the phenylpyri(mi)dinylazoles according to the invention or mixtures thereof for the control of undesired microorganisms and for the reduction of mycotoxins in plants and plant parts.

A further subject of the invention relates to an agent for the control of undesired microorganisms and for the reduction of mycotoxins in plants and plant parts, comprising at least one phenyl-pyri(mi)dinylazole according to the present invention.

In addition, the invention relates to a method for the control of undesired microorganisms and for the reduction of mycotoxins in plants and plant parts, characterized in that the phenylpyri(mi)dinylazoles according to the invention are applied onto the microorganisms and/or in their habitat.

The substances according to the invention exhibit a strong microbicidal action and can be used for the control of undesired microorganisms, such as fungi and bacteria, in plant protection and in material protection.

The phenylpyri(mi)dinylazoles according to the invention of the formula (Ia) and (Ib) possess very good fungicidal properties and can be used in plant protection for example for the control of Plasmodiophoro-mycetes, Oomycetes, Chytridiomycetes, Zygomycetes, Ascomycetes, Basidiomycetes and Deuteromycetes.

Bactericides can be used in plant protection for example for the control of Pseudomonadaceae, Rhizobiaceae, Enterobacteriaceae, Corynebacteriaceae and Streptomycetaceae.

The fungicidal agents according to the invention can be used curatively or protectively for the control of phytopathogenic fungi. The invention therefore also relates to curative and protective methods for the control of phytopathogenic fungi through the use of the active substances or agents according to the invention, which is applied onto the seeds, the plant or plant parts, the fruit or the soil in which the plants grow.

The agents according to the invention for the control of phytopathogenic fungi in plant protection comprise an effective, but non-phytotoxic quantity of the active substances according to the invention. “Effective, but non-phytotoxic quantity” means a quantity of the agent according to the invention which is sufficient adequately to control or entirely kill the fungal disease of the plant and which at the same time does not bring with it any significant symptoms of phytotoxicity. This application dosage can in general vary over a considerable range. It depends on several factors, e.g. on the fungus to be controlled, the plant, the climatic conditions and the ingredients of the agents according to the invention.

According to the invention, all plants and plant parts can be treated. Here plants are understood to mean all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties protectable or not protectable by plant breeders' rights. Plant parts should be understood to mean all aboveground and underground parts and organs of the plants, such as shoot, leaf, flowers and root, wherein for example leaves, needles, stalks, stems, flowers, fruit bodies, fruit and seeds and roots, tubers and rhizomes are mentioned. Plant plants also includes harvested material and vegetative and generative reproductive material, for example cuttings, tubers, rhizomes, runners and seeds.

As plants which can be treated according to the invention, the following may be mentioned: cotton, flax, vine, fruit and vegetables, such as Rosaceae sp. (for example pomes such as apple and pear, but also drupes such as apricots, cherries, almonds and peaches and berry fruit such as strawberries), Ribesioidae sp., Juglandaceae sp., Betulaceae sp., Anacardiaceae sp., Fagaceae sp., moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, organs and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); main use plants, such as Gramineae sp. (for example maize, lawns, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pale choi, kohlrabi, radishes and rape, mustard, horseradish and cress), Fabacae sp. (for example bean, peanut), Papilionaceae sp. (for example soya bean), Solanaceae sp. (for example potatoes), Chenopodiaceae sp. (for example sugarbeet, fodder beet, mangold, beetroot); useful plants and ornamental plants in garden and woods; and genetically modified species of each of these plants. Preferably cereal plants are treated according to the invention.

For example, but without limitation, some pathogens of fungal diseases which can be treated according to the invention may be mentioned:

Diseases caused by pathogens of the true mildew such as for example Blumeria species, such as for example Blumeria graminis; Podosphaera species, such as for example Podosphaera leucotricha; Sphaerotheca species, such as for example Sphaerotheca fuliginea; Uncinula species, such as for example Uncinula necator;

Diseases caused by pathogens of rust diseases such as for example Gymnosporangium species, such as for example Gymnosporangium sabinae; Hemileia species, such as for example Hemileia vastatrix; Phakopsora species, such as for example Phakopsora pachyrhizi and Phakopsora meibomiae; Puccinia species, such as for example Puccinia recondita or Puccinia triticina; Uromyces species, such as for example Uromyces appendiculatus;

Diseases caused by pathogens of the Oomycetes group such as for example Bremia species, such as for example Bremia lactucae; Peronospora species, such as for example Peronospora pili or P. brassicae; Phytophthora species, such as for example Phytophthora infestans; Plasmopara species, such as for example Plasmopara viticola; Pseudoperonospora species, such as for example Pseudoperonospora humuli or Pseudoperonospora cubensis; Pythium species, such as for example Pythium ultimum;

Leaf spot diseases and leaf blight, e.g. caused by Alternaria species, such as for example Alternaria solani; Cercospora species, such as for example Cercospora beticola; Cladiosporum species, such as for example Cladiosporium cucumerinum; Cochliobolus species, such as for example Cochliobolus sativus (conidial form: Drechslera, Syn: Helminthosporium); Colletotrichum species, such as for example Colletotrichum lindemuthanium; Cycloconium species, such as for example Cycloconium oleaginum; Diaporthe species, such as for example Diaporthe citri; Elsinoe species, such as for example Elsinoe fawcettii; Gloeosporium species, such as for example Gloeosporium laeticolor; Glomerella species, such as for example Glomerella cingulata; Guignardia species, such as for example Guignardia bidwelli; Leptosphaeria species, such as for example Leptosphaeria maculans; Magnaporthe species, such as for example Magnaporthe grisea; Microdochium species, such as for example Microdochium nivale; Mycosphaerella species, such as for example Mycosphaerella graminicola and M. fijiensis; Phaeosphaeria species, such as for example Phaeosphaeria nodorum; Pyrenophora species, such as for example Pyrenophora teres; Ramularia species, such as for example Ramularia collo-cygni; Rhynchosporium species, such as for example Rhynchosporium secalis; Septoria species, such as for example Septoria apii; Typhula species, such as for example Typhula incamata; Venturia species, such as for example Venturia inaequalis;

Root and stem diseases, e.g. caused by Corticium species, such as for example Corticium graminearum; Fusarium species, such as for example Fusarium oxysporum; Gaeumannomyces species, such as for example Gaeumannomyces graminis; Rhizoctonia species, such as for example Rhizoctonia solani; Tapesia species, such as for example Tapesia acuformis; Thielaviopsis species, such as for example Thielaviopsis basicola;

Ear and panicle diseases (including maize cobs), e.g. caused by Alternaria species, such as for example Alternaria spp.; Aspergillus species, such as for example Aspergillus flavus; Cladosporium species, such as for example Cladosporium cladosporioides; Claviceps species, such as for example Claviceps purpurea; Fusarium species, such as for example Fusarium culmorum; Gibberella species, such as for example Gibberella zeae; Monographella species, such as for example Monographella nivalis; Septoria species, such as for example Septoria nodorum;

Diseases caused by smut fungi such as for example Sphacelotheca species, such as for example Sphacelotheca reiliana; Tilletia species, such as for example Tilletia caries, T. controversa; Urocystis species, such as for example Urocystis occulta; Ustilago species, such as for example Ustilago nuda, U. nuda tritici;

Fruit rot e.g. caused by Aspergillus species, such as for example Aspergillus flavus; Botrytis species, such as for example Botrytis cinerea; Penicillium species, such as for example Penicillium expansum and P. purpurogenum; Sclerotinia species, such as for example Sclerotinia sclerotiorum;

Verticilium species, such as for example Verticilium alboatrum;

Seed and soil-borne rots and blights and seedling diseases e.g. caused by Fusarium species, such as for example Fusarium culmorum; Phytophthora species, such as for example Phytophthora cactorum; Pythium species, such as for example Pythium ultimum; Rhizoctonia species, such as for example Rhizoctonia solani; Sclerotium species, such as for example Sclerotium rolfsii;

Canker diseases, galls and witches' broom, e.g. caused by Nectria species, such as for example Nectria galligena;

blight diseases e.g. caused by Monilinia species, such as for example Monilinia laxa;

Deformations of leaves, flowers and fruit, e.g. caused by Taphrina species, such as for example Taphrina deformans;

Degenerative diseases of woody plants, e.g. caused by Esca species, such as for example Phaemoniella clamydospora and Phaeoacremonium aleophilum and Fomitiporia mediterranea;

Flower and seed diseases e.g. caused by Botrytis species, such as for example Botrytis cinerea;

Diseases of plant tubers, e.g. caused by Rhizoctonia species, such as for example Rhizoctonia solani; Helminthosporium species, such as for example Helminthosporium solani;

Diseases caused by bacterial pathogens such as for example Xanthomonas species, such as for example Xanthomonas campestris pv. oryzae; Pseudomonas species, such as for example Pseudomonas syringae pv. lachrymans; Erwinia species, such as for example Erwinia amylovora;

Preferably, the following diseases of soya beans can be controlled:

Fungal diseases on leaves, stems, shoots and seeds e.g. caused by Alternaria leaf spot (Alternaria spec. atrans tenuissima), anthracnose (Colletotrichum gloeosporoides dematium var. truncatum), brown spot (Septoria glycines), Cercospora leaf spot and blight (Cercospora kikuchii), Choanephora leaf blight (Choanephora infundibulifera trispora (Syn.)), Dactuliophora leaf spot (Dactuliophora glycines), downy mildew (Peronospora manshurica), Drechslera blight (Drechslera glycini), frogeye leaf spot (Cercospora sojina), Leptosphaerulina leaf spot (Leptosphaerulina trifolii), Phyllostica leaf spot (Phyllosticta sojaecola), pod and stem blight (Phomopsis sojae), powdery mildew (Microsphaera diffusa), Pyrenochaeta leaf spot (Pyrenochaeta glycines), Rhizoctonia aerial, foliage, and web blight (Rhizoctonia solani), rust (Phakopsora pachyrhizi, Phakopsora meibomiae), scab (Sphaceloma glycines), Stemphylium leaf blight (Stemphylium botryosum), target spot (Corynespora cassiicola).

Fungal diseases on roots and the stem base e.g. caused by black root rot (Calonectria crotalariae), charcoal rot (Macrophomina phaseolina), Fusarium blight or wilt, root rot, and pod and collar rot (Fusarium oxysporum, Fusarium orthoceras, Fusarium semitectum, Fusarium equiseti), Mycoleptodiscus root rot (Mycoleptodiscus terrestris), Neocosmospora (Neocosmopspora vasinfecta), pod and stem blight (Diaporthe phaseolorum), stem canker (Diaporthe phaseolorum var. caulivora), Phytophthora rot (Phytophthora megasperma), brown stem rot (Phialophora gregata), Pythium rot (Pythium aphanidermatum, Pythium irregulare, Pythium debaryanum, Pythium myriotylum, Pythium ultimum), Rhizoctonia root rot, stem decay, and damping-off (Rhizoctonia solani), Sclerotinia stem decay (Sclerotinia sclerotiorum), Sclerotinia Southern blight (Sclerotinia rolfsii) and Thielaviopsis root rot (Thielaviopsis basicola).

In the present case, undesired microorganisms are understood to mean phytopathogenic fungi and bacteria. The substances according to the invention can thus be used to protect plants within a certain period after the treatment against infection from the said pathogen pests. The period during which their protection is effected in general extends from 1 to 10 days, preferably 1 to 7 days after the treatment of the plants with the active substances.

The good plant tolerability of the active substances in the concentrations necessary for the control of plant diseases allows the treatment of aboveground plant parts, of plant and seed material, and of the soil.

At the same time, the active substances according to the invention can be used with particularly good results for the control of cereal diseases, such as for example against Erysiphe species, against Puccinia and against Fusaria species, of rice diseases, such as for example against Pyricularia and Rhizoctonia and of diseases in viticulture, fruit-growing and vegetable cultivation, such as for example against Botrytis-, Venturia-, Sphaerotheca- and Podosphaera species.

The active substances according to the invention are also suitable for increasing the harvest yield. Moreover, they are of low toxicity and display good plant tolerability.

The compounds according to the invention can optionally also be used at certain concentrations or application dosages as herbicides, safeners, growth regulators or agents for improvement of the plant properties, or as microbicides, for example as fungicides, antimycotics, bactericides, viricides (including agents against viroids) or as agents against MLO (mycoplasma-like organism) and RLO (Rickettsia-like organism). They can optionally also be used as insecticides. They can optionally also be used as intermediate or precursor products for the synthesis of further active substances.

The active substances according to the invention can also optionally be used at certain concentrations and application dosages as herbicides, for influencing plant growth, and for the control of animal pests. They can optionally also be used as intermediates or precursors for the synthesis of further active substances.

The active substances according to the invention, with good plant tolerability, low mammalian toxicity and good environmental tolerability, are suitable for the protection of plants and plant organs, for increasing the harvest yield, and improving the quality of the harvested material. They can preferably be used as pesticides. They are active against normally sensitive and resistant species and against all or some developmental stages.

The treatment of the plants and plant parts with the active substances or agents according to the invention is effected directly or by acting on their environment, habitat or storage space by the usual treatment methods, e.g. by dipping, sprinlding, spraying, irrigation, vaporization, dusting, misting, scattering, foaming, coating, spreading, drenching, droplet irrigation and also, for reproductive material, in particular for seeds, by dry dressing, wet dressing, slurry dressing, incrustation, single- or multilayer coating etc. It is also possible to apply the active substances by the ultra-low volume process or to inject the active substance preparation or the active substance itself into the soil.

The quantity of active substance applied can vary over a considerable range. It essentially depends on the nature of the desired effect. In general, the application dosages lie between 1 g and 10 kg active substance per hectare soil area, preferably between 5 g and 5 kg per ha.

The advantageous effect of the crop plant tolerability of the active substances according to the invention is particularly marked with certain concentration ratios. However, the weight ratios of the active substances in the active substance combinations can be varied over relatively large ranges. In general, 0.001 to 1000 parts by weight, preferably 0.01 to 100 parts by weight, particularly preferably 0.05 to 20 parts by weight, of one of the crop plant tolerability-improving compounds (antidotes/safeners) named above under (b′) are used for 1 part by weight of active substance of the formula (I).

The active substances according to the invention are generally used in the form of finished formulations. However, the active substances contained in the active substance combinations can also be mixed in single formulations on application, i.e. applied in the foam of tank mixtures.

In addition, through the treatment according to the invention, the mycotoxin content in the harvested material and the foods and feedstuffs produced therefrom can be reduced. Here, the following mycotoxins are particularly, but not exclusively, to be named: deoxynivalenol (DON), nivalenol, 15-Ac-DON, 3-Ac-DON, T2- and HT2-toxin, fumonisine, zearalenone, moniliformin, fusarin, diaceotoxyscirpenol (DAS), beauvericin, enniatin, fusaroproliferin, fusarenol, echratoxine, patulin, ergot alkaloids and aflatoxins, which can for example be caused by the following fungi: Fusarium spp., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides inter alia and also by Aspergillus spp., Penicillium spp., Claviceps purpurea or Stachybotrys spp. inter alia

The active substances or agents according to the invention can moreover be used in material protection for the protection of industrial materials against infection and destruction by undesired microorganisms, such as for example fungi.

In the present connection, industrial materials should be understood to mean nonliving materials which are prepared for use in industry. For example, technical materials which are intended to be protected by active substances according to the invention against microbial spoilage or destruction can be adhesives, glues, paper and cardboard, textiles, leather, wood, coating materials and plastic articles, cooling lubricants and other materials which can be infected or degraded by microorganisms. In the context of the materials to be protected, parts of production plants, for example cooling water loops may be mentioned, which can be impaired by multiplication of microorganisms. In the context of the present invention, preferably adhesives, glues, papers and cardboard, leather, wood, coating materials, cooling lubricants and heat transfer fluids, particularly preferably wood, may be mentioned as industrial materials. The active substances or agents according to the invention can prevent adverse effects such as rotting, decay, discolouration, decolourization or mouldiness.

The method according to the invention for the control of undesired fungi can also be used for the protection of so-called storage goods. Here “storage goods” is understood to mean natural substances or plant or animal origin, or processed products therefrom, which have been taken from nature, and for which long-term protection is desired. Storage goods of plant origin, such as for example plants or plant parts, such as stalks, leaves, tubers, seeds, fruit, or grain, can be protected in the freshly harvested state or after processing by (pre-)drying, moistening, grinding, milling, pressing or roasting. Storage goods also comprises timber, whether it is unprocessed, like whole timber, power line masts and boxes or in the form of finished products such as furniture. Storage goods of animal origin are for example pelts, leather, fleeces and hair. The active substances according to the invention prevent adverse effects such as rotting, decay, discolouration, decolourization or mouldiness.

As microorganisms which can cause a degradation or alteration in the industrial materials, for example bacteria, fungi, yeasts, algae and slime organisms may be named. Preferably the active substances according to the invention act against fungi, in particular mould fungi, wood-discolouring and wood-destroying fungi (Basidiomycetes) and against slime organisms and algae. For example microorganisms of the following genera may be named: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; polyporus, such as polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

The present invention further relates to an agent for the control of undesired microorganisms, comprising at least one of the thienylaminopyrimidines according to the invention. These are preferably fungicidal agents which contain agriculturally usable additives, solvents, carrier substances, surface-active substances or thinners.

According to the invention, carrier substance means a natural or synthetic, organic or inorganic substance, with which the active substances are mixed or combined for better applicability, above all for the application onto plants or plant parts or seeds. The carrier substance, which can be solid or liquid, is in general inert and should be usable in agriculture.

Possible carrier substances are for example: ammonium salts and natural mineral powders, such as kaolins, aluminas, talc, chalk, quartz, attapulgite, montmorillonite or diatomaceous earth and synthetic mineral powders, such as high disperse silica, aluminium oxides and silicates, possible carrier substances for granules are for example: broken and fractionated natural minerals such as calcite, marble, pumice, meerschaum, dolomite and synthetic granules from inorganic and organic powders and granules from organic material such as paper, sawdust, coconut shells, maize cobs and tobacco stalks; possible emulsifying or foaming agents are for example: nonionogenic and anionic emulsifiers, such as polyoxyethylene fatty acid esters, polyoxyethylene fatty alcohol ethers, e.g. alkylaryl polyglycol ethers, alkylsulphonates, alkyl sulphates, arylsulphonates and protein hydrolysates; possible dispersants are nonionic and/or ionic substances, e.g. from the classes of the alcohol POE and/or POP ethers, acid and/or POP-POE esters, alkyl-aryl and/or POP POE ethers, fatty and/or POP POE adducts, POE and/or POP polyol derivatives, POE and/or POP sorbitan or sugar adducts, alkyl or aryl sulphates, sulphonates and phosphates or the corresponding PO ether adducts. Also suitable oligo- or polymers, e.g. starting from vinylic monomers, from acrylic acid, from EO and/or PO alone or in combination with e.g. (poly-) alcohols or (poly-) amines. Further, lignin and sulphonic acid derivatives thereof, simple and modified celluloses, aromatic and/or aliphatic sulphonic acids and adducts thereof with formaldehyde, can be used.

The active substances can be converted into the usual formulations, such as solutions, emulsions, wettable powders, water- and oil-based suspensions, powders, dusting agents, pastes, soluble powders, soluble granules, granules for spreading, suspension emulsion concentrates, active substance-impregnated natural substances, active substance-impregnated synthetic substances, fertilizers and superfine encapsulations in polymeric substances.

The active substances can be applied as such, in the form of formulations thereof or the use forms prepared therefrom, such as ready-for-use solutions, emulsions, water- or oil-based suspensions, powders, wettable powders, pastes, soluble powders, dusting agents, soluble granules, granules for spreading, suspension emulsion concentrates, active substance-impregnated natural substances, active substance-impregnated synthetic substances, fertilizers and superfine encapsulations in polymeric substances. The application is effected in a usual manner, for example by drenching, sprinkling, spraying, scattering, dusting, foaming, coating etc. It is also possible to apply the active substances by the ultra-low volume process or to inject the active substance preparation or the active substance itself into the soil. The seeds of the plants can also be treated.

The said formulations can be prepared in a manner in itself known, e.g. by mixing of the active substances with at least one usual thinner, solvent or diluent, emulsifier, dispersing and/or binding or fixing agent, wetting agent, water-repellant, if necessary desiccants and UV stabilizers and if necessary dyes and pigments, defoamants, preservatives, secondary thickeners, glues, gibberellins and other processing additives.

The agents according to the invention comprise not only formulations which are already ready for use and can be applied onto the plant or the seeds with a suitable apparatus, but also commercial concentrates which must be diluted with water before use.

The active substances according to the invention can be present as such or in their (normal commercial) formulations and in the use forms prepared from these formulations mixed with other (known) active substances, such as insecticides, attractants, sterilants, bactericides, acaricides, nematicides, fungicides, growth regulators, herbicides, fertilizers, safeners or semiochemicals.

As additives, substances can be used which are suitable for imparting particular properties to the agent itself and/or preparations derived therefrom (e.g. wettable powders, seed dressings), such as certain technical properties and/or even particular biological properties. Thinners, solvents and carrier substances are typical possible additives.

Water, polar and nonpolar organic chemical liquids e.g. from the classes of the aromatic and nonaromatic hydrocarbons (such as paraffins, alkylbenzenes, alkylnaphthalenes, chlorobenzenes), the alcohols and polyols (which can also optionally also be substituted, etherified and/or esterified), the ketones (such as acetone, cyclohexanone), esters (also fats and oils) and (poly-)ethers, the simple and substituted amines, amides, lactams (such as N-alkylpyrrolidone) and lactones, the sulphones and sulphoxides (such as dimethyl sulphoxide), are for example suitable as thinners.

By liquefied gaseous thinners or carrier substances are meant those liquids which are gaseous at normal temperature and under normal pressure, e.g. aerosol propellant gases such as halohydrocarbons and butane, propane, nitrogen and carbon dioxide.

In the formulations, adhesive agents such as carboxymethylcellulose, natural and synthetic powder, granular or latex polymers, such as gum arabic, polyvinyl alcohol, polyvinyl acetate, and natural phospholipids, such as cephalins and lecithins, and synthetic phospholipids can be used. Other additives can be mineral and vegetable oils.

In case of the use of water as a thinner, for example organic solvents can also be used as auxiliary solvents. Essentially, possible liquid solvents are: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, e.g. petroleum fractions, alcohols, such as butanol or glycol and ethers and esters thereof, ketones, such as acetone, methyl ethyl ketone, methyl isobutyl ketone or cyclohexanone, strongly polar solvents, such as dimethylformamide and dimethyl sulphoxide, and water.

The agents according to the invention can additionally contain other components, such as for example surface-active substances. Possible surface-active substances are emulsifying and/or foaming agents, dispersants or wetting agents with ionic or nonionic properties or mixtures of these surface-active substances. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic acid esters, taurine derivatives (preferably alkyl taurates), phosphate esters of polyethoxylated alcohols or phenols, fatty acid esters of polyols, and derivatives of the compounds containing sulphates, sulphonates and phosphates, e.g. alkylaryl polyglycol ethers, alkylsulphonates, alkyl-sulphates, arylsulphonates, protein hydrolysates, lignin sulphite waste liquor and methylcellulose. The presence of a surface-active substance is necessary when one of the active substances and/or one of the inert carrier substances is not soluble in water and when the application is effected in water. The proportion of surface-active substances lies between 5 and 40 weight percent of the agent according to the invention.

Colorants such as inorganic pigments, e.g. iron oxide, titanium oxide, prussian blue and organic dyes such as alizarin, azo and metal phthalocyanine dyes and trace nutrients such as salts of iron, manganese, boron, copper, cobalt, molybdenum and zinc can be used.

Further additives can be perfumes, mineral or optionally modified vegetable oils, waxes and nutrients (including trace nutrients) such as salts of iron, manganese, boron, copper, cobalt, molybdenum and zinc.

Stabilizers such as cold stabilizers, preservatives, antioxidants, light protection agents or other agents improving the chemical and / or physical stability can also be contained.

Optionally, other additional components can also be contained, e.g. protective colloids, binders, adhesives, thickeners, thixotropic substances, penetration enhancers, stabilizers, sequestering agents and complexing agents. In general, the active substances can be combined with any solid or liquid additive which is commonly used for formulation purposes.

The formulations in general contain between 0.05 and 99 wt. %, 0.01 and 98 wt. %, preferably between 0.1 and 95 wt. %, particularly preferably between 0.5 and 90% of active substance, quite particularly preferably between 10 and 70 weight percent.

The formulations described above can be used in a method according to the invention for the control of undesired microorganisms, wherein the thienylaminopyrimidines according to the invention are applied onto the microorganisms and/or in their habitat.

The active substances according to the invention can also be used as such or in formulations thereof mixed with known fungicides, bactericides, acaricides, nematicides or insecticides, in order thus for example to broaden the activity spectrum or avoid the development of resistances.

Possible mixing partners are for example known fungicides, insecticides, acaricides, nematicides or also bactericides (see also Pesticide Manual, 13th ed.).

A mixture with other known active substances, such as herbicides, or with fertilizers and growth regulators, safeners or semiochemicals is also possible.

The application is effected in a manner suited to the use forms.

The control of plant pathogenic noxious fungi is effected first and foremost by the treatment of the soil and the aboveground plant parts with pesticides. Because of the concerns regarding possible effects of the pesticide on the environment and the health of people and animals, there are efforts to reduce the quantity of the active substances applied.

The active substances can be applied as such, in the form of formulations thereof or the use forms prepared therefrom, such as ready-for-use solutions, suspensions, wettable powders, pastes, soluble powders, dusting agents and granules. The application is effected in a usual manner, for example by drenching, sprinkling, spraying, scattering, dusting, foaming, coating, etc. It is also possible to apply the active substance preparation or the active substance itself by the ultra-low volume process or to inject the active substance preparation or the active substance itself into the soil. The seeds of the plants can also be treated.

In the use of the active substances according to the invention as fungicides, depending on the mode of application, the application dosages can be varied within a considerable range. The application dosage of the active substances according to the invention is:
in the treatment of plant parts, e.g. leaves: from 0.1 to 10,000 g/ha, preferably from 10 to 1,000 g/ha, particularly preferably from 50 to 300 g/ha (for application by drenching or dripping, the application dosage can even be reduced, particularly when inert substrates such as rock wool or perlite are used);
in seed treatment: from 2 to 200 g per 100 kg seeds, preferably from 3 to 150 g per 100 kg seeds, particularly preferably from 2.5 to 25 g per 100 kg seeds, quite particularly preferably from 2.5 to 12.5 g per 100 kg seeds;
In soil treatment: from 0.1 to 10,000 g/ha, preferably from 1 to 5,000 g/ha.

These application dosages are stated only for example and non-restrictively in the sense of the invention.

At the same time, the compounds according to the invention can be used for protection against growth on objects, in particular on ship hulls, sieves, nets, buildings, wharves and signal installations which come into contact with sea water or brackish water.

Further, the compounds according to the invention can be used alone or in combination with other active substances as antifouling agents.

The treatment method according to the invention can be used for the treatment of genetically modified organisms (GMOs), e.g. plants or seeds. Genetically modified plants (or transgenic plants) are plants in which a heterologous gene has been stably integrated into the genome. The term “heterologous gene” essentially means a gene which is prepared or assembled outside the plant and which on introduction into the cell nucleus genome, the chloroplast genome or the hypochondrial genome thereby imparts to the transformed plant new or improved agronomic or other properties, that it expresses a protein or polypeptide of interest or that it down-regulates or switches off another gene which is present in the plant, or other genes which are present in the plant (for example by means of antisense technology, cosuppression technology or RNAi technology [RNA Interference]). A heterologous gene which is present in the genome is also described as a transgene. A transgene which is defined by its specific presence in the plant genome is described as a transformation or transgenic event.

Depending on the plant species or plant varieties, their location and their growth conditions (soils, climate, vegetation periods, nutrition) the treatment according to the invention can also lead to super-additive (“synergistic”) effects. Thus for example the following effects are possible, which go beyond the effects strictly speaking to be expected: decreased application dosages and/or extended activity spectrum and/or increased effectiveness of the active substances and compositions which can be used according to the invention, better plant growth, increased tolerance against high or low temperatures, increased tolerance against drought or water or soil salt content, increased flowering, greater ease of harvesting, accelerated ripening, higher yields, larger fruit, greater plant height, more intense green colour of leaf, earlier flowering, higher quality and/or higher nutritional value of harvested products, higher sugar concentration in the fruit, and better storability and/or processability of the harvested products.

In the present case, undesired phytopathogenic fungi and/or microorganisms and/or viruses are understood to mean phytopathogenic fungi, bacteria and viruses. The substances according to the invention can therefore be used for the protection of plants against infection by the said pathogens within a certain period after the treatment. The period over which a protective action is achieved in general extends from 1 to 10 days, preferably 1 to 7 days after the treatment of the plants with the active substances.

Plants and plant varieties which are preferably treated according to the invention include all plants which have genetic material which imparts to these plants particularly advantageous, useful features (irrespective of whether this was achieved by breeding and/or biotechnology).

Plants and plant varieties which likewise are preferably treated according to the invention are resistant against one or more biotic stress factors, i.e. these plants have improved defences against animal and microbial pests such as nematodes, insects, mites, phytopathogenic fungi, bacteria, viruses and/or viroids.

Plants and plant varieties which can also be treated according to the invention are plants which are resistant against one or more abiotic stress factors. The abiotic stress factors can for example include aridity, cold and heat conditions, osmotic stress, waterlogging, increased soil salt content, increased exposure to minerals, ozone conditions, strong light conditions, limited availability of nitrogenous nutrients, limited availability of phosphorus nutrients or avoidance of shade.

Plants and plant varieties which can also be treated according to the invention are plants which are characterized by increased yield properties. In these plants, an increased yield can for example be due to improved plant physiology, improved plant growth and improved plant development, such as water utilization efficiency, water retention efficiency, improved nitrogen utilization, increased carbon assimilation, improved photosynthesis, strengthened vitality and accelerated ripening. The yield can moreover be influenced (under stress and non-stress conditions) by improved plant architecture, including early flowering, control of flowering for the production of hybrid seed, seedling vigour, plant size, internode number and spacing, root growth, seed size, fruit size, pod size, number of pods or ears, seed mass, intensified seed filling, decreased seed loss, decreased pod burst and lodging resistance. Further yield characteristics include seed composition such as carbohydrate content, protein content, oil content and oil composition, nutritional value, reduction in antinutrient compounds, improved processability and improved storability.

Plants which can be treated according to the invention are hybrid plants which already express the properties of the heterosis or hybrid effect, which in general results in higher yield, greater vigour, better health and better resistance against biotic and abiotic stress factors. Such plants are typically created by crossing an inbred pollen sterile parent line (the female crossing partner) with another inbred pollen fertile parent line (the male crossing partner). The hybrid seed is typically harvested from the pollen sterile plants and sold to growers. Pollen sterile plants can sometimes (e.g. for maize) be produced by detassling (i.e. mechanical removal of the male sex organs or the male flowers); it is however more usual for the pollen sterility to be due to genetic determinants in the plant genome. In this case, in particular when the desired product is the seeds, since it is desired to harvest from the hybrid plants, it is usually beneficial to ensure that the pollen fertility is fully restored in hybrid plants which contain the genetic determinants responsible for the pollen sterility. This can be achieved by ensuring that the male crossing partners possess corresponding fertility restorer genes which are capable of restoring the pollen fertility in hybrid plants which contain the genetic determinants responsible for the pollen sterility. Genetic determinants for pollen sterility can be located in the cytoplasm. Examples of cytoplasmic pollen sterility (CMS) have for example been described for Brassica species. However, genetic determinants for pollen sterility can also be located in the cell nucleus genome. Pollen sterile plants can also be obtained with plant biotechnology methods, such as genetic engineering. A particularly favourable means for the creation of pollen sterile plants is described in WO 89/10396, wherein for example a ribonuclease such as a barnase is selectively expressed in the tapetum cells in the stamens. The fertility can be restored by expression of a ribonuclease inhibitor such as barstar in the tapetum cells.

Plants or plant varieties (which are obtained by plant biotechnology methods, such as genetic engineering) which can be treated according to the invention are herbicide-tolerant plants, i.e. plants which have been made tolerant to one or more specified herbicides. Such plants can be obtained either by genetic transformation or by selection of plants which contain a mutation which imparts such herbicide tolerance.

Herbicide-tolerant plants are for example glyphosate-tolerant plants, i.e. plants which have been made tolerant to the herbicide glyphosate or salts thereof. Thus for example glyphosate-tolerant plants can be obtained by transformation of the plant with a gene which codes for the enzyme 5-enol-pyruvylshikimate 3-phosphate synthase (EPSPS). Examples of such EPSPS genes are the AroA gene (mutant CT7) of the bacterium Salmonella typhimurium, the CP4 gene of the bacterium Agrobacterium sp., and the genes which code for an EPSPS from the petunia, for an EPSPS from the tomato or for an EPSPS from eleusine. It can also be a mutated EPSPS. Glyphosate-tolerant plants can also be obtained by expressing a gene which codes for a glyphosate oxidoreductase enzyme. Glyphosate-tolerant plants can be obtained by expressing a gene which codes for a glyphosate acetyltransferase enzyme. Glyphosate-tolerant plants can be obtained by selecting plants which naturally occurring mutations of the aforesaid genes.

Other herbicide-resistant plants are for example plants which have been made tolerant towards herbicides which inhibit the enzyme glutamine synthase, such as bialaphos, phosphinotricin or glufosinate. Such plants can be obtained by expressing an enzyme which detoxifies the herbicide or is a mutant of the enzyme glutamine synthase which is resistant to inhibition. Such an effective detoxifying enzyme is for example an enzyme which codes for a phosphinotricin acetyltransferase (such as for example the bar- or pat-protein from Streptomyces species). Plants which express an exogeneous phosphinotricin acetyltransferase have been described.

Further herbicide-tolerant plants are also plants which have been made tolerant towards the herbicides which inhibit the enzyme hydroxyphenylpyruvate dioxygenase (HPPD). The hydroxyphenyl-pyruvate dioxygenases are enzymes which catalyse the reaction wherein para-hydroxyphenylpyruvate (HPP) is converted to homogentisate. Plants which are tolerant towards HPPD inhibitors can be transformed with a gene, which codes for a naturally occurring resistant HPPD, or a gene which codes for a mutated HPPD enzyme. A tolerance towards HPPD inhibitors can also be achieved by transforming plants with genes which code for certain enzymes which enable the formation of homogentisate in spite of inhibition of the native HPPD enzyme by the HPPD inhibitor. The tolerance of plants towards HPPD inhibitors can also be improved by transfot ming plants with a gene which codes for a prephenate dehydrogenase enzyme in addition to a gene which codes for an HPPD tolerant enzyme.

Other herbicide-resistant plants are plants which have been made tolerant towards acetolactate synthase (ALS) inhibitors. Known ALS inhibitors for example include sulphonylurea, imidazolinone, triazolopyrimidines, pyrimidinyloxy(thio)benzoates and/or sulphonylaminocarbonyltriazolinone herbicides. It is known that various mutations in the enzyme ALS (also known as acetohydroxy acid synthase, AHAS) impart a tolerance towards different herbicides or groups of herbicides. The production of sulphonylurea-tolerant plants and imidazolinone-tolerant plants is described in the international publication WO 96/033270. Other sulphonylurea- and imidazolinone-tolerant plants are also described for example in WO 07/024782.

Other plants which are tolerant towards imidazolinone and/or sulphonylurea tolerant can be obtained by induced mutagenesis, selection in cell cultures in the presence of the herbicide or by mutation breeding.

Plants or plant varieties (which were obtained by plant biotechnology methods, such as genetic engineering) which can also be treated according to the invention, are insect-resistant transgenic plants, i.e. plants which have been made resistant against infection by certain target insects. Such plants can be obtained by genetic transformation or by selection of plants which contain a mutation which imparts such an insect resistance.

In the present connection, the term “insect-resistant transgenic plant” comprises any plant which contains at least one transgene which contains a coding sequence which codes for the following:
1) an insecticidal crystalline protein from Bacillus thuringiensis or an insecticidal part thereof, such as the insecticidal crystalline proteins which have been described, were compiled online at: http://www.lifesci.sussex.ac.uk/Home/Neil_Crickmore/Bt/, or insecticidal parts thereof, e.g. proteins of the Cry protein classes Cry1Ab, Cry1Ac, Cry1F, Cry2Ab, Cry3Ae or Cry3Bb or insecticidal parts thereof; or
2) a crystalline protein from Bacillus thuringiensis or a part thereof, which in the presence of a second, other crystalline protein than Bacillus thuringiensis or a part thereof has insecticidal action, such as the binary toxin, which consists of the crystalline proteins Cy34 and Cy35; or
3) an insecticidal hybrid protein, which comprises parts of two different insecticidal crystalline proteins from Bacillus thuringiensis, such as for example a hybrid of the proteins from 1) above or a hybrid of the proteins from 2) above, e.g. the protein Cry1A.105, which is produced by the maize event MON98034 (WO 07/027777); or
4) A protein according to one of the points 1) to 3) above, wherein some, in particular 1 to 10, amino acids have been replaced by another amino acid in order to achieve higher insecticidal activity against a target insect species and/or in order to broaden the spectrum of the relevant target insect species and/or because of changes which were induced in the coding DNA during the cloning or transformation, such as the protein Cry3Bb1 in maize events MON863 or MON88017 or the protein Cry3A in the maize event MIR 604;

5) an insecticidal secreted protein from Bacillus thuringiensis or Bacillus cereus or an insecticidal part thereof, such as the vegetatively acting insect-toxic proteins (vegetative insecticidal proteins, VIP), which are listed under http://www.lifesci.sussex.ac.uk/Home/Neil_Crickmore/Bt/vip.html, e.g. proteins of the protein class VIP3Aa; or
6) a secreted protein from Bacillus thuringiensis or Bacillus cereus which in the presence of a second secreted protein from Bacillus thuringiensis or B. cereus has insecticidal action, such as the binary toxin which consists of the proteins VLP1A and VIP2A.
7) an insecticidal hybrid protein which comprises parts of various secreted proteins from Bacillus thuringiensis or Bacillus cereus, such as a hybrid of the proteins from 1) or a hybrid of the proteins from 2) above; or
8) a protein according to one of the points 1) to 3) above, wherein some, in particular 1 to 10, amino acids have been replaced by another amino acid in order to achieve higher insecticidal activity against a target insect species and/or in order to broaden the spectrum of the relevant target insect species and/or because of changes which were induced in the coding DNA during the cloning or transformation (wherein the coding for an insecticidal protein is retained), such as the protein VIP3Aa in the cotton event COT 102.

Naturally, the insect-resistant transgenic plants in the present connection also include any plant which contains a combination of genes which code for the proteins from one of the aforesaid classes 1 to 8. In one embodiment an insect-resistant plant contains more than one transgene which codes for a protein according to one of the aforesaid 1 to 8, in order to broaden the spectrum of the relevant target insect species or in order to retard the development of a resistance of the insects against the plants by inserting various proteins which are insecticidal for the same target insect species, but have a different mode of action, such as binding to different receptor binding sites in the insect.

Plants or plant varieties (which were obtained by plant biotechnology methods, such as genetic engineering) which can also be treated according to the invention are tolerant towards abiotic stress factors. Such plants can be obtained by genetic transformation or by selection of plants which contain a mutation which imparts such stress resistance. Particularly useful plants with stress tolerance include the following:
a. Plants which contain a transgene which is able to reduce the expression and/or activity of the gene for the poly(ADP-ribose) polymerase (PARP) in the plant cells or plants.
b. Plants which contain a stress tolerance-promoting transgene which is able to reduce the expression and/or activity of the genes of the plants or plant cells coding for PARD;
c. Plants which contain a stress tolerance-promoting transgene which codes for an enzyme of the nicotinamide adenine dinucleotide salvage biosynthesis pathway functional in plants, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant varieties (which were obtained by plant biotechnology methods, such as genetic engineering) which can also be treated according to the invention exhibit a modified quantity, quality and/or storability of the harvested product and/or modified properties of certain components of the harvested product, such as for example:
1) Transgenic plants which synthesize a modified starch which is modified as regards its chemical and physical properties, in particular the amylose content or the amylose/amylopectin ratio, the degree of branching, the average chain length, the distribution of the side-chains, the viscosity behaviour, the gel strength, the starch grain size and/or starch morphology compared with the starch synthesized in wild type cells or plants, so that this modified starch is better suited for certain applications.
2) Transgenic plants, which synthesize non-starch carbohydrate polymers, or non-starch carbohydrate polymers whose properties are modified compared to wild type plants with no genetic modification. Examples are plants which produce polyfructose, in particular of the inulin and levan type, plants which produce alpha-1,4-glucans, plants which produce alpha-1,6-branched alpha-1,4-glucans and plants which produce alteman.
3) Transgenic plants which produce hyaluronan.

Plants or plant varieties (which were obtained by plant biotechnology methods, such as genetic engineering) which can also be treated according to the invention are plants such as cotton plants with modified fibre properties. Such plants can be obtained by genetic transformation or by selection of plants which contain a mutation which imparts such modified fibre properties; these include:
a) Plants such as cotton plants which contain a modified form or cellulose synthase genes,
b) Plants such as cotton plants which contain a modified form of rsw2- or rsw3-homologous nucleic acids;
c) Plants such as cotton plants with increased expression of the saccharose phosphate synthase;
d) Plants such as cotton plants with increased expression of the saccharose synthase;
e) Plants such as cotton plants in which the timing of the permeability control of the plasmodesmata is modified on the basis of the fibre cell, e.g. by down-regulation of the fiber-selective β-1,3-glucanase;
f) Plants such as cotton plants with fibres with modified reactivity, e.g. by expression of the N-acetylglucosamine transferase gene, also including nodC, and of chitin synthase genes.

Plants or plant varieties (which were obtained by plant biotechnology methods, such as genetic engineering) which can also be treated according to the invention are plants such as rape or related Brassica plants with modified oil composition properties. Such plants can be obtained by genetic transfolination or by selection of plants which contain a mutation which imparts such modified oil properties; they include:
a) Plants such as rape plants which produce oil with a high oleic acid content;
b) Plants such as rape plants which produce oil with a low linolenic acid content.
c) Plants such as rape plants which produce oil with a low saturated fatty acid content.

Particularly useful transgenic plants which can be treated according to the invention are plants with one or more genes which code for one or more toxins, are the transgenic plants which are sold under the following trade names: YIELD GARD® (for example maize, cotton, soya beans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Herbicide-tolerant plants which are to be mentioned are for example maize varieties, cotton varieties and soya bean varieties which are sold under the following trade names: Roundup Ready® (glyphosate tolerance, for example maize, cotton, soya bean), Liberty Link® (phosphinotricin tolerance, for example rape), IMI® (imidazolinone tolerance) and SCS® (Sylfonylurea tolerance), for example maize. The herbicide-resistant plants (plants bred traditionally for herbicide tolerance) which are to be mentioned include the varieties sold under the name Clearfield® (for example maize).

Particularly useful transgenic plants which can be treated according to the invention are plants which contain transformation events, or a combination of transformation events, and which are for example listed in the databases of various national or regional authorities (see for example http://gmoinfo.jrc.it/gmp_browse.aspx and http://www.agbios.com/dbase.php).

The listed plants can be particularly advantageously treated according to the invention with den compounds of the general formula (I). The preference ranges stated above for the active substances or mixtures also apply for the treatment of these plants. The treatment of plants with the compounds or mixtures specifically listed in the present text may be particularly emphasized.

The active substances or agents according to the invention can also be used to protect plants against infection by the said pests within a certain period after the treatment. The period within which protection is imparted in general extends to 1 to 28 days, preferably to 1 to 14 days, particularly preferably to 1 to 10 days and quite particularly preferably to 1 to 7 days after the treatment of the plants with the active substances or to up to 200 days after a seed treatment.

The production and the use of the active substances according to the invention of the formulae [I] and [I-c] follows from the following examples. However, the invention is not restricted to these examples.

Production of Starting Materials of the Formula [VII-1]:

### 4-Bromo-3-(4-fluorophenyl)-1H-pyrazole [VII-I]

14.9 g (92 mmol) of 3-(4-fluorophenyl)-1H-pyrazole (synthesis described in EP-A-1 382 603) are dissolved in 45 mL acetic acid. To this is added a solution of 5.7 mL bromine (110 mmol) in 9 mL acetic acid at 3-5° C. A precipitate is formed to which a further 130 mL acetic acid are added. After this, the reaction mixture is stirred for a further 4 hrs at room temperature. Next all volatile components are removed under high vacuum. The residue is dissolved in 1 molar sodium carbonate solution and extracted several times with ethyl acetate. Next the organic phase is dried (Na₂SO₄) and concentrated. 21.8 g of 4-bromo-3-(4-fluorophenyl)-1H-pyrazole (yield 98%) are obtained as a colourless solid. The product is reacted further without further purification.

logP (pH 2.7): 2.29

MS (ESI): 241.0 ([M+H]⁺)

1H-NMR (400 MHz, d₆-DMSO): δ=13.3 (s, 1H, br), 7.82 (m, 3H), 7.30 (m, 2H) ppm

Production of Starting Materials of the Formula [VI]:

### Mixture of 4-bromo-3-(4-fluorophenyl)-1-isopropyl-1H-pyrazole and 4-bromo-5-(4-fluoro-phenyl)-1-isopropyl-1H-pyrazole [VI-1]

3.6 g (14.9 mmol) of 4-bromo-3-(4-fluorophenyl)-1H-pyrazole are dissolved in 6 mL N,N-dimethylformamide. 0.43 g of sodium hydride (17.9 mmol) as a 60% suspension in oil are added to this and the mixture is stirred for 20 mins at 25° C. Next, 3.8 g of isopropyl iodide (22.4 mmol) are added and the reaction mixture is stirred overnight at 25° C. For the workup, acetic acid (concentrated) is slowly added (0.2 eq) and then all volatile components are removed under high vacuum. The residue is dissolved in water and extracted several times with ethyl acetate. Next the organic phase is dried (Na₂SO₄) and concentrated. Purification is effected by silica gel chromatography with the eluent cyclohexane (A)/ethyl acetate (B) (0% B up to 40% B). 3.54 g of a (84:16) mixture of 4-bromo-3-(4-fluorophenyl)-1-isopropyl-1H-pyrazole and 4-bromo-5-(4-fluorophenyl)-1-isopropyl-1H-pyrazole (minor) are obtained as a colourless solid. The product is reacted further without further purification.

logP (pH 2.7): 3.96 and 3.68^{minor}

MS (ESI): 285.0 ([M+H]⁺)

1H-NMR (400 MHz, d6-DMSO): δ=8.04 (s, 1H), 7.85 (dd, 2H), 7.64 (s, 1H^{minor}), 7.43 (dd, 2H^{minor}), 7.36 (t, 2H^{minor}), 7.25 (t, 2H), 4.51 (m, 1H), 4.36 (m, 1H^{minor}), 1.45 (d, 6H), 1.33 (d, 6H^{minor}) ppm

### 4-Bromo-1-ethyl-3-(4-fluorophenyl)-1H-pyrazole [VI-2]

### 4-Bromo-1-ethyl-5-(4-fluorophenyl)-1H-pyrazole [VI-3]

3.0 g (12.3 mmol) of 4-bromo-3-(4-fluorophenyl)-1H-pyrazole are dissolved in 6 mL N,N-dimethylformamide. 0.59 g of sodium hydride (14.7 mmol) as a 60% suspension in oil are added to this and the mixture is stirred for 20 mins at 25° C. Next, 2.9 g of iodoethane (18.4 mmol) are added and the reaction mixture is stirred overnight at 25° C. For the workup, acetic acid (concentrated) is slowly added (0.2 eq) and then all volatile components are removed under high vacuum. The residue is dissolved in water and extracted several times with ethyl acetate. Next the organic phase is dried (Na₂SO₄) and concentrated. Purification is effected by silica gel chromatography with the eluent cyclohexane (A)/ethyl acetate (B) (0% B up to 40% B). 2.52 g of a (75:25) mixture of the pyrazole isomers are obtained as a colourless solid. The mixture is separated by preparative HPLC (Kromasil 100 C18 16 μm 250*100 mm, 60/40 methanol/H₂O isocratic, flow rate 800 ml/min) and 1.58 g (48% yield) of 4-bromo-1-ethyl-3-(4-fluorophenyl)-1H-pyrazole and 0.41 g (12% yield) of 4-bromo-1-ethyl-5-(4-fluorophenyl)-1H-pyrazole are obtained.

### Main isomer: 4-bromo-1-ethyl-3-(4-fluorophenyl)-1H-pyrazole [VI-2]

logP (pH 2.7): 3.37

MS (ESI): 269.0 ([M+H]⁺)

¹H-NMR (400 MHz, d₆-DMSO): δ=8.02 (s, 1H), 7.85 (dd, 2H), 7.25 (t, 2H), 4.16 (q, 2H), 1.41 (t, 3H) ppm

### Minor isomer: 4-bromo-1-ethyl-5-(4-fluorophenyl)-1H-pyrazole [VI-3]

logP (pH 2.7): 3.15

MS (ESI): 269.0 ([M+H]⁺)

¹H-NMR (400 MHz, d₆-DMSO): δ=7.62 (s, 1H), 7.48 (dd, 2H), 7.36 (t, 2H), 4.02 (q, 2H), 1.24 (t, 3H) ppm

### 4-Bromo-3-(4-fluorophenyl)-1-isobutyl-1H-pyrazole [VI-4]

### 4-Bromo-5-(4-fluorophenyl)-1-isobutyl-1H-pyrazole [VI-5]

3.62 g (14.9 mmol) of 4-bromo-3-(4-fluorophenyl)-1H-pyrazole are dissolved in 6 mL N,N-dimethylformamide. 0.43 g of sodium hydride (17.9 mmol) as a 60% suspension in oil is added to this and the mixture is stirred for 20 mins at 25° C. Next, 4.1 g of 1-iodo-2-methylpropane (22.4 mmol) are added to this and the reaction mixture is stirred overnight at 25° C. For the workup, acetic acid (concentrated) is slowly added (0.2 eq) and then all volatile components are removed under high vacuum. The residue is dissolved in water and extracted several times with ethyl acetate. Next the organic phase is dried (Na₂SO₄) and concentrated. Purification is effected by silica gel chromatography with the eluent cyclohexane (A)/ethyl acetate (B) (0% B up to 40% B). 3.74 g of a (71:29) mixture of the pyrazole isomers are obtained as a colourless solid. The mixture is separated by preparative HPLC (Kromasil 100 C18 16 μm 250*100 mm, 70/30 methanol/H₂O isocratic, flow rate 800 ml/min) and 2.51 g (56%) of 4-bromo-3 -(4-fluorophenyl)-1-isobutyl-1H-pyrazole and 0.59 g (13% yield) of 4-bromo-5-(4-fluorophenyl)-1-isobutyl-1H-pyrazole are obtained.

### Main isomer: 4-bromo-3-(4-fluorophenyl)-1-isobutyl-1H-pyrazole [VI-4]

logP (pH 2.7): 4.34

MS (ESI): 299.0 ([M+H]⁺)

¹H-NMR (400 MHz, d₆-DMSO): δ=7.99 (s, 1H), 7.85 (dd, 2H), 7.25 (t, 2H), 3.94 (d, 2H), 2.18 (m, 1H), 0.88 (d, 6H) ppm

### Minor isomer: 4-bromo-5-(4-fluorophenyl)-1-isobutyl-1H-pyrazole [VI-5]

logP (pH 2.7): 4.04

MS (ESI): 299.0 ([M+H]⁺)

¹H-NMR (400 MHz, d₆-DMSO): δ=7.64 (s, 1H), 7.48 (dd, 2H), 7.36 (t, 2H), 3.84 (d, 2H), 1.98 (m, 1H), 0.69 (d, 6H) ppm

### 4-Bromo-3-(4-fluorophenyl)-1-(2-methoxyethyl)-1H-pyrazole [VI-6]

### 4-Bromo-5-(4-fluorophenyl)-1-(2-methoxyethyl)-1H-pyrazole [VI-7]

3.62 g (14.9 mmol) of 4-bromo-3-(4-fluorophenyl)-1H-pyrazole are dissolved in 6 mL N,N-dimethyl-formamide. 0.43 g of sodium hydride (17.9 mmol) as a 60% suspension in oil are added to this and the mixture is stirred for 20 mins at 25° C. Next, 3.1 g of 1-bromo-2-methoxyethane (22.4 mmol) is added and the reaction mixture is stirred overnight at 25° C. For the workup, acetic acid (concentrated) is slowly added (0.2 eq) and then all volatile components are removed under high vacuum. The residue is dissolved in water and extracted several times with ethyl acetate. Next the organic phase is dried (Na₂SO₄) and concentrated. Purification is effected by silica gel chromatography with the eluent cyclohexane (A)/ethyl acetate (B) (0% B up to 40% B). 2.91 g of a (76:23) mixture of the pyrazole isomers are obtained as a colourless solid. The mixture is separated by preparative HPLC (Kromasil 100 C18 16 μm 250*100 mm, 62/38 methanol/H₂O isocratic, flow rate 800 ml/min) and 2.92 g (61% yield) of 4-bromo-3-(4-fluorophenyl)-1-(2-methoxyethyl)-1H-pyrazole and 0.43 g (9% yield) of 4-bromo-5-(4-fluorophenyl)-1-(2-methoxyethyl)-1H-pyrazole are obtained.

### Main isomer: 4-bromo-3-(4-fluorophenyl)-1-(2-methoxyethyl)-1H-pyrazole [VI-6]

logP (pH 2.7): 3.08

MS (ESI): 299.0 ([M+H]⁺)¹H-NMR (400 MHz, d₆-DMSO): δ=7.98 (s, 1H), 7.85 (dd, 2H), 7.23 (t, 2H), 4.29 (t, 2H), 3.73 (t, 2H), 3.26 (s, 3H) ppm

### Minor isomer: 4-bromo-5-(4-fluorophenyl)-1-(2-methoxyethyl)-1H-pyrazole [VI-7]

logP (pH 2.7): 2.90

MS (ESI): 299.0 ([M+H]⁺)

¹H-NMR (400 MHz, d₆-DMSO): δ=7.65 (s, 1H), 7.48 (dd, 2H), 7.36 (t, 2H), 4.13 (t, 2H), 3.63 (t, 2H), 3.10 (s, 3H) ppm

The following can be produced by the same process

### 4-Bromo-3-(4-fluorophenyl)-1-methyl-1H-pyrazole [V1-8]

logP (pH 2.7): 2.86

MS (ESI): 257.0 ([M+H]⁺)

¹H-NMR (400 MHz, CD₃CN): δ=7.88-7.84 (m, 2H), 7.65 (s, 1H), 7.21-7.16 (m, 2H), 3.87 (s, 3H) ppm

### 4-Bromo-3-(4-fluorophenyl)-1H-(2-fluorophenyl)ethyl1-1H-pyrazole [VI-9]

logP (pH 2.7): 3.63

MS (ESI): 477.0 ([M+H]⁺)

¹H-NMR (400 MHz, CD3CN): δ=8.15 (s, 1H), 7.88-7.78 (m, 2H), 7.38-7.28 (m, 2H), 4.37 (dd, 1H), 4.31 (dd, 1H), 2.33 (m, 1H), 1.87 (dd, 1H), 1.65 (t, 1H) ppm

### 4-Bromo-1-[(2,2-diehlorocyclopropyl)methyl]-3-(4-fluorophenyl)-1H-pyrazole [VI-10]

logP (pH 2.7): 4.43

MS (ESI): 364.9 ([M+H]⁺)

¹H-NMR (400 MHz, CD₃CN): δ=7.88-7.83 (m, 2H), 7.82 (s, 1H), 7.31-7.29 (m, 2H), 7.27-7.10 (m, 4H), 5.86 (q, 1H), 1.88 (d, 3H) ppm

### 5-(4-Bromo-1-isobutyl-1H-pyrazol-3-yl)-2-fluorobenzonitrile [VI-11]

logP (pH 2.7): 4.15

MS (ESI): 324.1 ([M+H]⁺)

¹H-NMR (400 MHz, d6-DMSO): δ=8.23-8.22 (m, 1H), 8.20-8.17 (m, 1H), 8.16 (s, 1H), 7.65 (t, 1H), 3.97 (d, 2H), 2.15 (q, 1H) ppm

### 3-{[4-Bromo-3-(4-fluorophenyl)-1H-pyrazol-1-yl]methyl}benzonitrile [VI-12]

logP (pH 2.7): 3.93

MS (ESI): 358.0 ([M+H]⁺)

¹H-NMR (400 MHz, CD₃CN): δ=7.88-7.81 (m, 2H), 7.78 (s, 1H), 7.70-7.65 (m, 2H), 7.60-7.51 (m, 2H), 7.22-7.16 (m, 2H), 5.35 (s, 2H) ppm

### 4-Bromo-1-(2-fluorobenzyl)-3-(4-fluorophenyl)-1H-pyrazole [V1-13]

logP (pH 2.7): 4.39

MS (ESI): 349.0 ([M+H]⁺)

¹H-NMR (400 MHz, d6-DMSO): δ=8.18 (s, 1H), 7.83-7.43 (m, 2H), 7.42-7.33 (m, 1H), 7.31-7.19 (m, 5H), 5.43 (s, 2H) ppm

### 4-Bromo-3-(4-fluorophenyl)-1-propyl-1H-pyrazole [VI-14]

logP (pH 2.7): 3.89

MS (ESI): 283.0 ([M+H]⁺)

¹H-NMR (400 MHz, CD₃CN): δ=7.89-7.84 (m, 2H), 7.69 (s, 1H), 7.21-7.15 (m, 2H), 4.08 (t, 2H), 1.90-1.81 (m, 2H), 0.89 (t, 3H) ppm

### 4-Bromo-3-(4-fluorophenyl)-1-[2-(methylsulphanyl)ethyl]-1H-pyrazole [VI-15]

logP (pH 2.7): 3.63

MS (ESI): 315.0 ([M+H]⁺)

¹H-NMR (400 MHz, CD₃CN): δ=7.89-7.84 (m, 2H), 7.75 (s, 1H), 7.22-7.16 (m, 2H), 4.31 (t, 2H), 2.95 (t, 2H), 2.04 (s, 3H) ppm

### Methyl 2-[4-bromo-3-(4-fluorophenyl)-1H-pyrazol-1-yl]-3-methylbutanoate [VI-16]

logP (pH 2.7): 4.27

MS (ESI): 357.0 ([M+H]⁺)

¹H-NMR (400 MHz, CD₃CN): δ=7.88 (s, 1H), 7.87-7.84 (m, 2H), 7.22-7.17 (m, 2H), 4.70 (d, 1H), 3.73 (s, 1H), 2.53 (m, 1H), 1.01 (d, 3H), 0.86 (d, 3.03) ppm

### 4-Bromo-1-(1,3-dioxolan-2-ylmethyl)-3-(4-fluorophenyl)-1H-pyrazole [VI-17]

logP (pH 2.7): 3.01

MS (ESI): 329.0 ([M+H]⁺)

¹H-NMR (400 MHz, CD₃CN): δ=7.89-7.84 (m, 2H), 7.73 (s, 1H), 7.22-7.16 (m, 2H), 5.20 (t, 1H), 4.26 (d, 2H), 3.87 (m, 4H) ppm

### 4-Bromo-1-(cyclopropylmethyl)-3-(4-fluorophenyl)-1H-pyrazole [VI-18]

logP (pH 2.7): 3.90

MS (ESI): 297.0 ([M+H]⁺)

¹H-NMR (400 MHz, CD₃CN): δ=7.90-7.85 (m, 2H), 7.78 (s, 1H), 7.28-7.16 (m, 2H), 3.98 (d, 2H), 1.27 (m,1H), 0.62 (m, 2H), 0.40 (m, 2H) ppm

### 4-Bromo-1-sec-butyl-3-(4-fluorophenyl)-1H-pyrazole [VI-19]

logP (pH 2.7): 4.39

MS (ESI): 299.0 ([M+H]⁺)

¹H-NMR (400 MHz, CD₃CN): δ=7.90-7.85 (m, 2H), 7.71 (s, 1H), 7.21-7.15 (m, 2H), 4.2 (m, 1H), 1.94-1.86 (m, 1H), 1.84-1.74 (m, 1H), 1.45 (d, 3H), 0.70 (t, 3H) ppm

### 4-Bromo-1-(2-ethoxyethyl)-3-(4-fluorophenyl)-1H-pyrazole [VI-20]

logP (pH 2.7): 3.51

MS (ESI): 313.0 ([M+H]⁺)

¹H-NMR (400 MHz, d6-DMSO): δ=8.04 (s, 1H), 7.87-7.82 (m, 2H), 7.32-7.26 (m, 2H), 4.28 (t, 2H), 3.78 (t, 2H), 3.44 (q, 2H), 1.08 (t, 3H) ppm

### 3-[4-Bromo-3-(4-fluorophenyl)-1H-pyrazol-1-yl]butanonitrile [VI-21]

logP (pH 2.7): 3.08

MS (ESI): 310.1 ([M+H]⁺)

¹H-NMR (400 MHz, CD₃CN): δ=7.91-7.88 (m, 2H), 7.83 (s, 1H), 7.23-7.19 (m, 2H), 4.72-4.69 (m, 1H), 3.03-2.95 (m, 2H), 1.60-1.59 (d, 3H) ppm

### Tert-butyl 4-[4-bromo-3-(4-fluorophenyl)-1H-pyrazol-1-yl]piperidin-1-carboxylate [VI-27]

logP (pH 2.7): 4.77

MS (ESI): 368.0 ([M−C₄H₉]⁺)

¹H-NMR (400 MHz, CD₃CN): δ=7.88-7.86 (m, 2H), 7.75 (s, 1H), 7.20-7.17 (m, 2H), 4.35-4.30 (m, 1H), 4.20-4.10 (m, 2H), 3.00-2.85 (m, 2H, br), 2.08-2.05 (m, 2H), 1.88-1.83 (m, 2H), 1.44 (s, 9H) ppm

Other Methods for the Production of Starting Materials of the Formula [VI]:

### 4-Bromo-1-(1-cyclopropylethyl)-3-(4-fluorophenyl)-1H-pyrazole [VI-22]

21.7 g (0.082 mol, 3 eq) of triphenylphosphine are dissolved in 70 mL tetrahydrofuran and cooled to 0° C. by ice-cooling. Under argon 25 mL of a solution of 23.8 g (2 eq, 0.055 mol) of diethyl azodicarboxylate (DEAD) in toluene are added slowly, during which the internal temperature does not exceed 20° C. After 10 mins' stirring, 6.9 g (1 eq, 0.027 mol) of 4-bromo-3-(4-fluorophenyl)-1H-pyrazole and 4.9 g (2 eq, 0.055 mol) of cyclopropylmethylcarbinol, dissolved in 20 mL tetrahydrofuran, are added slowly at 0° C. The reaction mixture is stirred overnight at RT, then evaporated and purified by column chromatography. 1.92 g (22.7%) of 4-bromo-1-(1-cyclo-propylethyl)-3-(4-fluorophenyl)-1H-pyrazole are obtained.

logP (pH 2.7): 4.43

MS (ESI): 309.0 ([M+H]⁺)

¹H-NMR (400 MHz, CD₃CN): δ=7.91-7.85 (m, 2H), 7.80 (s, 1H), 7.22-7.16 (m, 2H), 3.64 (m, 1H), 1.57 (d, 3H), 1.25 (m, 1H) 0.67 (m, 1H), 0.50 (m, 1H), 0.44 (m, 2H) ppm

### 3-[4-Bromo-3-(4-fluorophenyt)-1H-pyrazol-1-yl]propanonitrile [VI-23]

To a solution of 2.5 g of 4-bromo-5-(4-fluorophenyl)-1H-pyrazole (10.4 mmol) in 25 mL DMF are added 5.07 g of Cs₂CO₃ (15.6 mmol) and 3-bromopropanonitrile (2.08 g, 15.6 mmol) and the reaction mixture is stirred overnight at 70° C. After this, the mixture is cooled to room temperature, poured into water and extracted with ethyl acetate. The organic phase is dried, evaporated and purified by preparative HPLC. 2.60 g (85%) of 3-[4-bromo-3-(4-fluorophenyl)-1H-pyrazol-1-yl]propanonitrile are obtained.

logP (pH 2.7): 2.69

MS (ESI): 296.0 ([M+H]⁺)

¹H-NMR (400 MHz, CD₃CN): δ=7.91-7.86 (m, 2H), 7.79 (s, 1H), 7.23-7.17 (m, 2H), 4.38 (t, 2H), 3.73 (t, 2H) ppm

### 4-Bromo-3-(4-fluorophenyl)-1-isopropyl-5-(trifluoromethyl)-1H-pyrazole [VI-24]

To a solution of 1.4 g (4.5 mmol) of 4-bromo-3-(4-fluorophenyl)-5-(trifluoromethyl)-1H-pyrazole in 5 mL DMF are added 0.63 g (4.5 mmol) of K₂CO₃ and 0.66 g (5.4 mmol) of 3-bromopropanonitrile and the reaction mixture is stirred overnight at 80° C. After this, the mixture is cooled to room temperature, poured into water and extracted with diethyl ether. The organic phase is dried, evaporated and purified by chromatography on silica gel (eluent petroleum ether). 0.5 g (32%) of 4-bromo-3-(4-fluorophenyl)-1-isopropyl-5-(trifluoromethyl)-1H-pyrazole are obtained.

logP (pH 2.7): 5.51

MS (ESI): 353.1 ([M+H]⁺)

¹H-NMR (400 MHz, CD₃CN): δ=7.84-7.82 (m, 2H), 7.25-7.22 (m, 2H), 4.78 (q, 1H), 1.51 (d, 6H) ppm

### 4-Bromo-3-(4-fluorophenyl)-1-isopropoxy-1H-pyrazole [VI-25]

To a suspension of 1.26 g (52.7 mmol) of sodium hydride in DMF, 7.5 g (29.3 mmol) of 4-bromo-3-(4-fluorophenyl)-1H-pyrazol-1-ol dissolved in 30 mL DMF are added at 0° C. After the addition, the reaction mixture is stirred for 20 mins at room temperature. Then the mixture is cooled to 0° C. and 4.1 mL (43.9 mmol) of 2-bromopropane are added. After this, the reaction mixture is stirred for 15 hrs at room temperature, then poured into 500 mL of ice-water and extracted 3× with 150 mL ethyl acetate. The combined organic phases are washed with water, dried over Na₂SO₄ and evaporated under vacuum. The crude material is purified by column chromatography on silica gel (eluent 2% ethyl acetate/petroleum ether) and then by preparative HPLC. 1.2 g of 4-bromo-3-(4-fluorophenyl)-1-isopropoxy-1H-pyrazole (13.7%) are obtained.

logP (pH 2.7): 4.11

MS (ESI): 301.1 ([M+H]⁺)

¹H-NMR (400 MHz, d6-DMSO): δ=8.23 (s, 1H), 7.84-7.80 (m, 2H), 7.32-7.28 (m, 2H), 4.68 (q, 1H), 1.27 (d, 6H) ppm

### 4-Bromo-1-cyclopropyl-3-(4-fluorophenyl)-1H-pyrazole [VI-26]

To a suspension of 4.8 g (27.2 mmol) of N-bromosuccinimide in 250 mL dichloromethane, 5 g (20.2 mmol) of 1-cyclopropyl-3-(4-fluorophenyl)-1H-pyrazole are added at 10° C. After the addition, the reaction mixture is stirred for 1 hr at room temperature. After this, the reaction mixture is treated with water and extracted with dichloromethane. The combined organic phases are washed with water, dried over Na₂SO₄ and evaporated under vacuum. The crude material is purified by column chromatography on silica gel. 5 g of 4-bromo-1-cyclopropyl-3-(4-fluorophenyl)-1H-pyrazole (73%) are obtained.

logP (pH 2.7): 3.59

MS (ESI): 281.0 ([M+H]⁺)

¹H-NMR (400 MHz, CD₃CN): δ=7.88-7.84 (m, 2H), 7.75 (s, 1H), 7.20-7.16 (m, 2H), 3.68-3.65 (m, 1H), 1.12-1.09 (m, 2H), 1.04-1.01 (m, 2H) ppm

Production of Starting Materials of the Formula [V] by process V2:

### 3-(4-Fluorophenyl)-1-isopropyl-4-(4,4,5,5-tetramethyl-1,3,2-dioxaborolan-2-yl)-1H-pyrazole [V-1]

3.0 g (10.5 mmol) of 4-bromo-3-(4-fluorophenyl)-1-isopropyl-1H-pyrazole and 5.38 g (21.1 mmol) of bis-(pinacolato)-diborane are dissolved in 30 mL dimethyl sulphoxide. To this are added 3.1 g of potassium acetate (31.8 mmol) and 0.86 g (1.06 mmol) of 1,1′-bis(diphenylphosphino)ferrocene]-diehloropalladium(II) *CH₂Cl₂ and the reaction mixture is heated under a current of argon for 5 hrs at 85° C. After renewed heating for 2 hrs at 80° C. the reaction mixture is cooled and the dimethyl sulphoxide removed under high vacuum. The residue is dissolved in water and extracted several times with ethyl acetate. Next the organic phase is dried (Na₂SO₄) and concentrated. Purification is effected by silica gel chromatography with the eluent cyclohexane (A)/20% ethyl acetate in cyclohexane (B) (0% B up to 70% B). 3.85 g of 3-(4-fluorophenyl)-1-isopropyl-4-(4,4,5,5-tetramethyl-1,3,2-dioxaborolan-2-yl)-1H-pyrazole are obtained as a colourless solid (40% purity by NMR). The compound is reacted further without further purification.

logP (pH 2.7): 4.51

MS (ESI): 331.20 ([M+H]⁺)

¹H-NMR (400 MHz, d₃-CD₃CN): δ=7.90 (dd, 2H), 7.78 (s, 1H), 7.10 (dd, 1H), 4.52 (m, 1H), 1.49 (d, 6H), 1.25 (s, 12H) ppm

### 3-(4-Fluorophenyl)-1-(2-methoxyethyl)-4-(4,4,5,5-tetramethyl-1,3,2-dioxaborolan-2-yl)-1H-pyrazole [V-2]

1.0 g (3.3 mmol) of 4-bromo-3-(4-fluorophenyl)-1-(2-methoxyethyl)-1H-pyrazole and 1.69 g (2 eq, 6.7 mmol) of bis-(pinacolato)-diborane are dissolved in 15 mL dimethyl sulphoxide. To this are added 0.98 g of potassium acetate (10 mmol) and 0.27 g (0.3 mmol) of 1,1′-bis(diphenylphosphino)-ferrocene]dichloro-palladium(II)*CH₂Cl₂ and the reaction mixture is heated under a current of argon for 7 hrs at 85° C. After this, the reaction mixture is cooled and the dimethyl sulphoxide removed under high vacuum. The residue is dissolved in water and extracted several times with ethyl acetate. Next the organic phase is dried (Na₂SO₄) and concentrated. Purification is effected by silica gel chromatography with the eluent cyclohexane (A)/20% ethyl acetate in cyclohexane (B) (0% B up to 70% B). 0.39 g of 3-(4-fluorophenyl)-1-(2-methoxyethyl)-4-(4,4,5,5-tetramethyl-1,3,2-dioxaborolan-2-yl)-1H-pyrazole is obtained as a colourless solid (60% purity by NMR). The compound is reacted further without further purification.

logP (pH 2.7): 3.58

MS (ESI): 347.21 ([M+H]⁺)

¹H-NMR (400 MHz, d₃-CD₃CN): δ=7.80 (dd, 2H), 7.58 (s, 1H), 7.15 (dd, 1H), 4.30 (m, 2H), 3.75 (m, 2H), 3.30 (s, 3H), 1.28 (s, 12H) ppm

### 3-(4-Fluorophenyl)-1-isobutyl-4-(4,4,5,5-tetramethyl-1,3,2-dioxaborolan-2-yl)-1H-pyrazole [V-3]

2.0 g (6.7 mmol) of 4-bromo-3-(4-fluorophenyl)-1-isobutyl-1H-pyrazole and 3.4 g (13.4 mmol) of bis-(pinacolato)-diborane are dissolved in 30 mL dimethyl sulphoxide. To this are added 1.98 g of potassium acetate (20 mmol) and 0.55 g (0.67 mmol) of 1,1′-bis(diphenylphosphino)-ferrocene]dichloropalladium(H)*CH₂Cl₂ and the reaction mixture is heated under a current of argon for 7 hrs at 85° C. After this, the reaction mixture is cooled and the dimethyl sulphoxide removed under high vacuum. The residue is dissolved in water and extracted several times with ethyl acetate. Next the organic phase is dried (Na₂SO₄) and concentrated. Purification is effected by silica gel chromatography with the eluent cyclohexane (A)/20% ethyl acetate in cyclohexane (B) (0% B up to 70% B). 1.1 g of 3-(4-fluorophenyl)-1-isobutyl-4-(4,4,5,5-tetramethyl-1,3,2-dioxaborolan-2-yl)-1H-pyrazole is obtained as a colourless solid (23% purity by NMR). The compound is reacted further without further purification.

logP (pH 2.7): 4.76

MS (ESI): 345.17 ([M+H]⁺)

¹H-NMR (400 MHz, d3-CD₃CN): δ=7.90 (dd, 2H), 7.72 (s, 1H), 7.10 (dd, 1H), 3.95 (d, 2H), 2.20 (m, 1H), 1.30 (s, 12H), 0.90 (d, 6H) ppm

Analogously to the method described above, the following compounds of the type [III] can also be prepared:

### 1-(2,2-Ditluoroethyl)-3-(4-fluorophenyl)-4-(4,4,5,5-tetramethyl-1,3,2-dioxaborolan-2-yl)-1H-pyrazole [V-4]

logP (pH 2.7): 3.84

MS (ESI): 353.2 ([M+H]⁺)

¹H-NMR (400 MHz, d6-DMSO): δ=8.06 (s, 1H), 7.90-7.87 (m, 2H), 7.21 (m, 2H), 6.41 (m, 1H), 4.68 (m, 2H), 1.27 (s, 12H) ppm

### 3-(4-Fluorophenyl)-1-isopropoxy-4-(4,4,5,5-tetramethyl-1,3,2-dioxaborolan-2-yl)-1H-pyrazole [V-5]

logP (pH 2.7): 4.81

MS (ESI): 347.2 ([M+H]⁺)

¹H-NMR (400 MHz, CD₃CN): δ=7.89-7.85 (m, 2H), 7.68 (s, 1H), 7.15-7.10 (m, 2H), 4.71 (m, 1H), 1.29 (m, 18H) ppm

### 1-(Cyclopentyloxy)-3-(4-fluorophenyl)-4-(4,4,5,5-tetramethyl-1,3,2-dioxaborolan-2-yl)-1H-pyrazole [V-6]

logP (pH 2.7): 5.51

MS (ESI): 373.2 ([M+H]⁺)

¹H-NMR (400 MHz, CD₃CN): δ=7.89-7.85 (m, 2H), 7.67 (s, 1H), 7.15-7.10 (m, 2H), 1.95-1.93 (m, 7H), 1.80-1.79(m, 2H) ppm

### 1-Cyclopropyl-3-(4-fluorophenyl)-4-(4,4,5,5-tetramethyl-1,3,2-dioxaborolan-2-yl)-1H-pyrazole [V-7]

To a solution of 2 g (7.11 mmol) of 4-bromo-1-cyclopropyl-3-(4-fluorophenyl)-1H-pyrazole and 1.98 g (10.67 mmol) of 2-isopropoxy-4,4,5,5-tetramethyl-1,3,2-dioxaborolane in dry tetrahydrofuran (40 mL) under argon a solution of n-butyllithium in n-hexane (1 eq) is slowly added at −78° C. The reaction mixture is stirred for 5 mins at −78° C. and then treated with aqueous NH₄Cl solution. After warming of the reaction mixture to room temperature, the reaction mixture is extracted with ethyl acetate. The combined organic extracts are dried and evaporated under vacuum. The crude material obtained is purified by chromatography on silica gel (eluent n-hexane/dichloromethane 2:1). 900 mg (39%) of 1-cyclopropyl-3-(4-fluorophenyl)-4-(4,4,5,5-tetramethyl-1,3,2-dioxaborolan-2-yl)-1H-pyrazole are obtained.

logP (pH 2.7): 3.47

MS (ESI): 329.2 ([M+H]⁺)

¹H-NMR (400 MHz, CD₃CN): δ=7.89-7.86 (m, 2H), 7.81 (s, 1H), 7.13-7.09 (m, 2H), 3.65 (m, 1H), 1.28 (s, 12H), 1.10 (m, 2H), 1.00 (m, 2H) ppm

Analogously to the method described above, the following compounds of the type [III] can also be prepared by metallation of the pyrazole:

### 1-(Cyclopropylmethyl)-3-(4-fluorophenyl)-4-(4,4,5,5-tetramethyl-1,3,2-dioxaborolan-2-yl)-1H-pyrazole [V-8]

logP (pH 2.7): 4.42

MS (ESI): 343.2 ([M+H]⁺)

¹H-NMR (400 MHz, d6-DMSO): δ=8.01 (s, 1H), 7.90-7.87 (m, 2H), 7.19 (m, 2H), 4.00 (d, 2H), 0.39-0.55 (m, 5H) ppm

### 3-(4-Fluorophenyl)-1-methyl-4-(4,4,5,5-tetramethyl-1,3,2-dioxaborolan-2-yl)-1H-pyrazole [V-9]

logP (pH 2.7): 3.47

MS (ESI): 303.2 ([M+H]⁺)

¹H-NMR (400 MHz, d6-DMSO): δ=7.95 (s, 1H), 7.90-7.86 (m, 2H), 7.19 (m, 2H), 3.87 (s, 3H), 1.26 (s, 12H) ppm

### 1-(2-Chloroethyl)-3-(4-fluorophenyl)-4-(4,4,5,5-tetramethyl-1,3,2-dioxaborolan-2-yl)-1H-pyrazole [V-10]

logP (pH 2.7): 4.06

MS (ESI): 351.1 ([M+H]⁺)

¹H-NMR (400 MHz, d6-DMSO): δ=8.06 (s, 1H), 7.91-7.87 (m, 2H), 7.22-7.18 (m, 2H), 4.49 (t, 2H), 4.03 (t, 2H), 1.27 (s, 12H) ppm

Production of Starting Materials of the Formula [IV-c]:

### 4-[3-(4-Fluorophenyl)-1-isopropyl-1H-pyrazol-4-yl]pyridin-2-amine [IV-c-1]

500 mg (2.9 mmol) of 4-bromopyridin-2-amine and 450 μL (3.2 mmol) of triethylamine are dissolved in 25 mL tetrahydrofuran. To this are added 338 μL of 2-methylpropanoyl chloride (2.9 mmol) and the reaction mixture is stirred for 16 hrs at room temperature. Next, the volatile components are removed under vacuum and the crude material treated with 3 mL NH₃ in methanol (7 molar). The mixture is stirred for 16 hrs at room temperature and then evaporated. The crude product is purified by silica gel chromatography (eluent cyclohexane/ethyl acetate). 382 mg (47% yield) of N-(4-bromopyridin-2-yl)-2-methylpropanamide are obtained as a colourless solid.

logP (pH 2.7): 2.09

MS (ESI): 244.9 ([M+H]⁺)

¹H-NMR (400 MHz, d₃-CD₃CN): δ=8.70 (s, 1H, br), 8.40 (d, 1H), 8.12 (d, 1H), 7.25 (dd, 1H), 2.65 (m, 1H), 1.15 (d, 6H) ppm

Production of Starting Materials of the Formula [III] by Process (V3):

### 4-[3-(4-Fluorophenyl)-1-isopropyl-1H-pyrazol-4-yl]pyridin-2-amine [III-1]

200 mg (0.6 mmol) of 3-(4-fluorophenyl)-1-isopropyl-4-(4,4,5,5-tetramethyl-1,3,2-dioxaborolan-2-yl)-1H-pyrazole and 166 mg (0.72 mmol) of tert-butyl (4-chloropyridin-2-yl)carbamate are dissolved in 3 mL 1,4-dioxan. To this are added 44.7 mg of bis(tricyclohexylphosphine)palladium(II) dichloride (0.06 mmol) and 2 mL sodium carbonate solution (2 molar). The reaction mixture is flushed with argon for 5 mins and then sealed. Next the mixture is heated for 12 mins at 150° C. in the microwave (CEM Explorer). After cooling, insoluble components are filtered off and the salt residue washed with 1,4-dioxan. The organic phase is evaporated and the crude product purified by silica gel chromatography (eluent cyclohexane/ethyl acetate). 45.4 mg (25% yield) of 4-[3-(4-fluorophenyl)-1-isopropyl-1H-pyrazol-4-yl]pyridin-2-amine are obtained as a colourless solid.

logP (pH 2.7): 1.22

MS (ESI): 297.13 ([M+H]⁺)

¹H-NMR (400 MHz, d₃-CD₃CN): δ=7.80 (m, 2H), 7.50 (dd, 2H), 7.10 (dd, 1H), 6.47 (d, 1H), 6.39 (s, 1H), 5.10 (s, 2H, br), 4.53 (m, 1H), 1.20 (d, 6H) ppm

Production of Starting Materials of the Formula [III] by Process (V26):

### 4-[3-(4-Fluorophenyl)-1-(2-methoxyethyl)-1H-pyrazol-4-yl]pyridin-2-amine [III-2]

257 mg (0.86 mmol) of 4-bromo-3-(4-fluorophenyl)-1-(2-methoxyethyl)-1H-pyrazole and 303 mg (0.94 mmol) of tert-butyl-[4-(4,4,5,5-tetramethyl-1,3,2-dioxaborolan-2-yl)pyridin-2-yl]carbamate are dissolved in 4 mL 1,4-dioxan. To this are added 50.8 mg of bis(tricyclohexylphosphine)-palladium(II) dichloride (0.04 mmol) and 2 mL sodium carbonate solution (2 M in H2O). The reaction mixture is flushed for 5 mins with argon and then sealed. Next the mixture is heated for 12 mins at 150° C. in the microwave (CEM Explorer). After cooling, insoluble components are filtered off and the salt residue washed with 1,4-dioxan. The organic phase is evaporated and the crude product purified by silica gel chromatography (eluent dichloromethane/10% methanol-dichloromethane). 255 mg (86% yield) of 4-[3-(4-fluorophenyl)-1-(2-methoxyethyl)-1H-pyrazol-4-yl]pyridin-2-amine are obtained as a colourless solid.

logP (pH 2.7): 0.98

MS (ESI): 313.15 ([M+H]⁺)

¹H-NMR (400 MHz, d3-CD₃CN): δ=7.85 (d, 1H), 7.77 (s, 1H), 7.48-7.46 (m, 2H), 7.12-7.09 (m, 2H), 6.44 (dd, 1H), 6.37 (s, 1H), 4.79 (s, 2H, br), 4.29 (t, 21-1), 3.77 (t, 2H ), 3.31 (s, 3H) ppm

Analogously to the method described, the following compounds of the type [III] can also be prepared:

### 4-[1-Ethyl-3-(4-fluorophenyl)-1H-pyrazol-4-yl]pyridin-2-amine [III-3]

logP (pH 2.7): 0.97

MS (ESI): 283.32 ([M+H]⁺)

¹H-NMR (400 MHz, d3-CD₃CN): δ=7.85 (m, 1H), 7.77 (s, 1H), 7.48-7.46 (m, 2H), 7.12-7.09 (m, 2H), 6.45 (dd, 1H), 6.37 (s, 1H), 4.82 (s, 2H, br), 4.20 (q, 211), 1.49 (t, 3H) ppm

### 4-[1-(2,2-Difluoroethyl)-3-(4-fluorophenyl)-1H-pyrazol-4-yl]pyridin-2-amine [III-4]

logP (pH 2.7): 1.03

MS (ESI): 319.48 ([M+H]⁺)

¹H-NMR (400 MHz, d3-CD₃CN): δ=7.86 (d, 1H), 7.82 (s, 1H), 7.48-7.46 (m, 2H), 7.12-7.09 (m, 2H), 6.44 (dd, 1H), 6.37 (s, 1H), 6.26 (td, 1H), 4.87 (s, 1H), br), 4.57 (dt, 2H) ppm

### 4-[3-(4-Fluorophenyl)-1-methyl-1H-pyrazol-4-yl]pyridin-2-amine [III-5]

logP (pH 2.7): 0.71

MS (ESI): 269.2 ([M+H]⁺)

¹H-NMR (400 MHz, d6-DMSO): δ=7.95 (s, 1H), 7.81 (m, 1H), 7.45-7.42 (m, 2H), 7.21-7.18 (m, 2H), 6.31-6.29 (m, 2H), 5.80 (s, 2H, br), 3.90 (s, 3H) ppm

Production of Intermediates of the Formula [XV-a]:

### 2-Methoxy-N-[4-(4,4,5,5-tetramethyl-1,3,2-dioxaborolan-2-yl)pyridin-2-yl]acetamide [XV-a-1]

2.0 g (8.5 mmol) of N-(4-bromopyridin-2-yl)-2-methoxyacetamide and 2.4 g (9.3 mmol) of bis-(pinacolato)-diborane are dissolved in 50 mI, dry dioxan. To this are added 2.50 g of potassium acetate (25.5 mmol) and 0.31 g (0.38 mmol) of 1,1′-bis(diphenylphosphino)ferrocene]dichloro-palladium(H)*CH₂Cl₂ and the reaction mixture is heated under a current of argon for 3 hrs at 80° C. After this, the reaction mixture is cooled, water added and extracted several times with ethyl acetate. Next the organic phase is dried (Na₂SO₄) and concentrated. Purification is effected by silica gel chromatography with the eluent hexane/ether (3:1). 1.32 g (53% yield) of 2-methoxy-N-[4-(4,4,5,5-tetramethyl-1,3,2-dioxaborolan-2-yl)pyridin-2-yl]acetamide are obtained as a colourless solid.

logP (pH 2.7): −0.18

MS (ESI): 211.13 ([M(-pinacol)+H]⁺)

¹H-NMR (400 MHz, d₃-CD₃CN): δ=8.75 (s, 1H, br), 8.38 (s, 1H), 8.33 (d, 1H), 7.33 (m, 1H), 4.00 (s, 2H), 3.46 (s, 3H), 1.34 (s, 12H) ppm

### N-[4-(4,4,5,5-Tetramethyl-1,3,2-dioxaborolan-2-yl)pyridin-2-yl]propanamide [XV-a-2]

1.80 g (7.9 mmol) of N-(4-bromopyridin-2-yl)propanamide and 2.19 g (8.6 mmol) of bis-(pinacolato)-diborane are dissolved in 50 mL dry dioxan. To this are added 2.31 g of potassium acetate (23.6 mmol) and 0.35g (0.43 mmol) of 1,1′-bis(diphenylphosphino)ferrocene]dichloro-palladium(II)*CH₂Cl₂ and the reaction mixture is heated under a current of argon for 3 hrs at 80° C. After this, the reaction mixture is cooled, water added and extracted several times with ethyl acetate. Next the organic phase is dried (Na₂SO₄) and concentrated. Purification is effected by silica gel chromatography with the eluent hexane/ether (3:1). 0.870 g (36% yield) of N-[4-(4,4,5,5-tetramethyl-1,3,2-dioxaborolan-2-yl)pyridin-2-yl]propanamide are obtained as a colourless solid.

¹H-NMR (400 MHz, d₃-CD₃CN): δ=8.55 (s, 1H, br), 8.37 (s, 1H), 8.28 (d, 1H), 7.27 (m, 1H), 2.43 (q, 2H), 1.34 (s, 12H), 1.15 (t, 3H) ppm

### 2-Phenyl-N-[4-(4,4,5,5-tetramethyl-1,3,2-dioxaborolan-2-yl)pyridin-2-yl]acetamide [XV-a-3]

1.40 g (4.81 mmol) of N-(4-bromopyridin-2-yl)-2-phenylacetamide and 1.34 g (5.3 mmol) of bis-(pinacolato)-diborane are dissolved in 50 mL dry dioxan. To this are added 1.42 g of potassium acetate (14.3 mmol) and 0.18 g (0.22 mmol) of 1,1′-bis(diphenylphosphino)ferrocene]dichloro-palladium-(II)*CH₂Cl₂ and the reaction mixture is heated under a current of argon for 3 hrs at 80° C. After this, the reaction mixture is cooled, water added and extracted several times with ethyl acetate. Next the organic phase is dried (Na₂SO₄) and concentrated. Purification is effected by trituration of the product with hexane/ether (3:1). 0.87 g (54% yield) of 2-phenyl-N-[4-(4,4,5,5-tetramethyl-1,3,2-dioxaborolan-2-yl)pyridin-2-yl]acetamide are obtained as a colourless solid.

¹H-NMR (400 MHz, d₃-CD₃CN): δ=8.68 (s, 1H, br), 8.33 (s, 1H), 8.28 (d, 1H), 7.36 (m, 5H), 7.27 (m, 1H), 3.72 (s, 2H), 1.32 (s, 12H) ppm

The following intermediates of the type [XV-a] can also be produced analogously:

### 2-Methyl-N-[4-(4,4,5,5-tetramethyl-1,3,2-dioxaborolan-2-yl)pyridin-2-yl]propanamide [XV-a-4]

logP (pH 2.7): 0.04

MS (ESI): 209.1 ([M−C6H12]⁺)

¹H-NMR (400 MHz, CD₃CN): δ=8.39 (s, 1H), 8.29 (d, 1H), 7.28 (d, 1H), 1.94 (m, 1H), 1.34 (s, 12H), 1.17 (d, 6H) ppm

### 2-Cyclopropyl-N-[4-(4,4,5,5-tetramethyl-1,3,2-dioxaborolan-2-yl)pyridin-2-yl]acetamide [XV-a-5]

logP (pH 2.7): 0.27

MS (ESI): 221.1 ([M−C6H12]⁺)

¹H-NMR (400 MHz, CD₃CN): δ=8.60 (s, 1H, br), 8.39 (s, 1H), 8.29 (d, 1H), 7.28 (d, 1H) 2.29 (d, 2H), 1.34 (s, 12H), 1.10 (m, 1H), 0.57 (m, 2H), 0.25 (m, 2H) ppm

### Ethyl [4-(4,4,5,5-tetramethyl-1,3,2-dioxaborolan-2-yl)pyridin-2-yl]carbamate [XV-a-6]

logP (pH 2.7): 0.00

MS (ESI): 211.1 ([M−C6H12]⁺)

¹H-NMR (400 MHz, CD₃CN): δ=8.28 (m, 2H), 8.18 (s, 1H), 7.24 (d, 1H), 1.94 (m, 1H), 4.21 (q, 2H), 1.34 (s, 12H), 1.29 (t, 3H) ppm

N-[4-(4,4,5,5-Tetramethyl-1,3,2-dioxaborolan-2-yl)pyridin-2-yl]cyclopropanecarboxamide [XV-a-7]

logP (pH 2.7): 0.00

MS (ESI): 207.1 ([M−C6H12]⁺)

¹H-NMR (400 MHz, CD₃CN): δ=8.36 (s, 1H), 8.29 (d, 1H), 7.27 (d, 1H), 1.80 (m, 1H), 1.33 (s, 12H), 0.93 (m, 3H), 0.84 (m, 2H) ppm

Production of Intermediates of the Formula [XIII]:

### 4-Bromo-1-(tetrahydro-2H-pyran-2-yl)-1H-pyrazole [XIII-1]

234.7 g of bromine (75.26 ml, 1.473 mol) are added dropwise to a solution of pyrazole (100 g, 1.47 mol) in H₂O (400 ml) preheated to 40° C. The reaction solution is heated and stirred for 30 mins under reflux (TLC, hexane:EtOAc 1:1, R_{f}=0.6). After cooling of the reaction solution (pH=3) to room temperature, conc. NaOH_{(aq)} is added dropwise and the pH adjusted to 8 (deposition of a white precipitate). The suspension obtained is filtered, and the residue washed with ice-cold H₂O (150 ml) and then dried under vacuum. 195.47 g of the intermediate 4-bromo-1H-pyrazole (91% yield, purity level 99%) are obtained as a white solid which is reacted further without further purification.

A suspension of 4-bromo-1H-pyrazole (181 g, 1.23 mol), 3,4-dihydro-2H-pyran (155.5 g, 168.6 ml, 1.85 mol) and trifluoroacetic acid (0.84 g, 0.57 ml, 7.40 mmol) is heated and stirred under reflux for 5 hrs. Next, the crude product obtained after addition of NaH (1.18 g, 0.05 mol) is fractionally distilled. 253 g of 4-bromo-1-(tetrahydro-2H-pyran-2-yl)-1H-pyrazole (89%) are obtained as a colourless liquid (B.Pt. 88-90° C. at a pressure of 0.02 mm Hg).

The spectroscopic data correspond to the data described in the literature (Acta Chem. Scand. Series B: Organic Chemistry and Biochemistry 1982, 36, 2, 101-108)

1H-NMR (400 MHz, d₃-CD₃CN): δ=7.78 (s, 1H), 7.47 (s, 1H), 5.33 (dd, 1H), 3.95 (m, 1H), 3.65 (m, 1H), 2.10-2.00 (m, 2H), 1.70-1.50 (m, 4H) ppm

Production of Intermediates of the Formula [XII]:

### 4-[1-(Tetrahydro-2H-pyran-2-yl)-1H-pyrazol-4-yl]pyridine [XII-1]

4.88 g of Pd(PPh₃)₄ (4.22 mmol, 2.5 mol %) are added to a suspension of 39.0 g of 4-bromo-1-(tetrahydro-2H-pyran-2-yl)-1H-pyrazole (0.17 mol), 675 mL aqueous Na₂CO₃ (2.0 molar in H₂O, 1.35 mol) and 25.1 of 4-pyridineboronic acid (0.21 mol) in dioxan (2000 mL). The reaction mixture is heated at 80° C. under a blanket gas atmosphere and under reflux and stirred for 41 hrs. Next, the reaction was treated with H₂O (50 mL). The reaction solution is concentrated to ¼ of the volume and extracted with ethyl acetate (3×300 mL). The combined organic phases are washed with saturated NaCl solution and then dried with MgSO₄. The crude product obtained is purified by Kugelrohr distillation (B.Pt. 130-135° C. at p=0.02 mm Hg). 26.37 g are obtained (, up to a purity level of 97.3% could be achieved. 26.37 g of 4-[1-(tetrahydro-2H-pyran-2-yl)-1H-pyrazol-4-yl]pyridine (68%) were obtained as a yellow highly viscous oil.

logP (pH 2.7): 0.38

MS (ESI): 230.1 ([M+H]⁺)

1H-NMR (400 MHz, d₃-CD₃CN): δ=8.49 (d, 2H), 8.20 (s, 1H), 7.94 (s, 1H), 7.48 (d, 2H), 5.39 (dd, 1H), 4.00 (m, 1H), 3.69 (m, 1H), 2.10-2.00 (m, 211), 1.70-1.50 (m, 4H) ppm

Production of Intermediates of the Formula [XI]:

### 4-[1-(Tetrahydro-2H-pyran-2-yl)-5-(tributylstannyl)-1H-pyrazol-4-yl]pyridine [XI-1]

13.5 mL of n-butyllithium (2.5 molar in n-hexane, 33.75 mmol) are added to a solution of 7.0 g of 4-[1-(tetrahydro-2H-pyran-2-yl)-1H-pyrazol-4-yl]pyridine (30.5 mmol) in dry THF (200 mL) at −70° C. under a blanket gas atmosphere and with stirring. After completion of the addition, the mixture is stirred for a further hour at this temperature. After this, 9.5 g of tri-n-butyltin chloride (29.2 mmol) are added. Next, the reaction mixture is allowed to warm to room temperature and then stirred for a further 15 mins at this temperature. All volatile components are evaporated under vacuum and the residue is distilled under high vacuum (<0.1 mbar). The fraction with a boiling point over 130° C. is isolated and purified further by chromatography (n-hexane/diethyl ether=1:4 eluent). 7.5 g of 4-[1-(tetrahydro-2H-pyran-2-yl)-5-(tributylstannyl)-1H-pyrazol-4-yl]pyridine (45%) are obtained.

logP (pH 2.7): 5.09

MS (ESI): 520.1 ([M+H]⁺)

¹H-NMR (400 MHz, d₃-CD₃CN): δ=8.50 (d, 2H), 8.20 (s, 1H), 7.94 (s, 1H), 7.48 (d, 2H), 5.40 (dd, 1H), 4.00 (m, 1H), 3.68 (m, 1H), 2.10-2.00 (m, 2H), 1.70-1.50 (m, 10H), 1.40-1.30 (m, 6H), 1.10-1.00 (m, 6H), 0.89 (t, 9H) ppm

Production of Intermediates of the Formula [X]:

### 4-[5-(4-Fluorophenyl)-1-(tetrahydro-2H-pyran-2-yl)-1H-pyrazol-4-yl]pyridine [X-1]

250 mg (0.48 mmol) of 4-[1-(tetrahydro-2H-pyran-2-yl)-5-(tributylstannyl)-1H-pyrazol-4-yl]pyridine and 126 mg (0.72 mmol) of 4-bromofluorobenzene are stirred in 3 mL dimethylformamide. To this are added 146 mg of caesium fluoride (0.96 mmol), 84 mg of tetrakis(triphenylphosphine)-palladium(0) (0.07 mmol, 15 mol %) and 9 mg of copper(I) iodide (0.05 mmol, 10 mol. %) and the mixture is degassed for 5 mins with blanket gas. After this the mixture is heated at 150° C. for 20 mins in the microwave (CEM Discover). Next, the crude mixture is filtered through a cartridge with Celite and the volatile components removed under vacuum. The crude product is purified by chromatography on silica gel (cyclohexane/ethyl acetate) and 47.4 mg of 4-[5-(4-fluorophenyl)-1-(tetrahydro-2H-pyran-2-yl)-1H-pyrazol-4-yl]pyridine (30%) and 41 mg of the cleaved product 4-[3-(4-fluorophenyl)-1H-pyrazol-4-yl]pyridine [IX-1] (35%) are obtained as a colourless oil.

### 4-[5-(4-fluorophenyl)-1-(tetrahydro-2H-pyran-2-yl)-1H-pyrazol-4-yl]pyridine [X-1]

logP (pH 2.7): 1.23

MS (ESI): 324.18 ([M+H]⁺)

¹H-NMR (400 MHz, d₃-CD₃CN): δ=8.36 (dd, 2H), 7.90 (s, 1H), 7.42 (dd, 2H), 7.26 (dd, 2H), 7.09 (dd, 2H), 5.01 (dd, 1H), 3.96 (m, 1H), 2.40 (m, 1H), 1.82 (m, 1H), 1.70-1.45 (m, 3H), 1.35-1.25 (m, 1H) ppm

Production of Intermediates of the Formula [IX]:

### 4-[3-(4-Fluorophenyl)-1H-pyrazol-4-yl]pyridine [IX-1]

The intermediates produced in the general procedure V11 can also be used in the deprotection reaction without further purification.

Analogously to the procedure described above (V11), 750 mg (1.45 mmol) of 4-[1-(tetrahydro-2H-pyran-2-yl)-5-(tributylstannyl)-1H-pyrazol-4-yl]pyridine, 380 mg (2.17 mmol) of 4-bromofluoro-benzene, 440 mg of caesium fluoride (2.89 mmol), 250 mg of tetrakis(triphenylphosphine)-palladium(0) (0.28 mmol, 15 mol %) and 28 mg of copper(I) iodide (0.15 mmol, 10 mol %) are reacted. After removal of the insoluble components by filtration over Celite and removal of the volatile components under vacuum, 950 mg of a crude product are obtained.

The crude product is dissolved in 5 mL methanol and treated with 5.8 mL of HCl in dioxin (4 molar). The solution is stirred at room temperature for 1.5 hrs and then concentrated. The solid obtained is triturated several times with diethyl ether and 387 mg of 4-[3-(4-fluorophenyl)-1H-pyrazol-4-yl]pyridine hydrochloride (75%) are obtained as a white solid. From this, the free 4-[3-(4-fluorophenyl)-1H-pyrazol-4-yl]pyridine can be obtained in the form of the salt-free pyrazole by dissolving the hydrochloride in ethyl acetate and washing with sodium carbonate.

### 4-[3-(4-Fluorophenyl)-1H-pyrazol-4-yl]pyridine hydrochloride [IX-1]

logP (pH 2.7): 0.65

MS (ESI): 240.11 ([M+H]⁺)

¹H-NMR (400 MHz, d₃-CD₃CN): δ=11.3 (s, 0.5H), 8.44 (dd, 2H), 7.89 (s, 1H, br), 7.45 (dd, 2H), 7.40 (s, 0.5H, br), 7.23 (dd, 2H), 7.15 (m, 2H) ppm

Production of Compounds of the Formula [I] by Process (V7):

### 4-[3-(4-Fluorophenyl)-1-isopropyl-1H-pyrazol-4-yl]quinoline [I-1]

79 mg (0.24 mmol) of 3-(4-fluorophenyl)-1-isopropyl-4-(4,4,5,5-tetramethyl-1,3,2-dioxaborolan-2-yl)-1H)-pyrazole and 59 mg (0.36 mmol) of 4-chloroquinoline are dissolved in 2.5 mL 1,4-dioxan. To this are added 17.7 mg of 1,1′-bis(diphenylphosphino)ferrocene]dichloropalladium(II)*CH₂Cl₂ (0.01 mmol) and 0.5 mL sodium carbonate solution (2 molar). The reaction mixture is flushed with argon for 5 mins and then sealed. Next the mixture is heated for 12 mins at 150° C. in the microwave (CEM Explorer). After cooling, insoluble components are filtered off over Celite and the residue washed with 1,4-dioxan. The organic phase is evaporated and the crude product purified by preparative HPLC (XTerra 125×19 mm, 5 μm, gradient: 0-1.5 mins 80% water, 15% methanol, 5% aqueous 10% NH₄HCO₃-soln, 1.5-10.0 mins linear gradient up to 0% water, 95% methanol, 5% aqueous 10% NH₄HCO₃-soln, 10.0-15.0 mins 0% water, 95% methanol, 5% aqueous 10% NH₄HCO₃-soln). 25 mg (22%) of 4-[3-(4-fluorophenyl)-1-isopropyl-1H-pyrazol-4-yl]quinoline are obtained as a colourless solid.

logP (pH 2.7): 2.23

MS (ESI): 332.07 ([M+H]⁺)

¹H-NMR (400 MHz, d₆-DMSO): δ=8.86 (d, 1H), 8.15 (s, 1H), 8.05 (d, 1H), 7.75-7.70 (m, 2H), 7.48 (m, 1H), 7.35 (d, 1H), 7.28 (dd, 2H), 7.03 (t, 2H), 4.65 (m, 1H), 1.56 (d, 6H) ppm

Production of Compounds of the Formula [I] by Process (V6):

### N-{4-[3-(4-Fluorophenyl)-1-(2-methoxyethyl)-1H-pyrazol-4-yl]pyridin-2-yl}propanamide [I-2]

50 mg (0.18 mmol) of N-[4-(4,4,5,5-tetramethyl-1,3,2-dioxaborolan-2-yl)pyridin-2-yl]propanamide and 42 mg (0.13 mmol) of 4-bromo-3-(4-fluorophenyl)-1-(2-methoxyethyl)-1H-pyrazole are dissolved in 2.5 mL 1,4-dioxan. To this are added 11.3 mg of 1,1′-bis(diphenylphosphino)ferrocene]-dichloropalladium(II)*CH₂Cl₂ (0.01 mmol) and 1 mL caesium carbonate solution (2 molar). The reaction mixture is flushed for 5 mins with argon and then sealed. Next the mixture is heated for 25 mins at 90° C. in the microwave (CEM Explorer). After cooling, insoluble components are filtered off over Celite and the residue washed with 1,4-dioxan. The organic phase is evaporated and the crude product purified by preparative HPLC (Macherey Nagel, Nucleodur C18 100-5 ec, VP50×21 mm, gradient: 0-1.5 mins 90% water, 10% methanol, 1.5-10.0 mins linear gradient up to 5% water, 95% methanol, 10.0-15.0 mins 0% water, 95% methanol, modifier 20% HCOOH in H2O, addition of the modifier at 2.0 mL/min throughout the separation). 46 mg (69%) of N-{4-[3-(4-fluorophenyl)-1-(2-methoxyethyl)-1H-pyrazol-4-yl]pyridin-2-yl}propanamide are obtained as a colourless solid.

logP (pH 2.7): 1.61

MS (ESI): 369.22 ([M+H]⁺)

1H-NMR (400 MHz, d₃-CD₃CN): δ=8.54 (s, 1H, br), 8.10 (m, 2H), 7.86 (s, 1H), 7.46 (dd, 2H), 7.09 (t, 2H), 6.87 (dd, 1H), 4.31 (t, 2H), 3.78 (t, 2H), 3.32 (s, 3H), 2.38 (q, 2H), 1.11 (t, 2H) ppm

Production of Compounds of the Formula [I] by process (V13):

### 3-{[3-(4-Fluorophenyl)-4-(pyridin-4-yl)-1H-pyrazol-1-yl]methyl}benzonitrile [I-3]

### 3-{[5-(4-Fluorophenyl)-4-(pyridin-4-yl)-1H-pyrazol-1-yl]methyl}benzonitrile [I-4]

60 mg (0.25 mmol) of 4-[3-(4-fluorophenyl)-1H-pyrazol-4-yl]pyridine and dissolved in 2 mL dimethylformamide. To this are added 12.7 mg of sodium hydride (0.32 mol) as a 60% suspension in mineral oil and it is stirred for 10 mins at room temperature. Next, 74 mg (0.38 mmol) of 3-(bromomethyl)benzonitrile are added and the reaction mixture is stirred for 1 hr at room temperature. For the workup, ca. 2 μL acetic acid (0.03 mmol) are added. The suspension obtained is filtered and the crude product is purified by preparative HPLC (XTerra 125×19 mm, 5 μm, gradient: 0-1.5 mins 80% water, 15% methanol, 5% aqueous 10% NH₄HCO₃-soln, 1.5-10.0 mins linear gradient up to 15% water, 80% methanol, 5% aqueous 10% NH₄HCO₃-soln, 10.0-15.0 mins 15% water, 80% methanol, 5% aqueous 10% NH₄HCO₃-soln). 27 mg (30%) of the main isomer 3-{[3-(4-fluorophenyl)-4-(pyridin-4-yl)-1H-pyrazol-1-yl]methyl}benzonitrile [I-3] is obtained as a mixture (in the ratio 58:37) with the minor regioisomer 3-{[5-(4-fluorophenyl)-4-(pyridin-4-yl)-1H-pyrazol-1 -yl]methyl}benzonitrile [1-4] colourless solid.

logP (pH 2.7): 1.51 main isomer

logP (pH 2.7): 1.38 minor isomer

MS (ESI): 355.2 ([M+H]⁺) for both isomers

¹H-NMR (400 MHz, d₃-CD₃CN): δ=8.47 (dd), 8.37 (m), 8.16 (s), 7.86 (s), 7.81 (d), 7.70 (m), 7.50 (t), 7.45-7.30 (m), 7.25-7.10 (m), 7.12 (dd), 5.47 (s, 2H, CH₂ main isomer), 5.27 (s, 2H, CH₂ side isomer) ppm

Analogously to the above example and according to the general descriptions of the process according to the invention, the compounds of the formula [I] named in the following Table 1 can be obtained. These can be formed in the form of an isomer mixture, wherein the proportion of the main and minor isomer can differ depending on the substrate used.

Production of Compounds of the Formula [I-c] by Process (V4):

### N-{4-[3-(4-Fluorophenyl)-1-isopropyl-1H-pyrazol-4-yl]pyridin-2-yl}cyclopropanecarboxamide [I-c-1]

22 mg (0.077 mmol) of 4-[3-(4-fluorophenyl)-1-isopropyl-1H-pyrazol-4-yl]pyridin-2-amine and 12 μL (0.084 mmol) triethylamine are dissolved in 2 mL tetrahydrofuran. To this are added 8.8 mg of cyclopropanecarboxylic acid chloride (0.084 mmol) and the reaction mixture is stirred at room temperature for 2 days. Next, the volatile components are removed under vacuum and the crude material treated with 3 mL NH₃ in methanol (7 molar). The mixture is stirred for 2 hrs at room temperature and then evaporated. The crude product is purified by silica gel chromatography (eluent cyclohexane/ethyl acetate). 11.2 mg (40%) of N-{4-[3-(4 -fluorophenyl)-1-isopropyl-1H-pyrazol-4-yl]pyridin-2-yl}cyclopropanecarboxamide are obtained as a colourless solid.

logP (pH 2.7): 2.07

MS (ESI): 365.13 ([M+H]⁺)

1H-NMR (400 MHz, d₃-CD₃CN): δ=8.82 (s, 1H, br), 8.11 (d, 1H), 8.07 (s, 1H), 7.85 (s, 1H), 7.44 (dd, 2H), 7.06 (t, 2H), 6.86 (dd, 1H), 4.54 (m, 1H), 1.78 (m, 1H), 1.51 (d, 6H), 0.90-0.80 (m, 4H) ppm

Production of Compounds of the Formula [I-c] by Process (V5):

### 4-[3-(4-Fluorophenyl)-1-isopropyl-1H-pyrazol-4-yl]quinoline [I-c-2]

80 mg (0.18 mmol) of 3-(4-fluorophenyl)-1-isobutyl-4-(4,4,5,5 -tetramethyl -1,3,2-dioxaborolan-2-yl)-1H-pyrazole and 68 mg (0.27 mmol) of N-(4-bromopyridin-2-yl)-2-methylpropanamide are dissolved in 2.5 mL 1,4-dioxan. To this are added 15 mg of 1,1′-bis(diphenylphosphino)ferrocene]-dichloro-palladium-(II)*CH₂Cl₂ (0.01 mmol) and 0.5 mL sodium carbonate solution (2 molar). The reaction mixture is flushed for 5 mins with argon and then sealed. Next the mixture is heated for 25 mins at 80° C. in the microwave (CEM Explorer). After cooling, insoluble components are filtered off over Celite and the residue washed with 1,4-dioxan. The organic phase is evaporated and the crude product purified by silica gel chromatography (eluent cyclohexane/ethyl acetate). 29 mg (40% yield) of N-{4-[3-(4-fluorophenyl)-1-isobutyl-1H-pyrazol-4-yl]pyridin-2-yl}-2-methylpropanamide are obtained as a colourless solid.

logP (pH 2.7): 2.89

MS (ESI): 381.19 ([M+H]⁺)

1H-NMR (400 MHz, d₃-CD₃CN): δ=8.59 (s, 1H, br), 8.10 (m, 2H), 7.83 (s, 1H), 7.46 (dd, 2H), 7.09 (dd, 2H), 6.86 (m, 1H), 3.96 (d, 2H), 2.62 (m, 1H), 2.25 (m, 1H), 1.13 (d, 6H), 0.93 (d, 6H) ppm

Production of Compounds of the Formula [I-d] by Process (V17):

### 4-[3-(2,6-Difluorophenyl)-1-isopropyl-1H-pyrazol-4-yl]pyridine [I-d-1] and

### 4-[5-(2,6-Difluorophenyl)-1-isopropyl-1H-pyrazol-4-yl]pyridine [I-d-2]

A mixture of 1-(2,6-difluorophenyl)-3-(dimethylamino)-2-(pyridin-4-yl)prop-2-en-1-one (0.86 mmol), isopropylhydrazine (1.3 mmol) and triethylamine (1.3 mmol) in 5 ml ethanol is irradiated for 15 mins at 120° C. in the microwave. The solvent is evaporated under vacuum and the residue chromatographed over silica gel (gradient heptane/EA 20:1 to 5:1). 69 mg of 4-[3-(2,6-difluoro-phenyl)-1-isopropyl-1H-pyrazol-4-yl]pyridine (25% yield) and 34 mg (12% yield) of 4-[5-(2,6-difluorophenyl)-1-isopropyl-1H-pyrazol-4-yl]pyridine are obtained.

### 4-[3-(2,6-Difluorophenyl)-1-isopropyl-1H-pyrazol-4-yl]pyridine

logP (pH 2.7): 1.40

MS (ESI): 300.2 ([M+H]⁺)

¹H-NMR (400 MHz, d₆-DMSO): δ=8.43 (d, 2H), 8.21 (s, 1H), 7.78 (m, 1H), 7.30 (t, 2H), 7.10 (d, 2H), 4.19 (m, 1H), 1.38 (d, 6H) ppm

### 4-[5-(2,6-Difluorophenyl)-1-isopropyl-1H-pyrazol-4-yl]pyridine

logP (pH 2.7): 1.54

MS (ESI): 300.3 ([M+H]⁺)

¹H-NMR (400 MHz, d₆-DMSO): δ=8.53 (s, 1H), 8.42 (d, 2H), 7.60 (m, 1H), 7.24 (t, 2H), 7.15 (d 2H), 4.60 (m, 1H), 1.51 (d, 6H) ppm

Production of Compounds of the Formula [XX] by Process (V18):

### 1-Cyclopropyl-3-(4-fluorophenyl)-1H-pyrazole [XX-1]

A mixture of 10 g of 3-(4-fluorophenyl)-1H-pyrazole (62 mmol), 10.59 g of cyclopropylboronic acid (123 mmol), 44 mL triethylamine (308 mmol) and 40 mL pyridine (493 mmol) in dry THF is heated under reflux for 18 hrs. Next the reaction mixture is cooled, filtered over Celite and concentrated. The residue is taken up in ethyl acetate, washed with Na2CO3 solution, dried and evaporated under vacuum. The crude product is chromatographed over silica gel and 5 g (40%) of 1-cyclopropyl-3-(4-fluorophenyl)-1H-pyrazole are obtained.

MS (ESI): 203.0 ([M+H]+)

1H-NMR (400MHz, CDCl3) δ=7.76-7.73 (m, 2H) 7.435 (d, J=2.04 Hz, 1H), 7.05 (t, J=8.6 Hz, 2H), 6.44 (s, 1H), 3.64-3.58 (m, 1H), 1.24-1.14 (m, 2H), 1.06-1.01 (m, 2H) ppm

Production of Compounds of the Formula [XXVII] by Process (V16):

### 4-[3-(2,6-Difluorophenyl)-1H-pyrazol-4-yl]pyridine [XXVII-1]

A mixture of 1-(2,6-difluorophenyl)-3-(dimethylamino)-2-(pyridin-4-yl)prop-2-en-1-one (0.86 mmol), hydrazine hydrate (1.3 mmol) and triethylamine (1.3 mmol) in 5 ml ethanol is irradiated for 15 mins at 120° C. in the microwave. The solvent is evaporated under vacuum, the residue taken up in dichloromethane (DCM) and the suspension filtered. The solid is dried in the vacuum oven at 50° C. 0.18 g (76% yield) of 4-[3-(2,6-difluorophenyl)-1H-pyrazol-4-yl]pyridine is obtained.

logP (pH 2.7): 1.54

MS (ESI): 258.1 ([M+H]⁺)

¹H-NMR (400 MHz, d₆-DMSO): δ=13.59 (bs, 1H), 8.46 (m, 3H), 7.60 (m, 1H), 7.29 (m, 2H), 7.18 (m, 2H) ppm

Production of Compounds of the Formula [XXVI] by Process (V15):

### 1-(2,6-Difluorophenyl)-3-(dimethylamino)-2-(pyridin-4-yl)prop-2-en-1-one [XXVI-1]

A suspension of 4-(2,6-difluorophenyl)-2-pyrid-4-ylethanone (17.7 mmol) in 20 ml N,N-dimethyl-formamide (DMF) is treated with N,N-dimethylformamide dimethyl acetal (60.3 mmol) and heated for 3 hrs under reflux. After cooling to room temperature the solvent is evaporated under vacuum, the residue taken up in ethyl acetate and the aqueous phase extracted three times with EA. The combined extracts are dried over MgSO₄ and evaporated under vacuum. dried and evaporated under vacuum. The residue is chromatographed over silica gel (gradient heptane/EA 2:1 to 0:1). 3.1 g (58%) of 1-(2,6-difluorophenyl)-3- (dimethylamino)-2-(pyridin-4-yl)prop-2-en-1-one are obtained.

logP (pH 2.7): 0.60

MS (ESI): 289.2 ([M+H]⁺)

Production of Compounds of the Formula [XXV] by Process (V14):

### 1-(2,6-Difluorophenyl)-2-(pyridin-4-yl)ethanone [XXV-1]

A solution of 4-methylpyridine (24.6 mmol) and ethyl 2,6-difluorobenzoate (27.1 mmol) in 58 ml anhydrous tetrahydrofuran (THF) is cooled to 0° C. and treated dropwise with 24.6 ml lithium bistrimethylsilylamide (LiHMDS, 1 molar solution in hexane). After 3 hours at 5-10° C., water is added and the mixture extracted with ethyl acetate (acetic acid ethyl ester). The organic phase is washed with saturated sodium chloride solution (saturated NaCl), dried over magnesium sulphate (MgSO₄) and evaporated under vacuum. The residue is chromatographed over silica gel (gradient heptane/EA 3:1 to 1:1). 4.1 g (54% yield) of 4-(2,6-difluorophenyl)-2-pyrid-4-ylethanone are obtained

logP (pH 2.7): 0.62

MS (ESI): 234.1 ([M+H]⁺)

¹H-NMR (400 MHz, d₆-DMSO): δ=8.53 (d, 2H), 7.65 (m, 1H), 7.27 (m, 4H), 4.34 (s, 2H) ppm

Production of Compounds of the Formula [XXVIII] by Process (V19):

### 4-[5-Bromo-3-(4-fluorophenyl)-1H-pyrazol-4-yl]pyridine [XXVIII-1]

A solution of 4-[3-(4-fluorophenyl)-1H-pyrazol-4-yl]pyridine (0.58 mmol) in 2 mL N,N-dimethyl-formamide and N-bromosuccinimide (0.58 mmol) is heated for 2 hrs at 80° C. After cooling to room temperature, this is treated with water and ethyl acetate. The organic phase is washed with water, dried over MgSO₄ and after filtration evaporated under vacuum. The residue is suspended in diisopropyl ether, filtered and dried in the vacuum oven at 50° C. 0.13 g (64% yield) of 4-[5-bromo-3-(4-fluorophenyl)-1H-pyrazol-4-yl]pyridine were obtained

logP (pH 2.7): 0.92

MS (ESI): 318.0 ([M+H]⁺)

¹H-NMR (400 MHz, d₆-DMSO): δ=13.88 (bs, 1H), 8.60 (d, 2H), 7.38 (m, 2H), 7.29 (m, 4H) ppm

Production of Compounds of the Formula [IX-b] by Process (V20):

### 4-[3-(4-Fluorophenyl)-5-methyl-1H-pyrazol-4-yl]pyridine [IX-b-1]

Under argon, a degassed solution of 4-[5-bromo-3-(4-fluorophenyl)-1-(4-methoxybenzyl)-1H-pyrazol-4-yl]pyridine and 4-[3-bromo-5-(4-fluorophenyl)-1-(4-methoxybenzyl)-1H-pyrazol-4-yl]-pyridine (mixture of two regioisomers, 1:1, 0.68 mmol) in 10.5 ml dimethoxyethane and 3 ml water is added to a solution of sodium hydrogen carbonate (NaHCO₃, 2.1 mmol) and dichloro[1.1′-ferrocenylbis(diphenylphosphane)]palladium(II)dichloromethane (0.03 mmol). This followed by the addition of a 50% solution of trimethylboroxine (1.36 mmol) in THF. The mixture is heated for 3 hrs at 90° C., cooled to room temperature and treated with water and ethyl acetate. The aqueous solution is extracted with ethyl acetate, and the organic phase washed with saturated aqueous NaC1 solution, dried over MgSO₄ and evaporated under vacuum.

Removal of the N-substituent on the pyrazole: the residue is taken up in 3 ml trifluoroacetic acid (TFA) and stirred for 2 hrs at 65° C. After addition of water and ethyl acetate, the organic phase is extracted with ethyl acetate, washed with saturated aqueous NaCl solution, dried over MgSO₄ and evaporated under vacuum.

The residue is chromatographed on silica gel (gradient DCM/methanol (MeOH) 20:1 to 10:1). 88 mg (43% yield) of 4-[3-(4-fluorophenyl)-5-methyl-1H-pyrazol-4-yl]pyridine are obtained.

logP (pH 2.7): 0.60

MS (ESI): 254.1 ([M+H]⁺)

¹H-NMR (400 MHz, d₆-DMSO): δ=13.03 (s, 1H), 8.52 (d, 2H), 7.35 (m, 2H), 7.16 (m, 4H), 2.29 (s, 3H) ppm

### 4-[3-(4-Fluorophenyl)-5-cyclopropyl-1H-pyrazol-4-yl]pyridine [IX-b-2]

Under argon, a degassed solution of 4-[5-bromo-3-(4-fluorophenyl)-1-(4-methoxybenzyl)-1H-pyrazol-4-yl]pyridine and 4-[3-bromo-5-(4-fluorophenyl)-1-(4-methoxybenzyl)-1H-pyrazol-4-yl]-pyridine (mixture of two regioisomers, 1:1, 0.68 mmol) in 10.5 ml dimethoxyethane and 3 ml water is added to a solution of sodium hydrogen carbonate (NaHCO₃, 2.1 mmol), dichloro[1.1′-ferrocenyl-bis(diphenylphosphane)]palladium(II)dichloromethane (0.03 mmol) and cyclopropylboronic acid (1.63 mmol). The mixture is heated for 3 hrs at 90° C. and 16 hrs at 65° C., cooled to room temperature and treated with water and ethyl acetate. The aqueous solution is extracted with ethyl acetate, the organic phase washed with satd. NaCl, over MgSO₄ dried and evaporated under vacuum.

Removal of the N-substituent on the pyrazole: The residue is taken up in 3 ml trifluoroacetic acid (TFA) and stirred for 2 hrs at 65° C. After addition of water and EA, the organic phase is extracted with ethyl acetate, washed with satd. NaCl, over MgSO₄, dried and evaporated under vacuum.

The residue is chromatographed on silica gel (gradient DCM/methanol (MeOH) 20:1 to 10:1). 75.8 mg (38% yield) of 4-[3-(4-fluorophenyl)-5-cyclopropyl-1H-pyrazol-4-yl]pyridine were obtained.

logP (pH 2.7): 0.920

MS (ESI): 280 ([M+H]⁺)

Production of Compounds of the Formula [XXXII] by Process (V22):

### 5-(4-Fluorophenyl)-2-methyl-2,4-dihydro-3H-pyrazol-31-one [XXXII-1]

To a solution of 8.00 g of methyl 3-(4-fluorophenyl)-3-oxopropanoate (40.8 mol) in 45 mL ethyl acetate, 2.43 g (53.0 mmol) of methylhydrazine are slowly added. Next the reaction mixture is heated under reflux until complete reaction of the starting material. After cooling to room temperature, the reaction mixture is treated with diethyl ether and water. The precipitate formed is filtered off at the pump, washed with a petroleum ether/diethyl ether mixture (60 mL, 1:1) and dried. 5.33 g (68%) of 5-(4-fluorophenyl)-2-methyl-2,4-dihydro-3H-pyrazol-3-one are obtained.

¹H-NMR (400 MHz, CDCl₃): δ=7.69-7.63 (m, 2 H); 7.12 (t, 2H); 3.59 (s, 2H), 3.41 (s, 3H) ppm

Production of Compounds of the Formula [XXXIII] by process (V23):

### 5-(Difluoromethoxy)-3-(4-fluorophenyl)-1-methyl-1H-pyrazole [XXXIII-1]

To a solution of 4.40 g (22.9 mmol) of 5-(4-fluorophenyl)-2-methyl-2,4-dihydro-3H-pyrazol-3-one in anhydrous acetonitrile (100 mL) are added 3.16 g (22.9 mmol) of K₂CO₃ and 4.19 g (27.5 mmol) of sodium chlorodifluoracetate and the mixture is heated under reflux for 5 hrs in a N₂ atmosphere under reflux. After cooling of the reaction mixture, aqueous NH4Cl solution (85 mL) is added and the organic phase is extracted with ethyl acetate (3×50 mL). The combined organic extracts are washed with NaCl solution, dried and evaporated under vacuum. The crude product is purified by chromatography on silica gel (eluent petroleum ether/ethyl acetate 8/2). 1.26 g (23%) of 5-(difluoro-methoxy)-3-(4-fluorophenyl)-1-methyl-1H-pyrazole are obtained.

¹H-NMR (400 MHz, CDCl₃): δ=7.74-7.68 (m, 2H); 7.08 (t, 2H); 6.56 (t, 2JHF=72.2 Hz, 1H, CHF2); 6.14 (s, 1H); 3.78 (s, 3H) ppm

Production of Compounds of the Formula [VI-b] by Process (V24):

### 4-Bromo-5-(difluoromethoxy)-3-(4-fluorophenyl)-1-methyl-1H-pyrazole [VI-b-1]

0.914 g (5.72 mmol) of bromine is added dropwise to a solution of 1.16 g (7.28 mmol) of 5-(difluoro-methoxy)-3-(4-fluorophenyl)-1-methyl-1H-pyrazole in dichloromethane (14 mL). The reaction mixture is stirred for 26 hrs at room temperature. Next the reaction mixture is washed with Na₂S₂O₃ solution (3×20 mL) and with NaHCO3 solution (3×30 mL). The organic phase is dried and evaporated under vacuum. 1.34 g (80%) of 4-bromo-5-(difluoromethoxy)-3-(4-fluorophenyl)-1-methyl-1H-pyrazole are obtained.

logP (pH 2.7): 3.52

MS (ESI): 321.1 ([M+H]⁺)

¹H-NMR (400 MHz, d6-DMSO): δ=7.86-7.81 (m, 2H), 7.50-7.15 (m, 3H), 3.80 (s, 3H) ppm

Analogously to the above example the following compounds of the formula [VI-b] can also be obtained:

### 4-Bromo-5-(difluoromethoxy)-3-(4-fluorophenyl)-1-isopropyl-1H-pyrazole [VI-b-2]

logP (pH 2.7): 4.61

MS (ESI): 351.0 ([M+H]⁺)

¹H-NMR (400 MHz, d6-DMSO): δ=7.87-7.82 (m, 2H), 7.52-7.16 (m, 3H), 4.64-4.57 (m, 1H), 1.42 (d, 6H) ppm

### 4-Bromo-5-(difluoromethoxy)-3-(4-fluorophenyl)-1-isobutyl-1H-pyrazole [VI-b-3]

logP (pH 2.7): 4.91

MS (ESI): 365.0 ([M+H]⁺)

¹H-NMR (400 MHz, d6-DMSO): δ=7.87-7.83 (m, 2H), 7.51-7.16 (m, 3H), 3.90 (d, 2H), 2.22-2.15 (m, 6H) ppm

### 4-Bromo-5-(difluoromethoxy)-3-(4-fluoro-2-methoxyphenyl)-1-methyl-1H-pyrazole [VI-b-4]

logP (pH 2.7): 3.15

MS (ESI): 353.0 ([M+H]⁺)

¹H-NMR (400 MHz, d6-DMSO): δ=7.50-7.13 (m, 3H), 7.05-7.02 (m, 1H), 6.86-6.81 (m, 1H) 3.80 (s, 3H), 3.78 (s, 3H) ppm

Production of Compounds of the Formula [VI-c] by Process (V25):

### 2-[4-Bromo-5-(difluoromethoxy)-1-methyl-1H-pyrazol-3-yl]-5-fluorophenol [VI-c-1]

5.8 mL of BBr₃ (1M solution in dichloromethane, 5.8 mmol) are added dropwise at 0° C. to a solution of 3.0 g (8.6 mmol) of 4-bromo-5-(difluoromethoxy)-3-(4-fluoro-2-methoxyphenyl)-1-methyl-1H-pyrazole in dichloromethane (68 mL). The reaction mixture is slowly warmed to room temperature and stirred for 23 hrs. Next 150 mL of diethyl ether are added and the mixture obtained is partitioned between saturated NaHCO₃ solution (100 mL) and ethyl acetate (200 mL). The precipitate obtained is dissolved by addition of 100 mL water and the phases separated. The aqueous phase is extracted with ethyl acetate (3×200 mL). The combined organic extracts are washed with water and saturated NaCl solution and dried. After removal of the solvent under vacuum, the crude product obtained is purified by chromatography on silica gel (eluent petroleum ether/ethyl acetate 95/5). 1.6 g (55%) of 2-[4-bromo-5-(difluoromethoxy)-1-methyl-1H-pyrazol-3-yl]-5-fluorophenol are obtained.

logP (pH 2.7): 3.48

MS (ESI): 336.9 ([M+H]⁺)

1H-NMR (400 MHz, d6-DMSO): δ=7.49-7.13 (m, 2H), 6.74-6.68 (m, 2H), 3.78 (s, 3H) ppm

Production of Starting Materials of the Formula [XXXVII] by Process V14:

### 1-(4-Fluorophenyl)-2-[2-(methylsulphanyl)pyrimidin-4-yl]ethanone [XXXVII-1]

A solution of 4-methyl-2-(methylsulphanyl)pyrimidine (1 eq, 41 mmol) and ethyl-4-fluorobenzoate (1.1 eq, 45 mmol) in 50 mL anhydrous tetrahydrofuran (THF) is cooled to 0° C. and treated dropwise with lithium bistrimethylsilylamide (2 eq, 82 mmol, 1 molar solution of LiHMDS in n-hexane). After 3 hours at 5-10° C., water is added and the mixture extracted with ethyl acetate (acetic acid ethyl ester). The organic phase is washed with saturated sodium chloride solution (NaCl), dried over magnesium sulphate (MgSO₄) and evaporated under vacuum. The residue is purified by crystallization from 100 mL cyclohexane and dried under vacuum. 8.9 g (83% yield) of 1-(4-fluoro-phenyl)-2-[2-(methylsulphanyl)pyrimidin-4-yl]ethanone are obtained.

Production of Starting Materials of the Formula [XXXVIII] by Process V15:

### 3-(Dimethylamino)-1-(4-fluorophenyl)-2-[2-(methylsulphanyl)pyrimidin-4-yl]prop-2-en-1-one [XXXVIII-1]

A solution of 1-(4-fluorophenyl)-2-[2-(methylsulphanyl)pyrimidin-4-yl]ethanone (1 eq, 30 mmol) in 40 mL N,N-dimethylformamide dimethyl acetal is heated for 1 hr at 75-80° C. After cooling to room temperature, the solvent is removed under vacuum and the residue purified by chromatography on silica gel (gradient heptane/ethyl acetate 1:1 to 2:8). 9.3 g (97%) of (2Z)-3-(dimethylamino)-1-(4-fluorophenyl)-2-[2-(methylsulphanyl)pyrimidin-4-yl]prop-2-en-1-one are obtained.

Production of Starting Materials of the Formula [XXXIX] by Process V16:

### 4-[3-(4-Fluorophenyl)-1H-pyrazol-4-yl]-2-(methylsulphanyl)pyrimidine [XXXIX-1]

A mixture of 3-(dimethylamino)-1-(4-fluorophenyl)-2-[2-(methylsulphanyl)pyrimidin-4-yl]prop-2-en-1-one (1 eq, 29 mmol), hydrazine hydrate (1.5 eq, 44 mmol) and triethylamine (1.5 eq, 44 mmol) in 186 mL ethanol is heated for 3 hrs under reflux. The solvent is evaporated under vacuum, water is added and the mixture extracted with ethyl acetate. The organic phase is separated, dried (MgSO₄) and evaporated under vacuum. 7.9 g (94% yield) of 4-[3-(4-fluorophenyl)-1H-pyrazol-4-yl]-2-(methylsulphanyl)pyrimidine are obtained.

logP (pH 2.7): 2.28

MS (ESI): 287.0 ([M+H]⁺)

¹H-NMR (400 MHz, d₆-DMSO): δ=13.48 (bs, 1H), 8.44 (d, 1H), 8.38 (bs, 1H), 7.56 (m, 2H), 7.27 (t, 2H), 7.12 (d, 1H), 2.21 (s, 3H) ppm

Production of Starting Materials of the Formula [XL] by Process V13:

### 4-[3-(4-Fluorophenyl)-1-isopropyl-1H-pyrazol-4-yl]-2-(methylsulphanyl)pyrimidine [XL-1]

Cs₂CO₃ (2.5 eq, 73.3 mmol) and 2-iodopropane (1.5 eq, 44 mmol) are added to a solution of 4-[3-(4-fluorophenyl)-1H-pyrazol-4-yl]-2-(methylsulphanyl)pyrimidine (1 eq, 29.3 mol) in 75 mL N,N-dimethylformamide and the reaction mixture is stirred overnight at room temperature. After this, the mixture is treated with water and extracted with ethyl acetate. The organic phase is dried, evaporated and purified by chromatography on silica gel (gradient dichloromethane/ethyl acetate 20:1 to 5:1). 6.5 g (64%) of 4-[3-(4-fluorophenyl)-1-isopropyl-1H-pyrazol-4-yl]-2-(methylsulphanyl)-pyrimidine are obtained.

logP (pH 2.7): 3.62

MS (ESI): 329.0 ([M+H]⁺)

¹H-NMR (400 MHz, CDCl₃): δ=8.28 (d, 1H), 8.10 (s, 1H), 7.52 (m, 2H), 7.11 (t, 2H), 6.73 (d, 1H), 4.57 (m, 1H), 2.50 (s, 3H), 1.60 (d, 6H) ppm

Production of Starting Materials of the Formula [XLI] by Process V27:

### 4-[3-(4-Fluorophenyl)-1-isopropyl-1H-pyrazol-4-yl]-2-(methylsulphonyl)pyrimidine [XLI-1]

A solution of 4-[3-(4-fluorophenyl)-1-isopropyl-1H-pyrazol-4-yl]-2-(methylsulphanyl)pyrimidine (1 eq, 19.7 mmol) and m-chloroperbenzoic acid (2 eq, 40 mmol, 70%) in 520 mL dichloromethane is stirred overnight at room temperature. After this, the reaction mixture is treated with water and sodium sulphite (2.1 eq, 41.5 mmol) and the phases separated. The organic phase is washed 2× with a 2M K₂CO₃ solution, dried and evaporated under vacuum. 6.3 g (84%) of 4-[3-(4-fluorophenyl)-1-isopropyl-1H-pyrazol-4-yl]-2-(methylsulphonyppyrimidine are obtained.

logP (pH 2.7): 2.90

MS (ESI): 375.1 ([M+H]+)

¹H-NMR (400 MHz, d₆-DMSO): δ=8.87 (d, 1H), 8.70 (s, 1H), 7.62 (d, 1H), 7.61 (m, 2H), 7.25 (t, 2H), 4.05 (d, 2H), 3.17 (s, 3H), 2.22 (m, 1H), 0.92 (d, 6H) ppm

Production of Compounds of the Formula [I-f] by Process V28:

### N-Benzyl-4-[3-(4-fluorophenyl)-1-isopropyl-1H-pyrazol-4-yl]pyrimidin-2-amine [I-f-1]

A mixture of 4-[3-(4-fluorophenyl)-1-isopropyl-1H-pyrazol-4-yl]-2-(methylsulphonyl)pyrimidine (1 eq, 9.15 mmol) in 43 mL benzylamine is stirred for 4 hrs at room temperature. Next, the benzylamine is removed under vacuum and the crude product purified by chromatography on silica gel (gradient dichloromethane/ethyl acetate 20:1 to 5:1). 1.77 g (47%) of N-benzyl-4-[3-(4-fluoro-phenyl)-1-isopropyl-1H-pyrazol-4-yl]pyrimidin-2-amine are obtained.

logP (pH 2.7): 3.31

MS (ESI): 385.1 ([M+H]⁺)

Production of Starting Materials of the Formula by Process V29:

### 4-[3-(4-Fluorophenyl)-1-isopropyl-1H-pyrazol-4-yl]pyrimidin-2-amine [III-a-1]

A solution of N-benzyl-4-[3-(4-fluorophenyl)-1-isopropyl-1H-pyrazol-4-yl]pyrimidin-2-amine (1 eq, 5.21 mmol) in sulphuric acid (100 eq, 521 mmol) is stirred overnight at room temperature. Next the reaction mixture is treated first with ice, then with water and cautiously neutralized to pH=9 with 30% NaOH. The aqueous phase is extracted several times with dichloromethane. The combined organic extracts are dried and evaporated under vacuum. 1.1 g (67%) of 4-[3-(4-fluorophenyl)-1-isopropyl-1H-pyrazol-4-yl]pyrimidin-2-amine are obtained.

logP (pH 2.7): 1.54

MS (ESI): 298.2 ([M+H]+)

¹H-NMR (400 MHz, d₆-DMSO): δ=8.23 (s, 1H), 8.08 (d, 1H), 7.55 (m, 2H), 7.23 (t, 2H), 6.47 (s, 1H) 6.76 (d, 1H), 6.35 (d, 1H), 4.57 (m, 1H), 1.48 (d, 6H) ppm

Production of Compounds of the Formula [I-g] by Process V4:

### N-{4-[3-(4-Fluorophenyl)-1-isopropyl-1H-pyrazol-4-yl]pyrimidin-2-yl}propanamide [I-g-1]

Propionyl chloride (2 eq, 0.67 mmol) is added to a solution of 4-[3-(4-fluorophenyl)-1-isopropyl-1H-pyrazol-4-yl]pyrimidin-2-amine (4 eq, 1.34 mmol) and triethylamine (4 eq, 1.34 mmol) in 6 mL tetrahydrofuran. The reaction mixture is stirred overnight at room temperature. Next, the volatile components are removed under vacuum and the crude material is treated with 6 mL NH₃ in methanol (7 molar). The mixture is stirred for 2 hrs at room temperature and then evaporated. The crude product is treated with water and extracted 2× with dichloromethane. The organic extracts are dried and evaporated under vacuum. The crude product is purified by chromatography on silica gel (eluent heptane/ethyl acetate). 70 mg (59%) of N-{4-[3-(4-fluorophenyl)-1-isopropyl-1H-pyrazol-4-yl]-pyrimidin-2-yl}propanamide are obtained.

logP (pH 2.7): 2.35

MS (ESI): 354.2 ([M+H]⁺)

¹H-NMR (400 MHz, d₆-DMSO): δ=10.27 (bs, 1H), 8.46 (d, 1H), 8.38 (s, 1H), 7.60 (m, 2H), 7.23 (t, 2H), 6.94 (m, 1H), 4.61 (m, 1H), 2.40 (q, 2H), 1.50 (d, 6H), 0.98 (t, 3H) ppm

Production of Starting Materials of the Formula [XLIII] by Process V14:

### 2-(2-Chloropyrimidin-4-yl)-1-(4-fluorophenyl)ethanone [XLIII-1]

A solution of 2-chloro-4-methylpyrimidine (1 eq, 15.5 mmol) and ethyl 4-fluorobenzoate (1.1 eq, 17.1 mmol) in 17 mL anhydrous tetrahydrofuran (THF) is cooled to 0° C. and treated dropwise with lithium bistrimethylsilylamide (2 eq, 31 mmol, 1 molar solution of LiHMDS in n-hexane). After 3 hours at 5-10° C., water is added and the mixture extracted with dichloromethane. The organic phase is washed with saturated sodium chloride solution (NaCl), dried over magnesium sulphate (MgSO₄) and evaporated under vacuum. 3.8 g (83% yield) of 2-(2-chloropyrimidin-4-yl)-1-(4-fluorophenyl)-ethanone (4/9 mixture of keto and enol form) are obtained.

logP (pH 2.7): 2.27

MS (ESI): 251.0 ([M+H]⁺)

¹H-NMR (400 MHz, d₆-DMSO): δ=13.58 (s, 1H, enol), 8.75 (d, 1H), 8.57 (bs, 1H, enol), 8.11 (m, 2H), 7.92 (m, 2H, enol), 7.60 (d, 1H), 7.41 (m, 2H), 7.34 (m, 3H, enol), 6.51 (bs, 1H, enol), 4.70 (s, 2H) ppm

Production of Starting Materials of the Formula [XLIV] by Process V28:

### N-Benzyl-4-[3-(4-fluorophenyl)-1-isopropyl-1H-pyrazol-4-yl]pyrimidin-2-amine [XLIV-1]

A mixture of 2-(2-chloropyrimidin-4-yl)-1-(4-fluorophenyl)ethanone (1 eq, 16 mmol) in 27 mL isopropylamine is heated for 10 mins at 110° C. in the microwave oven (CEM Explorer). Next, the amine is removed under vacuum and the crude product treated with dichloromethane (25 mL) and 1M HCl (7 mL). The solution is stirred overnight at room temperature and then neutralized with 1M NaOH. The phases are separated and the aqueous phase is extracted 2× with dichloromethane. The combined organic extracts are dried (MgSO4) and evaporated under vacuum. 4.0 g (77%) of 1-(4-fluorophenyl)-2-[2-(isopropylamino)pyrimidin-4-yl]ethanone are obtained.

logP (pH 2.7): 2.05

MS (ESI): 274.2 ([M+H]⁺)

Production of Starting Materials of the Formula [XLV] by Process V15:

### 3-(Dimethylamino)-1-(4-fluorophenyl)-2-[2-(isopropylamino)pyrimidin-4-yl]prop-2-en-1-one [XLV-1]

A solution of 1-(4-fluorophenyl)-2-[2-(isopropylamino)pyrimidin-4-yl]ethanone (1 eq, 14.6 mmol) in a mixture of 6.5 mL N,N-dimethylformamide and 6.5 mL N,N-dimethylformamide dimethyl acetal is heated for 2.5 hrs at 100° C. After cooling to room temperature, the solvent is removed under vacuum. 5.4 g (65%) of 3-(dimethylamino)-1-(4-fluorophenyl)-2-[2-(isopropylamino)pyrimidin-4-yl]prop-2-en-1-one are obtained.

logP (pH 2.7): 1.45

MS (ESI): 329.2 ([M+H]⁺)

Production of Starting Materials of the Formula [XLVI] by Process V16:

### 4-[3-(4-fluorophenyl)-1H-pyrazol-4-yl]-N-isopropylpyrimidin-2-amine [XLVI-1]

A mixture of 3-(dimethylamino)-1-(4-fluorophenyl)-2-[2-(isopropylamino)pyrimidin-4-yl]prop-2-en-1-one (1 eq, 16.4 mmol), hydrazine hydrate (1.1 eq, 18 mmol) in 100 mL ethanol is stirred for 16 hrs at room temperature. The solvent is evaporated under vacuum and the residue purified by chromatography on silica gel (gradient heptane/ethyl acetate 1:0 to 6:4). 3.5 g (65% yield) of 4-[3-(4-fluorophenyl)-1H-pyrazol-4-yl]-N-isopropylpyrimidin-2-amine are obtained.

logP (pH 2.7): 1.37

MS (ESI): 298.2 ([M+H]⁺)

¹H-NMR (400 MHz, d₆-DMSO): δ=13.28 (bs, 1H), 8.12 (d, 1H), 7.59 (bs, 2H), 7.28 (bs, 2H), 6.77 (d, 1H), 6.52 (bs, 1H), 3.81 (bs, 1H), 1.09 (bs, 6H) ppm.

Production of Compounds of the Formula [I-h] by Process V13:

### 4-[3-(4-fluorophenyl)-1-isobutyl-1H-pyrazol-4-yl]-N-isopropylpyrimidin-2-amine [I-h-1]

Cs₂CO₃ (1.1 eq, 0.9 mmol) and 2-iodopropane (1.5 eq, 1.23 mmol) are added to a solution of 4-[3-(4-fluorophenyl)-1H-pyrazol-4-yl]-N-isopropylpyrimidin-2-amine (1 eq, 0.82 mmol) in 8 mL N,N-dimethylformamide and the reaction mixture is stirred overnight at room temperature and then heated for 4 hrs at 80° C. After this, the solvent is removed, and the crude product treated with water and extracted 3× min dichloromethane. The organic phase is dried, evaporated and purified by chromatography on silica gel (gradient heptane/ethyl acetate 1:0 to 1:1). 124 mg (40%) of 4-[3-(4-fluorophenyl)-1-isobutyl-1H-pyrazol-4-yl]-N-isopropylpyrimidin-2-amine are obtained.

logP (pH 2.7): 1.81

MS (ESI): 312.2 ([M+H]⁺) p ¹H-NMR (400 MHz, CDCl₃): δ=8.01 (d, 1H), 7.89 (s, 1H), 7.45 (m, 2H), 7.02 (t, 2H), 6.36 (d, 1H), 4.94 (bs, 2H), 3.89 (d, 2H), 2.23 (m, 1H), 0.91 (d, 6H) ppm

The compounds of the formula [I-a] and [I-b] named in the following Tables I-III are also obtained by the aforesaid methods.

TABLE 1
[I-a]

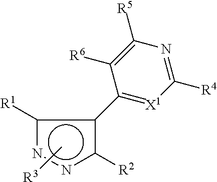

[I-b]

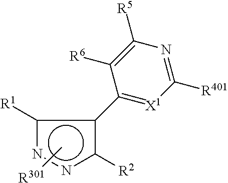

| Ex. | X¹ | R¹ | R² | R^{3/301} | R^{4/401} | R⁵ | R⁶ |
|---|---|---|---|---|---|---|---|
| 1 | CH | 4-fluorophenyl | H | 2-methylpropyl | H | H | H |
| 2 | CH | 2,6-difluorophenyl | H | 2-methylpropyl | H | H | H |
| 3 | CH | 2,6-difluorophenyl | H | butan-2-yl | H | H | H |
| 4^{[s]} | CH | 4-fluorophenyl | H | H | H | H | H |
| 5 | N | 3-chlorophenyl | H | 2-methylpropyl | H | H | H |
| 6 | CH | 2,6-difluorophenyl | H | 2,2-dimethylpropyl | H | H | H |
| 7 | CH | 2,6-difluorophenyl | H | 3-methylbut-2-en-1-yl | H | H | H |
| 8 | CH | 2,6-difluorophenyl | H | cyclobutyl | H | H | H |
| 9 | CH | 4-fluorophenyl | H | tetrahydro-2H-pyran-2-yl | H | H | H |
| 10 | CH | 2,6-difluorophenyl | H | propan-2-yl | H | H | H |
| 11 | CH | 2,6-difluorophenyl | H | 2,2-dimethylpropyl | H | H | H |
| 12 | CH | 2,6-difluorophenyl | H | 2,2,2-trifluoroethyl | H | H | H |
| 13 | CH | 2,6-difluorophenyl | H | 2-methylpropyl | H | H | H |
| 14 | CH | 2,6-difluorophenyl | H | propan-2-yl | H | H | H |
| 15 | CH | 2,6-difluorophenyl | H | cyclobutyl | H | H | H |
| 16 | CH | 2,6-difluorophenyl | H | cyclopentyl | H | H | H |
| 17 | CH | 2,6-difluorophenyl | H | cyclopentyl | H | H | H |
| 18^{[s]} | N | 4-fluorophenyl | H | H | CH₃ | H | H |
| 19 | CH | 4-fluorophenyl | H | tetrahydro-2H-pyran-2-yl | propanoylamino | H | H |
| 20 | CH | 4-fluorophenyl | H | tetrahydro-2H-pyran-2-yl | H | —NHCH═CH— | |
| 21 | CH | 4-fluorophenyl | H | tetrahydro-2H-pyran-2-yl | (2-methylpropanoyl)- | H | H |
| | | | | | amino | | |
| 22 | CH | 2,6-difluorophenyl | H | 3-methylbut-2-en-1-yl | H | H | H |
| 23 | CH | 4-fluorophenyl | H | tetrahydro-2H-pyran-2-yl | (methoxyacetyl)- | H | H |
| | | | | | amino | | |
| 24 | CH | 4-fluorophenyl | H | 2-methylpropyl | H | H | H |
| 25 | CH | 4-fluorophenyl | H | 3-methylbut-2-en-1-yl | H | H | H |
| 26 | CH | 2,6-difluorophenyl | H | tetrahydrofuran-2-ylmethyl | H | H | H |
| 27 | CH | 2,6-difluorophenyl | H | tetrahydrofuran-2-ylmethyl | H | H | H |
| 28 | CH | 3-chlorophenyl | H | tetrahydro-2H-pyran-2-yl | H | H | H |
| 29 | N | 4-fluorophenyl | H | tetrahydro-2H-pyran-2-yl | CH₃ | H | H |
| 30 | CH | 4-fluorophenyl | H | 3-methylbut-2-en-1-yl | H | H | H |
| 31 | CH | 4-fluorophenyl | H | tetrahydro-2H-pyran-2-yl | CH₃ | H | H |
| 32 | CH | 3-chlorophenyl | H | tetrahydro-2H-pyran-2-yl | H | —NHCH═CH— | |
| 33 | CH | 2,2-difluoro-1,3- | H | tetrahydro-2H-pyran-2-yl | H | H | H |
| | | benzodioxol-4-yl | | | | | |
| 34 | N | 3-chlorophenyl | H | 3-methylbut-2-en-1-yl | H | H | H |
| 35 | CH | 4-fluorophenyl | bromo | tetrahydro-2H-pyran-2-yl | H | H | H |
| 36 | N | 3-(trifluoromethyl)- | H | tetrahydro-2H-pyran-2-yl | morpholin-4-yl | H | H |
| | | phenyl | | | | | |
| 37 | CH | 4-fluoro-2-methyl- | H | tetrahydro-2H-pyran-2-yl | H | H | H |
| | | phenyl | | | | | |
| 38 | CH | 2,6-difluorophenyl | H | butan-2-yl | H | H | H |
| 39 | CH | 4-fluorophenyl | H | tetrahydro-2H-pyran-2-yl | amino | H | H |
| 40 | CH | 4-fluorophenyl | H | propan-2-yl | (methoxyacetyl)- | H | H |
| | | | | | amino | | |
| 41 | CH | 4-fluorophenyl | H | tetrahydro-2H-pyran-2-yl | (2,2-dimethyl- | H | H |
| | | | | | propanoyl)amino | | |
| 42 | CH | 3-(trifluoromethyl)- | H | tetrahydro-2H-pyran-2-yl | H | H | H |
| | | phenyl | | | | | |
| 43 | CH | 4-methoxyphenyl | H | tetrahydro-2H-pyran-2-yl | H | H | H |
| 44 | CH | 4-fluorophenyl | H | propan-2-yl | H | (1Z,3Z)-buta-1,3-dien-1,4-diyl | |
| 45 | CH | 4-methoxynaphthalen- | H | tetrahydro-2H-pyran-2-yl | H | H | H |
| | | 1-yl | | | | | |
| 46 | CH | 4-fluorophenyl | H | propan-2-yl | amino | H | H |
| 47 | CH | 4-fluorophenyl | H | propan-2-yl | H | -(prop-2-yn-1-yl)N—CH═CH— | |
| 48 | CH | 3-(trifluoromethyl)- | H | tetrahydro-2H-pyran-2-yl | (2,2-dimethyl- | H | H |
| | | phenyl | | | propanoyl)amino | | |
| 49 | CH | 4-fluorophenyl | H | tetrahydro-2H-pyran-2-yl | H | -(prop-2-yn-1-yl)N—CH═CH— | |
| 50 | CH | 3-(trifluoromethyl)- | H | tetrahydro-2H-pyran-2-yl | H | —NHCH═CH— | |
| | | phenyl | | | | | |
| 51 | CH | 3-chloro-4-fluorophenyl | H | tetrahydro-2H-pyran-2-yl | H | H | H |
| 52 | N | 4-fluorophenyl | H | tetrahydro-2H-pyran-2-yl | morpholin-4-yl | H | H |
| 53 | CH | 4-fluorophenyl | H | propan-2-yl | methylcarbamoyl | H | H |
| 54 | CH | 4-fluorophenyl | H | tetrahydro-2H-pyran-2-yl | H | (1Z,3Z)-buta-1,3-dien-1,4-diyl | |
| 55 | CH | 4-fluoro-3-methyl- | H | tetrahydro-2H-pyran-2-yl | H | H | H |
| | | phenyl | | | | | |
| 56 | CH | 2,4-difluorophenyl | H | tetrahydro-2H-pyran-2-yl | H | H | H |
| 57 | CH | 4-tert-butylphenyl | H | tetrahydro-2H-pyran-2-yl | H | H | H |
| 58 | CH | 4-fluorophenyl | H | tert-butyl | H | H | H |
| 59 | CH | 4-phenoxyphenyl | H | tetrahydro-2H-pyran-2-yl | H | H | H |
| 60 | CH | 3-cyanophenyl | H | tetrahydro-2H-pyran-2-yl | H | H | H |
| 61 | CH | 3-cyano-4-fluorophenyl | H | tetrahydro-2H-pyran-2-yl | H | H | H |
| 62 | N | 4-methoxyphenyl | H | tetrahydro-2H-pyran-2-yl | 1H-pyrazol-1-yl | H | H |
| 63 | N | 4-fluorophenyl | H | tetrahydro-2H-pyran-2-yl | ethylamino | H | H |
| 64 | CH | 4-fluoro-2-methoxy- | H | tetrahydro-2H-pyran-2-yl | H | H | H |
| | | phenyl | | | | | |
| 65 | N | 4-methoxyphenyl | H | tetrahydro-2H-pyran-2-yl | H | —NHCH═CH— | |
| 66 | N | 4-fluorophenyl | H | tetrahydro-2H-pyran-2-yl | H | —NHCH═CH— | |
| 67 | CH | 3-phenoxyphenyl | H | tetrahydro-2H-pyran-2-yl | H | H | H |
| 68 | CH | 4-(methylsulphonyl)- | H | tetrahydro-2H-pyran-2-yl | H | H | H |
| | | phenyl | | | | | |
| 69 | CH | 4-methoxyphenyl | H | tetrahydro-2H-pyran-2-yl | amino | H | H |
| 70 | CH | 4-chlorophenyl | H | tetrahydro-2H-pyran-2-yl | H | H | H |
| 71 | CH | 4-(trifluoromethoxy)- | H | tetrahydro-2H-pyran-2-yl | H | H | H |
| | | phenyl | | | | | |
| 72 | CH | 4-fluorophenyl | H | propan-2-yl | (2,2-dimethyl- | H | H |
| | | | | | propanoyl)amino | | |
| 73 | CH | 4-methoxyphenyl | H | tetrahydro-2H-pyran-2-yl | CH₃ | H | H |
| 74 | N | 4-fluorophenyl | H | tetrahydro-2H-pyran-2-yl | methylsulphanyl | H | H |
| 75 | N | 4-fluorophenyl | H | tetrahydro-2H-pyran-2-yl | 1H-pyrazol-1-yl | H | H |
| 76 | CH | 2,3-dichlorophenyl | H | tetrahydro-2H-pyran-2-yl | H | H | H |
| 77 | N | 4-methoxyphenyl | H | tetrahydro-2H-pyran-2-yl | CH₃ | H | H |
| 78 | N | 4-methoxyphenyl | H | tetrahydro-2H-pyran-2-yl | ethylamino | H | H |
| 79 | CH | 4-fluorophenyl | H | 5-(trifluoromethyl)pyridin-2-yl | H | H | H |
| 80 | CH | 4-methoxyphenyl | H | tetrahydro-2H-pyran-2-yl | H | —NHCH═CH— | |
| 81 | CH | 4-fluorophenyl | H | tetrahydro-2H-pyran-2-yl | H | —N(COCH₃)—CH═CH— | |
| 82 | CH | 3-(trifluoromethyl)- | H | tetrahydro-2H-pyran-2-yl | (2,2-dimethyl- | H | H |
| | | phenyl | | | propanoyl)amino | | |
| 83 | CH | 3-chlorophenyl | H | tetrahydro-2H-pyran-2-yl | methylcarbamoyl | H | H |
| 84 | CH | 4-fluorophenyl | H | propan-2-yl | (2-methylpropanoyl)- | H | H |
| | | | | | amino | | |
| 85 | CH | 4-fluorophenyl | H | propan-2-yl | propanoylamino | H | H |
| 86 | CH | 4-fluorophenyl | H | propan-2-yl | (cyclopropyl- | H | H |
| | | | | | carbonyl)amino | | |
| 87 | CH | 4-fluorophenyl | H | 2-methylpropyl | (2-methylpropanoyl)- | H | H |
| | | | | | amino | | |
| 88 | CH | 4-fluorophenyl | H | 2-methoxyethyl | H | H | H |
| 89 | CH | 4-fluorophenyl | H | propan-2-yl | H | H | H |
| 90 | CH | 4-fluorophenyl | H | tetrahydro-2H-pyran-2-yl | methylcarbamoyl | H | H |
| 91 | CH | 4-fluorophenyl | H | butan-2-yl | H | H | H |
| 92 | CH | 4-fluorophenyl | H | cyclopentyl | H | H | H |
| 93 | CH | 4-fluorophenyl | H | tetrahydro-2H-pyran-2-yl | carbamoyl | H | H |
| 94 | CH | 4-fluorophenyl | H | tetrahydro-2H-pyran-2-yl | phenylcarbamoyl | H | H |
| 95 | CH | 3-chlorophenyl | H | tetrahydro-2H-pyran-2-yl | carbamoyl | H | H |
| 96 | CH | 3-chlorophenyl | H | tetrahydro-2H-pyran-2-yl | phenylcarbamoyl | H | H |
| 97 | CH | 4-fluorophenyl | H | propan-2-yl | amino | H | H |
| 98 | CH | 2-(benzyloxy)phenyl | H | tetrahydro-2H-pyran-2-yl | H | H | H |
| 99 | CH | quinolin-8-yl | H | tetrahydro-2H-pyran-2-yl | H | H | H |
| 100 | CH | 4-fluorophenyl | H | pyridine-3-ylmethyl | H | H | H |
| 101 | CH | 4-fluorophenyl | H | 1-(3-chlorophenyl)ethyl | H | H | H |
| 102 | CH | 4-fluorophenyl | H | 1-(2-chlorophenyl)ethyl | H | H | H |
| 103 | CH | 4-fluorophenyl | H | 2,2,2-trifluoroethyl | H | H | H |
| 104 | CH | 4-fluorophenyl | H | 2,3-difluorobenzyl | H | H | H |
| 105 | CH | 4-fluorophenyl | H | 2-tert-butoxy-2-oxoethyl | H | H | H |
| 106 | CH | 4-fluorophenyl | H | 1-methoxy-3-methyl-1- | H | H | H |
| | | | | oxobutan-2-yl | | | |
| 107 | CH | 4-fluorophenyl | H | 1-methoxy-1-oxopropan-2-yl | H | H | H |
| 108 | CH | 4-fluorophenyl | H | 2-chloro-6-fluorobenzyl | H | H | H |
| 109 | CH | 4-fluorophenyl | H | 4-(difluoromethoxy)benzyl | H | H | H |
| 110 | CH | 4-fluorophenyl | H | (2-chloro-1,3-thiazol-5-yl)- | H | H | H |
| | | | | methyl | | | |
| 111 | CH | 4-fluorophenyl | H | 5-(trifluoromethyl)-1,3,4- | H | H | H |
| | | | | thiadiazol-2-yl | | | |
| 112 | CH | 4-fluorophenyl | H | 6-(trifluoromethyl)pyrimidin-4- | H | H | H |
| | | | | yl | | | |
| 113 | CH | 4-fluorophenyl | H | 2-cyclohexyl-2-oxoethyl | H | H | H |
| 114 | CH | 4-fluorophenyl | H | 2-cyclopentyl-2-oxoethyl | H | H | H |
| 115 | CH | 4-fluorophenyl | H | pyridin-2-ylmethyl | H | H | H |
| 116 | CH | 4-fluorophenyl | H | (2,2-dichlorocyclopropyl)- | H | H | H |
| | | | | methyl | | | |
| 117 | CH | 4-fluorophenyl | H | 2-(methylsulphanyl)ethyl | H | H | H |
| 118 | CH | 4-fluorophenyl | H | 2-(3-chlorophenyl)ethyl | H | H | H |
| 119 | CH | 4-fluorophenyl | H | 2-(2-chlorophenyl)ethyl | H | H | H |
| 120 | CH | 4-fluorophenyl | H | 1-(4-chlorophenyl)ethyl | H | H | H |
| 121 | CH | 4-fluorophenyl | H | pyridin-4-ylmethyl | H | H | H |
| 122 | CH | 4-fluorophenyl | H | 2-(trifluoromethoxy)ethyl | H | H | H |
| 123 | CH | 4-fluorophenyl | H | tetrahydrofuran-2-ylmethyl | H | H | H |
| 124 | CH | 4-fluorophenyl | H | 2,3-dihydroxypropyl | H | H | H |
| 125 | CH | 4-fluorophenyl | H | biphenyl-3-ylmethyl | H | H | H |
| 126 | CH | 4-fluorophenyl | H | cyclopropylmethyl | H | H | H |
| 127 | CH | 4-fluorophenyl | H | benzyl | H | H | H |
| 128 | CH | 4-fluorophenyl | H | 2-fluorobenzyl | H | H | H |
| 129 | CH | 4-fluorophenyl | H | 3-fluorobenzyl | H | H | H |
| 130 | CH | 4-fluorophenyl | H | 4-fluorobenzyl | H | H | H |
| 131 | CH | 4-fluorophenyl | H | biphenyl-4-ylmethyl | H | H | H |
| 132 | CH | 4-fluorophenyl | H | 3-ethoxy-3-oxopropyl | H | H | H |
| 133 | CH | 4-fluorophenyl | H | 2-[2-(2-methoxyethoxy)- | H | H | H |
| | | | | ethoxy]ethyl | | | |
| 134 | CH | 4-fluorophenyl | H | 2-(2-methoxyethoxy)ethyl | H | H | H |
| 135 | CH | 4-fluorophenyl | H | biphenyl-2-ylmethyl | H | H | H |
| 136 | CH | 4-fluorophenyl | H | 2-cyanobenzyl | H | H | H |
| 137 | CH | 4-fluorophenyl | H | naphthalen-1-ylmethyl | H | H | H |
| 138 | CH | 4-fluorophenyl | H | 3-phenoxybenzyl | H | H | H |
| 139 | CH | 4-fluorophenyl | H | 4-fluoro-3-phenoxybenzyl | H | H | H |
| 140 | CH | 4-fluorophenyl | H | 3-cyanobenzyl | H | H | H |
| 141 | CH | 4-fluorophenyl | H | 4-cyanobenzyl | H | H | H |
| 142 | CH | 4-fluorophenyl | H | 2-oxotetrahydrofuran-3-yl | H | H | H |
| 143 | CH | 4-fluorophenyl | H | 1H-imidazol-2-ylmethyl | H | H | H |
| 144 | CH | 4-fluorophenyl | H | 2-(1,5-dimethyl-1H-pyrazol-3- | H | H | H |
| | | | | yl)-2-oxoethyl | | | |
| 145 | CH | 4-fluorophenyl | H | 2-(acetyloxy)ethyl | H | H | H |
| 146 | CH | 4-fluorophenyl | H | 2-cyanoethyl | H | H | H |
| 147 | CH | 4-fluorophenyl | H | 2-methylpropyl | H | —NHCOCH₂— | |
| 148 | CH | 4-fluorophenyl | H | 2-methylpropyl | H | —N(CH₃)COC(CH₃)₂— | |
| 149 | N | 4-fluorophenyl | H | 2-methylpropyl | prop-2-en-1-ylamino | H | H |
| 150 | N | 4-fluorophenyl | H | 2-methylpropyl | prop-2-en-1-ylamino | H | H |
| 151^{[s]} | CH | 4-methoxyphenyl | H | H | H | H | H |
| 152^{[s]} | CH | 3-(trifluoromethyl)- | H | H | H | H | H |
| | | phenyl | | | | | |
| 153 | CH | 4-fluorophenyl | H | 2-methoxyethyl | propanoylamino | H | H |
| 154 | CH | 3-cyano-4-fluorophenyl | H | 2-methylpropyl | H | H | H |
| 155 | CH | 4-fluorophenyl | H | 2-methylpropyl | propanoylamino | H | H |
| 156 | CH | 4-fluorophenyl | H | propan-2-yl | (cyclobutylcarbonyl)- | H | H |
| | | | | | amino | | |
| 157 | CH | 4-fluorophenyl | H | butan-2-yl | (2-methylpropanoyl)- | H | H |
| | | | | | amino | | |
| 158 | CH | 4-fluorophenyl | H | 2-methylpropyl | (cyclopropylacetyl)- | H | H |
| | | | | | amino | | |
| 159 | CH | 4-fluorophenyl | H | butan-2-yl | (cyclopropylacetyl)- | H | H |
| | | | | | amino | | |
| 160 | CH | 4-fluorophenyl | H | propan-2-yl | (3-methylbutanoyl)- | H | H |
| | | | | | amino | | |
| 161 | CH | 4-fluorophenyl | H | propan-2-yl | acetylamino | H | H |
| 162 | CH | 4-fluorophenyl | H | butan-2-yl | (methoxyacetyl)- | H | H |
| | | | | | amino | | |
| 163 | CH | 4-fluorophenyl | H | 2-methoxyethyl | (methoxyacetyl)- | H | H |
| | | | | | amino | | |
| 164 | CH | 4-fluorophenyl | H | butan-2-yl | propanoylamino | H | H |
| 165 | CH | 4-fluorophenyl | H | 2-methylpropyl | (cyclopropyl- | H | H |
| | | | | | carbonyl)amino | | |
| 166 | CH | 4-fluorophenyl | H | 2-methylpropyl | (methoxyacetyl)- | H | H |
| | | | | | amino | | |
| 167 | CH | 4-fluorophenyl | H | butan-2-yl | (cyclopropyl- | H | H |
| | | | | | carbonyl)amino | | |
| 168 | CH | 4-fluorophenyl | H | cyclohexyl | H | H | H |
| 169 | CH | 4-fluorophenyl | H | 2,2-difluoroethyl | H | H | H |
| 170 | CH | 4-fluorophenyl | H | 2-ethoxy-2-oxoethyl | H | H | H |
| 171 | CH | 4-fluorophenyl | CH₃ | prop-2-yn-1-yl | H | H | H |
| 172 | N | 4-fluorophenyl | H | H | propan-2-ylamino | H | H |
| 173 | N | 4-fluorophenyl | H | H | prop-2-yn-1-ylamino | H | H |
| 174 | N | 4-fluorophenyl | H | 2-methylpropyl | propan-2-ylamino | H | H |
| 175 | CH | 4-fluorophenyl | cyano | H | H | H | H |
| 176 | CH | 4-fluorophenyl | H | 1-(2-chloro-1,3-thiazol-5-yl)- | H | H | H |
| | | | | ethyl | | | |
| 177 | CH | 4-fluorophenyl | H | 1,3-difluoropropan-2-yl | H | H | H |
| 178 | CH | 4-fluorophenyl | H | 3-methyl-2-oxobutyl | H | H | H |
| 179 | CH | 4-fluorophenyl | H | 2-ethoxyethyl | H | H | H |
| 180 | CH | 4-fluorophenyl | H | cyanomethyl | H | H | H |
| 181 | CH | 4-fluorophenyl | H | propan-2-yl | [(propan-2-yloxy)- | H | H |
| | | | | | carbonyl]amino | | |
| 182 | N | 4-fluorophenyl | H | 2-methylpropyl | benzylamino | H | H |
| 183 | N | 4-fluorophenyl | H | 2-methylpropyl | prop-2-yn-1-ylamino | H | H |
| 184 | N | 4-fluorophenyl | H | 2-methylpropyl | amino | H | H |
| 185 | N | 4-fluorophenyl | H | 2-methylpropyl | (2-methylpropanoyl)- | H | H |
| | | | | | (propan-2-yl)amino | | |
| 186 | N | 4-fluorophenyl | H | 2-methylpropyl | propan-2-yl(2,2,4- | H | H |
| | | | | | trimethyl-3-oxo- | | |
| | | | | | pentanoyl)amino | | |
| 187 | CH | 4-fluorophenyl | H | 2-methylpropyl | 2-oxopyrrolidin-1-yl | H | H |
| 188 | N | 4-fluorophenyl | H | 2-methylpropyl | bis(2-methyl- | H | H |
| | | | | | propanoyl)amino | | |
| 189 | N | 4-fluorophenyl | H | 2-methylpropyl | (2-methyl- | H | H |
| | | | | | propanoyl)amino | | |
| 190 | CH | 4-fluorophenyl | H | ethyl | (phenylacetyl)amino | H | H |
| 191 | CH | 4-fluorophenyl | H | 2-methylpropyl | (phenylacetyl)amino | H | H |
| 192 | CH | 4-fluorophenyl | H | propan-2-yl | [(2S)-2-hydroxy- | H | H |
| | | | | | propanoyl]amino | | |
| 193 | CH | 4-fluorophenyl | H | 1-(tert-butoxycarbonyl)- | propanoylamino | H | H |
| | | | | piperidin-4-yl | | | |
| 194 | CH | 4-cyano-2,5- | difluoromethoxy | CH₃ | propanoylamino | H | H |
| | | difluorophenyl | | | | | |
| 195 | CH | 4-chloro-2- | methylsulphanyl | CH₃ | propanoylamino | H | H |
| | | fluorophenyl | | | | | |
| 196 | CH | 4-fluoro-2- | difluoromethoxy | CH₃ | propanoylamino | H | H |
| | | hydroxyphenyl | | | | | |
| 197 | CH | 4-chloro-2- | CF₃ | CH₃ | propanoylamino | H | H |
| | | fluorophenyl | | | | | |
| 198 | CH | 4-fluorophenyl | H | ethyl | propanoylamino | H | H |
| 199 | CH | 4-cyano-2,5- | difluoromethoxy | CH₃ | (methoxyacetyl)- | H | H |
| | | difluorophenyl | | | amino | | |
| 200 | CH | 4-chloro-2- | methylsulphanyl | CH₃ | (methoxyacetyl)- | H | H |
| | | fluorophenyl | | | amino | | |
| 201 | CH | 4-fluoro-2- | difluoromethoxy | CH₃ | (methoxyacetyl)- | H | H |
| | | hydroxyphenyl | | | amino | | |
| 202 | CH | 4-chloro-2- | CF₃ | CH₃ | (methoxyacetyl)- | H | H |
| | | fluorophenyl | | | amino | | |
| 203 | CH | 4-fluorophenyl | H | ethyl | (methoxyacetyl)- | H | H |
| | | | | | amino | | |
| 204 | CH | 4-fluorophenyl | H | 1-(tert-butoxycarbonyl)- | (methoxyacetyl)- | H | H |
| | | | | piperidin-4-yl | amino | | |
| 205 | CH | 4-fluorophenyl | H | propan-2-yl | benzoylamino | H | H |
| 206 | CH | 4-fluorophenyl | H | propan-2-yl | (thiophen-2-yl- | H | H |
| | | | | | carbonyl)amino | | |
| 207 | CH | 4-fluorophenyl | H | propan-2-yl | (thiophen-3-yl- | H | H |
| | | | | | carbonyl)amino | | |
| 208 | CH | 4-fluorophenyl | H | propan-2-yl | [(2-methoxyethoxy)- | H | H |
| | | | | | acetyl]amino | | |
| 209 | CH | 4-fluorophenyl | H | propan-2-yl | (2-hydroxy-2-methyl- | H | H |
| | | | | | propanoyl)amino | | |
| 210 | CH | 4-fluorophenyl | H | propan-2-yl | (2,3-dihydroxy- | H | H |
| | | | | | propanoyl)amino | | |
| 211 | CH | 4-fluorophenyl | H | propan-2-yl | (ethylcarbamoyl)- | H | H |
| | | | | | amino | | |
| 212 | CH | 4-fluorophenyl | H | 2-methoxyethyl | (phenylacetyl)amino | H | H |
| 213 | CH | 4-fluorophenyl | H | 2-fluorobenzyl | propanoylamino | H | H |
| 214 | CH | 4-fluorophenyl | H | cyclopropylmethyl | propanoylamino | H | H |
| 215 | CH | 4-fluorophenyl | H | 2,2-difluoroethyl | propanoylamino | H | H |
| 216 | CH | 4-fluorophenyl | H | 2,2-difluoroethyl | (methoxyacetyl)- | H | H |
| | | | | | amino | | |
| 217 | CH | 4-fluorophenyl | H | 2,2-difluoroethyl | (cyclopropyl- | H | H |
| | | | | | carbonyl)amino | | |
| 218 | CH | 4-fluorophenyl | H | 2,2-difluoroethyl | (2-methylpropanoyl)- | H | H |
| | | | | | amino | | |
| 219 | CH | 4-fluorophenyl | H | 2-chloroethyl | propanoylamino | H | H |
| 220 | CH | 4-fluorophenyl | H | 2-chloroethyl | (methoxyacetyl)- | H | H |
| | | | | | amino | | |
| 221 | CH | 4-fluorophenyl | H | 2-chloroethyl | (cyclopropyl- | H | H |
| | | | | | carbonyl)amino | | |
| 222 | CH | 4-fluorophenyl | H | 2-chloroethyl | (2-methylpropanoyl)- | H | H |
| | | | | | amino | | |
| 223 | CH | 4-fluorophenyl | H | cyclopropylmethyl | (methoxyacetyl)- | H | H |
| | | | | | amino | | |
| 224 | CH | 4-fluorophenyl | H | cyclopropylmethyl | (cyclopropyl- | H | H |
| | | | | | carbonyl)amino | | |
| 225 | CH | 4-fluorophenyl | H | cyclopropylmethyl | (2-methylpropanoyl)- | H | H |
| | | | | | amino | | |
| 226 | CH | 4-fluorophenyl | H | propan-2-yloxy | propanoylamino | H | H |
| 227 | CH | 4-fluorophenyl | H | propan-2-yloxy | H | H | H |
| 228 | CH | 4-fluorophenyl | H | 2-methylpropyl | H | H | 4-fluoro-phenyl |
| 229 | CH | 4-fluorophenyl | H | 2-methylpropyl | H | H | 3-fluoro-phenyl |
| 230 | CH | 3-cyano-4-fluorophenyl | H | 2-methylpropyl | propanoylamino | H | H |
| 231 | CH | 4-fluorophenyl | H | 2,2-difluoroethyl | (ethylcarbamoyl)- | H | H |
| | | | | | amino | | |
| 232 | CH | 4-fluorophenyl | H | 1-(2-fluorophenyl)ethyl | (2-methylpropanoyl)- | H | H |
| | | | | | amino | | |
| 233 | CH | 4-fluorophenyl | H | propan-2-yl | (dimethylcarbamoyl) | H | H |
| | | | | | amino | | |
| 234 | CH | 4-fluorophenyl | H | 2,2-difluoroethyl | (propan-2-yl- | H | H |
| | | | | | sulphonyl)amino | | |
| 235 | CH | 4-fluorophenyl | H | 2,2-difluoroethyl | (thiophen-3-yl- | H | H |
| | | | | | carbonyl)amino | | |
| 236 | CH | 4-fluorophenyl | H | 2,2-difluoroethyl | (thiophen-2-yl- | H | H |
| | | | | | carbonyl)amino | | |
| 237 | CH | 4-fluorophenyl | H | 2,2-difluoroethyl | (phenylcarbonyl)- | H | H |
| | | | | | amino | | |
| 238 | CH | 4-fluorophenyl | H | 2,2-difluoroethyl | (3-methylbutanoyl)- | H | H |
| | | | | | amino | | |
| 239 | CH | 4-fluorophenyl | H | 2,2-difluoroethyl | (2-methylbutanoyl)- | H | H |
| | | | | | amino | | |
| 240 | CH | 4-fluorophenyl | H | 2,2-difluoroethyl | (cyclobutylcarbonyl)- | H | H |
| | | | | | amino | | |
| 241 | CH | 4-fluorophenyl | H | 2,2-difluoroethyl | (ethoxycarbonyl)- | H | H |
| | | | | | amino | | |
| 242 | CH | 4-fluorophenyl | H | 2,2-difluoroethyl | acetylamino | H | H |
| 243 | CH | 4-fluorophenyl | H | 2,2-difluoroethyl | (2-hydroxy-2-methyl- | H | H |
| | | | | | propanoyl)amino | | |
| 244 | CH | 4-fluorophenyl | H | 1-(2-fluorophenyl)ethyl | (methoxyacetyl)- | H | H |
| | | | | | amino | | |
| 245 | CH | 4-fluorophenyl | H | (2,2-dichlorocyclopropyl)- | (methoxyacetyl)- | H | H |
| | | | | methyl | amino | | |
| 246 | CH | 3-cyano-4-fluorophenyl | H | 2-methylpropyl | (methoxyacetyl)- | H | H |
| | | | | | amino | | |
| 247 | CH | 4-fluorophenyl | H | 2-fluorobenzyl | (methoxyacetyl)- | H | H |
| | | | | | amino | | |
| 248 | CH | 4-fluorophenyl | H | 1-(2-fluorophenyl)ethyl | propanoylamino | H | H |
| 249 | CH | 4-fluorophenyl | H | (2,2-dichlorocyclopropyl)- | propanoylamino | H | H |
| | | | | methyl | | | |
| 250 | CH | 4-fluorophenyl | H | 1-(2-fluorophenyl)ethyl | (cyclopropylacetyl)- | H | H |
| | | | | | amino | | |
| 251 | CH | 4-fluorophenyl | H | (2,2-dichlorocyclopropyl)- | (cyclopropylacetyl)- | H | H |
| | | | | methyl | amino | | |
| 252 | CH | 3-cyano-4-fluorophenyl | H | 2-methylpropyl | (cyclopropylacetyl)- | H | H |
| | | | | | amino | | |
| 253 | CH | 4-fluorophenyl | H | 2-fluorobenzyl | (cyclopropylacetyl)- | H | H |
| | | | | | amino | | |
| 254 | CH | 4-fluorophenyl | H | (2,2-dichlorocyclopropyl)- | (2-methylpropanoyl)- | H | H |
| | | | | methyl | amino | | |
| 255 | CH | 3-cyano-4-fluorophenyl | H | 2-methylpropyl | (2-methylpropanoyl)- | H | H |
| | | | | | amino | | |
| 256 | CH | 4-fluorophenyl | H | 2-fluorobenzyl | (2-methylpropanoyl)- | H | H |
| | | | | | amino | | |
| 257 | CH | 4-fluoro-2- | difluoromethoxy | CH₃ | (2-methylpropanoyl)- | H | H |
| | | hydroxyphenyl | | | amino | | |
| 258 | CH | 4-fluoro-2- | difluoromethoxy | CH₃ | (cyclopropyl- | H | H |
| | | hydroxyphenyl | | | carbonyl)amino | | |
| 259 | CH | 4-fluorophenyl | H | 1-(2-fluorophenyl)ethyl | (cyclopropyl- | H | H |
| | | | | | carbonyl)amino | | |
| 260 | CH | 4-fluorophenyl | H | (2,2-dichlorocyclopropyl)- | (cyclopropyl- | H | H |
| | | | | methyl | carbonyl)amino | | |
| 261 | CH | 3-cyano-4-fluorophenyl | H | 2-methylpropyl | (cyclopropyl- | H | H |
| | | | | | carbonyl)amino | | |
| 262 | CH | 4-fluorophenyl | H | 2-fluorobenzyl | (cyclopropyl- | H | H |
| | | | | | carbonyl)amino | | |
| 263 | CH | 4-fluorophenyl | H | 1-(2-fluorophenyl)ethyl | (ethoxycarbonyl)- | H | H |
| | | | | | amino | | |
| 264 | CH | 4-fluorophenyl | H | (2,2-dichlorocyclopropyl)- | (ethoxycarbonyl)- | H | H |
| | | | | methyl | amino | | |
| 265 | CH | 3-cyano-4-fluorophenyl | H | 2-methylpropyl | (ethoxycarbonyl)- | H | H |
| | | | | | amino | | |
| 266 | CH | 4-fluorophenyl | H | 2-fluorobenzyl | (ethoxycarbonyl)- | H | H |
| | | | | | amino | | |
| 267 | CH | 4-fluorophenyl | H | 2-methylpropyl | (ethoxycarbonyl)- | H | H |
| | | | | | amino | | |
| 268 | CH | 4-fluorophenyl | H | butan-2-yl | (ethoxycarbonyl)- | H | H |
| | | | | | amino | | |
| 269 | CH | 4-fluorophenyl | H | cyclopentyloxy | (cyclopropyl- | H | H |
| | | | | | carbonyl)amino | | |
| 270 | CH | 4-fluorophenyl | H | cyclopentyloxy | (2-methylpropanoyl)- | H | H |
| | | | | | amino | | |
| 271 | CH | 4-fluorophenyl | H | propan-2-yloxy | (cyclopropyl- | H | H |
| | | | | | carbonyl)amino | | |
| 272 | CH | 4-fluorophenyl | H | propan-2-yloxy | (2-methylpropanoyl)- | H | H |
| | | | | | amino | | |
| 273 | N | 4-fluorophenyl | H | 2-methylpropyl | (cyclopropylmethyl)- | H | H |
| | | | | | amino | | |
| 274 | CH | 4-fluorophenyl | H | 1-methoxy-3-methyl-1- | propanoylamino | H | H |
| | | | | oxobutan-2-yl | | | |
| 275 | CH | 4-fluorophenyl | H | 1,3-dioxolan-2-ylmethyl | H | H | H |
| 276 | CH | 4-fluorophenyl | H | 1,3-dioxolan-2-ylmethyl | (2-methylpropanoyl)- | H | H |
| | | | | | amino | | |
| 277 | CH | 4-fluorophenyl | H | 1-methoxy-3-methyl-1- | (2-methylpropanoyl)- | H | H |
| | | | | oxobutan-2-yl | amino | | |
| 278 | CH | 4-fluorophenyl | H | 1,3-dioxolan-2-ylmethyl | propanoylamino | H | H |
| 279 | N | 4-fluorophenyl | H | 2-methylpropyl | oxetan-3-ylamino | H | H |
| 280 | CH | 4-fluorophenyl | H | propan-2-yl | H | —NHCH═N— | |
| 281 | N | 4-fluorophenyl | H | 2-methylpropyl | cyclopropylamino | H | H |
| 282 | N | 4-fluorophenyl | H | propan-2-yl | H | —NHCH═N— | |
| 283 | CH | 4-fluorophenyl | H | propan-2-yl | 4-methyl-1H- | H | H |
| | | | | | imidazol-1-yl | | |
| 284 | N | 4-fluorophenyl | H | 2-methylpropyl | tert-butylamino | H | H |
| 285 | N | 4-fluorophenyl | H | 2-methylpropyl | (1-methoxypropan-2- | H | H |
| | | | | | yl)amino | | |
| 286 | N | 4-fluorophenyl | H | 2-methylpropyl | butan-2-ylamino | H | H |
| 287 | N | 4-fluorophenyl | H | 2-methylpropyl | methylsulphonyl | H | H |
| 288 | N | 4-fluorophenyl | H | 2-methylpropyl | H | —C(CH₃)═CHS— | |
| 289 | N | 4-fluorophenyl | H | cyclopropylmethyl | H | —C(CH₃)═CHS— | |
| 290 | CH | 4-fluorophenyl | H | ethyl | (2-methylpropanoyl)- | H | H |
| | | | | | amino | | |
| 291 | CH | 4-fluorophenyl | H | ethyl | (cyclopropylacetyl)- | H | H |
| | | | | | amino | | |
| 292 | CH | 4-fluorophenyl | H | propyl | propanoylamino | H | H |
| 293 | CH | 4-fluorophenyl | H | propyl | (methoxyacetyl)- | H | H |
| | | | | | amino | | |
| 294 | CH | 4-fluorophenyl | H | propyl | (2-methylpropanoyl)- | H | H |
| | | | | | amino | | |
| 295 | CH | 4-fluorophenyl | H | propyl | (cyclopropylacetyl)- | H | H |
| | | | | | amino | | |
| 296 | CH | 4-fluorophenyl | H | CH₃ | propanoylamino | H | H |
| 297 | CH | 4-fluorophenyl | H | CH₃ | (methoxyacetyl)- | H | H |
| | | | | | amino | | |
| 298 | CH | 4-fluorophenyl | H | CH₃ | (2-methylpropanoyl)- | H | H |
| | | | | | amino | | |
| 299 | CH | 4-fluorophenyl | H | CH₃ | (cyclopropylacetyl)- | H | H |
| | | | | | amino | | |
| 300 | CH | 4-fluorophenyl | H | 2-(methylsulphanyl)ethyl | propanoylamino | H | H |
| 301 | CH | 4-fluorophenyl | H | 1-cyclopropylethyl | propanoylamino | H | H |
| 302 | CH | 4-fluorophenyl | H | 3-cyanobenzyl | propanoylamino | H | H |
| 303 | CH | 4-fluorophenyl | H | 2-cyanoethyl | propanoylamino | H | H |
| 304 | CH | 4-fluorophenyl | CF₃ | propan-2-yl | propanoylamino | H | H |
| 305 | CH | 4-fluorophenyl | H | 1-cyclopropylethyl | H | H | H |
| 306 | CH | 4-fluorophenyl | H | 2-(methylsulphanyl)ethyl | (2-methylpropanoyl)- | H | H |
| | | | | | amino | | |
| 307 | CH | 4-fluorophenyl | H | 1-cyclopropylethyl | (2-methylpropanoyl)- | H | H |
| | | | | | amino | | |
| 308 | CH | 4-fluorophenyl | H | 3-cyanobenzyl | (2-methylpropanoyl)- | H | H |
| | | | | | amino | | |
| 309 | CH | 4-fluorophenyl | H | 2-cyanoethyl | (2-methylpropanoyl)- | H | H |
| | | | | | amino | | |
| 310 | N | 4-fluorophenyl | H | propan-2-yl | H | —NHCH═CH— | |
| 311 | N | 4-fluorophenyl | H | propan-2-yl | H | —NHCH₂CH₂CH₂— | |
| 312 | CH | 4-fluorophenyl | H | 1-cyanopropan-2-yl | propanoylamino | H | H |
| 313 | CH | 4-fluorophenyl | H | 1-cyanopropan-2-yl | (methoxyacetyl)- | H | H |
| | | | | | amino | | |
| 314 | CH | 4-fluorophenyl | H | 1-cyanopropan-2-yl | (2-methylpropanoyl)- | H | H |
| | | | | | amino | | |
| 315 | CH | 4-fluorophenyl | H | 1-cyanopropan-2-yl | (cyclopropylacetyl)- | H | H |
| | | | | | amino | | |
| 316 | CH | 4-fluorophenyl | difluoromethoxy | CH₃ | propanoylamino | H | H |
| 317 | CH | 4-fluorophenyl | H | cyclopropylmethyl | (1,1,1-trifluoro- | H | H |
| | | | | | propan-2-yl)amino | | |
| 318 | CH | 4-fluorophenyl | H | propan-2-yl | (1,1,1-trifluoro- | H | H |
| | | | | | propan-2-yl)amino | | |
| 319 | CH | 4-fluorophenyl | H | CH₃ | (1,1,1-trifluoro- | H | H |
| | | | | | propan-2-yl)amino | | |
| 320 | CH | 4-fluorophenyl | H | cyclopropyl | propanoylamino | H | H |
| 321 | CH | 4-fluorophenyl | H | cyclopropyl | (methoxyacetyl)- | H | H |
| | | | | | amino | | |
| 322 | CH | 4-fluorophenyl | H | cyclopropyl | (2-methylpropanoyl)- | H | H |
| | | | | | amino | | |
| 323 | CH | 4-fluorophenyl | H | cyclopropyl | (cyclopropylacetyl)- | H | H |
| | | | | | amino | | |
| 324 | CH | 4-fluorophenyl | H | propan-2-yl | [2,2,2-trifluoro-1-(4- | H | H |
| | | | | | fluorophenyl)ethyl]- | | |
| | | | | | amino | | |
| 325 | CH | 4-fluorophenyl | H | propan-2-yl | (2,2,2-trifluoro-1- | H | H |
| | | | | | phenylethyl)amino | | |
| 326 | CH | 4-fluorophenyl | H | 2-ethoxyethyl | propanoylamino | H | H |
| 327 | CH | 4-fluorophenyl | H | 2-ethoxyethyl | (methoxyacetyl)- | H | H |
| | | | | | amino | | |
| 328 | CH | 4-fluorophenyl | H | 2-ethoxyethyl | (2-methylpropanoyl)- | H | H |
| | | | | | amino | | |
| 329 | CH | 4-fluorophenyl | H | 2-ethoxyethyl | (cyclopropylacetyl)- | H | H |
| | | | | | amino | | |
| 330 | CH | 4-fluorophenyl | H | ethyl | (6-methoxy-6-oxo- | H | H |
| | | | | | hexanoyl)amino | | |
| 331 | CH | 4-fluorophenyl | H | ethyl | (5-methoxy-5-oxo- | H | H |
| | | | | | pentanoyl)amino | | |
| 332 | CH | 4-fluorophenyl | H | ethyl | (5-ethoxy-3-ethyl-5- | H | H |
| | | | | | oxopentanoyl)amino | | |
| 333 | CH | 4-fluorophenyl | H | ethyl | {[3-(difluoromethyl)- | H | H |
| | | | | | 1-methyl-1H-pyrazol- | | |
| | | | | | 4-yl]carbonyl}amino | | |
| 334 | CH | 4-fluorophenyl | H | ethyl | {[2-(difluoromethyl)- | H | H |
| | | | | | thiophen-3-yl]- | | |
| | | | | | carbonyl}amino | | |
| 335 | CH | 4-fluorophenyl | H | ethyl | [(4-methyl-1,2,3- | H | H |
| | | | | | thiadiazol-5-yl)- | | |
| | | | | | carbonyl]amino | | |
| 336 | CH | 4-fluorophenyl | H | ethyl | [3-(4-chlorophenyl)- | H | H |
| | | | | | propanoyl]amino | | |
| 337 | CH | 4-fluorophenyl | H | ethyl | {[(1R,2R)-2-phenyl- | H | H |
| | | | | | cyclopropyl]- | | |
| | | | | | carbonyl}amino | | |
| 338 | CH | 4-fluorophenyl | H | ethyl | (cyclopentylacetyl)- | H | H |
| | | | | | amino | | |
| 339 | CH | 4-fluorophenyl | H | ethyl | (3-methylbutanoyl)- | H | H |
| | | | | | amino | | |
| 340 | CH | 4-fluorophenyl | H | ethyl | (2-methylbutanoyl)- | H | H |
| | | | | | amino | | |
| 341 | CH | 4-fluorophenyl | H | ethyl | [(2-methylcyclo- | H | H |
| | | | | | propyl)carbonyl]- | | |
| | | | | | amino | | |
| 342 | CH | 4-fluorophenyl | H | ethyl | acetylamino | H | H |
| 343 | CH | 4-fluorophenyl | H | CH₃ | (3-phenylpropanoyl)- | H | H |
| | | | | | amino | | |
| 344 | CH | 4-fluorophenyl | H | CH₃ | (4-fluorobenzoyl)- | H | H |
| | | | | | amino | | |
| 345 | CH | 4-fluorophenyl | H | CH₃ | [(2-methoxyethoxy)- | H | H |
| | | | | | acetyl]amino | | |
| 346 | CH | 4-fluorophenyl | H | CH₃ | (phenylcarbonyl)- | H | H |
| | | | | | amino | | |
| 347 | CH | 4-fluorophenyl | H | CH₃ | (3-methylbutanoyl)- | H | H |
| | | | | | amino | | |
| 348 | CH | 4-fluorophenyl | H | CH₃ | (2-methylbutanoyl)- | H | H |
| | | | | | amino | | |
| 349 | CH | 4-fluorophenyl | H | CH₃ | (cyclobutylcarbonyl)- | H | H |
| | | | | | amino | | |
| 350 | CH | 4-fluorophenyl | H | CH₃ | acetylamino | H | H |
| 351 | CH | 4-fluorophenyl | H | ethyl | [amino(oxo)acetyl]- | H | H |
| | | | | | amino | | |
| 352 | CH | 4-fluorophenyl | H | ethyl | [(2S)-2-hydroxy- | H | H |
| | | | | | propanoyl]amino | | |
| 353 | CH | 4-fluorophenyl | H | CH₃ | [(2S)-2-hydroxy- | H | H |
| | | | | | propanoyl]amino | | |
| 354 | N | 4-fluorophenyl | H | propan-2-yl | (methoxyacetyl)- | H | H |
| | | | | | amino | | |
| 355 | N | 4-fluorophenyl | H | propan-2-yl | (2-methylpropanoyl)- | H | H |
| | | | | | amino | | |
| 356 | N | 4-fluorophenyl | H | propan-2-yl | (cyclopropyl- | H | H |
| | | | | | carbonyl)amino | | |
| 357 | N | 4-fluorophenyl | H | 2-methylpropyl | acetylamino | H | H |
| 358 | N | 4-fluorophenyl | H | 2-methoxyethyl | propan-2-ylamino | H | H |
| 359 | N | 4-fluorophenyl | H | 2-chloroethyl | propan-2-ylamino | H | H |
| 360 | N | 4-fluorophenyl | H | butan-2-yl | propan-2-ylamino | H | H |
| 361 | N | 4-fluorophenyl | H | CH₃ | propan-2-ylamino | H | H |
| 362 | N | 4-fluorophenyl | H | 2-cyanoethyl | propan-2-ylamino | H | H |
| 363 | N | 4-fluorophenyl | H | prop-2-yn-1-yl | propan-2-ylamino | H | H |
| 364 | N | 4-fluorophenyl | H | 2,2-difluoroethyl | propan-2-ylamino | H | H |
| 365 | N | 4-fluorophenyl | H | 2-methylpropyl | propanoylamino | H | H |
| 366 | N | 4-fluorophenyl | H | propan-2-yl | amino | H | H |
| 367 | CH | 4-fluorophenyl | CH₃ | 2,2-difluoroethyl | H | H | H |
| 368 | CH | 4-fluorophenyl | CH₃ | 2-methylpropyl | H | H | H |
| 369 | N | 4-fluorophenyl | H | propan-2-yl | benzylamino | H | H |
| 370 | N | 4-fluorophenyl | H | ethyl | propan-2-ylamino | H | H |
| 371 | N | 4-fluorophenyl | H | propan-2-yl | propan-2-ylamino | H | H |
| 372 | N | 4-fluorophenyl | H | 2-methylpropyl | (1-cyclopropylethyl)- | H | H |
| | | | | | amino | | |

^{[s]}The following examples are present in the form of the following salts: Example 4: hydrochloride; Example 18: trihydrochloride; Example 151: hydrochloride; Example 152: hydrochloride.

TABLE 2
NMR data of compounds of the type [I-a] and [I-b]

| | | | Isomer |
|---|---|---|---|
| No. | Name | NMR | ratio^{[1]} |
| 1 | 4-[3-(4-fluorophenyl)-1-(2- | 1H-NMR (400 MHz, d₆-DMSO): δ = 8.47 (d, 2H), 8.22 (s, 1H), 7.43 (m, | — |
| | methylpropyl)-1H-pyrazol-4- | 2H), 7.22 (m, 4H), 3.99 (d, 2H), 2.20 (m, 1H), 0.92 (d, 6H) ppm | |
| | yl]pyridine | | |
| 2 | 4-[5-(2,6-difluorophenyl)-1-(2- | 1H-NMR (400 MHz, (d₆-DMSO): δ = 8.47 (s, 1H), 8.42 (d, 2H), 7.59 (m, | — |
| | methylpropyl)-1H-pyrazol-4- | 1H), 7.24 (m, 2H), 7.13 (m, 2H), 4.03 (d, 2H), 2.19 (m, 1H), 0.91 (d, 6H) | |
| | yl]pyridine | ppm | |
| 3 | 4-[1-(butan-2-yl)-3-(2,6- | 1H-NMR (400 MHz, d₆-DMSO): δ = 8.53 (s, 1H), 8.42 (d, 2H), 7.59 (m, | — |
| | difluorophenyl)-1H-pyrazol-4- | 1H), 7.25 (m, 2H), 7.14 (d, 2H), 4.36 (m, 1H), 1.84 (m, 2H), 1.50 (d, 3H), | |
| | yl]pyridine | 0.79 (d, 3H) ppm | |
| 5 | 4-[3-(3-chlorophenyl)-1-isobutyl-1H- | 1H-NMR (400 MHz, CDCl₃): δ = 9.12 (s, 1H), 8.48 (s, 1H), 8.08 (s, 1H), | — |
| | pyrazol-4-yl]pyrimidine | 7.58 (s, 1H), 7.36 (m, 3H), 7.10 (d, 1H), 3.99 (d, 2H), 2.32 (m, 1H), | |
| | | 0.99 (d, 6H) ppm | |
| 6 | 4-[5-(2,6-difluorophenyl)-1-(2,2- | 1H-NMR (400 MHz, d₆-DMSO): δ = 8.43 (s, 1H), 8.42 (d, 2H), 7.58 (m, | — |
| | dimethylpropyl)-1H-pyrazol-4- | 1H), 7.23 (m, 2H), 7.13 (d, 2H), 4.02 (s, 2H), 0.96 (s, 9H) ppm | |
| | yl]pyridine | | |
| 7 | 4-[3-(2,6-difluorophenyl)-1-(3- | 1H-NMR (400 MHz, d₆-DMSO): δ = 8.42 (d, 2H), 8.17 (s, 1H), 7.75 (m, | — |
| | methylbut-2-en-1-yl)-1H-pyrazol-4- | 1H), 7.38 (m, 2H), 7.13 (m, 2H), 5.14 (m, 1H), 4.59 (d, 2H), 1.59 (s, 3H), | |
| | yl]pyridine | 1.36 (s, 3H) ppm | |
| 8 | 4-[1-cyclobutyl-3-(2,6- | 1H-NMR (400 MHz, d₆-DMSO): δ = 8.42 (d, 2H), 8.24 (s, 1H), 7.76 (m, | — |
| | difluorophenyl)-1H-pyrazol-4- | 1H), 7.38 (m, 2H), 7.13 (d, 2H), 4.50 (m, 1H), 2.58 (m, 2H), 2.23 (m, 2H), | |
| | yl]pyridine | 1.75 (m, 2H) ppm | |
| 9 | 4-[3-(4-fluorophenyl)-1-(tetrahydro- | 1H-NMR (400 MHz, d3-CD3CN): δ = 8.45 (dd, 2H), 8.02 (s, 1H), 7.44 (dd, | — |
| | 2H-pyran-2-yl)-1H-pyrazol-4- | 2H), 7.20 (dd, 2H), 7.10 (t, 2H), 5.44 (dd, 1H), 4.05-4.00 (m, 1H), | |
| | yl]pyridine | 3.75-3.70 (m, 1H), 2.20-2.10 (m, 1H), 2.05-1.95 (m, 2H), 1.70-1.50 (m, 3H) ppm | |
| 10 | 4-[5-(2,6-difluorophenyl)-1-(propan- | 1H-NMR (400 MHz, d₆-DMSO): δ = 8.53 (s, 1H), 8.42 (d, 2H), 7.59 (m, | — |
| | 2-yl)-1H-pyrazol-4-yl]pyridine | 1H), 7.24 (m, 2H), 7.15 (m, 2H), 4.60 (m, 1H), 1.51 (d, 6H) ppm | |
| 11 | 4-[3-(2,6-difluorophenyl)-1-(2,2- | 1H-NMR (400 MHz, d₆-DMSO): δ = 8.42 (d, 2H), 8.20 (s, 1H), 7.74 (m, | — |
| | dimethylpropyl)-1H-pyrazol-4- | 1H), 7.38 (m, 2H), 7.12 (d, 2H), 3.33 (s, 2H), 0.79 (s, 9H) ppm | |
| | yl]pyridine | | |
| 13 | 4-[3-(2,6-difluorophenyl)-1-(2- | 1H-NMR (400 MHz, d₆-DMSO): δ = 8.42 (d, 2H), 8.21 (s, 1H), 7.77 (m, | — |
| | methylpropyl)-1H-pyrazol-4- | 1H), 7.39 (m, 2H), 7.13 (d, 2H), 3.75 (d, 2H), 2.05 (m, 1H), 0.73 (d, 6H) | |
| | yl]pyridine | ppm | |
| 15 | 4-[1-cyclobutyl-5-(2,6- | 1H-NMR (400 MHz, d₆-DMSO): δ = 8.59 (s, 1H), 8.42 (d, 2H), 7.60 (m, | — |
| | difluorophenyl)-1H-pyrazol-4- | 1H), 7.24 (m, 2H), 7.14 (d, 2H), 4.94 (m, 1H), 2.60-2.45 (m, 4H), | |
| | yl]pyridine | 1.83 (m, 2H) ppm | |
| 16 | 4-[1-cyclopentyl-3-(2,6- | 1H-NMR (400 MHz, d₆-DMSO): δ = 8.42 (d, 2H), 8.20 (s, 1H), 7.76 (m, | — |
| | difluorophenyl)-1H-pyrazol-4- | 1H), 7.39 (m, 2H), 7.11 (d, 2H), 4.37 (m, 1H), 1.95 (m, 4H), 1.84 (m, 2H), | |
| | yl]pyridine | 1.58 (m, 2H) ppm | |
| 19 | N-{4-[3-(4-fluorophenyl)-1- | 1H-NMR (400 MHz, d₆-DMSO): δ = 10.38 (s, 1H), 8.29 (s, 1H), 8.18 (d, | — |
| | (tetrahydro-2H-pyran-2-yl)-1H- | 1H), 8.07 (s, 1H), 7.44 (dd, 2H), 7.20 (t, 2H), 6.85 (dd, 1H), 5.49 (dd, 1H), | |
| | pyrazol-4-yl]pyridin-2- | 3.96 (m, 1H), 3.68 (m, 1H), 2.35 (q, 2H), 2.18 (m, 2H), 2.00-1.90 (m, 2H), | |
| | yl}propanamide | 1.70 (m, 1H), 1.58 (m, 1H), 1.03 (t, 3H) ppm | |
| 20 | 4-[3-(4-fluorophenyl)-1-(tetrahydro- | 1H-NMR (400 MHz, d₃-CD3CN): δ = 9.70 (s, 1H, br), 8.16 (d, 1H), 8.02 (s, | — |
| | 2H-pyran-2-yl)-1H-pyrazol-4-yl]-1H- | 1H), 7.42 (dd, 2H), 7.25 (m, 1H), 6.99 (t, 2H), 6.89 (d, 1H), 6.15 (d, 1H), | |
| | pyrrolo[2,3-b]pyridine | 5.46 (dd, 1H), 4.04 (m, 1H), 3.76 (m, 1H), 2.25-2.15 (m, 1H), 2.10-2.00 (m, | |
| | | 2H), 1.80-1.60 (m, 3H) ppm | |
| 33 | 4-[5-(2,2-difluoro-1,3-benzodioxol-4- | 1H-NMR (400 MHz, d₃-CD3CN): δ = 8.39 (m, 2H), 7.97 (s, 1H), | — |
| | yl)-1-(tetrahydro-2H-pyran-2-yl)-1H- | 7.70-7.60 (m, 1H), 7.53-7.50 (m, 1H), 7.35-7.30 (m, 2H), 7.12 (m, 2H), 5.10 (m, 1H), | |
| | pyrazol-4-yl]pyridine | 3.90 (m, 1H), 3.40 (m, 1H), 2.50-2.40 (m, 1H), 2.00 (m, 1H), 1.90 (m, 1H), | |
| | | 1.60-1.40 (m, 3H) ppm | |
| 38 | 4-[1-(butan-2-yl)-5-(2,6- | 1H-NMR (400 MHz, d₆-DMSO): δ = 8.42 (d, 2H), 8.23 (s, 1H), 7.77 (m, | |
| | difluorophenyl)-1H-pyrazol-4- | 1H), 7.41 (t, 2H), 7.13 (d, 2H), 3.85 (m, 1H), 1.89 (m, 1H), 1.68 (m, 1H), | |
| | yl]pyridine | 1.38 (d, 3H), 0.59 (t, 3H) ppm | |
| 39 | 4-[3-(4-fluorophenyl)-1-(tetrahydro- | 1H-NMR (400 MHz, d₃-CD₃CN): δ = 7.91 (s, 1H), 7.87 (d, 1H), 7.47 (dd, | — |
| | 2H-pyran-2-yl)-1H-pyrazol-4- | 2H), 7.10 (t, 2H), 6.45 (dd, 1H), 6.37 (s, 1H), 5.42 (dd, 1H), 4.79 (s, 2H, | |
| | yl]pyridin-2-amine | br), 4.05-4.00 (m, 1H), 3.75-3.65 (m, 1H), 2.20-2.10 (m, 1H), 2.05-1.95 (m, | |
| | | 2H), 1.70-1.50 (m, 3H) ppm | |
| 40 | N-{4-[3-(4-fluorophenyl)-1-(propan- | 1H-NMR (400 MHz, d₆-DMSO): δ = 9.90 (s, 1H), 8.22 (s, 1H), 8.19 (d, | — |
| | 2-yl)-1H-pyrazol-4-yl]pyridin-2-yl}-2- | 1H), 8.02 (s, 1H), 7.44 (dd, 2H), 7.20 (t, 2H), 6.93 (d, 1H), 4.58 (m, 1H), | |
| | methoxyacetamide | 4.02 (s, 3H), 3.30 (m, 2H), 1.50 (d, 6H) ppm | |
| 43 | 4-[3-(4-methoxyphenyl)-1- | 1H-NMR (400 MHz, d₃-CD₃CN): δ = 8.44 (dd, 2H), 8.00 (s, 1H), 7.34 (d, | — |
| | (tetrahydro-2H-pyran-2-yl)-1H- | 2H), 7.20 (dd, 2H), 6.92 (d, 2H), 5.43 (dd, 1H), 4.04 (m, 1H), 3.80 (s, 3H), | |
| | pyrazol-4-yl]pyridine | 3.60 (m, 1H), 2.15 (m, 1H), 2.05-2.00 (m, 2H), 1.70-1.50 (m, 3H) ppm | |
| 44 | 4-[3-(4-fluorophenyl)-1-(propan-2-yl)- | 1H-NMR (400 MHz, d₆-DMSO): δ = 8.86 (d, 1H), 8.15 (s, 1H), 8.05 (d, | — |
| | 1H-pyrazol-4-yl]quinoline | 1H), 7.75-7.70 (m, 2H), 7.48 (m, 1H), 7.35 (d, 1H), 7.28 (dd, 2H), 7.03 (t, | |
| | | 2H), 4.65 (m, 1H), 1.56 (d, 6H) ppm | |
| 46 | 4-[3-(4-fluorophenyl)-1-(propan-2-yl)- | 1H-NMR (400 MHz, d₃-CD₃CN): δ = 7.80 (m, 2H), 7.50 (dd, 2H), 7.10 (dd, | — |
| | 1H-pyrazol-4-yllpyridin-2-amine | 1H), 6.47 (d, 1H), 6.39 (s, 1H), 5.10 (s, 2H, br), 4.53 (m, 1H), 1.20 (d, 6H) | |
| | | ppm | |
| 47 | 4-[3-(4-fluorophenyl)-1-(propan-2-yl)- | 1H-NMR (400 MHz, d₃-CD₃CN): δ = 8.18 (d, 1H), 8.05 (d, 1H, isomer), | 80:20 |
| | 1H-pyrazol-4-yl]-1-(prop-2-yn-1-yl)- | 7.96 (s, 1H, isomer), 7.93 (s, 1H), 7.43 (m, 3H), 7.38 (dd, 2H, isomer), | |
| | 1H-pyrrolo[2,3-b]pyridine | 7.19 (t, 2H, isomer), 7.01 (t, 2H), 6.89 (d, 1H), 6.64 (d, 1H, isomer), 6.48 (d, 1H, | |
| | | isomer), 6.29 (d, 1H), 5.08 (d, 1H), 5.04 (d, 1H, isomer), 4.60 (d, 1H, | |
| | | 4.48 (m, 1H, isomer), 2.63 (t, 1H), 2.60 (t, 1H, isomer), 1.56 (d, 6H), 1.42 (d, | |
| | | 6H, isomer) ppm | |
| 53 | 4-[3-(4-fluorophenyl)-1-(propan-2-yl)- | 1H-NMR (400 MHz, d₆-DMSO): δ = 8.74 (d, 1H), 8.50 (d, 1H), 8.41 (s, | — |
| | 1H-pyrazol-4-yl]-N-methylpyridine-2- | 1H), 7.90 (s, 1H), 7.45-7.40 (dd, 2H), 7.37 (d, 1H), 7.25-7.20 (t, 2H), | |
| | carboxamide | 4.57 (m, 1H), 2.79 (d, 3H), 1.51 (d, 6H) ppm | |
| 70 | 4-[5-(4-chlorophenyl)-1-(tetrahydro- | 1H-NMR (400 MHz, d₃-CD₃CN): δ = 8.37 (m, 2H), 7.90 (s, 1H), 7.53 (d, | — |
| | 2H-pyran-2-yl)-1H-pyrazol-4- | 2H), 7.37 (d, 2H), 7.09 (m, 2H), 5.02 (dd, 1H), 3.96 (m, 1H), 3.46 (m, 1H), | |
| | yl]pyridine | 2.50-2.35 (m, 1H), 2.00 (m, 1H), 1.80 (m, 1H), 1.60-1.40 (m, 3H) ppm | |
| 76 | 4-[5-(2,3-dichlorophenyl)-1- | 1H-NMR (400 MHz, d₃-CD₃CN): δ = 8.37 (m, 2H), 8.01 (s, 1H), 7.75 (m, | 40:60 |
| | (tetrahydro-2H-pyran-2-yl)-1H- | 1H), 7.50-7.45 (m, 1H), 7.43-7.38 (m, 1H), 7.05 (m, 2H), 5.08 (dd, 1H), | |
| | pyrazol-4-yl]pyridine | 4.90 (dd, 1H, isomer), 3.90-3.80 (m, 1H), 3.40-3.30 (m, 1H), 2.50-2.40 (m, | |
| | | 1H), 2.00 (m, 1H), 1.80 (m, 1H), 1.60-1.40 (m, 3H) ppm | |
| 81 | 1-{4-[3-(4-fluorophenyl)-1- | 1H-NMR (400 MHz, d₃-CD₃CN): δ = 8.29 (d, 1H), 8.03 (s, 1H), 7.85 (d, | — |
| | (tetrahydro-2H-pyran-2-yl)-1H- | 1H), 7.40-7.35 (dd, 2H), 7.09 (d, 1H), 7.02 (t, 2H), 6.34 (d, 1H), 5.49 (dd, | |
| | pyrazol-4-yl]-1H-pyrrolo[2,3- | 1H), 4.05-4.00 (m, 1H), 3.75-3.70 (m, 1H), 3.00 (s, 3H), 2.30-2.00 (m, 3H), | |
| | b]pyridin-1-yl}ethanone | 1.80-1.60 (m, 3H) ppm | |
| 84 | N-{4-[3-(4-fluorophenyl)-1-(propan- | 1H-NMR (400 MHz, d₆-DMSO): δ = 10.36 (s, 1H), 8.18 (s, 1H), 8.16 (d, | — |
| | 2-yl)-1H-pyrazol-4-yl]pyridin-2-yl}-2- | 1H), 8.10 (s, 1H), 7.44 (dd, 2H), 7.19 (t, 2H), 6.80 (dd, 1H), 4.57 (m, 1H), | |
| | methylpropanamide | 2.73 (m, 1H), 1.50 (d, 6H), 1.06 (d, 6H) ppm | |
| 85 | N-{4-[3-(4-fluorophenyl)-1-(propan- | 1H-NMR (400 MHz, d₃-CD₃CN): δ = 8.55 (s, 1H, br), 8.10 (m, 2H), | — |
| | 2-yl)-1H-pyrazol-4-yl]pyridin-2- | 7.88 (s, 1H), 7.46 (dd, 2H), 7.09 (t, 2H), 6.87 (dd, 1H), 4.55 (m, 1H), 2.38 (q, | |
| | yl}propanamide | 2H), 1.51 (d, 6H), 1.10 (t, 2H) ppm | |
| 86 | N-{4-[3-(4-fluorophenyl)-1-(propan- | 1H-NMR (400 MHz, d₃-CD₃CN): δ = 8.82 (s, 1H, br), 8.11 (d, 1H), 8.07 (s, | — |
| | 2-yl)-1H-pyrazol-4-yl]pyridin-2- | 1H), 7.85 (s, 1H), 7.44 (dd, 2H), 7.06 (t, 2H), 6.86 (dd, 1H), 4.54 (m, 1H), | |
| | yl}cyclopropanecarboxamide | 1.78 (m, 1H), 1.51 (d, 6H), 0.90-0.80 (m, 4H) ppm | |
| 87 | N-{4-[3-(4-fluorophenyl)-1-(2- | 1H-NMR (400 MHz, d₃-CD₃CN): δ = 8.59 (s, 1H, br), 8.12 (m, 2H), | — |
| | methylpropyl)-1H-pyrazol-4- | 7.83 (s, 1H), 7.45 (dd, 2H), 7.09 (t, 2H), 6.86 (dd, 1H), 3.96 (d, 2H), 2.62 (m, | |
| | yl]pyridin-2-yl}-2- | 1H), 2.21 (m, 1H), 1.14 (d, 6H), 0.93 (d, 6H) ppm | |
| | methylpropanamide | | |
| 88 | 4-[3-(4-fluorophenyl)-1-(2- | 1H-NMR (400 MHz, d₆-DMSO): δ = 8.45 (d, 2H), 8.37 (d, 2H, isomer), | 70:30 |
| | methoxyethyl)-1H-pyrazol-4- | 8.20 (s, 1H), 8.08 (s, 1H, isomer), 7.48-7.38 (m, 2H), 7.25-7.20 (m, 4H), | |
| | yl]pyridine | 7.08 (d, 2H, isomer), 4.33 (t, 2H), 4.06 (t, 2H, isomer), 3.77 (t, 2H), 3.65 (t, | |
| | | 2H, isomer), 3.27 (s, 3H), 3.11 (s, 3H, isomer) ppm | |
| 92 | 4-[1-cyclopentyl-3-(4-fluorophenyl)- | 1H-NMR (400 MHz, d₆-DMSO): δ = 8.45 (d, 2H), 8.36 (d, 2H, isomer), | 85:15 |
| | 1H-pyrazol-4-yl]pyridine | 8.28 (s, 1H), 8.07 (s, 1H, isomer), 7.48-7.38 (m, 2H), 7.25-7.20 (m, 4H), | |
| | | 7.07 (d, 2H, isomer), 4.75 (m, 1H), 4.38 (m, 1H, isomer), 2.15-2.10 (m, | |
| | | 2H), 2.05-2.00 (m, 2H), 2.00-1.90 (m, 2H, isomer), 1.80 (m, 2H), | |
| | | 1.70-1.65 (m, 2H), 1.55 (m, 2H, isomer) ppm | |
| 92 | 4-[1-cyclopentyl-3-(4-fluorophenyl)- | 1H-NMR (400 MHz, d₆-DMSO): δ = 8.45 (d, 2H), 8.36 (d, 2H, isomer), | 80:20 |
| | 1H-pyrazol-4-yl]pyridine | 8.28 (s, 1H), 8.07 (s, 1H, isomer), 7.48-7.38 (m, 2H), 7.25-7.20 (m, 4H), | |
| | | 7.07 (d, 2H, isomer), 4.75 (m, 1H), 4.38 (m, 1H, isomer), 2.15-2.10 (m, | |
| | | 2H), 2.05-2.00 (m, 2H), 2.00-1.90 (m, 2H, isomer), 1.80 (m, 2H), | |
| | | 1.70-1.65 (m, 2H), 1.55 (m, 2H, isomer) ppm | |
| 100 | 3-{[3-(4-fluorophenyl)-4-(pyridin-4- | 1H-NMR (400 MHz, d₆-DMSO): δ = 8.63 (s, 1H), 8.54 (d, 1H), 8.47 (d, | — |
| | yl)-1H-pyrazol-1-yl]methyl}pyridine | 2H), 8.38 (s, 1H), 7.78 (d, 1H), 7.40 (m, 3H), 7.25-7.20 (m, 4H), 5.46 (s, | |
| | | 2H) ppm | |
| 101 | 4-{1-[1-(3-chlorophenyl)ethyl]-3-(4- | 1H-NMR (400 MHz, d₆-DMSO): δ = 8.47 (d, 2H), 8.38 (d, 2H, isomer), | 80:20 |
| | fluorophenyl)-1H-pyrazol-4- | 7.50-7.10 (m, 11H), 7.11 (m, 2H, isomer), 5.72 (m, 1H), 5.30 (m, 1H, | |
| | yl}pyridine | isomer), 1.88 (d, 3H), 1.79 (d, 3H, isomer) ppm | |
| 102 | 4-{1-[1-(2-chlorophenyl)ethyl]-3-(4- | 1H-NMR (400 MHz, d₆-DMSO): δ = 8.46 (m, 3H), 8.37 (d, 2H, isomer), | 91:9 |
| | fluorophenyl)-1H-pyrazol-4- | 7.50 (d, 1H), 7.40-7.20 (m, 10H), 7.11 (d, 2H, isomer), 6.03 (q, 2H), | |
| | yl}pyridine | 5.65 (q, 2H, isomer), 1.88 (d, 3H), 1.79 (d, 3H, isomer) ppm | |
| 103 | 4-[3-(4-fluorophenyl)-1-(2,2,2- | 1H-NMR (400 MHz, d₆-DMSO): δ = 8.50 (d, 2H), 8.40 (d, 2H, isomer), | 91:9 |
| | trifluoroethyl)-1H-pyrazol-4- | 8.33 (s, 1H), 8.23 (s, 1H, isomer), 7.48-7.38 (m, 2H), 7.25-7.20 (m, 4H), | |
| | yl]pyridine | 7.11 (d, 2H, isomer), 5.25 (q, 2H), 4.89 (q, 2H, isomer) ppm | |
| 104 | 4-[1-(2,3-difluorobenzyl)-3-(4- | 1H-NMR (400 MHz, d₆-DMSO): δ = 8.47 (d, 2H), 8.38 (d, 2H, isomer), | 75:25 |
| | fluorophenyl)-1H-pyrazol-4- | 8.35 (s, 1H), 8.14 (s, 1H, isomer), 7.45-7.30 (m, 4H), 7.25-7.20 (m, 5H), | |
| | yl]pyridine | 7.11 (d, 2H, isomer), 5.52 (s, 2H), 5.29 (s, 1H, isomer) ppm | |
| 105 | tert-butyl [3-(4-fluorophenyl)-4- | 1H-NMR (400 MHz, d₆-DMSO): δ = 8.48 (d, 2H), 8.39 (d, 2H, isomer), | 78:22 |
| | (pyridin-4-yl)-1H-pyrazol-1-yl]acetate | 8.21 (s, 1H), 8.10 (s, 1H, isomer), 7.40 (m, 2H), 7.25-7.20 (m, 4H), 7.12 (d, | |
| | | 2H, isomer), 5.04 (s, 2H), 4.79 (s, 2H, isomer), 1.45 (s, 9H), 1.30 (s, 9H, | |
| | | isomer) ppm | |
| 106 | methyl 2-[3-(4-fluorophenyl)-4- | 1H-NMR (400 MHz, d₆-DMSO): δ = 8.48 (d, 2H), 8.37 (d, 2H, isomer), | 93:7 |
| | (pyridin-4-yl)-1H-pyrazol-1-yl]-3- | 8.36 (s, 1H), 8.17 (s, 1H, isomer), 7.43 (m, 2H), 7.25-7.20 (m, 4H), 7.09 (d, | |
| | methylbutanoate | 2H, isomer), 4.89 (d, 1H), 4.30 (d, 1H, isomer), 3.72 (s, 3H), 3.64 (s, 3H, | |
| | | isomer), 1.00 (d, 3H), 0.87 (d, 3H), 0.71 (d, 3H, isomer) ppm | |
| 107 | methyl 2-[3-(4-fluorophenyl)-4- | 1H-NMR (400 MHz, d₆-DMSO): δ = 8.48 (d, 2H), 8.36 (s, 1H), 7.43 (dd, | — |
| | (pyridin-4-yl)-1H-pyrazol-1- | 2H), 7.25-7.20 (m, 4H), 5.36 (q, 1H), 3.70 (s, 3H), 1.75 (d, 3H) ppm | |
| | yl]propanoate | | |
| 108 | 4-[1-(2-chloro-6-fluorobenzyl)-3-(4- | 1H-NMR (400 MHz, d₆-DMSO): δ = 8.46 (d, 2H), 8.37 (d, 2H, isomer), | 87:13 |
| | fluorophenyl)-1H-pyrazol-4- | 8.29 (s, 1H), 8.00 (s, 1H, isomer), 7.55-7.45 (m, 1H), 7.40-7.30 (m, 3H), | |
| | yl]pyridine | 7.25-7.20 (m, 4H), 7.09 (d, 2H, isomer), 5.53 (s, 2H), 5.26 (s, 1H, isomer) | |
| | | ppm | |
| 109 | 4-{1-[4-(difluoromethoxy)benzyl]-3- | 1H-NMR (400 MHz, d₆-DMSO): δ = 8.46 (d, 2H), 8.38 (d, 2H, isomer), | 74:26 |
| | (4-fluorophenyl)-1H-pyrazol-4- | 8.35 (s, 1H), 8.14 (s, 1H, isomer), 7.45-7.35 (m, 4H), 7.25-7.20 (m, 6H), | |
| | yl}pyridine | 7.15 (m, 1H), 7.05 (d, 2H, isomer), 5.39 (s, 2H), 5.18 (s, 1H, isomer) ppm | |
| 110 | 4-{1-[(2-chloro-1,3-thiazol-5- | 1H-NMR (400 MHz, d₆-DMSO): δ = 8.47 (d, 2H), 8.32 (s, 1H), 7.78 (s, | — |
| | yl)methyl]-3-(4-fluorophenyl)-1H- | 1H), 7.45-7.40 (dd, 2H), 7.25-7.20 (m, 4H), 5.66 (s, 2H) ppm | |
| | pyrazol-4-yl}pyridine | | |
| 111 | 4-{3-(4-fluorophenyl)-1-[5- | 1H-NMR (400 MHz, d₆-DMSO): δ = 9.32 (s, 1H), 8.58 (d, 2H), 7.53 (dd, | — |
| | (trifluoromethyl)-1,3,4-thiadiazol-2- | 2H), 7.37 (d, 2H), 7.34 (t, 2H) ppm | |
| | yl]-1H-pyrazol-4-yl}pyridine | | |
| 112 | 4-[3-(4-fluorophenyl)-4-(pyridin-4- | 1H-NMR (400 MHz, d₆-DMSO): δ = 9.39 (s, 1H), 9.26 (s, 1H), 8.57 (d, | — |
| | yl)-1H-pyrazol-1-yl]-6- | 2H), 8.35 (s, 1H), 7.59 (dd, 2H), 7.38 (d, 2H), 7.33 (t, 2H) ppm | |
| | (trifluoromethyl)pyrimidine | | |
| 113 | 1-cyclohexyl-2-[3-(4-fluorophenyl)-4- | 1H-NMR (400 MHz, d₆-DMSO): δ = 8.47 (d, 2H), 8.38 (d, 2H, isomer), | 80:20 |
| | (pyridin-4-yl)-1H-pyrazol-1- | 8.12 (s, 1H), 8.09 (s, 1H, isomer), 7.43-7.35 (m, 2H), 7.25-7.20 (m, 4H), | |
| | yl]ethanone | 7.11 (d, 2H, isomer), 5.32 (s, 2H), 5.09 (s, 1H, isomer), 2.60 (m, 1H), | |
| | | 1.90 (m, 2H), 1.70 (m, 2H), 1.65-1.60 (m, 2H), 1.30-1.10 (m, 4H) ppm | |
| 114 | 1-cyclopentyl-2-[3-(4-fluorophenyl)- | 1H-NMR (400 MHz, d₆-DMSO): δ = 8.47 (d, 2H), 8.38 (d, 2H, isomer), | 75:25 |
| | 4-(pyridin-4-yl)-1H-pyrazol-1- | 8.15 (s, 1H), 8.10 (s, 1H, isomer), 7.43-7.35 (m, 2H), 7.25-7.20 (m, 4H), | |
| | yl]ethanone | 7.11 (d, 2H, isomer), 5.30 (s, 2H), 5.08 (s, 1H, isomer), 3.05 (m, 1H), | |
| | | 2.90 (m, 1H, isomer), 1.90-1.20 (m, 8H) ppm | |
| 115 | 2-{[3-(4-fluorophenyl)-4-(pyridin-4- | 1H-NMR (400 MHz, d₆-DMSO): δ = 8.56 (d, 1H), 8.47 (d, 2H), 8.22 (s, | — |
| | yl)-1H-pyrazol-1-yl]methyl}pyridine | 1H), 7.88 (t, 1H), 7.58 (d, 1H), 7.52 (d, 1H), 7.46 (dd, 2H), 7.38 (m, 1H), | |
| | | 7.25-7.20 (m, 2H), 5.63 (s, 2H) ppm | |
| 115 | 2-{[3-(4-fluorophenyl)-4-(pyridin-4- | 1H-NMR (400 MHz, d₆-DMSO): δ = 8.57 (d, 1H), 8.47 (d, 2H), 8.38 (m, | 75:25 |
| | yl)-1H-pyrazol-1-yl]methyl}pyridine | 1H), 7.88 (m, 1H), 7.72 (m, 1H, isomer), 7.45-7.40 (m, 3H), 7.30 (m, 1H), | |
| | | 7.25 (m, 1H), 7.20 (m, 2H), 7.11 (d, 2H, isomer), 5.51 (s, 2H), 5.27 (s, 1H, | |
| | | isomer) ppm | |
| 116 | 4-{1-[(2,2- | 1H-NMR (400 MHz, d₆-DMSO): δ = 8.48 (d, 2H), 8.39 (d, 2H, isomer), | 75:25 |
| | dichlorocyclopropyl)methyl]-3-(4- | 8.30 (s, 1H), 8.15 (s, 1H, isomer), 7.50 (m, 2H, isomer), 7.48-7.40 (m, 2H), | |
| | fluorophenyl)-1H-pyrazol-4- | 7.25-7.20 (m, 4H), 7.11 (d, 2H, isomer), 4.44 (dd, 1H), 4.34 (dd, 1H), | |
| | yl}pyridine | 4.13 (m, 2H, isomer), 2.38 (m, 1H), 2.21 (m, 1H, isomer), 1.89 (dd, 1H), | |
| | | 1.77 (dd, 1H, isomer), 1.70 (dd, 1H), 1.37 (dd, 1H, isomer) ppm | |
| 117 | 4-{3-(4-fluorophenyl)-1-[2- | 1H-NMR (400 MHz, d₆-DMSO): δ = 8.46 (d, 2H), 8.38 (d, 2H, isomer), | 80:20 |
| | (methylsulphanyl)ethyl]-1H-pyrazol- | 8.28 (s, 1H), 8.10 (s, 1H, isomer), 7.48-7.38 (m, 2H), 7.25-7.20 (m, 4H), | |
| | 4-yl}pyridine | 7.08 (d, 2H, isomer), 4.37 (t, 2H), 4.10 (t, 2H, isomer), 2.98 (t, 2H), 2.82 (t, | |
| | | 2H, isomer), 2.07 (s, 3H), 1.81 (s, 3H, isomer) ppm | |
| 120 | 4-{1-[1-(4-chlorophenyl)ethyl]-3-(4- | 1H-NMR (400 MHz, d₆-DMSO): δ = 8.46 (d, 2H), 8.42 (s, 1H), 8.37 (d, | 80:20 |
| | fluorophenyl)-1H-pyrazol-4- | 2H, isomer), 8.17 (s, 1H, isomer), 7.45-7.35 (m, 6H), 7.25-7.20 (m, 4H), | |
| | yl}pyridine | 7.08 (m, 2H, isomer), 5.70 (q, 1H), 5.30 (q, 1H, isomer), 1.86 (d, 3H), | |
| | | 1.78 (d, 3H, isomer) ppm | |
| 121 | 4-[3-(4-fluorophenyl)-1-(pyridin-4- | 1H-NMR (400 MHz, d₆-DMSO): δ = 8.62 (d, 2H), 8.59 (d, 2H), 8.32 (s, | — |
| | ylmethyl)-1H-pyrazol-4-yl]pyridine | 1H), 7.67 (d, 2H), 7.50 (m, 2H), 7.30 (d, 2H), 7.27 (t, 2H), 5.59 (s, 2H) ppm | |
| 122 | 4-{3-(4-fluorophenyl)-1-[2- | 1H-NMR (400 MHz, d₆-DMSO): δ = 8.48 (d, 2H), 8.38 (d, 2H, isomer), | 88:12 |
| | (trifluoromethoxy)ethyl]-1H-pyrazol- | 8.29 (s, 1H), 8.16 (s, 1H, isomer), 7.43 (m, 2H), 7.25-7.20 (m, 4H), 7.10 (d, | |
| | 4-yl}pyridine | 2H, isomer), 4.51 (m, 4H), 4.40 (t, 2H, isomer), 4.24 (t, 2H, isomer) ppm | |
| 122 | 4-{3-(4-fluorophenyl)-1-[2- | 1H-NMR (400 MHz, d₆-DMSO): δ = 8.48 (d, 2H), 8.38 (d, 2H, isomer), | 66:33 |
| | (trifluoromethoxy)ethyl]-1H-pyrazol- | 8.29 (s, 1H), 8.16 (s, 1H, isomer), 7.43 (m, 2H), 7.25-7.20 (m, 4H), 7.10 (d, | |
| | 4-yl}pyridine | 2H, isomer), 4.51 (m, 4H), 4.40 (t, 2H, isomer), 4.24 (t, 2H, isomer) ppm | |
| 123 | 4-[3-(4-fluorophenyl)-1- | 1H-NMR (400 MHz, d₆-DMSO): δ = 8.45 (d, 2H), 8.37 (d, 2H, isomer), | 89:11 |
| | (tetrahydrofuran-2-ylmethyl)-1H- | 8.18 (s, 1H), 8.07 (s, 1H, isomer), 7.40 (m, 2H), 7.25-7.20 (m, 4H), 7.08 (d, | |
| | pyrazol-4-yl]pyridine | 2H, isomer), 4.25 (m, 2H), 4.20 (m, 1H), 3.99 (dd, 1H, isomer), 3.89 (dd, | |
| | | 1H, isomer), 3.79 (m, 1H), 3.66 (m, 1H), 3.55 (m, 1H, isomer), 2.00 (m, | |
| | | 1H), 1.83 (m, 2H), 1.56 (m, 1H) ppm | |
| 124 | 3-[3-(4-fluorophenyl)-4-(pyridin-4- | 1H-NMR (400 MHz, d₆-DMSO): δ = 8.65 (d, 2H), 8.51 (s, 1H), 8.47 (s, | — |
| | yl)-1H-pyrazol-1-yl]propan-1,2-diol | 1H), 7.78 (d, 2H), 7.53 (m, 2H), 7.30 (t, 2H), 5.70 (s, 1H, br), 5.15 (s, 1H, | |
| | | br), 4.57 (dd, 1H), 4.28 (dd, 1H), 3.80 (m, 1H), 3.48 (m, 2H) ppm | |
| 128 | 4-[1-(2-fluorobenzyl)-3-(4- | 1H-NMR (400 MHz, d₆-DMSO): δ = 8.47 (d, 2H), 8.38 (d, 2H, isomer), | 80:20 |
| | fluorophenyl)-1H-pyrazol-4- | 8.32 (s, 1H), 8.13 (s, 1H, isomer), 7.40-7.30 (m, 5H), 7.25-7.20 (m, 5H), | |
| | yl]pyridine | 7.12 (m, 2H, isomer), 5.47 (s, 2H), 5.24 (s, 2H, isomer) ppm | |
| 134 | 4-{3-(4-fluorophenyl)-1-[2-(2- | 1H-NMR (400 MHz, d₃-CD₃CN): δ = 8.44 (d, 2H), 7.91 (s, 1H), 7.45 (m, | — |
| | methoxyethoxy)ethyl]-1H-pyrazol-4- | 2H), 7.18 (d, 2H), 7.12 (t, 2H), 4.31 (t, 2H), 3.86 (t, 2H), 3.57 (t, 2H), | |
| | yl}pyridine | 3.46 (t, 2H), 3.26 (s, 3H) ppm | |
| 140 | 3-{[3-(4-fluorophenyl)-4-(pyridin-4- | 1H-NMR (400 MHz, d₃-CD₃CN): δ = 8.47 (dd), 8.37 (m), 8.16 (s), 7.86 (s), | 60:40 |
| | yl)-1H-pyrazol-1- | 7.81 (d), 7.70 (m), 7.50 (t), 7.45-7.30 (m), 7.25-7.10 (m), 7.12 (dd), 5.47 (s, | |
| | yl]methyl}benzonitrile | 2H), 5.27 (s, 2H, isomer) ppm | |
| 141 | 4-{[3-(4-fluorophenyl)-4-(pyridin-4- | 1H-NMR (400 MHz, d₆-DMSO): δ = 8.48 (d, 2H), 8.39 (d, 2H, isomer), | 70:30 |
| | yl)-1H-pyrazol-1- | 8.37 (s, 1H), 8.17 (s, 1H, isomer), 7.84 (d, 2H), 7.74 (d, 2H, isomer), | |
| | yl]methyl}benzonitrile | 7.50 (d, 2H), 7.40 (m, 2H), 7.33 (m, 1H), 7.25-7.20 (m, 2H), 7.08 (m, 2H, | |
| | | isomer), 5.52 (s, 2H), 5.30 (s, 2H, isomer) ppm | |
| 142 | 3-[3-(4-fluorophenyl)-4-(pyridin-4- | 1H-NMR (400 MHz, d₃-CD₃CN): δ = 8.46 (m, 2H), 8.03 (s, 1H), 7.46 (dd, | — |
| | yl)-1H-pyrazol-1-yl]dihydrofuran- | 2H), 7.24 (dd, 2H), 7.10 (t, 2H), 5.33 (t, 1H), 4.60 (m, 1H), 4.40 (m, 1H), | |
| | 2(3H)-one | 2.90-2.80 (m, 2H) ppm | |
| 143 | 4-[3-(4-fluorophenyl)-1-(1H-imidazol- | 1H-NMR (400 MHz, d₃-CD₃CN): δ = 8.43 (d, 2H), 7.94 (s, 1H), 7.44 (dd, | — |
| | 2-ylmethyl)-1H-pyrazol-4-yl]pyridine | 2H), 7.17 (d, 2H), 7.10 (t, 2H), 6.99 (s, 2H), 5.40 (s, 2H), 2.90 (s, 1H, br) | |
| | | ppm | |
| 144 | 1-(1,5-dimethyl-1H-pyrazol-3-yl)-2- | 1H-NMR (400 MHz, d₃-CD₃CN): δ = 8.45 (d, 2H), 8.35 (d, 2H, isomer), | 62:38 |
| | [3-(4-fluorophenyl)-4-(pyridin-4-yl)- | 7.94 (s, 1H, isomer), 7.91 (s, 1H), 7.44 (dd, 2H), 7.36 (dd, 2H, isomer), | |
| | 1H-pyrazol-1-yl]ethanone | 7.20 (m, 2H), 7.10 (m, 2H), 6.57 (s, 1H), 6.47 (s, 1H, isomer), 5.62 (s, 2H), | |
| | | 5.40 (s, 2H, isomer), 3.85 (s, 3H), 3.77 (s, 3H, isomer), 2.31 (s, 3H), 2.26 (s, 3H, | |
| | | isomer) ppm | |
| 144 | 1-(1,5-dimethyl-1H-pyrazol-3-yl)-2- | 1H-NMR (400 MHz, d₃-CD₃CN): δ = 8.45 (d, 2H), 7.91 (s, 1H), 7.44 (dd, | — |
| | [3-(4-fluorophenyl)-4-(pyridin-4-yl)- | 2H), 7.20 (d, 2H), 7.10 (t, 2H), 6.57 (s, 1H), 5.62 (s, 2H), 3.85 (s, 3H), | |
| | 1H-pyrazol-1-yl]ethanone | 2.31 (s, 3H) ppm | |
| 145 | 2-[3-(4-fluorophenyl)-4-(pyridin-4- | 1H-NMR (400 MHz, d₃-CD₃CN): δ = 8.50 (m, 2H, br), 7.89 (s, 1H), | 91:9 |
| | yl)-1H-pyrazol-1-yl]ethyl acetate | 7.45 (dd, 2H), 7.28 (m, 2H, br), 7.10 (t, 2H), 4.46 (m, 2H), 4.39 (m, 2H), 4.28 (t, | |
| | | 2H, isomer), 3.90 (t, 2H), 2.00 (s, 3H), 1.90 (s, 3H, isomer) ppm | |
| 146 | 3-[3-(4-fluorophenyl)-4-(pyridin-4- | 1H-NMR (400 MHz, d₃-CD₃CN): δ = 8.50 (m, 2H, br), 7.94 (s, 1H), | — |
| | yl)-1H-pyrazol-1-yl]propanonitrile | 7.46 (dd, 2H), 7.28 (m, 2H, br), 7.14 (t, 2H), 4.45 (t, 2H), 3.02 (t, 2H) ppm | |
| 147 | 4-[3-(4-fluorophenyl)-1-isobutyl-1H- | ¹H-NMR (400 MHz, d₆-DMSO): δ = 11.02 (s, 1H), 8.21 (s, 1H), 7.91 (d, | — |
| | pyrazol-4-yl]-1,3-dihydro-2H- | 1H), 7.42 (m, 2H), 7.21 (m, 2H), 6.60 (d, 1H), 4.00 (d, 2H), 3.42 (s, 2H), | |
| | pyrrolo[2,3-b]pyridine-2-one | 2.20 (m, 1H), 0.92 (d, 6H) ppm | |
| 148 | 4-[3-(4-fluorophenyl)-1-isobutyl-1H- | ¹H-NMR (400 MHz, d₆-DMSO): δ = 8.06 (d, 1H), 7.30 (m, 3H), 6.85 (t, | — |
| | pyrazol-4-yl]-1,3,3-trimethyl-1,3- | 2H), 6.67 (d, 1H), 3.92 (d, 2H), 3.27 (s, 3H), 2.26 (m, 1H), 1.13 (s, 6H), | |
| | dihydro-2H-pyrrolo[2,3-b]pyridine-2- | 0.92 (d, 6H) ppm | |
| | one | | |
| 149 | 4-[5-(4-fluorophenyl)-1-(2- | 1H-NMR (400 MHz, CDCl₃): δ = 8.11 (s, 1H), 8.03 (d, 1H), 7.32 (m, 2H), | — |
| | methylpropyl)-1H-pyrazol-4-yl]-N- | 7.19 (t, 2H), 6.18 (bs, 1H), 5.87 (m, 1H), 5.20 (d, 1H), 5.09 (d, 1H), | |
| | (prop-2-en-1-yl)pyrimidin-2-amine | 5.02 (m, 1H), 3.85 (bs, 2H), 3.74 (d, 2H), 2.18 (m, 1H), 0.79 (d, 6H) ppm | |
| 150 | 4-[3-(4-fluorophenyl)-1-(2- | 1H-NMR (400 MHz, CDCl₃): δ = 8.10 (d, 1H), 7.93 (s, 1H), 7.54 (m, 2H), | — |
| | methylpropyl)-1H-pyrazol-4-yl]-N- | 7.07 (t, 2H), 6.39 (d, 1H), 5.95 (m, 1H), 5.26 (d, 1H), 5.12 (d, 2H), | |
| | (prop-2-en-1-yl)pyrimidin-2-amine | 4.04 (bs 1H), 3.96 (d, 2H), 2.30 (m, 1H), 0.98 (d, 6H) ppm | |
| 153 | N-{4-[3-(4-fluorophenyl)-1-(2- | 1H-NMR (400 MHz, d₃-CD₃CN): δ = 8.54 (s, 1H, br), 8.10 (m, 2H), | — |
| | methoxyethyl)-1H-pyrazol-4- | 7.86 (s, 1H), 7.46 (dd, 2H), 7.09 (t, 2H), 6.87 (dd, 1H), 4.31 (t, 2H), 3.78 (t, 2H), | |
| | yl]pyridin-2-yl}propanamide | 3.32 (s, 3H), 2.38 (q, 2H), 1.11 (t, 2H) ppm | |
| 154 | 2-fluoro-5-[1-(2-methylpropyl)-4- | 1H-NMR (400 MHz, d₃-CD3CN): δ = 8.47-8.46 (m, 2H), 7.86 (s, 1H), | — |
| | (pyridin-4-yl)-1H-pyrazol-3- | 7.81-7.79 (m, 1H), 7.73-7.71 (m, 1H), 7.33-7.30 (m, 1H), 7.19-7.18 (m, | |
| | yl]benzonitrile | 2H), 3.99 (d, 2H), 2.23 (m, 1H), 0.94 (d, 6H) ppm | |
| 155 | N-{4-[3-(4-fluorophenyl)-1-(2- | 1H-NMR (400 MHz, d₆-DMSO): δ = 10.40 (s, 1H), 8.16 (d, 1H), 8.14 (s, | — |
| | methylpropyl)-1H-pyrazol-4- | 1H), 8.08 (s, 1H), 7.42-7.40 (m, 2H), 7.20-7.18 (m, 2H), 6.82 (dd, 1H), | |
| | yl]pyridin-2-yl}propanamide | 3.98 (d, 2H), 2.38 (q, 2H), 2.20 (m, 1H), 1.05 (t, 3H), 0.92 (d, 6H) ppm | |
| 156 | N-{4-[3-(4-fluorophenyl)-1-(propan- | 1H-NMR (400 MHz, d₃-CD3CN): δ = 8.35 (s, 1H, br), 8.12 (s, 1H), | — |
| | 2-yl)-1H-pyrazol-4-yl]pyridin-2- | 8.10 (m, 1H), 7.88 (s, 1H), 7.48-7.45 (m, 2H), 7.12-7.08 (m, 2H), 6.86 (dd, 1H), | |
| | yl}cyclobutancarboxamide | 4.55 (m, 1H), 3.25 (m, 1H), 2.03-2.15 (m, 6H), 1.53 (d, 6H) ppm | |
| 157 | N-{4-[1-(butan-2-yl)-3-(4- | 1H-NMR (400 MHz, d₆-DMSO): δ = 10.39 (s, 1H), 8.19 (s, 1H), 8.15 (m, | — |
| | fluorophenyl)-1H-pyrazol-4-yl]- | 1H), 8.11 (s, 1H), 7.44-7.42 (m, 2H), 7.20-7.18 (m, 2H), 6.82-6.81 (dd, 1H), | |
| | pyridin-2-yl}-2-methylpropanamide | 4.35 (m, 1H), 2.74 (m, 1H), 1.90-1.80 (m, 1H), 1.48 (d, 2H), 1.06 (d, 6H), | |
| | | 0.80 (t, 3H) ppm | |
| 158 | 2-cyclopropyl-N-{4-[3-(4- | 1H-NMR (400 MHz, d₆-DMSO): δ = 10.32 (s, 1H), 8.16-8.14 (m, 2H), | — |
| | fluorophenyl)-1-(2-methylpropyl)-1H- | 8.09 (s, 1H), 7.44-7.42 (m, 2H), 7.20-7.18 (m, 2H), 6.83-6.82 (dd, 1H), 3.99 (d, | |
| | pyrazol-4-yl]pyridin-2-yl}acetamide | 2H), 2.24 (d, 2H), 2.20 (m, 1H), 1.05 (m, 1H), 0.91 (d, 6H), 0.45 (m, 2H), | |
| | | 0.16 (m, 2H) ppm | |
| 159 | N-{4-[1-(butan-2-yl)-3-(4- | 1H-NMR (400 MHz, d₆-DMSO): δ = 10.31 (s, 1H), 8.19 (s, 1H), 8.16 (d, | — |
| | fluorophenyl)-1H-pyrazol-4- | 1H), 8.09 (s, 1H), 7.44-7.42 (dd, 2H), 7.20-7.18 (m, 21-1), 6.84-6.83 (dd, | |
| | yl]pyridin-2-yl}-2- | 1H), 4.33 (m, 1H), 2.23 (d, 2H), 1.90 (m, 1H), 1.80 (m, 1H), 1.48 (d, 3H), | |
| | cyclopropylacetamide | 1.05 (m, 1H), 0.80 (t, 3H), 0.45 (m, 2H), 0.17 (m, 2H) ppm | |
| 160 | N-{4-[3-(4-fluorophenyl)-1-(propan- | 1H-NMR (400 MHz, d₃-CD3CN): δ = 8.50 (s, 1H, br), 8.10 (m, 2H), | — |
| | 2-yl)-1H-pyrazol-4-yl]pyridin-2-yl}-3- | 7.89 (s, 1H), 7.47-7.45 (m, 2H), 7.09-7.08 (m, 2H), 6.88 (dd, 1H), 4.55 (m, 1H), | |
| | methylbutanamide | 2.21 (d, 2H), 2.10 (m, 1H), 1.53 (d, 6H), 0.95 (d, 6H) ppm | |
| 161 | N-{4-[3-(4-fluorophenyl)-1-(propan- | 1H-NMR (400 MHz, d₃-CD3CN): δ = 8.59 (s, 1H, br), 8.11 (m, 1H), | — |
| | 2-yl)-1H-pyrazol-4-yl]pyridin-2- | 8.05 (s, 1H), 7.88 (s, 1H), 7.47-7.45 (m, 2H), 7.11-7.08 (m, 2H), 6.88 (dd, 1H), | |
| | yl}acetamide | 4.55 (m, 1H), 2.08 (s, 3H), 1.52 (d, 6H) ppm | |
| 162 | N-{4-[1-(butan-2-yl)-3-(4- | 1H-NMR (400 MHz, d₆-DMSO): δ = 9.93 (s, 1H), 8.23 (s, 1H), 8.20 (d, | — |
| | fluorophenyl)-1H-pyrazol-4- | 1H), 8.03 (s, 1H), 7.44-7.40 (m, 2H), 7.22-7.19 (m, 2H), 6.94 (d, 1H), | |
| | yl]pyridin-2-yl}-2-methoxyacetamide | 4.35 (m, 1H), 4.03 (s, 2H), 1.90 (m, 1H), 1.80 (m, 1H), 1.49 (d, 3H), 1.04 (t, | |
| | | 3H), 0.80 (t, 3H) ppm | |
| 163 | N-{4-[3-(4-fluorophenyl)-1-(2- | 1H-NMR (400 MHz, d₃-CD3CN): δ = 8.70 (s, 1H, br), 8.15 (d, 1H), 8.08 (s, | — |
| | methoxyethyl)-1H-pyrazol-4- | 1H), 7.88 (s, 1H), 7.47-7.45 (m, 2H), 7.12-7.10 (m, 2H), 6.95 (dd, 1H), | |
| | yl]pyridin-2-yl}-2-methoxyacetamide | 4.32 (t, 2H), 3.96 (s, 2H), 3.78 (t, 2H), 3.45 (s, 3H), 3.32 (s, 3H) ppm | |
| 164 | N-{4-[1-(butan-2-yl)-3-(4- | 1H-NMR (400 MHz, d₆-DMSO): δ = 10.39 (s, 1H), 8.19 (s, 1H), 8.17 (d, | — |
| | fluorophenyl)-1H-pyrazol-4- | 1H), 8.09 (s, 1H), 7.44-7.42 (m, 2H), 7.22-7.19 (m, 2H), 6.84-6.83 (dd, 1H), | |
| | yl]pyridin-2-yl}propanamide | 4.34 (m, 1H), 2.38 (q, 2H), 1.90 (m, 1H), 1.80 (m, 1H), 1.49 (d, 3H), | |
| | | 1.04 (t, 3H), 0.80 (t, 3H) ppm | |
| 165 | N-{4-[3-(4-fluorophenyl)-1-(2- | 1H-NMR (400 MHz, d₆-DMSO): δ = 10.76 (s, 1H), 8.17 (d, 1H), 8.13 (s, | — |
| | methylpropyl)-1H-pyrazol-4- | 1H), 8.08 (s, 1H), 7.44-7.42 (m, 2H), 7.22-7.19 (m, 2H), 6.83-6.81 (dd, 1H), | |
| | yl]pyridin-2- | 3.99 (d, 2H), 2.20 (m, 1H), 1.98 (m, 1H), 0.91 (m, 6H), 0.78 (m, 4H) ppm | |
| | yl}cyclopropanecarboxamide | | |
| 166 | N-{4-[3-(4-fluorophenyl)-1-(2- | 1H-NMR (400 MHz, d₆-DMSO): δ = 9.94 (s, 1H), 8.21-8.19 (m, 1H), | — |
| | methylpropyl)-1H-pyrazol-4- | 8.04 (s, 1H), 7.45-7.43 (m, 2H), 7.22 (m, 2H), 6.83-6.81 (dd, 1H), 4.03 (s, 2H), | |
| | yl]pyridin-2-yl}-2-methoxyacetamide | 3.99 (d, 2H), 2.20 (m, 1H), 0.91 (m, 6H) ppm | |
| 167 | N-{4-[1-(butan-2-yl)-3-(4- | 1H-NMR (400 MHz, d₆-DMSO): δ = 10.76 (s, 1H), 8.17 (m, 2H), 8.07 (s, | — |
| | fluorophenyl)-1H-pyrazol-4- | 1H), 7.44-7.42 (dd, 2H), 7.24-7.20 (m, 2H), 6.83-6.82 (dd, 1H), 4.35 (m, | |
| | yl]pyridin-2- | 1H), 2.00 (m, 1H), 1.90 (m, 1H), 1.80 (m, 1H), 1.40 (d, 3H), 0.80 (m, 7H) | |
| | yl}cyclopropanecarboxamide | ppm | |
| 169 | 4-[1-(2,2-difluoroethyl)-3-(4- | 1H-NMR (400 MHz, d₃-CD3CN): δ = 8.46 (dd, 1H), 7.93 (s, 1H), | — |
| | fluorophenyl)-1H-pyrazol-4- | 7.46-7.44 (m, 2H), 7.19 (m, 1H), 7.14-7.10 (m, 2H), 6.27 (tt, 2J (H, F) = 55 Hz, | |
| | yl]pyridine | 1H), 4.59 (dt, 3J (H, F) = 15 Hz, 2H) ppm | |
| 170 | ethyl [3-(4-fluorophenyl)-4-(pyridin- | 1H-NMR (400 MHz, d₃-CD3CN): δ = 8.45 (d, 2H), 7.91 (s, 1H), 7.45 (m, | — |
| | 4-yl)-1H-pyrazol-1-yl] acetate | 2H), 7.21 (d, 2H), 7.14-7.08 (m, 2H), 4.99 (s, 2H), 4.21 (q, 2H), 1.28 (t, 3H) | |
| | | ppm | |
| 171 | 4-[3-(4-fluorophenyl)-5-methyl-1- | 1H-NMR (400 MHz, d₆-DMSO): δ = 8.56 (d, 2H), 8.46 (d, 2H, isomer), | 62/19 |
| | (prop-2-yn-1-yl)-1H-pyrazol-4- | 7.58 (t, 2H), 7.32 (m, 2H), 7.17 (m, 2H), 5.77 (d, 2H), 5.50 (d, 2H, isomer), | |
| | yl]pyridine | 2.34 (s, 3H), 2.30 (s, 1H) ppm | |
| 172 | 4-[3-(4-fluorophenyl)-1H-pyrazol-4- | ¹H-NMR (400 MHz, d₆-DMSO): δ = 13.28 (bs, 1H), 8.12 (d, 1H), 7.59 (bs, | — |
| | yl]-N-(propan-2-yl)pyrimidin-2-amine | 2H), 7.28 (bs, 2H), 6.77 (d, 1H), 6.52 (bs, 1H), 3.81 (bs, 1H), 1.09 (bs, 6H) | |
| | | ppm. | |
| 173 | 4-[3-(4-fluorophenyl)-1H-pyrazol-4- | 1H-NMR (400 MHz, CDCl₃): δ = 8.15 (s, 1H), 8.06 (d, 1H), 7.46 (m, 2H), | — |
| | yl]-N-(prop-2-yn-1-yl)pyrimidin-2- | 7.09 (t, 2H), 6.49 (d, 1H), 4.05 (bs, 2H), 2.15 (s, 1H) ppm | |
| | amine | | |
| 174 | 4-[3-(4-fluorophenyl)-1-(2- | 1H-NMR (400 MHz, d₆-DMSO): δ = 8.11 (s, 1H), 8.00 (d, 1H), 7.43 (m, | — |
| | methylpropyl)-1H-pyrazol-4-yl]-N- | 2H), 7.33 (t, 2H), 6.65 (d, 1H), 6.40 (bs, 1H), 3.70 (d, 2H), 2.02 (m, 1H), | |
| | (propan-2-yl)pyrimidin-2-amine | 0.98 (bs, 6H), 0.72 (d, 6H) ppm | |
| 175 | 3-(4-fluorophenyl)-4-(pyridin-4-yl)- | 1H-NMR (400 MHz, d₆-DMSO): δ = 8.62 (d, 2H), 7.46 (m, 2H), 7.631 (m, | — |
| | 1H-pyrazol-5-carbonitrile | 4H) ppm | |
| 176 | 4-{1-[1-(2-chloro-1,3-thiazol-5- | 1H-NMR (400 MHz, d₃-CD3CN): δ = 8.45 (m, 2H), 7.98 (s, 1H), 7.57 (s, | — |
| | yl)ethyl]-3-(4-fluorophenyl)-1H- | 1H), 7.48-7.43 (m, 2H), 7.18 (m, 2H), 7.14-7.08 (m, 2H), 5.88 (q, 1H), | |
| | pyrazol-4-yl}pyridine | 2.15 (m, 3H) ppm | |
| 178 | 1-[3-(4-fluorophenyl)-4-(pyridin-4- | 1H-NMR (400 MHz, d₃-CD3CN): δ = 8.45 (m, 2H), 8.10 (s, 1H), 7.45 (m, | — |
| | yl)-1H-pyrazol-1-yl]-3-methylbutan- | 2H), 7.25 (m, 2H), 7.15 (m, 2H), 1.75 (d, 6H) ppm | |
| | 2-one | | |
| 179 | 4-[1-(2-ethoxyethyl)-3-(4- | 1H-NMR (400 MHz, d₃-CD3CN): δ = 8.45 (d, 2H), 7.89 (s, 1H), 7.45 (m, | — |
| | fluorophenyl)-1H-pyrazol-4- | 2H), 7.18 (d, 2H), 7.14-7.08 (m, 2H), 4.30 (t, 2H), 3.82 (t, 2H), 3.49 (q, | |
| | yl]pyridine | 2H), 1.12 (t, 3H) ppm | |
| 180 | [3-(4-fluorophenyl)-4-(pyridin-4-yl)- | 1H-NMR (400 MHz, d₃-CD3CN): δ = 8.46 (dd, 2H), 7.96 (s, 1H), | — |
| | 1H-pyrazol-1-yl]acetonitrile | 7.47-7.44 (m, 2H), 7.20 (d, 2H), 7.14-7.11 (m, 2H), 5.23 (s, 2H) ppm | |
| 181 | propan-2-yl-{4-[3-(4-fluorophenyl)-1- | 1H-NMR (400 MHz, d7-DMF): δ = 8.16 (d, 1H), 8.02 (m, 2H), 7.93 (s, | — |
| | (propan-2-yl)-1H-pyrazol-4- | 1H), 7.57-7.53 (m, 2H), 7.25-7.21 (m, 2H), 6.92 (dd, 1H), 4.90 (m, 1H), | |
| | yl]pyridin-2-yl}carbamate | 4.67 (m, 1H), 3.20 (m, 6H), 1.57 (d, 6H) ppm | |
| 182 | N-benzyl-4-[3-(4-fluorophenyl)-1-(2- | 1H-NMR (400 MHz, d₆-DMSO): δ = 8.30 (bs, 1H), 8.12 (d, 1H), 7.56 (m, | — |
| | methylpropyl)-1H-pyrazol-4- | 3H), 7.25 (t, 7H), 6.49 (bs, 1H), 4.37 (bs, 2H), 4.01 (d, 2H), 2.18 (m, 1H), | |
| | yl]pyrimidin-2-amine | 0.89 (d, 6H) ppm | |
| 184 | 4-[3-(4-fluorophenyl)-1-(2- | 1H-NMR (400 MHz, CDCl₃): δ = 8.01 (d, 1H), 7.89 (s, 1H), 7.45 (m, 2H), | — |
| | methylpropyl)-1H-pyrazol-4- | 7.02 (t, 2H), 6.36 (d, 1H), 4.94 (bs, 2H), 3.89 (d, 2H), 2.23 (m, 1H), 0.91 (d, | |
| | yl]pyrimidin-2-amine | 6H) ppm | |
| 185 | N-{4-[3-(4-fluorophenyl)-1-(2- | 1H-NMR (400 MHz, CDCl₃): δ = 8.37 (d, 2H), 7.98 (s, 1H), 7.44 (m, 2H), | — |
| | methylpropyl)-1H-pyrazol-4- | 7.07 (d, 2H), 6.87 (d, 1H), 4.85 (m, 1H), 3.92 (d, 2H), 2.48 (m, 1H), | |
| | yl]pyrimidin-2-yl}-2-methyl-N- | 2.24 (m, 1H), 1.18 (d, 6H), 1.03 (d, 6H), 0.92 (d, 6H) ppm | |
| | (propan-2-yl)propanamide | | |
| 186 | N-{4-[3-(4-fluorophenyl)-1-(2- | 1H-NMR (400 MHz, CDCl₃): δ = 8.09 (bs, 1H), 7.93 (s, 1H), 7.41 (m, 2H), | — |
| | methylpropyl)-1H-pyrazol-4- | 7.05 (t, 2H), 6.8 (d, 1H), 4.85 (m, 1H), 3.92 (d, 2H), 3.05 (m, 1H), 2.25 (m, | |
| | yl]pyrimidin-2-yl}-2,2,4-trimethyl-3- | 1H), 1.23 (d, 6H), 1.1 (d, 6H), 1.04 (s, 6H), 0.93 (d, 6H) ppm | |
| | oxo-N-(propan-2-yl)pentanamide | | |
| 187 | 1-{4-[3-(4-fluorophenyl)-1-(2- | 1H-NMR (400 MHz, d₆-DMSO): δ = 8.26 (d, 2H), 8.16 (s, 1H), 7.43 (m, | — |
| | methylpropyl)-1H-pyrazol-4- | 2H), 7.20 (t, 2H), 6.93 (m, 1H), 3.98 (m, 4H), 2.54 (m, 2H), 2.20 (m, 1H), | |
| | yl]pyridin-2-yl}pyrrolidin-2-one | 2.02 (m, 2H), 0.91 (d, 6H) ppm | |
| 188 | N-{4-[3-(4-fluorophenyl)-1-(2- | 1H-NMR (400 MHz, CDCl₃): δ = 8.44 (d, 1H), 7.98 (s, 1H), 7.44 (m, 2H), | — |
| | methylpropyl)-1H-pyrazol-4- | 7.06 (m, 2H), 6.95 (d, 1H), 3.90 (d, 2H), 2.87 (m, 2H), 2.23 (m, 1H), | |
| | yl]pyrimidin-2-yl}-2-methyl-N-(2- | 1.14 (d, 12H), 0.91 (d, 6H) ppm | |
| | methylpropanoyl)propanamide | | |
| 189 | N-{4-[3-(4-fluorophenyl)-1-(2- | 1H-NMR (400 MHz, CDCl₃): δ = 8.25 (d, 2H), 8.05 (s, 1H), 8.00 (s, 1H), | — |
| | methylpropyl)-1H-pyrazol-4- | 7.42 (m, 2H), 7.04 (d, 2H), 6.98 (d, 1H), 3.90 (d, 2H), 2.9 (m, 1H), 2.24 (m, | |
| | yl]pyrimidin-2-yl}-2- | 1H), 1.2 (d, 6H), 0.91 (d, 6H) ppm | |
| | methylpropanamide | | |
| 191 | N-{4-[3-(4-fluorophenyl)-1-(2- | 1H-NMR (400 MHz, d₃-CD3CN): δ = 8.61 (s, 1H, br), 8.10 (d, 1H), 8.09 (s, | — |
| | methylpropyl)-1H-pyrazol-4- | 1H), 7.82 (s, 1H), 7.45-7.43 (m, 2H), 7.35-7.28 (m, 5H), 7.09-7.06 (m, 2H), | |
| | yl]pyridin-2-yl}-2-phenylacetamide | 6.88 (d, 1H), 3.96 (d, 2H), 3.69 (s, 2H), 2.20 (m, 1H), 0.93 (d, 6H) ppm | |
| 192 | (2S)—N-{4-[3-(4-fluorophenyl)-1- | 1H-NMR (400 MHz, d₃-CD3CN): δ = 8.98 (s, 1H, br), 8.15-8.10 (m, 2H), | — |
| | (propan-2-yl)-1H-pyrazol-4- | 7.90 (s, 1H), 7.48-7.45 (m, 2H), 7.11-7.08 (m, 2H), 6.92 (dd, 1H), 4.56 (m, | |
| | yl]pyridin-2-yl}-2- | 1H), 4.22 (m, 1H), 4.05 (s, 1H), 1.53 (d, 6H) ppm | |
| | hydroxypropanamide | | |
| 193 | tert-butyl 4-{3-(4-fluorophenyl)-4-[2- | 1H-NMR (400 MHz, d₃-CD3CN): δ = 8.50 (s, 1H, br), 8.10 (m, 2H), | — |
| | (propanoylamino)pyridin-4-yl]-1H- | 7.89 (s, 1H), 7.47-7.45 (m, 2H), 7.11-7.08 (m, 2H), 6.92 (dd, 1H), 4.38 (m, 1H), | |
| | pyrazol-1-yl}piperidin-1-carboxylate | 4.18 (m, 2H), 2.90 (m, 2H), 2.37 (q, 2H), 2.10 (m, 2H), 1.90 (m, 2H), | |
| | | 1.45 (s, 9H), 1.10 (t, 3H) ppm | |
| 195 | N-{4-[3-(4-chloro-2-fluorophenyl)-1- | 1H-NMR (400 MHz, d₃-CD3CN): δ = 8.53 (s, 1H, br), 8.15-8.13 (m, 3H), | — |
| | methyl-5-(methylsulphanyl)-1H- | 7.42-7.40 (m, 2H), 7.25 (d, 1H), 7.15 (d, 1H), 6.85 (dd, 1H), 4.05 (s, 3H), | |
| | pyrazol-4-yl]pyridin-2- | 2.38 (q, 2H), 2.30 (s, 3H), 1.10 (t, 3H) ppm | |
| | yl}propanamide | | |
| 196 | N-{4-[5-(difluoromethoxy)-3-(4- | 1H-NMR (400 MHz, d₃-CD3CN): δ = 10.2 (s, 1H), 8.63 (s, 1H, br), 8.26 (d, | — |
| | fluoro-2-hydroxyphenyl)-1-methyl- | 1H), 8.13 (s, 1H), 7.03 (dd, 1H), 6.95 (m, 1H), 6.70 (m, 1H), 6.55 (t, 1H), | |
| | 1H-pyrazol-4-yl]pyridin-2- | 6.45 (m, 1H), 3.82 (s, 3H), 2.41 (q, 2H), 1.12 (t, 3H) ppm | |
| | yl}propanamide | | |
| 197 | N-{4-[3-(4-chloro-2-fluorophenyl)-1- | 1H-NMR (400 MHz, d₃-CD3CN): δ = 8.55 (s, 1H, br), 8.17 (d, 1H), 8.01 (s, | — |
| | methyl-5-(trifluoromethyl)-1H- | 1H), 7.36 (dd, 1H), 7.21 (d, 1H), 7.18 (d, 1H), 6.82 (dd, 1H), 4.10 (s, 3H), | |
| | pyrazol-4-yl]pyridin-2- | 2.37 (q, 2H), 1.09 (t, 3H) ppm | |
| | yl}propanamide | | |
| 198 | N-{4-[1-ethyl-3-(4-fluorophenyl)-1H- | 1H-NMR (400 MHz, d₃-CD3CN): δ = 8.50 (s, 1H, br), 8.10 (m, 2H), | — |
| | pyrazol-4-yl]pyridin-2- | 7.86 (s, 1H), 7.47-7.45 (m, 2H), 7.11-7.08 (m, 2H), 6.86 (dd, 1H), 4.21 (q, 2H), | |
| | yl}propanamide | 2.38 (q, 2H), 1.49 (t, 3H), 1.10 (t, 3H) ppm | |
| 199 | N-{4-[3-(4-cyano-2,5- | 1H-NMR (400 MHz, d₃-CD3CN): δ = 8.70 (s, 1H, br), 8.20 (d, 1H), 7.99 (s, | — |
| | difluorophenyl)-5-(difluoromethoxy)- | 1H), 7.50-7.45 (m, 2H), 6.94 (dd, 1H), 6.65 (t, 2J (H, F), 1H), 3.96 (s, 2H), | |
| | 1-methyl-1H-pyrazol-4-yl]pyridin-2- | 3.86 (s, 3H), 3.44 (s, 3H) ppm | |
| | yl}-2-methoxyacetamide | | |
| 200 | N-{4-[3-(4-chloro-2-fluorophenyl)-1- | 1H-NMR (400 MHz, d₃-CD3CN): δ = 8.73 (s, 1H, br), 8.18-8.15 (m, 2H), | — |
| | methyl-5-(methylsulphanyl)-1H- | 7.40 (m, 1H), 7.25 (d, 1H), 7.15 (d, 1H), 6.90 (m, 1H), 4.03 (s, 3H), 3.97 (s, | |
| | pyrazol-4-yl]pyridin-2-yl}-2- | 2H), 3.45 (s, 3H), 2.30 (s, 3H) ppm | |
| | methoxyacetamide | | |
| 201 | N-{4-[5-(difluoromethoxy)-3-(4- | 1H-NMR (400 MHz, d₃-CD3CN): δ = 8.80 (s, 1H, br), 8.30 (d, 1H), 8.12 (s, | — |
| | fluoro-2-hydroxyphenyl)-1-methyl- | 1H), 7.03-7.00 (m, 2H), 6.70 (m, 1H), 6.58 (t, 2J(H, F), 1H), 6.48 (m, 1H), | |
| | 1H-pyrazol-4-yl]pyridin-2-yl}-2- | 3.99 (s, 2H), 3.84 (s, 3H), 3.46 (s, 3H) ppm | |
| | methoxyacetamide | | |
| 202 | N-{4-[3-(4-chloro-2-fluorophenyl)-1- | 1H-NMR (400 MHz, d₃-CD3CN): δ = 8.78 (s, 1H, br), 8.21 (d, 1H), 8.01 (s, | — |
| | methyl-5-(trifluoromethyl)-1H- | 1H), 7.36 (dd, 1H), 7.21 (d, 1H), 7.16 (d, 1H), 6.89 (dd, 1H), 4.10 (s, 3H), | |
| | pyrazol-4-yl]pyridin-2-yl}-2- | 3.96 (s, 2H), 3.45 (s, 3H) ppm | |
| | methoxyacetamide | | |
| 203 | N-{4-[1-ethyl-3-(4-fluorophenyl)-1H- | 1H-NMR (400 MHz, d₃-CD3CN): δ = 8.70 (s, 1H, br), 8.14 (d, 1H), 8.08 (s, | — |
| | pyrazol-4-yl]pyridin-2-yl}-2- | 1H), 7.88 (s, 1H), 7.47-7.45 (m, 2H), 7.12-7.10 (m, 2H), 6.94 (dd, 1H), | |
| | methoxyacetamide | 4.21 (q, 2H), 3.96 (s, 2H), 3.45 (s, 3H), 1.50 (t, 3H) ppm | |
| 204 | tert-butyl 4-[3-(4-fluorophenyl)-4-{2- | 1H-NMR (400 MHz, d₃-CD3CN): δ = 8.70 (s, 1H, br), 8.12 (d, 1H), 8.07 (s, | — |
| | [(methoxyacetyl)amino]pyridin-4-yl}- | 1H), 7.92 (s, 1H), 7.45-7.41 (m, 2H), 7.08 (m, 2H), 6.95 (d, 1H), 4.38 (m, | |
| | 1H-pyrazol-1-yl]piperidin-1- | 1H), 4.15 (m, 2H), 3.96 (s, 2H), 3.45 (s, 3H), 2.1 (m, 2H), 1.90 (m, 2H), | |
| | carboxylate | 1.45 (s, 9H) ppm | |
| 205 | N-{4-[3-(4-fluorophenyl)-1-(propan- | 1H-NMR (400 MHz, d₃-CD3CN): δ = 8.95 (s, 1H, br), 8.26 (s, 1H), 8.18 (d, | — |
| | 2-yl)-1H-pyrazol-4-yl]pyridin-2- | 1H), 7.93 (m, 3H), 7.60 (m, 1H), 7.51-7.49 (m, 4H), 7.13-7.10 (m, 1H), | |
| | yl }benzamide | 6.96-6.95 (dd, 1H), 4.57 (m, 1H), 1.54 (d, 6H) ppm | |
| 206 | N-{4-[3-(4-fluorophenyl)-1-(propan- | 1H-NMR (400 MHz, d₃-CD3CN): δ = 8.93 (s, 1H, br), 8.18 (m, 2H), | — |
| | 2-yl)-1H-pyrazol-4-yl]pyridin-2- | 7.93 (s, 1H), 7.84 (m, 1H), 7.70 (dd, 1H), 7.50-7.48 (m, 2H), 7.18-7.16 (dd, 1H), | |
| | yl}thiophen-2-carboxamide | 7.12-7.10 (m, 2H), 6.95 (dd, 1H), 4.56 (m, 1H), 1.54 (d, 6H) ppm | |
| 207 | N-{4-[3-(4-fluorophenyl)-1-(propan- | 1H-NMR (400 MHz, d₃-CD3CN): δ = 8.80 (s, 1H br), 8.21 (s, 1H), | — |
| | 2-yl)-1H-pyrazol-4-yl]pyridin-2- | 8.18 (m, 1H), 7.93 (s, 1H), 7.56 (m, 1H), 7.50-7.48 (m, 3H), 7.12-7.09 (m, 2H), | |
| | yl}thiophen-3-carboxamide | 6.94 (dd, 1H), 4.55 (m, 1H), 1.53 (d, 6H) ppm | |
| 208 | N-{4-[3-(4-fluorophenyl)-1-(propan- | 1H-NMR (400 MHz, d₃-CD3CN): δ = 9.18 (s, 1H, br), 8.16 (d, 1H), 8.11 (s, | — |
| | 2-yl)-1H-pyrazol-4-yl]pyridin-2-yl}-2- | 1H), 7.90 (s, 1H), 7.48-7.46 (m, 2H), 7.11-7.08 (m, 2H), 6.94 (dd, 1H), | |
| | (2-methoxyethoxy)acetamide | 4.55 (m, 1H), 4.04 (s, 2H), 3.71 (t, 2H), 3.56 (t, 2H), 3.38 (s, 3H), 1.53 (d, 6H) | |
| | | ppm | |
| 209 | N-{4-[3-(4-fluorophenyl)-1-(propan- | 1H-NMR (400 MHz, d₃-CD3CN): δ = 9.12 (s, 1H, br), 8.13 (m, 2H), | — |
| | 2-yl)-1H-pyrazol-4-yl]pyridin-2-yl}-2- | 7.90 (s, 1H), 7.48-7.46 (m, 2H), 7.11-7.09 (m, 2H), 6.91 (dd, 1H), 4.55 (m, 1H), | |
| | hydroxy-2-methylpropanamide | 3.90 (s, 1H, br), 1.53 (d, 6H), 1.40 (s, 6H) ppm | |
| 210 | N-{4-[3-(4-fluorophenyl)-1-(propan- | 1H-NMR (400 MHz, d₃-CD3CN): δ = 9.01 (s, 1H, br), 8.14 (m, 2H), | — |
| | 2-yl)-1H-pyrazol-4-yl]pyridin-2-yl}- | 7.91 (s, 1H), 7.48-7.46 (m, 2H), 7.11-7.09 (m, 2H), 6.93 (dd, 1H), 4.56 (m, 1H), | |
| | 2,3-dihydroxypropanamide | 4.15 (m, 1H), 4.10 (s, 1H, br), 3.74 (m, 2H), 3.10 (s, 1H, br), 1.53 (d, 6H) | |
| | | ppm | |
| 211 | 1-ethyl-3-{4-[3-(4-fluorophenyl)-1- | 1H-NMR (400 MHz, d₃-CD3CN): δ = 8.82 (s, 1H, br), 8.05 (d, 1H), 7.86 (s, | — |
| | (propan-2-yl)-1H-pyrazol-4- | 1H), 7.49-7.45 (m, 2H), 7.13-7.10 (m, 2H), 6.81 (s, 1H), 6.78 (m, 1H), | |
| | yl]pyridin-2-yl}urea | 4.56 (m, 1H), 3.25 (q, 2H), 1.52 (d, 6H), 1.15 (t, 3H) ppm | |
| 212 | N-{4-[3-(4-fluorophenyl)-1-(2- | 1H-NMR (400 MHz, d₃-CD3CN): δ = 8.60 (s, 1H, br), 8.10 (d, 1H), 8.09 (s, | — |
| | methoxyethyl)-1H-pyrazol-4- | 1H), 7.84 (s, 1H), 7.45 (dd, 2H), 7.38-7.30 (m, 5H), 7.10 (t, 2H), 6.88 (d, | |
| | yl]pyridin-2-yl}-2-phenylacetamide | 1H), 4.29 (t, 2H), 3.77 (t, 2H), 3.69 (s, 2H), 3.30 (s, 3H) ppm | |
| 213 | N-{4-[1-(2-fluorobenzyl)-3-(4- | 1H-NMR (400 MHz, d₆-DMSO): δ = 10.41 (s, 1H), 8.27 (s, 1H), 8.18 (d, | — |
| | fluorophenyl)-1H-pyrazol-4- | 1H), 8.09 (s, 1H), 7.43-7.37 (m, 4H), 7.30-7.18 (m, 4H), 6.83 (dd, 1H), | |
| | yl]pyridin-2-yl}propanamide | 5.48 (s, 2H), 2.38 (q, 2H), 1.05 (t, 3H) ppm | |
| 214 | N-{4-[1-(cyclopropylmethyl)-3-(4- | 1H-NMR (400 MHz, d₃-CD3CN): δ = 8.57 (s, 1H, br), 8.10 (m, 2H), | — |
| | fluorophenyl)-1H-pyrazol-4- | 7.93 (s, 1H), 7.48-7.45 (m, 2H), 7.12-7.09 (m, 2H), 6.87 (dd, 1H), 4.03 (d, 2H), | |
| | yl]pyridin-2-yl}propanamide | 2.38 (q, 2H), 1.36 (m, 1H), 1.11 (t, 3H), 0.62 (m, 2H), 0.44 (m, 2H) ppm | |
| 215 | N-{4-[1-(2,2-difluoroethyl)-3-(4- | 1H-NMR (400 MHz, d₃-CD3CN): δ = 8.60 (s, 1H, br), 8.12-8.10 (m, 2H), | — |
| | fluorophenyl)-1H-pyrazol-4- | 7.93 (s, 1H), 7.48-7.45 (m, 2H), 7.13-7.10 (m, 2H), 6.86 (dd, 1H), 6.28 (tt, | |
| | yl]pyridin-2-yl}propanamide | 2J (F, H) = 54 Hz, 1H), 4.59 (dt, 3J (F, H) = 14 Hz, 2H), 2.39 (q, 2H), 1.11 (t, | |
| | | 3H) ppm | |
| 216 | N-{4-[1-(2,2-difluoroethyl)-3-(4- | 1H-NMR (400 MHz, d₃-CD3CN): δ = 8.76 (s, 1H, br), 8.17 (d, 2H), 8.09 (s, | — |
| | fluorophenyl)-1H-pyrazol-4- | 1H), 7.96 (s, 1H), 7.48-7.46 (m, 2H), 7.13-7.10 (m, 2H), 6.95 (dd, 1H), | |
| | yl]pyridin-2-yl}2-methoxyacetamide | 6.28 (tt, 2J (F, H) = 54 Hz, 1H), 4.59 (dt, 3J (F, H) = 14 Hz, 2H), 3.97 (s, 2H), 3.45 (s, | |
| | | 3H) ppm | |
| 217 | N-{4-[1-(2,2-difluoroethyl)-3-(4- | 1H-NMR (400 MHz, d₃-CD3CN): δ = 8.91 (s, 1H, br), 8.13 (dd, 2H), | — |
| | fluorophenyl)-1H-pyrazol-4- | 8.09 (s, 1H), 7.91 (s, 1H), 7.47-7.44 (m, 2H), 7.11-7.08 (m, 2H), 6.85 (dd, 1H), | |
| | yl]pyridin-2- | 6.27 (tt, 2J (F, H) = 59 Hz, 1H), 4.59 (dt, 3J (F, H) = 14 Hz, 2H), 1.77 (m, 1H), | |
| | yl}cyclopropanecarboxamide | 0.90-0.80 (m, 4H) ppm | |
| 218 | N-{4-[1-(2,2-difluoroethyl)-3-(4- | 1H-NMR (400 MHz, d₃-CD3CN): δ = 8.58 (s, 1H, br), 8.12 (m, 2H), | — |
| | fluorophenyl)-1H-pyrazol-4- | 7.93 (s, 1H), 7.47-7.45 (m, 2H), 7.12-7.09 (m, 2H), 6.86 (dd, 1H), 6.27 (tt, | |
| | yl]pyridin-2-yl}-2- | 2J (F, H) = 55 Hz, 1H), 4.58 (dt, 3J (F, H) = 14 Hz, 2H), 2.62 (m, 1H), 1.14 (d, | |
| | methylpropanamide | 6H) ppm | |
| 219 | N-{4-[1-(2-chloroethyl)-3-(4- | 1H-NMR (400 MHz, d₃-CD3CN): δ = 8.58 (s, 1H, br), 8.11 (m, 2H), | — |
| | fluorophenyl)-1H-pyrazol-4- | 7.93 (s, 1H), 7.49-7.45 (m, 2H), 7.15-7.08 (m, 2H), 6.86 (dd, 1H), 4.48 (t, 2H), | |
| | yl]pyridin-2-yl}propanamide | 4.02 (t, 2H), 2.38 (q, 2H), 1.11 (t, 3H) ppm | |
| 220 | N-{4-[1-(2-chloroethyl)-3-(4- | 1H-NMR (400 MHz, d₃-CD3CN): δ = 8.74 (s, 1H, br), 8.16 (dd, 1H), | — |
| | fluorophenyl)-1H-pyrazol-4- | 8.09 (s, 1H), 7.95 (s, 1H), 7.49-7.46 (m, 2H), 7.12-7.09 (m, 2H), 6.95 (dd, 1H), | |
| | yl]pyridin-2-yl}-2-methoxyacetamide | 4.49 (t, 2H), 4.02 (t, 2H), 3.96 (s, 2H), 3.45 (s, 3H) ppm | |
| 221 | N-{4-[1-(2-chloroethyl)-3-(4- | 1H-NMR (400 MHz, d₃-CD3CN): δ = 8.90 (s, 1H, br), 8.12 (d, 1H), 8.09 (s, | — |
| | fluorophenyl)-1H-pyrazol-4- | 1H), 7.90 (s, 1H), 7.47-7.45 (m, 2H), 7.12-7.08 (m, 2H), 6.86 (dd, 1H), | |
| | yl]pyridin-2- | 4.48 (t, 2H), 4.01 (t, 2H), 1.77 (m, 1H), 0.88-0.81 (m, 4H) ppm | |
| | yl}cyclopropanecarboxamide | | |
| 222 | N-{4-[1-(2-chloroethyl)-3-(4- | 1H-NMR (400 MHz, d₃-CD3CN): δ = 8.58 (s, 1H, br), 8.13-8.11 (m, 2H), | — |
| | fluorophenyl)-1H-pyrazol-4- | 7.93 (s, 1H), 7.48-7.46 (m, 2H), 7.12-7.09 (m, 2H), 6.86 (dd, 1H), 4.49 (t, | |
| | yl]pyridin-2-yl}-2- | 2H), 4.02 (t, 2H), 2.62 (m, 1H), 1.14 (d, 6H) ppm | |
| | methylpropanamide | | |
| 223 | N-{4-[1-(2-cyclopropylmethyl)-3-(4- | 1H-NMR (400 MHz, d₃-CD3CN): δ = 8.73 (s, 1H, br), 8.15 (d, 1H), 8.09 (s, | — |
| | fluorophenyl)-1H-pyrazol-4- | 1H), 7.95 (s, 1H), 7.48-7.46 (m, 2H), 7.12-7.09 (m, 2H), 6.96 (dd, 1H), | |
| | yl]pyridin-2-yl}-2-methoxyacetamide | 4.03 (d, 2H), 3.97 (s, 2H), 3.45 (s, 3H), 1.36 (m, 1H) 0.63 (m, 2H), 0.43 (m, 2H) | |
| | | ppm | |
| 224 | N-{4-[1-(cyclopropylmethyl)-3-(4- | 1H-NMR (400 MHz, d₃-CD3CN): δ = 8.92 (s, 1H, br), 8.11 (d, 1H), 8.10 (s, | — |
| | fluorophenyl)-1H-pyrazol-4- | 1H), 7.91 (s, 1H), 7.47-7.45 (m, 2H), 7.11-7.08 (m, 2H), 6.86 (dd, 1H), | |
| | yl]pyridin-2- | 4.02 (d, 2H), 1.78 (m, 1H), 1.34 (m, 1H), 0.85-0.80 (m, 4H), 0.62 (m, 2H), | |
| | yl}cyclopropanecarboxamide | 0.43 (m, 2H) ppm | |
| 225 | N-{4-[1-(cyclopropylmethyl)-3-(4- | 1H-NMR (400 MHz, d₃-CD3CN): δ = 8.60 (s, 1H, br), 8.12 (s, 1H), 8.10 (d, | — |
| | fluorophenyl)-1H-pyrazol-4- | 1H), 7.93 (s, 1H), 7.48-7.45 (m, 2H), 7.11-7.08 (m, 2H), 6.86 (dd, 1H), | |
| | yl]pyridin-2-yl}-2- | 4.03 (d, 2H), 2.63 (m, 1H), 1.38 (m, 1H), 1.14 (d, 6H), 0.62 (m, 2H), 0.43 (m, | |
| | methylpropanamide | 2H) ppm | |
| 226 | N-{4-[3-(4-fluorophenyl)-1-(propan- | 1H-NMR (400 MHz, d₃-CD3CN): δ = 8.54 (s, 1H, br), 8.13 (d, 1H), 8.09 (s, | — |
| | 2-yloxy)-1H-pyrazol-4-yl]pyridin-2- | 1H), 7.80 (s, 1H), 7.47-7.44 (m, 2H), 7.11-7.08 (m, 2H), 6.89 (dd, 1H), | |
| | yl}propanamide | 4.74 (m, 1H), 2.38 (q, 2H), 1.33 (d, 6H), 1.10 (t, 3H) ppm | |
| 227 | 4-[3-(4-fluorophenyl)-1-(propan-2- | 1H-NMR (400 MHz, d₃-CD3CN): δ = 8.45 (dd, 2H), 7.82 (s, 1H), | — |
| | yloxy)-1H-pyrazol-4-yl]pyridine | 7.45-7.43 (m, 2H), 7.20-7.19 (dd, 2H), 7.13-7.10 (m, 2H), 4.77 (m, 1H), 1.33 (d, | |
| | | 6H) ppm | |
| 228 | 3-(4-fluorophenyl)-4-[3-(4- | 1H-NMR (400 MHz, d₆-DMSO): δ = 8.51 (d, 1H), 8.45 (s, 1H), 7.45 (s, | — |
| | fluorophenyl)-1-(2-methylpropyl)-1H- | 1H), 7.29 (d, 1H), 7.04-7.03 (dd, 2H), 6.94-6.91 (dd, 2H), 6.90-6.80 (m, | |
| | pyrazol-4-yl]pyridine | 4H), 3.89 (d, 2H), 2.20 (m, 1H), 0.87 (d, 6H) ppm | |
| 229 | 3-(3-fluorophenyl)-4-[3-(4- | 1H-NMR (400 MHz, d₆-DMSO): δ = 8.54-8.53 (d, 1H), 8.46 (s, 1H), | — |
| | fluorophenyl)-1-(2-methylpropyl)-1H- | 7.46 (s, 1H), 7.32 (d, 1H), 7.13 (m, 1H), 7.05 (m, 2H), 6.90-6.86 (m, 2H), | |
| | pyrazol-4-yl]pyridine | 6.75 (m, 1H), 6.65 (m, 1H), 3.89 (d, 2H), 2.20 (m, 1H), 0.87 (d, 6H) ppm | |
| 230 | N-{4-[3-(3-cyano-4-fluorophenyl)-1- | 1H-NMR (400 MHz, d₆-DMSO): δ = 10.41 (s, 1H), 8.24 (s, 1H), 8.20 (m, | — |
| | (2-methylpropyl)-1H-pyrazol-4- | 1H), 8.06 (s, 1H), 7.92 (m, 1H), 7.78 (m, 1H), 7.56 (t, 1H), 6.92 (m, 1H), | |
| | yl]pyridin-2-yl}propanamide | 4.02 (d, 2H), 2.39 (q, 2H), 2.20 (m, 1H), 1.05 (t, 3H), 0.92 (d, 6H) ppm | |
| 231 | 1-{4-[1-(2,2-difluoroethyl)-3-(4- | 1H-NMR (400 MHz, d₃-CD3CN): δ = 8.80 (s, 1H, br), 8.06 (m, 1H), | — |
| | fluorophenyl)-1H-pyrazol-4- | 7.88 (s, 1H), 7.53-7.45 (m, 3H), 7.14-7.10 (m, 2H), 6.84 (s, 1H), 6.77 (m, 1H), | |
| | yl]pyridin-2-yl}-3-ethylurea | 6.27 (tt, 2J (F, H) = 55 Hz, 1H), 4.59 (dt, 3J (F, H) = 14 Hz, 2H), 3.27 (q, 2H), | |
| | | 1.14 (t, 3H) ppm | |
| 232 | N-(4-{3-(4-fluorophenyl)-1-[1-(2- | 1H-NMR (400 MHz, d₆-DMSO): δ = 10.40 (s, 1H), 8.34 (s, 1H), 8.17 (d, | — |
| | fluorophenyl)ethyl]-1H-pyrazol-4- | 1H), 8.10 (s, 1H), 7.45-7.35 (m, 4H), 7.25-7.18 (m, 4H), 6.82 (dd, 1H), | |
| | yl}pyridin-2-yl)-2- | 5.96 (q, 1H), 2.72 (m, 1H), 1.06 (d, 6H) ppm | |
| | methylpropanamide | | |
| 233 | 3-{4-[3-(4-fluorophenyl)-1-(propan-2- | 1H-NMR (400 MHz, d₃-CD3CN): δ = 8.05 (m, 1H), 7.90 (m, 1H), 7.85 (s, | — |
| | yl)-1H-pyrazol-4-yl]pyridin-2-yl}-1,1- | 1H), 7.48-7.46 (m, 2H), 7.36 (s, 1H, br), 7.11-7.09 (m, 2H), 6.77 (dd, 1H), | |
| | dimethylurea | 4.55 (m, 1H), 2.95 (s, 6H), 1.53 (d, 6H) ppm | |
| 234 | N-{4-[1-(2,2-difluoroethyl)-3-(4- | 1H-NMR (400 MHz, d₃-CD3CN): δ = 8.01 (s, 1H), 7.90 (d, 1H), 7.85 (m, | — |
| | fluorophenyl)-1H-pyrazol-4- | 1H), 7.50-7.45 (m, 3H), 7.13-7.05 (m, 2H), 6.35 (m, 1H), 6.27 (tt, 1H), | |
| | yl]pyridin-2-yl}propan-2- | 4.60 (m, 2H), 3.05 (m, 1H), 1.34 (d, 6H) ppm | |
| | sulphonamide | | |
| 235 | N-{4-[1-(2,2-difluoroethyl)-3-(4- | 1H-NMR (400 MHz, d₃-CD3CN): δ = 8.91 (s, 1H, br), 8.23-8.17 (m, 3H), | — |
| | fluorophenyl)-1H-pyrazol-4- | 7.97 (s, 1H), 7.57 (m, 1H), 7.49-7.48 (m, 2H), 7.13-7.11 (m, 2H), 6.94 (dd, | |
| | yl]pyridin-2-yl}thiophen-3- | 1H), 6.28 (tt, 2J (F, H) = 55 Hz, 1H), 4.60 (dt, 3J (F, H) = 14 Hz, 2H) ppm | |
| | carboxamide | | |
| 236 | N-{4-[1-(2,2-difluoroethyl)-3-(4- | 1H-NMR (400 MHz, d₃-CD3CN): δ = 9.04 (s, 1H, br), 8.24 (d, 1H), 8.20 (s, | — |
| | fluorophenyl)-1H-pyrazol-4- | 1H), 7.87 (dd, 1H), 7.74 (d, 1H), 7.53-7.51 (m, 2H), 7.21-7.20 (dd, 1H), | |
| | yl]pyridin-2-yl}thiophen-2- | 7.17-7.14 (m, 2H), 6.98 (dd, 1H), 6.31 (tt, 2J (F, H) = 55 Hz, 1H), 4.63 (dt, | |
| | carboxamide | 3J (F, H) = 14 Hz, 2H) ppm | |
| 237 | N-{4-[1-(2,2-difluoroethyl)-3-(4- | 1H-NMR (400 MHz, d₃-CD3CN): δ = 9.08 (s, 1H, br), 8.30 (m, 1H), | — |
| | fluorophenyl)-1H-pyrazol-4- | 8.24 (m, 1H), 8.01 (s, 1H), 7.97 (d, 2H), 7.64 (m, 1H), 7.56-7.50 (m, 4H), | |
| | yl]pyridin-2-yl}benzamide | 7.17-7.14 (m, 2H), 6.98 (dd, 1H), 6.32 (tt, 2J (F, H) = 55 Hz, 1H), 4.64 (dt, | |
| | | 3J (F, H) = 14 Hz, 2H) ppm | |
| 238 | N-{4-[1-(2,2-difluoroethyl)-3-(4- | 1H-NMR (400 MHz, d₃-CD3CN): δ = 8.57 (s, 1H, br), 8.13-8.10 (m, 2H), | — |
| | fluorophenyl)-1H-pyrazol-4- | 7.93 (s, 1H), 7.47-7.45 (m, 2H), 7.12-7.09 (m, 2H), 6.88 (dd, 1H), 6.27 (tt, | |
| | yl]pyridin-2-yl}-3-methylbutanamide | 2J (F, H) = 55 Hz, 1H), 4.58 (dt, 3J (F, H) = 14 Hz, 2H), 2.22 (m, 2H), 2.10 (m, | |
| | | 1H), 0.95 (d, 6H) ppm | |
| 239 | N-{4-[1-(2,2-difluoroethyl)-3-(4- | 1H-NMR (400 MHz, d₃-CD3CN): δ = 8.57 (s, 1H, br), 8.13-8.10 (m, 2H), | — |
| | fluorophenyl)-1H-pyrazol-4- | 7.93 (s, 1H), 7.47-7.45 (m, 2H), 7.12-7.09 (m, 2H), 6.86 (dd, 1H), 6.27 (tt, | |
| | yl]pyridin-2-yl}-2-methylbutanamide | 2J (F, H) = 55 Hz, 1H), 4.59 (dt, 3J (F, H) = 14 Hz, 2H), 2.42 (m, 1H), 1.65 (m, | |
| | | 1H), 1.45 (m, 1H), 1.12 (d, 3H), 0.89 (t, 3H) ppm | |
| 240 | N-{4-[1-(2,2-difluoroethyl)-3-(4- | 1H-NMR (400 MHz, d₃-CD3CN): δ = 8.44 (s, 1H, br), 8.16-8.14 (m, 2H), | — |
| | fluorophenyl)-1H-pyrazol-4- | 7.96 (s, 1H), 7.51-7.49 (m, 2H), 7.16-7.13 (m, 2H), 6.89 (dd, 1H), 6.31 (tt, | |
| | yl]pyridin-2- | 2J (F, H) = 55 Hz, 1H), 4.62 (dt, 3J (F, H) = 14 Hz, 2H), 2.30-2.20 (m, 6H), | |
| | yl}cyclobutancarboxamide | 1.85 (m, 1H) ppm | |
| 241 | ethyl {4-[1-(2,2-difluoroethyl)-3-(4- | 1H-NMR (400 MHz, d₃-CD3CN): δ = 8.16 (s, 1H, br), 8.12 (d, 1H), 7.92 (s, | — |
| | fluorophenyl)-1H-pyrazol-4- | 1H), 7.85 (d, 1H), 7.48-7.46 (m, 2H), 7.12-7.10 (m, 2H), 6.86 (dd, 1H), | |
| | yl]pyridin-2-yl}carbamate | 6.27 (tt, 2J (F, H) = 55 Hz, 1H), 4.59 (dt, 3J (F, H) = 14 Hz, 2H), 4.14 (q, 2H), | |
| | | 1.24 (t, 3H) ppm | |
| 242 | N-{4-[1-(2,2-difluoroethyl)-3-(4- | 1H-NMR (400 MHz, d₃-CD3CN): δ = 8.63 (s, 1H, br), 8.13 (dd, 1H), | — |
| | fluorophenyl)-1H-pyrazol-4- | 8.06 (s, 1H), 7.92 (s, 1H), 7.48-7.46 (m, 2H), 7.13-7.09 (m, 2H), 6.88 (dd, 1H), | |
| | yl]pyridin-2-yl}acetamide | 6.27 (tt, 2J (F, H) = 55 Hz, 1H), 4.59 (dt, 3J (F, H) = 14 Hz, 2H), 2.10 (s, 3H) | |
| | | ppm | |
| 243 | N-{4-[1-(2,2-difluoroethyl)-3-(4- | 1H-NMR (400 MHz, d₃-CD3CN): δ = 9.20 (s, 1H, br), 8.12 (m, 2H), | — |
| | fluorophenyl)-1H-pyrazol-4- | 7.97 (s, 1H), 7.51-7.49 (m, 2H), 7.15-7.13 (m, 2H), 6.94 (dd, 1H), 6.30 (tt, | |
| | yl]pyridin-2-yl}-2-hydroxy-2- | 2J (F, H) = 55 Hz, 1H), 4.62 (dt, 2H), 1.43 (s, 6H) ppm | |
| | methylpropanamide | | |
| 244 | N-(4-{3-(4-fluorophenyl)-1-[1-(2- | 1H-NMR (400 MHz, d₆-DMSO): δ = 9.95 (s, 1H), 8.38 (s, 1H), 8.20 (dd, | — |
| | fluorophenyl)ethyl]-1H-pyrazol-4- | 1H), 8.07 (s, 1H), 7.43-7.36 (m, 4H), 7.25-7.15 (m, 4H), 6.96 (dd, 1H), | |
| | yl}pyridin-2-yl)-2-methoxyacetamide | 4.03 (s, 2H), 3.40 (s, 3H), 1.90 (d, 3H) ppm | |
| 245 | N-(4-{1-[(2,2- | 1H-NMR (400 MHz, d₆-DMSO): δ = 9.96 (s, 1H), 8.26 (s, 1H), 8.22 (d, | — |
| | dichlorocyclopropyl)methyl]-3-(4- | 1H), 8.06 (s, 1H), 7.47 (m, 2H), 7.23 (m, 2H), 6.91 (dd, 1H), 4.45 (dd, 1H), | |
| | fluorophenyl)-1H-pyrazol-4- | 4.34 (dd, 1H), 4.03 (s, 2H), 3.40 (s, 3H), 2.40 (m, 1H), 1.90 (dd, 1H), | |
| | yl}pyridin-2-yl)-2-methoxyacetamide | 1.70 (dd, 1H) ppm | |
| 246 | N-{4-[3-(3-cyano-4-fluorophenyl)-1- | 1H-NMR (400 MHz, d₆-DMSO): δ = 9.97 (s, 1H), 8.27 (s, 1H), 8.24 (d, | — |
| | (2-methylpropyl)-1H-pyrazol-4- | 1H), 7.98 (s, 1H), 7.92 (dd, 1H), 7.78 (m, 1H), 7.55 (t, 1H), 6.99 (d, 1H), | |
| | yl]pyridin-2-yl}-2-methoxyacetamide | 4.03 (m, 4H), 3.40 (s, 3H), 2.20 (m, 1H), 0.91 (d, 6H) ppm | |
| 247 | N-{4-[1-(2-fluorobenzyl)-3-(4- | 1H-NMR (400 MHz, d₆-DMSO): δ = 9.96 (s, 1H), 8.30 (s, 1H), 8.21 (d, | — |
| | fluorophenyl)-1H-pyrazol-4- | 1H), 8.04 (s, 1H), 7.45-7.38 (m, 4H), 7.30-7.18 (m, 4H), 6.92 (dd, 1H), | |
| | yl]pyridin-2-yl}-2-methoxyacetamide | 5.49 (s, 2H), 4.03 (s, 2H), 3.4 (s, 3H) ppm | |
| 248 | N-(4-{3-(4-fluorophenyl)-1-[1-(2- | 1H-NMR (400 MHz, d₆-DMSO): δ = 10.40 (s, 1H), 8.34 (s, 1H), 8.18 (d, | — |
| | fluorophenyl)ethyl]-1H-pyrazol-4- | 1H), 8.08 (s, 1H), 7.42-7.35 (m, 4H), 7.25-7.18 (m, 4H), 6.86 (dd, 1H), | |
| | yl}pyridin-2-yl)propanamide | 5.96 (q, 1H), 2.38 (q, 2H), 1.90 (d, 3H), 1.05 (t, 3H) ppm | |
| 249 | N-(4-{1-[(2,2- | 1H-NMR (400 MHz, d₆-DMSO): δ = 10.42 (s, 1H), 8.24 (s, 1H), 8.18 (d, | — |
| | dichlorocyclopropyl)methyl]-3-(4- | 1H), 8.11 (s, 1H), 7.47 (m, 2H), 7.23 (m, 2H), 6.84 (dd, 1H), 4.45 (dd, 1H), | |
| | fluorophenyl)-1H-pyrazol-4- | 4.33 (dd, 1H), 2.40-2.35 (m, 3H), 1.90 (dd, 1H), 1.70 (dd, 1H), 1.04 (q, 3H) | |
| | yl}pyridin-2-yl)propanamide | ppm | |
| 250 | 2-cyclopropyl-N-(4-{3-(4- | 1H-NMR (400 MHz, d₆-DMSO): δ = 10.33 (s, 1H), 8.33 (s, 1H), 8.16 (d, | — |
| | fluorophenyl)-1-[1-(2- | 1H), 8.09 (s), 7.42-7.35 (m, 4H), 7.25-7.15 (m, 4H), 6.84 (dd, 1H), 5.95 (q, | |
| | fluorophenyl)ethyl]-1H-pyrazol-4- | 1H), 2.23 (d, 2H), 1.90 (d, 3H), 1.00 (m, 1H), 0.45 (m, 2H), 0.16 (m, 2H) | |
| | yl}pyridin-2-yl)acetamide | ppm | |
| 251 | 2-cyclopropyl-N-(4-{1-[(2,2- | 1H-NMR (400 MHz, d₆-DMSO): δ = 10.35 (s, 1H), 8.24 (s, 1H), 8.18 (dd, | — |
| | dichlorocyclopropyl)methyl]-3-(4- | 1H), 8.11 (s, 1H), 7.45 (m, 2H), 7.22 (m, 2H), 6.83 (dd, 1H), 4.45 (dd, 1H), | |
| | fluorophenyl)-1H-pyrazol-4- | 4.33 (dd, 1H), 2.40 (m, 1H), 2.23 (d, 2H), 1.90 (dd, 1H), 1.70 (dd, 1H), | |
| | yl}pyridin-2-yl)acetamide | 1.00 (m, 1H), 0.45 (d, 2H), 0.17 (d, 2H) ppm | |
| 252 | N-{4-[3-(3-cyano-4-fluorophenyl)-1- | 1H-NMR (400 MHz, d₆-DMSO): δ = 10.34 (s, 1H), 8.24 (s, 1H), 8.20 (d, | — |
| | (2-methylpropyl)-1H-pyrazol-4- | 1H), 8.06 (s, 1H), 7.90 (dd, 1H), 7.78 (m, 1H), 7.56 (t, 1H), 6.92 (dd, 1H), | |
| | yl]pyridin-2-yl}-2- | 4.00 (d, 2H), 2.23 (d, 2H), 2.20 (m, 1H), 1.05 (m, 1H), 0.91 (d, 6H), | |
| | cyclopropylacetamide | 0.45 (d, 2H), 0.16 (d, 2H) ppm | |
| 253 | 2-cyclopropyl-N-{4-[1-(2- | 1H-NMR (400 MHz, d₆-DMSO): δ = 10.33 (s, 1H), 8.27 (s, 1H), 8.16 (dd, | — |
| | fluorobenzyl)-3-(4-fluorophenyl)-1H- | 1H), 8.09 (s, 1H), 7.42-7.35 (m, 4H), 7.25-7.15 (m, 4H), 6.82-6.80 (dd, 1H), | |
| | pyrazol-4-yl]pyridin-2-yl}acetamide | 5.47 (s, 2H), 2.24 (d, 2H), 1.00 (m, 1H), 0.44 (m, 2H), 0.18 (m, 2H) ppm | |
| 254 | N-(4-{1-[(2,2- | 1H-NMR (400 MHz, d₆-DMSO): δ = 10.41 (s, 1H), 8.24 (s, 1H), 8.18 (d, | — |
| | dichlorocyclopropyl)methyl]-3-(4- | 1H), 8.13 (s, 1H), 7.45 (dd, 2H), 7.22 (m, 2H), 6.81 (dd, 1H), 4.45 (dd, 1H), | |
| | fluorophenyl)-1H-pyrazol-4- | 4.35 (dd, 1H), 2.75 (m, 1H), 2.43 (m, 1H), 1.89 (dd, 1H), 1.70 (dd, 1H), | |
| | yl}pyridin-2-yl)-2- | 1.05 (d, 6H) ppm | |
| | methylpropanamide | | |
| 255 | N-{4-[3-(3-cyano-4-fluorophenyl)-1- | 1H-NMR (400 MHz, d₆-DMSO): δ = 10.41 (s, 1H), 8.25 (s, 1H), 8.20 (d, | — |
| | (2-methylpropyl)-1H-pyrazol-4- | 1H), 8.09 (s, 1H), 7.90 (dd, 1H), 7.78 (m, 1H), 7.55 (t, 1H), 6.90 (dd, 1H), | |
| | yl]pyridin-2-yl}-2- | 4.00 (d, 2H), 2.72 (m, 1H), 2.20 (m, 1H), 1.05 (d, 6H), 0.91 (d, 6H) ppm | |
| | methylpropanamide | | |
| 256 | N-{4-[1-(2-fluorobenzyl)-3-(4- | 1H-NMR (400 MHz, d₆-DMSO): δ = 10.40 (s, 1H), 8.27 (s, 1H), 8.16 (dd, | — |
| | fluorophenyl)-1H-pyrazol-4- | 1H), 8.11 (s, 1H), 7.45-7.35 (m, 3H), 7.25-7.15 (m, 5H), 6.80-6.79 (dd, 1H), | |
| | yl]pyridin-2-yl}-2- | 5.47 (s, 2H), 2.61 (m, 1H), 1.05 (d, 6H) ppm | |
| | methylpropanamide | | |
| 258 | N-{4-[5-(difluoromethoxy)-3-(4- | 1H-NMR (400 MHz, d₆-DMSO): δ = 10.69 (s, 1H), 9.92 (s, 1H), 8.15 (d, | — |
| | fluoro-2-hydroxyphenyl)-1-methyl- | 1H), 8.03 (s, 1H), 7.20 (m, 1H), 7.13 (t, 2J (H, F) = 71 Hz, 1H), 6.73 (dd, 1H), | |
| | 1H-pyrazol-4-yl]pyridin-2- | 6.67-6.64 (m, 1H), 6.58-6.55 (dd, 1H), 3.79 (s, 3H), 1.95 (m, 1H), 0.76 (m, | |
| | yl}cyclopropanecarboxamide | 4H) ppm | |
| 259 | N-(4-{3-(4-fluorophenyl)-1-[1-(2- | 1H-NMR (400 MHz, d₆-DMSO): δ = 10.76 (s, 1H), 8.32 (s, 1H), 8.18 (d, | — |
| | fluorophenyl)ethyl]-1H-pyrazol-4- | 1H), 8.07 (s), 7.44-7.42 (m, 4H), 7.25-7.15 (m, 4H), 6.83 (dd, 1H), 5.95 (q, | |
| | yl}pyridin-2- | 1H), 2.00 (m, 2H), 1.80 (d, 3H), 0.80 (m, 4H) ppm | |
| | yl)cyclopropanecarboxamide | | |
| 260 | N-(4-{1-[(2,2-dichlorocyclo- | 1H-NMR (400 MHz, d₆-DMSO): δ = 10.78 (s, 1H), 8.22 (s, 1H), 8.19 (d, | — |
| | propyl)methyl]-3-(4-fluorophenyl)- | 1H), 8.09 (s, 1H), 7.44 (dd, 2H), 7.23 (m, 2H), 6.82 (dd, 1H), 4.45 (dd, 1H), | |
| | 1H-pyrazol-4-yl}pyridin-2- | 4.34 (dd, 1H), 2.40 (m, 1H), 1.98 (m, 1H), 1.70 (dd, 1H), 0.78 (m, 4H) ppm | |
| | yl)cyclopropanecarboxamide | | |
| 261 | N-{4-[3-(3-cyano-4-fluorophenyl)-1- | 1H-NMR (400 MHz, d₆-DMSO): δ = 10.78 (s, 1H), 8.23 (s, 1H), 8.21 (d, | — |
| | (2-methylpropyl)-1H-pyrazol-4- | 1H), 8.05 (s, 1H), 7.90 (dd, 1H), 7.77 (m, 1H), 7.55 (t, 1H), 6.90 (dd, 1H), | |
| | yl]pyridin-2-yl}cyclopropane | 4.00 (d, 2H), 2.20 (m, 1H), 1.98 (m, 1H), 0.90 (d, 6H), 0.78 (m, 4H) ppm | |
| | carboxamide | | |
| 262 | N-{4-[1-(2-fluorobenzyl)-3-(4- | 1H-NMR (400 MHz, d₆-DMSO): δ = 10.78 (s, 1H), 8.25 (s, 1H), 8.17 (d, | — |
| | fluorophenyl)-1H-pyrazol-4- | 1H), 8.08 (s, 1H), 7.45-7.30 (m, 3H), 7.30-7.15 (m, 5H), 6.81 (m, 1H), | |
| | yl]pyridin-2- | 5.47 (s, 2H), 1.98 (m, 1H), 0.78 (m, 4H) ppm | |
| | yl}cyclopropanecarboxamide | | |
| 263 | ethyl (4-{3-(4-fluorophenyl)-1-[1-(2- | 1H-NMR (400 MHz, d₆-DMSO): δ = 10.05 (s, 1H), 8.35 (s, 1H), 8.15 (d, | — |
| | fluorophenyl)ethyl]-1H-pyrazol-4- | 1H), 7.77 (s, 1H), 7.45-7.35 (m, 3H), 7.25-7.15 (m, 5H), 6.83 (dd, 1H), | |
| | yl}pyridin-2-yl)carbamate | 5.94 (q, 1H), 1.90 (d, 3H), 1.22 (t, 3H) ppm | |
| 264 | ethyl (4-{1-[(2,2- | 1H-NMR (400 MHz, d₆-DMSO): δ = 10.07 (s, 1H), 8.24 (s, 1H), 8.15 (d, | — |
| | dichlorocyclopropyl)methyl]-3-(4- | 1H), 7.78 (s, 1H), 7.47-7.45 (dd, 2H), 7.22 (dd, 2H), 6.83 (dd, 1H), | |
| | fluorophenyl)-1H-pyrazol-4- | 4.47-4.44 (dd, 1H), 4.35-4.32 (dd, 1H), 4.09 (q, 2H), 2.40 (m, 1H), 1.90 (dd, 1H), | |
| | yl}pyridin-2-yl)carbamate | 1.70 (dd, 1H), 1.22 (t, 3H) ppm | |
| 265 | ethyl {4-[3-(3-cyano-4-fluorophenyl)- | 1H-NMR (400 MHz, d₆-DMSO): δ = 10.08 (s, 1H), 8.25 (s, 1H), 8.16 (m, | — |
| | 1-(2-methylpropyl)-1H-pyrazol-4- | 1H), 7.92 (m, 1H), 7.78-7.70 (m, 2H), 7.56-7.53 (m, 2H), 7.40 (m, 1H), | |
| | yl]pyridin-2-yl}carbamate | 6.90 (dd, 1H), 4.09 (q, 2H), 4.00 (d, 2H), 2.20 (m, 1H), 1.22 (m, 6H), | |
| | | 0.80 (d, 3H) ppm | |
| 266 | ethyl {4-[1-(2-fluorobenzyl)-3-(4- | 1H-NMR (400 MHz, d₆-DMSO): δ = 10.06 (s, 1H), 8.27 (s, 1H), | — |
| | fluorophenyl)-1H-pyrazol-4- | 8.14-8.13 (d, 1H), 7.78 (d, 1H), 7.44-7.42 (m, 4H), 7.35 (m, 1H), 7.25-7.15 (m, 4H), | |
| | yl]pyridin-2-yl}carbamate | 5.47 (s, 2H), 4.10 (q, 2H), 1.22 (t, 3H) ppm | |
| 267 | ethyl {4-[3-(4-fluorophenyl)-1-(2- | 1H-NMR (400 MHz, d₆-DMSO): δ = 10.05 (s, 1H), 8.19 (s, 1H), 8.13 (d, | — |
| | methylpropyl)-1H-pyrazol-4- | 1H), 7.78 (d, 1H), 7.44-7.42 (m, 2H), 7.22-7.19 (dd, 2H), 6.83-6.82 (dd, | |
| | yl]pyridin-2-yl}carbamate | 1H), 4.10 (q, 2H), 3.99 (d, 2H), 2.18 (m, 1H), 1.22 (m, 6H), 0.80 (d, 3H) | |
| | | ppm | |
| 268 | ethyl {4-[1-(butan-2-yl)-3-(4- | 1H-NMR (400 MHz, d₆-DMSO): δ = 10.04 (s, 1H), 8.19 (s, 1H), 8.13 (d, | — |
| | fluorophenyl)-1H-pyrazol-4- | 1H), 7.77 (s, 1H), 7.45-7.42 (m, 2H), 7.22 (dd, 2H), 6.84-6.83 (dd, 1H), | |
| | yl]pyridin-2-yl}carbamate | 4.35 (m, 1H), 4.09 (q, 2H), 1.90 (m, 1H), 1.80 (m, 1H), 1.22 (m, 6H), | |
| | | 0.80 (t, 3H) ppm | |
| 269 | N-{4-[1-(cyclopentyloxy)-3-(4- | 1H-NMR (400 MHz, d₃-CD3CN): δ = 8.86 (s, 1H, br), 8.14 (d, 1H), 8.07 (s, | — |
| | fluorophenyl)-1H-pyrazol-4- | 1H), 7.80 (s, 1H), 7.47-7.43 (m, 2H), 7.10-7.06 (m, 2H), 6.88 (dd, 1H), | |
| | yl]pyridin-2-yl}cyclopropane- | 5.09 (m, 1H), 1.85-1.78 (m, 5H), 1.70-1.63 (m, 2H), 0.85-0.75 (m, 4H) ppm | |
| | carboxamide | | |
| 270 | N-{4-[1-(cyclopentyloxy)-3-(4- | 1H-NMR (400 MHz, d₃-CD3CN): δ = 8.53 (s, 1H, br), 8.13-8.11 (m, 2H), | — |
| | fluorophenyl)-1H-pyrazol-4- | 7.81 (s, 1H), 7.47-7.44 (m, 2H), 7.11-7.08 (m, 2H), 6.87 (dd, 1H), 5.11 (m, | |
| | yl]pyridin-2-yl}-2- | 1H), 2.63 (m, 1H), 1.85-1.78 (m, 4H), 1.70-1.63 (m, 2H), 1.13 (d, 6H) ppm | |
| | methylpropanamide | | |
| 271 | N-{4-[3-(4-fluorophenyl)-1-(propan- | 1H-NMR (400 MHz, d₃-CD3CN): δ = 8.87 (s, 1H, br), 8.13-8.12 (d, 1H), | — |
| | 2-yloxy)-1H-pyrazol-4-yl]pyridin-2- | 8.07 (s, 1H), 7.78 (s, 1H), 7.46-7.44 (m, 2H), 7.10-7.08 (m, 2H), 6.87 (dd, | |
| | yl}cyclopropanecarboxamide | 1H), 4.75 (m, 1H), 1.76 (m, 1H), 1.32 (d, 6H), 0.87-0.81 (m, 4H) ppm | |
| 272 | N-{4-[3-(4-fluorophenyl)-1-(propan- | 1H-NMR (400 MHz, d₃-CD3CN): δ = 8.53 (s, 1H, br), 8.13-8.11 (m, 2H), | — |
| | 2-yloxy)-1H-pyrazol-4-yl]pyridin-2- | 7.80 (s, 1H), 7.47-7.44 (m, 2H), 7.10-7.08 (m, 2H), 6.87 (dd, 1H), 4.75 (m, | |
| | yl}-2-methylpropanamide | 1H), 2.60 (m, 1H), 1.32 (d, 6H), 1.13 (d, 6H) ppm | |
| 273 | N-(cyclopropylmethyl)-4-[3-(4- | 1H-NMR (400 MHz, d₆-DMSO): δ = 8.30 (bs, 1H), 8.11 (d, 1H), 7.56 (m, | — |
| | fluorophenyl)-1-(2-methylpropyl)-1H- | 2H), 7.21 (t, 2H), 7.00 (m, 1H), 6.47 (bs, 1H), 3.99 (d, 2H), 2.94 (bs, 2H), | |
| | pyrazol-4-yl]pyrimidin-2-amine | 2.18 (m, 1H), 0.90 (d, 6H), 0.35 (m, 2H), 0.10 (m, 2H) ppm | |
| 274 | methyl 2-{3-(4-fluorophenyl)-4-[2- | 1H-NMR (400 MHz, d₆-DMSO): δ = 10.41 (s, 1H), 8.27 (s, 1H), 8.18 (dd, | — |
| | (propionylamino)pyridin-4-yl]-1H- | 1H), 8.09 (s, 1H), 7.44-7.41 (m, 2H), 7.22-7.20 (m, 2H), 6.85 (dd, 1H), | |
| | pyrazol-1-yl}-3-methylbutanoate | 4.92 (d, 1H), 3.72 (s, 3H), 2.60 (m, 1H), 2.37 (q, 2H), 1.03 (t, 3H), 1.00 (d, 3H), | |
| | | 0.86 (d, 3H) ppm | |
| 275 | 4-[1-(1,3-dioxolan-2-ylmethyl)-3-(4- | 1H-NMR (400 MHz, d₆-DMSO): δ = 8.47 (dd, 2H), 8.19 (s, 1H), | — |
| | fluorophenyl)-1H-pyrazol-4- | 7.44-7.42 (m, 2H), 7.25-7.15 (m, 4H), 5.27 (t, 1H), 4.32 (d, 2H), 3.95-3.85 (m, 4H) | |
| | yl]pyridine | ppm | |
| 276 | N-{4-[1-(1,3-dioxolan-2-ylmethyl)-3- | 1H-NMR (400 MHz, d₆-DMSO): δ = 10.41 (s, 1H), 8.16 (dd, 1H), 8.12 (s, | — |
| | (4-fluorophenyl)-1H-pyrazol-4- | 2H), 7.44-7.42 (m, 2H), 7.22-7.19 (m, 2H), 6.78 (dd, 1H), 5.27 (t, 1H), | |
| | yl]pyridin-2-yl}-2- | 4.32 (d, 2H), 3.95-3.80 (m, 4H), 2.72 (m, 1H), 1.06 (d, 6H) ppm | |
| | methylpropanamide | | |
| 277 | methyl 2-{3-(4-fluorophenyl)-4-[2- | 1H-NMR (400 MHz, d₆-DMSO): δ = 10.42 (s, 1H), 8.27 (s, 1H), 8.18 (dd, | — |
| | (isobutyrylamino)pyridin-4-yl]-1H- | 1H), 8.12 (s, 1H), 7.44-7.41 (m, 2H), 7.22-7.20 (m, 2H), 6.83 (dd, 1H), | |
| | pyrazol-1-yl}-3-methylbutanoate | 4.92 (d, 1H), 3.72 (s, 3H), 2.73 (m, 1H), 2.62 (m, 1H), 1.06 (d, 6H), 1.00 (d, 3H), | |
| | | 0.86 (d, 3H) ppm | |
| 278 | N-{4-[1-(1,3-dioxolan-2-ylmethyl)-3- | 1H-NMR (400 MHz, d₆-DMSO): δ = 10.41 (s, 1H), 8.16 (dd, 1H), | — |
| | (4-fluorophenyl)-1H-pyrazol-4- | 8.11-8.09 (m, 2H), 7.44-7.42 (m, 2H), 7.22-7.19 (m, 2H), 6.81 (dd, 1H), 5.27 (t, | |
| | yl]pyridin-2-yl}propanamide | 1H), 4.32 (d, 2H), 3.95-3.80 (m, 4H), 2.37 (q, 2H), 1.04 (t, 3H) ppm | |
| 279 | 4-[3-(4-fluorophenyl)-1-(2- | 1H-NMR (400 MHz, d₆-DMSO): δ = 8.31 (bs, 1H), 8.16 (d, 1H), 7.73 (d, | — |
| | methylpropyl)-1H-pyrazol-4-yl]-N- | 1H), 7.54 (m, 2H), 7.27 (t, 2H), 6.52 (bs, 1H), 4.54 (bs, 2H), 4.43 (bs, 2H), | |
| | (oxetan-3-yl)pyrimidin-2-amine | 3.99 (d, 2H), 3.75 (bs, 1H), 2.18 (m, 1H), 0.90 (d, 6H) ppm | |
| 280 | 7-[3-(4-fluorophenyl)-1-isopropyl-1H- | 1H-NMR (400 MHz, CDCl₃): δ = 12.35 (bs, 1H), 8.78 (s, 1H), 8.69 (s, 1H), | — |
| | pyrazol-4-yl]-3H-imidazo[4,5- | 8.12 (s, 1H), 7.65 (m, 2H), 7.00 (t, 2H), 4.58 (m, 1H), 1.57 (d, 6H) ppm | |
| | b]pyridine | | |
| 281 | N cycloproyl-4-[3-(4-fluorophenyl)- | 1H-NMR (400 MHz, d₆-DMSO): δ = 8.26 (bs, 1H), 8.14 (d, 1H), 7.63 (m, | — |
| | 1-(2-methylpropyl)-1H-pyrazol-4- | 2H), 7.21 (t, 2H), 6.48 (d, 1H), 4.0 (d, 2H), 2.62 (m, 1H), 2.018 (m, 1H), | |
| | yl]pyrimidin-2-amine | 0.9 (d, 6H), 0.55 (m, 2H), 0.43 (m, 2H) ppm | |
| 282 | 6-[3-(4-fluorophenyl)-1-isopropyl-1H- | 1H-NMR (400 MHz, d₃-CD3CN): δ = 11.00 (s, 1H), 8.85 (s, 1H), 8.60 (s, | — |
| | pyrazol-4-yl]-9H-purine | 1H), 8.21 (s, 1H), 7.74-7.73 (m, 2H), 7.12-7.09 (m, 2H), 4.65 (m, 1H), | |
| | | 1.57 (d, 6H) ppm | |
| 283 | 4-[3-(4-fluorophenyl)-1-isopropyl-1H- | 1H-NMR (250 MHz, CDCl₃): δ = 8.24 (d, 1H), 8.04 (s, 1H), 7.65 (s, 1H), | — |
| | pyrazol-4-yl]-2-(4-methyl-1H- | 7.41 (m, 2H), 7.12 (d, 1H), 7.06-6.98 (m, 4H), 4.52 (m, 1H), 2.20 (s, 3H), | |
| | imidazol-1-yl)pyridine | 1.54 (d, 6H) ppm | |
| 284 | N-tert-butyl-4-[3-(4-fluorophenyl)-1- | 1H-NMR (400 MHz, d₆-DMSO): δ = 8.18 (s, 1H), 8.10 (d, 1H), 7.5 (m, | — |
| | (2-methylpropyl)-1H-pyrazol-4- | 2H), 7.22 (t, 2H), 6.45 (s, 1H), 6.33 (d, 1H), 4.0 (d, 2H), 2.17 (m, 1H), | |
| | yl]pyrimidin-2-amine | 1.3 (bs, 9H), 0.9 (d, 6H) ppm | |
| 285 | 4-[3-(4-fluorophenyl)-1-(2- | 1H-NMR (400 MHz, d₆-DMSO): δ = 8.04 (bs, 1H), 7.88 (d, 1H), 7.31 (m, | — |
| | methylpropyl)-1H-pyrazol-4-yl]-N-(1- | 2H), 6.97 (t, 2H), 6.48 (d, 1H), 6.19 (bs, 1H), 3.76 (m, 3H), 3.10 (s, 3H), | |
| | methoxypropan-2-yl)pyrimidin-2- | 2.96 (bs, 2H), 1.94 (m, 1H), 0.80 (bs, 3H), 0.66 (d, 6H) ppm | |
| | amine | | |
| 286 | N-(butan-2-yl)-4-[3-(4-fluorophenyl)- | 1H-NMR (400 MHz, d₆-DMSO): δ = 8.25 (bs, 1H), 8.10 (d, 1H), 7.55 (m, | — |
| | 1-(2-methylpropyl)-1H-pyrazol-4- | 2H), 7.20 (t, 2H), 6.72 (d, 1H), 3.99 (d, 2H), 2.18 (m, 1H), 1.00 (bs, 2H), | |
| | yl]pyrimidin-2-amine | 0.9 (d, 6H), 0.78 (bs, 2H) ppm | |
| 287 | 4-[3-(4-fluorophenyl)-1-(2- | 1H-NMR (400 MHz, d₆-DMSO): δ = 8.87 (d, 1H), 8.70 (s, 1H), 7.62 (d, | — |
| | methylpropyl)-1H-pyrazol-4-yl]-2- | 1H), 7.61 (m, 2H), 7.25 (t, 2H), 4.05 (d, 2H), 3.17 (s, 3H), 2.22 (m, 1H), | |
| | (methylsulphonyl)pyrimidine | 0.92 (d, 6H) ppm | |
| 288 | 4-[3-(4-fluorophenyl)-1-isobutyl-1H- | 1H-NMR (400 MHz, d₃-CD3CN): δ = 9.02 (s, 1H), 8.17 (s, 1H), 7.77 (s, | — |
| | pyrazol-4-yl]-7-methylthieno[3,2- | 1H), 7.52-7.50 (m, 2H), 7.05-7.02 (m, 2H), 4.06 (d, 2H), 2.46 (s, 3H), | |
| | d]pyrimidine | 2.27 (m, 1H), 0.96 (d, 6H) ppm | |
| 289 | 4-[1-(cyclopropylmethyl)-3-(4- | 1H-NMR (400 MHz, d₃-CD3CN): δ = 9.02 (s, 1H), 8.26 (s, 1H), 7.77 (m, | — |
| | fluorophenyl)-1H-pyrazol-4-yl]-7- | 1H), 7.53-7.50 (m, 2H), 7.06-7.02 (m, 2H), 4.10 (d, 2H), 2.46 (d, 3H), | |
| | methylthieno[3,2-d]pyrimidine | 1.42-1.37 (m, 1H), 0.66-0.64 (m, 2H), 0.48-0.43 (m, 2H) ppm | |
| 290 | N-{4-[1-ethyl-3-(4-fluorophenyl)-1H- | 1H-NMR (400 MHz, d₃-CD3CN): δ = 8.61 (s, 1H, br), 8.11-8.09 (m, 2H), | — |
| | pyrazol-4-yl]pyridin-2-yl}-2- | 7.87 (s, 1H), 7.47-7.45 (m, 2H), 7.11-7.09 (m, 2H), 6.86 (dd, 1H), 4.20 (q, | |
| | methylpropanamide | 2H), 2.62 (m, 1H), 1.50 (t, 3H), 1.14 (d, 6H) ppm | |
| 291 | 2-cyclopropyl-N-{4-[1-ethyl-3-(4- | 1H-NMR (400 MHz, d₃-CD3CN): δ = 8.60 (s, 1H, br), 8.11 (m, 2H), | — |
| | fluorophenyl)-1H-pyrazol-4- | 7.88 (s, 1H), 7.47-7.45 (m, 2H), 7.11-7.08 (m, 2H), 6.88 (dd, 1H), 4.21 (q, 2H), | |
| | yl]pyridin-2-yl}acetamide | 2.26 (d, 2H), 1.50 (t, 3H), 1.05 (m, 1H), 0.54 (m, 2H), 0.21 (m, 2H) ppm | |
| 292 | N-{4-[3-(4-fluorophenyl)-1-propyl- | 1H-NMR (400 MHz, d₃-CD3CN): δ = 8.61 (s, 1H, br), 8.10 (m, 2H), | — |
| | 1H-pyrazol-4-yl]pyridin-2- | 7.86 (s, 1H), 7.47-7.45 (m, 2H), 7.11-7.08 (m, 2H), 6.87 (dd, 1H), 4.12 (t, 2H), | |
| | yl}propanamide | 2.37 (q, 2H), 1.90 (m, 2H), 1.11 (t, 3H), 0.93 (t, 3H) ppm | |
| 293 | N-{4-[3-(4-fluorophenyl)-1-propyl- | 1H-NMR (400 MHz, d₃-CD3CN): δ = 8.72 (s, 1H, br), 8.14 (dd, 1H), | — |
| | 1H-pyrazol-4-yl]pyridin-2-yl}-2- | 8.08 (s, 1H), 7.88 (s, 1H), 7.47-7.45 (m, 2H), 7.11-7.09 (m, 2H), 6.93 (dd, 1H), | |
| | methoxyacetamide | 4.13 (t, 2H), 3.96 (s, 3H), 3.45 (s, 3H), 0.93 (t, 3H) ppm | |
| 294 | N-{4-[3-(4-fluorophenyl)-1-propyl- | 1H-NMR (400 MHz, d₃-CD3CN): δ = 8.60 (s, 1H, br), 8.12-8.10 (m, 2H), | — |
| | 1H-pyrazol-4-yl]pyridin-2-yl}-2- | 7.86 (s, 1H), 7.47-7.45 (m, 2H), 7.11-7.08 (m, 2H), 6.86 (dd, 1H), 4.12 (t, | |
| | methylpropanamide | 2H), 2.62 (m, 1H), 1.90 (m, 2H), 1.14 (d, 6H), 0.93 (t, 3H) ppm | |
| 295 | 2-cyclopropyl-N-{4-[3-(4- | 1H-NMR (400 MHz, d₃-CD3CN): δ = 8.60 (s, 1H, br), 8.11 (m, 2H), | — |
| | fluorophenyl)-1-propyl-1H-pyrazol-4- | 7.87 (s, 1H), 7.47-7.45 (m, 2H), 7.11-7.08 (m, 2H), 6.87 (dd, 1H), 4.13 (t, 2H), | |
| | yl]pyridin-2-yl}acetamide | 2.26 (d, 2H), 1.05 (m, 1H), 0.93 (t, 3H), 0.54 (m, 2H), 0.21 (m, 2H) ppm | |
| 296 | N-{4-[3-(4-fluorophenyl)-1-methyl- | 1H-NMR (400 MHz, d₃-CD3CN): δ = 8.61 (s, 1H, br), 8.10 (m, 2H), | — |
| | 1H-pyrazol-4-yl]pyridin-2- | 7.82 (s, 1H), 7.47-7.45 (m, 2H), 7.11-7.08 (m, 2H), 6.85 (dd, 1H), 3.91 (s, 3H), | |
| | yl}propanamide | 2.37 (q, 2H), 1.11 (t, 3H) ppm | |
| 297 | N-{4-[3-(4-fluorophenyl)-1-methyl- | 1H-NMR (400 MHz, d₃-CD3CN): δ = 8.72 (s, 1H, br), 8.14 (dd, 1H), | — |
| | 1H-pyrazol-4-yl]pyridin-2-yl}-2- | 8.08 (s, 1H), 7.84 (s, 1H), 7.47-7.45 (m, 2H), 7.11-7.09 (m, 2H), 6.93 (dd, 1H), | |
| | methoxyacetamide | 3.96 (s, 2H), 3.91 (s, 3H), 3.45 (s, 3H) ppm | |
| 298 | N-{4-[3-(4-fluorophenyl)-1-methyl- | 1H-NMR (400 MHz, d₃-CD3CN): δ = 8.60 (s, 1H, br), 8.10 (m, 2H), | — |
| | 1H-pyrazol-4-yl]pyridin-2-yl}-2- | 7.82 (s, 1H), 7.47-7.45 (m, 2H), 7.11-7.09 (m, 2H), 6.95 (dd, 1H), 3.91 (s, 3H), | |
| | methylpropanamide | 2.62 (m, 1H), 1.14 (d, 6H) ppm | |
| 299 | 2-cyclopropyl-N-{4-[3-(4- | 1H-NMR (400 MHz, d₃-CD3CN): δ = 8.60 (s, 1H, br), 8.11 (m, 2H), | — |
| | fluorophenyl)-1-methyl-1H-pyrazol-4- | 7.83 (s, 1H), 7.47-7.45 (m, 2H), 7.11-7.08 (m, 2H), 6.86 (dd, 1H), 3.91 (s, 3H), | |
| | yl]pyridin-2-yl}acetamide | 2.33 (d, 2H), 1.05 (m, 1H), 0.54 (m, 2H), 0.21 (m, 2H) ppm | |
| 300 | N-(4-{3-(4-fluorophenyl)-1-[2- | 1H-NMR (400 MHz, d₆-DMSO): δ = 10.40 (s, 1H), 8.21 (s, 1H), 8.16 (dd, | — |
| | (methylsulphanyl)ethyl]-1H-pyrazol- | 1H), 8.10 (s, 1H), 7.44-7.42 (m, 2H), 7.22-7.19 (m, 2H), 6.81-6.80 (dd, 1H), | |
| | 4-yl}pyridin-2-yl)propanamide | 4.37 (t, 2H), 2.98 (t, 2H), 2.36 (q, 2H), 2.07 (s, 3H), 1.03 (t, 3H) ppm | |
| 301 | N-{4-[1-(1-cyclopropylethyl)-3-(4- | 1H-NMR (400 MHz, d₆-DMSO): δ = 10.39 (s, 1H), 8.21 (s, 1H), 8.16 (dd, | — |
| | fluorophenyl)-1H-pyrazol-4- | 1H), 8.09 (s, 1H), 7.45-7.43 (m, 2H), 7.21-7.18 (m, 2H), 6.84-6.83 (dd, 1H), | |
| | yl]pyridin-2-yl}propanamide | 3.71 (m, 1H), 2.36 (q, 2H), 1.58 (d, 3H), 1.32 (m, 1H), 1.03 (t, 3H), | |
| | | 0.63 (m, 1H), 0.50 (m, 1H), 0.42-0.38 (m, 2H) ppm | |
| 302 | N-{4-[1-(3-cyanobenzyl)-3-(4- | 1H-NMR (400 MHz, d₆-DMSO): δ = 10.40 (s, 1H), 8.35 (s, 1H), 8.17 (dd, | — |
| | fluorophenyl)-1H-pyrazol-4- | 1H), 8.10 (s, 1H), 7.88 (d, 1H), 7.80 (m, 1H), 7.70 (d, 1H), 7.62-7.58 (m, | |
| | yl]pyridin-2-yl}propanamide | 1H), 7.43-7.41 (m, 2H), 7.21-7.19 (m, 2H), 6.83-6.82 (dd, 1H), 5.48 (s, 2H), | |
| | | 2.36 (q, 2H), 1.03 (t, 3H) ppm | |
| 303 | N-{4-[1-(2-cyanoethyl)-3-(4- | 1H-NMR (400 MHz, d₆-DMSO): δ = 10.42 (s, 1H), 8.25 (s, 1H), 8.18 (dd, | — |
| | fluorophenyl)-1H-pyrazol-4- | 1H), 8.11 (s, 1H), 7.46-7.43 (m, 2H), 7.25-7.21 (m, 2H), 6.82-6.81 (dd, 1H), | |
| | yl]pyridin-2-yl}propanamide | 4.48 (t, 2H), 3.13 (t, 2H), 2.38 (q, 2H), 1.04 (t, 3H) ppm | |
| 304 | N-{4-[3-(4-fluorophenyl)-1-isopropyl- | 1H-NMR (400 MHz, d₆-DMSO): δ = 10.58 (s, 1H), 8.32 (dd, 1H), 8.03 (s, | — |
| | 5-(trifluoromethyl)-1H-pyrazol-4- | 1H), 7.33-7.30 (m, 2H), 7.18-7.15 (m, 2H), 7.00 (dd, 1H), 4.75 (m, 1H), | |
| | yl]pyridin-2-yl}propanamide | 2.38 (q, 2H), 1.56 (d, 6H), 1.02 (t, 3H) ppm | |
| 305 | 4-[1-(1-cyclopropylethyl)-3-(4- | 1H-NMR (400 MHz, d₆-DMSO): δ = 8.46 (dd, 2H), 8.31 (s, 1H), | — |
| | fluorophenyl)-1H-pyrazol-4- | 7.45-7.43 (m, 2H), 7.24-7.21 (m, 4H), 3.71 (m, 1H), 1.57 (d, 3H), 1.32 (m, 1H), | |
| | yl]pyridine | 0.63 (m, 1H), 0.50 (m, 1H), 0.42-0.38 (m, 2H) ppm | |
| 306 | N-(4-{3-(4-fluorophenyl)-1-[2- | 1H-NMR (400 MHz, d₆-DMSO): δ = 10.41 (s, 1H), 8.22 (s, 1H), 8.16 (dd, | — |
| | (methylsulphanyl)ethyl]-1H-pyrazol- | 1H), 8.13 (s, 1H), 7.44-7.42 (m, 2H), 7.22-7.19 (m, 2H), 6.78 (dd, 1H), | |
| | 4-yl}pyridin-2-yl)-2- | 4.37 (t, 2H), 2.98 (t, 2H), 2.73 (m, 1H), 2.07 (s, 3H), 1.06 (d, 6H) ppm | |
| | methylpropanamide | | |
| 307 | N-{4-[1-(1-cyclopropylethyl)-3-(4- | 1H-NMR (400 MHz, d₆-DMSO): δ = 10.40 (s, 1H), 8.22 (s, 1H), 8.16 (dd, | — |
| | fluorophenyl)-1H-pyrazol-4- | 1H), 8.12 (s, 1H), 7.45-7.42 (m, 2H), 7.21-7.18 (m, 2H), 6.81 (dd, 1H), | |
| | yl]pyridin-2-yl}-2- | 3.71 (m, 1H), 2.73 (m, 1H), 1.58 (d, 3H), 1.32 (m, 1H), 1.05 (d, 6H), 0.63 (m, | |
| | methylpropanamide | 1H), 0.50 (m, 1H), 0.42-0.38 (m, 2H) ppm | |
| 308 | N-{4-[1-(3-cyanobenzyl)-3-(4- | 1H-NMR (400 MHz, d₆-DMSO): δ = 10.41 (s, 1H), 8.36 (s, 1H), 8.17 (dd, | — |
| | fluorophenyl)-1H-pyrazol-4- | 1H), 8.13 (s, 1H), 7.88 (s, 1H), 7.82 (m, 1H), 7.69 (m, 1H), 7.62-7.58 (m, | |
| | yl]pyridin-2-yl}-2- | 1H), 7.43-7.41 (m, 2H), 7.21-7.18 (m, 2H), 6.81-6.79 (dd, 1H), 5.48 (s, 2H), | |
| | methylpropanamide | 2.73 (m, 1H), 1.06 (d, 6H) ppm | |
| 309 | N-{4-[1-(2-cyanoethyl)-3-(4- | 1H-NMR (400 MHz, d₆-DMSO): δ = 10.42 (s, 1H), 8.26 (s, 1H), 8.18 (dd, | — |
| | fluorophenyl)-1H-pyrazol-4- | 1H), 8.14 (s, 1H), 7.46-7.44 (m, 2H), 7.24-7.21 (m, 2H), 6.79 (dd, 1H), | |
| | yl]pyridin-2-yl}-2- | 4.48 (t, 2H), 3.17 (t, 2H), 2.73 (m, 1H), 1.07 (d, 6H) ppm | |
| | methylpropanamide | | |
| 310 | 4-[3-(4-fluorophenyl)-1-isopropyl-1H- | 1H-NMR (400 MHz, d₃-CD3CN): δ = 9.90 (s, 1H), 8.63 (s, 1H), 8.11 (s, | — |
| | pyrazol-4-yl]-7H-pyrrolo[2,3- | 1H), 7.55-7.53 (m, 2H), 7.28 (dd, 1H), 7.05-7.02 (m, 2H), 6.27 (dd, 1H), | |
| | d]pyrimidine | 4.62 (m, 1H), 1.57 (d, 6H) ppm | |
| 311 | 4-[3-(4-fluorophenyl)-1-isopropyl-1H- | 1H-NMR (400 MHz, d₃-CD3CN): δ = 8.15 (s, 1H), 7.69 (s, 1H), | — |
| | pyrazol-4-yl]-5,6,7,8- | 7.47-7.44 (m, 2H), 7.05-7.02 (m, 2H), 5.89 (s, 1H), 4.55 (m, 1H), 3.28 (m, 1H), | |
| | tetrahydropyrido[2,3-d]pyrimidine | 2.35 (m, 2H), 1.67 (m, 2H), 1.52 (d, 6H) ppm | |
| 312 | N-{4-[1-(1-cyanopropan-2-yl)-3-(4- | 1H-NMR (400 MHz, d₃-CD3CN): δ = 8.54 (s, 1H, br), 8.12-8.10 (m, 2H), | — |
| | fluorophenyl)-1H-pyrazol-4- | 7.50-7.47 (m, 2H), 7.13-7.10 (m, 2H), 7.13-7.7.10 (m, 1H), 6.87 (dd, 1H), | |
| | yl]pyridin-2-yl}propanamide | 4.77-4.74 (m, 1H), 3.08-2.98 (m, 2H), 2.39 (q, 2H), 1.64 (d, 3H),1.10 (t, | |
| | | 3H) ppm | |
| 313 | N-{4-[1-(1-cyanopropan-2-yl)-3-(4- | 1H-NMR (400 MHz, d₃-CD3CN): δ = 8.71 (s, 1H, br), 8.17-8.10 (m, 1H), | — |
| | fluorophenyl)-1H-pyrazol-4- | 7.99 (s, 1H), 7.50-7.48 (m, 2H), 7.13-7.7.10 (m, 1H), 6.96 (dd, 1H), | |
| | yl]pyridin-2-yl}-2-methoxyacetamide | 4.78-4.75 (m, 1H), 3.08-2.99 (m, 2H), (m, 1H), 2.27 (d, 2H), 1.64 (d, 3H), | |
| | | 1.14 (d, 2H) ppm | |
| 314 | N-{4-[1-(1-cyanopropan-2-yl)-3-(4- | 1H-NMR (400 MHz, d₃-CD3CN): δ = 8.56 (s, 1H, br), 8.15-8.11 (m, 2H), | — |
| | fluorophenyl)-1H-pyrazol-4- | 7.96 (s, 1H), 7.50-7.47 (m, 2H), 7.13-7.10 (m, 1H), 6.86 (dd, 1H), 4.76 (m, | |
| | yl]pyridin-2-yl}-2- | 2H), 3.08-2.99 (m, 2H), 2.65-2.60 (m, 1H), 2.27 (d, 2H), 1.64 (d, 3H), | |
| | methylpropanamide | 1.19 (d, 1H), 0.56 (m, 6H) ppm | |
| 315 | N-{4-[1-(1-cyanopropan-2-yl)-3-(4- | 1H-NMR (400 MHz, d₃-CD3CN): δ = 8.56 (s, 1H, br), 8.15-8.13 (m, 1H), | — |
| | fluorophenyl)-1H-pyrazol-4- | 7.98 (s, 1H), 7.51-7.48 (m, 2H), 7.14-7.11 (m, 2H), 6.90 (dd, 1H), 5.29 (m, | |
| | yl]pyridin-2-yl}-2- | 1H), 4.78-4.75 (m, 1H), 3.09-3.00 (m, 2H), 2.27 (d, 2H), 1.65 (d, 3H), | |
| | cyclopropylacetamide | 1.12-1.05 (m, 1H), 0.56 (m, 2H), 0.22 (m, 2H) ppm | |
| 316 | N-{4-[5-(difluoromethoxy)-3-(4- | 1H-NMR (400 MHz, d₃-CD3CN): δ = 8.58 (s, 1H, br), 8.09 (s, 1H), | — |
| | fluorophenyl)-1-methyl-1H-pyrazol-4- | 7.42-7.40 (m, 2H), 7.08-7.05 (m, 2H), 6.89 (dd, 1H), 3.82 (s, 3H), 2.50 (s, 1H), | |
| | yl]pyridin-2-yl}propanamide | 2.39 (q, 2H), 1.11 (t, 2H) ppm | |
| 317 | 4-[1-(cyclopropylmethyl)-3-(4- | 1H-NMR (400 MHz, d₃-CD3CN): δ = 7.92 (d, 1H), 7.86 (s, 1H), | — |
| | fluorophenyl)-1H-pyrazol-4-yl]-N- | 7.49-7.44 (m, 2H), 7.10 (m, 2H), 6.53 (dd, 1H), 6.42 (m, 1H), 5.29 (m, 1H), 4.54 (d, | |
| | (1,1,1-trifluoropropan-2-yl)pyridin-2- | 1H, br), 4.95-4.88 (m, 1H), 4.01 (d, 2H), 1.36-1.29 (m, 4H), 0.63 (m, 2H), | |
| | amine | 0.42 (m, 2H) ppm | |
| 318 | 4-[3-(4-fluorophenyl)-1-isopropyl-1H- | 1H-NMR (400 MHz, d₃-CD3CN): δ = 7.92 (d, 1H), 7.48-7.46 (m, 1H), | — |
| | pyrazol-4-yl]-N-(1,1,1- | 7.13-7.10 (m, 2H), 6.52 (dd, 2H), 6.50 (dd, 1H), 6.41 (m, 1H), 5.28 (d, 1H, | |
| | trifluoropropan-2-yl)pyridin-2-amine | br), 4.90 (m, 1H), 4.54 (m, 1H), 1.50 (d, 6H), 1.31 (d, 3H) ppm | |
| 319 | 4-[3-(4-fluorophenyl)-1-methyl-1H- | 1H-NMR (400 MHz, d₃-CD3CN): δ = 7.92 (d, 1H), 7.74 (s, 1H), | — |
| | pyrazol-4-yl]-N-(1,1,1- | 7.47-7.44 (m, 2H), 7.12-7.10 (m, 2H), 6.50 (dd, 1H), 6.40 (m, 1H), 5.30 (d, 1H, br), | |
| | trifluoropropan-2-yl)pyridin-2-amine | 4.91 (m, 1H), 3.89 (s, 3H), 1.30 (d, 2H) ppm | |
| 320 | N-{4-[1-cyclopropyl-3-(4- | 1H-NMR (400 MHz, d₃-CD3CN): δ = 8.52 (s, 1H, br), 8.11 (d, 1H), 8.08 (s, | — |
| | fluorophenyl)-1H-pyrazol-4- | 1H), 7.90 (s, 1H), 7.46-7.44 (m, 2H), 7.11-7.08 (m, 2H), 6.86 (dd, 1H), | |
| | yl]pyridin-2-yl}propanamide | 3.71 (m, 1H), 2.37 (q, 2H), 1.16-1.15 (m, 2H), 1.10 (t, 3H), 1.04-1.02 (m, 2H) | |
| | | ppm | |
| 321 | N-{4-[1-cyclopropyl-3-(4- | 1H-NMR (400 MHz, d₃-CD3CN): δ = 8.70 (s, 1H, br), 8.15 (d, 1H), 8.07 (s, | — |
| | fluorophenyl)-1H-pyrazol-4- | 1H), 7.92 (m, 2H), 7.47-7.44 (m, 2H), 7.12-7.08 (m, 2H), 6.94 (dd, 1H), | |
| | yl]pyridin-2-yl}-2-methoxyacetamide | 3.72 (m, 1H), 3.45 (s, 3H), 1.17-1.14 (m, 2H), 1.05-1.02 (m, 2H) ppm | |
| 322 | N-{4-[1-cyclopropyl-3-(4- | 1H-NMR (400 MHz, d₃-CD3CN): δ = 8.51 (s, 1H, br), 8.11 (d, 2H), 7.90 (s, | — |
| | fluorophenyl)-1H-pyrazol-4- | 1H), 7.46-7.44 (m, 2H), 7.10-7.07 (m, 2H), 6.85 (dd, 1H), 3.71 (m, 1H), | |
| | yl]pyridin-2-yl}-2- | 2.61 (m, 1H), 1.17-1.13 (m, 8H), 1.05-1.02 (m, 2H) ppm | |
| | methylpropanamide | | |
| 323 | 2-cyclopropyl-N-{4-[1-cyclopropyl-3- | 1H-NMR (400 MHz, d₃-CD3CN): δ = 8.53 (s, 1H, br), 8.12-8.10 (m, 2H), | — |
| | (4-fluorophenyl)-1H-pyrazol-4- | 7.90 (s, 1H), 7.47-7.44 (m, 2H), 7.11-7.07 (m, 2H), 6.88 (dd, 1H), 3.71 (m, | |
| | yl]pyridin-2-yl}acetamide | 1H), 2.50 (d, 2H), 1.17-1.14 (m, 2H), 1.11-1.60 (m, 3H) 0.55 (m, 2H), | |
| | | 0.22 (m, 2H) ppm | |
| 324 | 4-[3-(4-fluorophenyl)-1-isopropyl-1H- | 1H-NMR (400 MHz, d₃-CD3CN): δ = 7.90 (d, 1H), 7.82 (s, 1H), | — |
| | pyrazol-4-yl]-N-[2,2,2-trifluoro-1-(4- | 7.53-7.50 (m, 2H), 7.46-7.44 (m, 2H), 7.17-7.09 (m, 4H), 6.56-6.53 (m, 2H), | |
| | fluorophenyl)ethyl]pyridin-2-amine | 6.09-6.04 (m, 1H), 4.53 (q, 1H), 1.51 (d, 6H) ppm | |
| 325 | 4-[3-(4-fluorophenyl)-1-isopropyl-1H- | 1H-NMR (400 MHz, d₃-CD3CN): δ = 7.91 (d, 1H), 7.82 (s, 1H), | — |
| | pyrazol-4-yl]-N-(2,2,2-trifluoro-1- | 7.50-7.38 (m, 7H), 7.12-7.09 (m, 2H), 6.54 (d, 2H), 6.09-6.01 (m, 2H), 4.53 (m, 1H), | |
| | phenylethyl)pyridin-2-amine | 1.51 (d, 6H), ppm | |
| 326 | N-{4-[1-(2-ethoxyethyl)-3-(4- | 1H-NMR (400 MHz, d₃-CD3CN): δ = 8.52 (s, 1H, br), 8.11 (m, 2H), | — |
| | fluorophenyl)-1H-pyrazol-4- | 7.87 (m, 1H), 7.90 (s, 1H), 7.48-7.43 (m, 2H), 7.12-7.08 (m, 2H), 6.74 (dd, 1H), | |
| | yl]pyridin-2-yl}propanamide | 4.30 (t, 2H), 3.83 (t, 2H), 3.83 (t, 2H), 3.47 (q, 2H), 2.37 (q, 2H), | |
| | | 1.14-1.04 (t, 6H) ppm | |
| 327 | N-{4-[1-(2-ethoxyethyl)-3-(4- | 1H-NMR (400 MHz, d₃-CD3CN): δ = 8.70 (s, 1H, br), 8.15 (d, 1H), 8.09 (s, | — |
| | fluorophenyl)-1H-pyrazol-4- | 1H), 7.90 (s, 1H), 7.48-7.46 (m, 2H), 7.12-7.09 (m, 2H), 6.94 (dd, 1H), | |
| | yl]pyridin-2-yl}-2-methoxyacetamide | 4.31 (t, 2H), 3.96 (s, 2H), 3.83 (t, 2H), 3.49 (q, 2H), 3.45 (s, 3H), 1.12 (t, 3H) | |
| | | ppm | |
| 328 | N-{4-[1-(2-ethoxyethyl)-3-(4- | 1H-NMR (400 MHz, d₃-CD3CN): δ = 8.51 (s, 1H, br), 8.11 (m, 2H), | — |
| | fluorophenyl)-1H-pyrazol-4- | 7.88 (s, 1H), 7.48-7.45 (m, 2H), 7.11-7.09 (m, 2H), 6.86 (dd, 1H), 4.30 (t, 2H), | |
| | yl]pyridin-2-yl}-2- | 3.83 (t, 2H), 3.49 (q, 2H), 1.14-1.10 (m, 10H) ppm | |
| | methylpropanamide | | |
| 329 | 2-cyclopropyl-N-{4-[1-(2- | 1H-NMR (400 MHz, d₃-CD3CN): δ = 8.54 (s, 1H, br), 8.11 (m, 2H), | — |
| | ethoxyethyl)-3-(4-fluorophenyl)-1H- | 7.88 (s, 1H), 7.48-7.41 (m, 2H), 7.12-7.09 (m, 2H), 6.77 (dd, 1H), 4.30 (t, 2H), | |
| | pyrazol-4-yl]pyridin-2-yl}acetamide | 4.07 (2H, isomer), 3.83 (t, 2H), 3.73 (2H, isomer), 3.49 (q, 2H), 3.32 (2H, | |
| | | isomer), 2.41 (d, 2H), 2.22 (2H, isomer), 1.13 (t, 3H), 1.07 (m, 1H), | |
| | | 0.54 (m, 2H), 0.21 (m, 2H) ppm | |
| 330 | methyl 6-({4-[1-ethyl-3-(4- | 1H-NMR (400 MHz, d₃-CD3CN): δ = 8.18-8.16 (m, 2H), 8.08 (s, 1H), | — |
| | fluorophenyl)-1H-pyrazol-4- | 7.44-7.42 (m, 2H), 7.21-7.18 (m, 2H), 6.77 (dd, 1H), 4.21 (q, 2H) 3.58 (s, | |
| | yl]pyridin-2-yl}amino)-6- | 1H), 2.39-2.31 (m, 4H), 1.56-1.52 (m, 4H), 1.45 (t, 3H) ppm | |
| | oxohexanoate | | |
| 331 | methyl 5-({4-[1-ethyl-3-(4- | 1H-NMR (400 MHz, d₃-CD3CN): δ = 8.18-8.16 (m, 2H), 8.07 (s, 1H), | — |
| | fluorophenyl)-1H-pyrazol-4- | 7.44-7.42 (m, 2H), 7.21-7.18 (m, 2H), 6.83 (dd, 1H), 4.21 (q, 2H), 3.59 (s, | |
| | yl]pyridin-2-yl}amino)-5- | 1H), 2.41-2.39 (m, 2H), 2.33 (t, 2H), 1.82-1.77 (t, 3H) ppm | |
| | oxopentanoate | | |
| 332 | ethyl 3-ethyl-5-({4-[1-ethyl-3-(4- | 1H-NMR (400 MHz, d₃-CD3CN): δ = 8.18-8.16 (m, 2H), 8.07 (s, 1H), | — |
| | fluorophenyl)-1H-pyrazol-4- | 7.44-7.41 (m, 2H), 7.20-7.17 (m, 2H), 6.84 (dd, 1H), 4.21 (q, 2H), 4.03 (q, | |
| | yl]pyridin-2-yl}amino)-5- | 2H), 2.33-2.19 (m, 4H), 1.45 (t, 3H), 1.34-1.29 (m, 2H), 1.16 (t, 3H), 0.84 (t, | |
| | oxopentanoate | 3H) ppm | |
| 333 | 3-(difluoromethyl)-N-{4-[1-ethyl-3- | 1H-NMR (400 MHz, d₃-CD3CN): δ = 8.67 (s, 1H, br), 8.23 (m, 2H), | — |
| | (4-fluorophenyl)-1H-pyrazol-4- | 8.14 (m, 1H), 7.46-7.43 (m, 2H), 7.23-7.20 (m, 2H), 6.89-6.88 (dd, 1H), 4.22 (q, | |
| | yl]pyridin-2-yl}-1-methyl-1H- | 2H), 3.64 (s, 3H), 1.46 (t, 3H) ppm | |
| | pyrazol-4-carboxamide | | |
| 334 | 2-(difluoromethyl)-N-{4-[1-ethyl-3- | 1H-NMR (400 MHz, d₃-CD3CN): δ = 8.28 (d, 1H), 8.24 (s, 1H), 8.22 (m, | — |
| | (4-fluorophenyl)-1H-pyrazol-4- | 1H), 8.13 (m, 1H), 7.55-7.54 (m, 1H), 7.47-7.44 (m, 2H), 7.24-7.21 (m, | |
| | yl]pyridin-2-yl}thiophen-3- | 2H), 6.94 (dd, 1H), 4.24 (q, 2H), 1.46 (t, 3H) ppm | |
| | carboxamide | | |
| 335 | N-{4-[1-ethyl-3-(4-fluorophenyl)-1H- | 1H-NMR (400 MHz, d₃-CD3CN): δ = 8.27 (m, 2H), 8.08 (s, 1H), | — |
| | pyrazol-4-yl]pyridin-2-yl}-4-methyl- | 7.47-7.44 (m, 2H), 7.24-7.21 (m, 2H), 7.00 (dd, 1H), 4.22 (q, 2H), 2.78 (s, 3H), | |
| | 1,2,3-thiadiazol-5-carboxamide | 1.46 (t, 3H) ppm | |
| 336 | 3-(4-chlorophenyl)-N-{4-[1-ethyl-3- | 1H-NMR (400 MHz, d₃-CD3CN): δ = 8.17 (m, 2H), 8.06 (s, 1H, br), | — |
| | (4-fluorophenyl)-1H-pyrazol-4- | 7.44-7.41 (m, 2H), 7.43-7.32 (m, 2H), 7.26 (m, 2H), 7.22-7.19 (m, 2H), 6.85 (dd, | |
| | yl]pyridin-2-yl}propanamide | 1H), 4.21 (q, 2H), 2.87 (t, 2H), 2.64 (t, 2H), 1.45 (t, 3H) ppm | |
| 337 | (1R,2R)—N-{4-[1-ethyl-3-(4- | 1H-NMR (400 MHz, d₆-DMSO): δ = 8.18-8.15 (m, 2H), 8.10 (s, 1H), | — |
| | fluorophenyl)-1H-pyrazol-4- | 7.44-7.41 (m, 2H), 7.32-7.28 (m, 2H), 7.23-7.15 (m, 5H), 6.82-6.81 (dd, 1H), | |
| | yl]pyridin-2-yl}-2- | 4.23-4.19 (q, 2H), 2.35 (m, 1H), 1.43 (t, 3H), 1.43 (m, 1H), 1.35 (m, 1H) | |
| | phenylcyclopropanecarboxamide | ppm | |
| 338 | 2-cyclopentyl-N-{4-[1-ethyl-3-(4- | 1H-NMR (400 MHz, d₆-DMSO): δ = 8.18 (s, 1H), 8.16-8.15 (m, 1H), | — |
| | fluorophenyl)-1H-pyrazol-4- | 8.07 (s, 1H), 7.43-7.41 (m, 2H), 7.21-7.18 (m, 2H), 6.83-6.82 (dd, 1H), | |
| | yl]pyridin-2-yl}acetamide | 4.22-4.18 (m, 2H), 2.32 (d, 2H), 2.18 (m, 1H), 1.70 (m, 1H), 1.60 (m, 1H), | |
| | | 1.50 (m, 1H), 1.43 (t, 3H), 1.15 (m, 1H) ppm | |
| 339 | N-{4-[1-ethyl-3-(4-fluorophenyl)-1H- | 1H-NMR (400 MHz, d₃-CD3CN): δ = 8.18-8.16 (m, 2H), 8.08 (s, 1H, br), | — |
| | pyrazol-4-yl]pyridin-2-yl}-3- | 7.44-7.41 (m, 2H), 7.21-7.18 (m, 2H), 6.84 (dd, 1H), 4.21 (m, 2H), 2.23 (d, | |
| | methylbutanamide | 2H), 2.05-2.00 (m, 1H), 1.45 (t, 3H), 0.90 (d, 6H) ppm | |
| 340 | N-{4-[1-ethyl-3-(4-fluorophenyl)-1H- | 1H-NMR (400 MHz, d₃-CD3CN): δ = 8.19-8.16 (m, 2H), 8.12 (s, 1H, br), | — |
| | pyrazol-4-yl]pyridin-2-yl}-2- | 7.44-7.41 (m, 2H), 7.22-7.18 (m, 2H), 6.82 (dd, 1H), 4.21 (m, 1H), 1.45 (t, | |
| | methylbutanamide | 3H), 1.38-1.34 (m, 1H), 1.21 (m, 1H), 1.04 (d, 3H), 0.83 (t, 3H) ppm | |
| 341 | N-{4-[1-ethyl-3-(4-fluorophenyl)-1H- | 1H-NMR (400 MHz, d₆-DMSO): δ = 10.67 (m, 1H), 8.17-8.14 (m, 2H), | — |
| | pyrazol-4-yl]pyridin-2-yl}-2- | 8.05 (s, 1H), 7.43-7.40 (m, 2H), 7.21-7.17 (m, 2H), 6.82-6.79 (dd, 1H), | |
| | methylcyclopropanecarboxamide | 4.22-4.18 (m, 2H), 1.72 (m, 1H), 1.45 (m, 2H), 1.20 (m, 1H), 1.07 (d, 3H), | |
| | | 0.90 (m, 1H), 0.60 (m, 1H) ppm | |
| 342 | N-{4-[1-ethyl-3-(4-fluorophenyl)-1H- | 1H-NMR (400 MHz, d₃-CD3CN): δ = 8.17 (m, 2H), 8.03 (s, 1H, br), | — |
| | pyrazol-4-yl]pyridin-2-yl}acetamide | 7.44-7.41 (m, 2H), 7.22-7.18 (m, 2H), 6.86 (dd, 1H), 4.21 (m, 1H), 4.21 (q, 2H), | |
| | | 2.05 (s, 3H), 1.45 (t, 3H) ppm | |
| 343 | N-{4-[3-(4-fluorophenyl)-1-methyl- | 1H-NMR (400 MHz, d₃-CD3CN): δ = 8.16 (d, 1H), 8.13 (s, 1H), 8.07 (s, | — |
| | 1H-pyrazol-4-yl]pyridin-2-yl}-3- | 1H, br), 7.44-7.41 (m, 2H), 7.29-7.26 (m, 2H), 7.23-7.17 (m, 5H), 6.82 (dd, | |
| | phenylpropanamide | 1H), 3.92 (s, 3H), 2.86 (t, 2H), 2.67 (t, 2H) ppm | |
| 344 | 4-fluoro-N-{4-[3-(4-fluorophenyl)-1- | 1H-NMR (400 MHz, d₃-CD3CN): δ = 8.27 (d, 1H), 8.19 (s, 1H), 8.16 (s, | — |
| | methyl-1H-pyrazol-4-yl]pyridin-2- | 1H), 8.09-8.06 (m, 2H), 7.47-7.45 (m, 2H), 7.35-7.32 (m, 2H), | |
| | yl}benzamide | 7.24-7.21 (m, 2H), 6.92 (dd, 1H), 3.94 (s, 3H) ppm | |
| 345 | N-{4-[3-(4-fluorophenyl)-1-methyl- | 1H-NMR (400 MHz, d₃-CD3CN): δ = 8.20 (d, 1H), 8.19 (s, 1H), 8.15 (s, | — |
| | 1H-pyrazol-4-yl]pyridin-2-yl}-2-(2- | 1H), 8.05 (s, 1H, br), 7.44-7.41 (m, 2H), 7.22-7.20 (m, 2H), 6.90 (dd, 1H), | |
| | methoxyethoxy)acetamide | 4.10 (s, 2H), 3.91 (s, 3H), 3.66-3.64 (m, 2H), 3.51-3.50 (m, 2H), 3.29 (s, | |
| | | 3H) ppm | |
| 346 | N-{4-[3-(4-fluorophenyl)-1-methyl- | 1H-NMR (400 MHz, d₃-CD3CN): δ = 8.24 (m, 1H), 8.18 (s, 2H), 8.00 (m, | — |
| | 1H-pyrazol-4-yl]pyridin-2- | 2H), 7.60-7.58 (m, 1H), 7.52-7.45 (m, 4H), 7.23-7.21 (m, 2H), 6.92 (dd, | |
| | yl}benzamide | 1H), 3.94 (s, 3H) ppm | |
| 347 | N-{4-[3-(4-fluorophenyl)-1-methyl- | 1H-NMR (400 MHz, d₃-CD3CN): δ = 8.17 (m, 1H), 8.14 (s, 1H), 8.07 (s, | — |
| | 1H-pyrazol-4-yl]pyridin-2-yl}-3- | 1H, br), 7.43-7.41 (m, 2H), 7.21-7.18 (m, 2H), 6.83 (dd, 1H), 3.92 (s, 3H), | |
| | methylbutanamide | 2.23 (d, 2H), 2.05-2.00 (m, 1H) ppm | |
| 348 | N-{4-[3-(4-fluorophenyl)-1-methyl- | 1H-NMR (400 MHz, d₃-CD3CN): δ = 8.16 (m, 1H), 8.14 (s, 1H), 8.11 (s, | — |
| | 1H-pyrazol-4-yl]pyridin-2-yl}-2- | 1H), 7.44-7.41 (m, 2H), 7.21-7.18 (m, 2H), 6.80 (dd, 1H), 3.92 (s, 3H), | |
| | methylbutanamide | 1.59-1.55 (m, 1H), 1.38-1.35 (m, 1H), 1.23 (s, 1H, br), 1.04 (d, 3H), 0.84 (t, | |
| | | 3H) ppm | |
| 349 | N-{4-[3-(4-fluorophenyl)-1-methyl- | 1H-NMR (400 MHz, d₆-DMSO): δ = 10.27 (s, 1H), 8.15-8.11 (m, 3H), | — |
| | 1H-pyrazol-4-yl]pyridin-2- | 7.43-7.41 (m, 2H), 7.22-7.19 (m, 2H), 6.80-6.78 (dd, 1H), 3.92 (s, 3H), | |
| | yl}cyclobutancarboxamide | 2.15 (m, 2H), 2.05 (m, 2H), 1.90 (m, 1H), 1.23 (m, 2H) ppm | |
| 350 | N-{4-[3-(4-fluorophenyl)-1-methyl- | 1H-NMR (400 MHz, d₃-CD3CN): δ = 8.12 (d, 1H), 8.12 (s, 1H), 8.03 (s, | — |
| | 1H-pyrazol-4-yl]pyridin-2- | 1H, br), 7.43-7.41 (m, 2H), 7.21-7.18 (m, 2H), 6.84 (dd, 1H), 3.92 (s, 3H), | |
| | yl}acetamide | 2.05 (s, 3H) ppm | |
| 351 | N-{4-[1-ethyl-3-(4-fluorophenyl)-1H- | 1H-NMR (400 MHz, d₃-CD3CN): δ = 8.42 (s, 1H, br), 8.28 (d, 1H), 8.24 (s, | — |
| | pyrazol-4-yl]pyridin-2- | 1H), 8.12 (s, 1H, br), 7.99 (s, 1H, br), 7.47-7.43 (m, 2H), 7.23-7.20 (m, 2H), | |
| | yl}ethandiamide | 7.05-7.04 (m, 1H), 4.22 (q, 2H), 1.46 (t, 3H) ppm | |
| 352 | (2S)—N-{4-[1-ethyl-3-(4- | 1H-NMR (400 MHz, d₃-CD3CN): δ = 9.56 (s, 1H), 8.21 (s, 1H), 8.19 (d, | — |
| | fluorophenyl)-1H-pyrazol-4- | 1H), 8.07 (s, 1H, br), 7.45-7.42 (m, 2H), 7.22-7.19 (m, 2H), 6.90 (dd, 1H), | |
| | yl]pyridin-2-yl}-2- | 5.90 (d, 1H), 4.23-4.16 (m, 3H), 1.45 (t, 3H), 1.28 (d, 3H) ppm | |
| | hydroxypropanamide | | |
| 353 | (2S)—N-{4-[3-(4-fluorophenyl)-1- | 1H-NMR (400 MHz, d₃-CD3CN): δ = 9.56 (s, 1H), 8.20 (d, 1H), 8.16 (s, | — |
| | methyl-1H-pyrazol-4-yl]pyridin-2- | 1H), 8.07 (s, 1H, br), 7.44-7.41 (m, 2H), 7.22-7.19 (m, 2H), 6.90 (dd, 1H), | |
| | yl}-2-hydroxypropanamide | 4.18 (m, 1H), 3.92 (s, 1H), 1.45 (t, 3H), 1.28 (d, 3H) ppm | |
| 354 | N-{4-[3-(4-fluorophenyl)-1-(propan- | 1H-NMR (400 MHz, d₆-DMSO): δ = 10.15 (bs, 1H), 8.49 (d, 1H), 8.44 (s, | — |
| | 2-yl)-1H-pyrazol-4-yl]pyrimidin-2- | 1H), 7.60 (m, 2H), 7.24 (t, 1H), 6.99 (d, 1H), 4.61 (m, 1H), 4.07 (s, 2H), | |
| | yl}-2-methoxyacetamide | 3.33 (s, 3H), 1.51 (d, 6H) ppm | |
| 355 | N-{4-[3-(4-fluorophenyl)-1-(propan- | 1H-NMR (400 MHz, d₆-DMSO): δ = 10.33 (bs, 1H), 8.47 (d, 1H), 8.37 (s, | — |
| | 2-yl)-1H-pyrazol-4-yl]pyrimidin-2- | 1H), 7.66 (m, 2H), 7.23 (t, 1H), 6.92 (d, 1H), 4.62 (m, 1H), 2.84 (m, 1H), | |
| | yl}-2-methylpropanamide | 1.50 (d, 6H), 1.05 (d, 6H) ppm | |
| 356 | N-{4-[3-(4-fluorophenyl)-1-(propan- | 1H-NMR (400 MHz, d₆-DMSO): δ = 10.63 (s, 1H), 8.47 (d, 1H), 8.36 (s, | — |
| | 2-yl)-1H-pyrazol-4-yl]pyrimidin-2- | 1H), 7.66 (m, 2H), 7.22 (t, 2H), 6.94 (d, 1H), 4.61 (m, 1H), 2.16 (m, 1H), | |
| | yl}cyclopropanecarboxamide | 1.5 (d, 6H), 0.8 (m, 2H), 0.74 (m, 2H) ppm | |
| 357 | N-{4-[3-(4-fluorophenyl)-1-(2- | 1H-NMR (400 MHz, d₃-CD3CN): δ = 8.25 (d, 2H), 7.96 (s, 1H), 7.43 (m, | — |
| | methylpropyl)-1H-pyrazol-4- | 2H), 7.05 (t, 2H), 6.69 (d, 1H), 3.92 (d, 2H), 2.39 (s, 3H), 2.24 (m, 1H), | |
| | yl]pyrimidin-2-yl}acetamide | 0.92 (d, 6H), ppm | |
| 358 | 4-[3-(4-fluorophenyl)-1-(2- | 1H-NMR (400 MHz, d₆-DMSO): δ = 8.26 (bs, 1H), 8.11 (d, 1H), 7.56 (m, | 76/23 |
| | methoxyethyl)-1H-pyrazol-4-yl]-N- | 2H), 7.22 (t, 2H), 6.78 (d, 1H), 6.41 (bs, 1H), 4.34 (t, 2H), 4.01 (t, 2H, | |
| | (propan-2-yl)pyrimidin-2-amine | isomer), 3.76 (t, 2H), 3.65 (t, 2H, isomer), 3.28 (s, 3H), 3.12 (s, 3H, | |
| | | isomer), 1.06 (bs, 6H), 0.96 (bs, 6H, isomer) ppm | |
| 359 | 4-[1-(2-chloroethyl)-3-(4- | 1H-NMR (400 MHz, d₆-DMSO): δ = 8.36 (bs, 1H), 8.12 (d, 1H), 7.56 (m, | 70/30 |
| | fluorophenyl)-1H-pyrazol-4-yl]-N- | 2H), 7.23 (t, 2H), 6.80 (d, 1H), 6.40 (bs, 1H), 4.54 (t, 2H), 4.20 (t, 2H, | |
| | (propan-2-yl)pyrimidin-2-amine | isomer), 4.08 (t, 2H), 3.97 (t, 2H, isomer), 1.06 (bs, 6H), 0.95 (bs, 6H, | |
| | | isomer) ppm | |
| 360 | 4-[1-(butan-2-yl)-3-(4-fluorophenyl)- | 1H-NMR (400 MHz, CDCl₃): δ = 7.99 (d, 1H), 7.89 (s, 1H), 7.46 (m, 2H), | — |
| | 1H-pyrazol-4-yl]-N-(propan-2- | 7.01 (t, 2H), 6.26 (d, 1H), 4.87 (bd, 1H), 4.19 (m, 1H), 4.03 (m, 1H), | |
| | yl)pyrimidin-2-amine | 1.79 (m, 2H), 1.49 (d, 3H), 1.15 (d, 6H), 0.83 (t, 3H) ppm | |
| 361 | 4-[3-(4-fluorophenyl)-1-methyl-1H- | 1H-NMR (400 MHz, d₆-DMSO): δ = 8.25 (bs, 1H), 7.55 (m, 2H), 7.21 (t, | — |
| | pyrazol-4-yl]-N-(propan-2- | 2H), 6.76 (d, 1H), 6.38 (bs, 1H), 3.23 (s, 3H), 1.06 (bs, 6H) ppm | |
| | yl)pyrimidin-2-amine | | |
| 362 | 3-{3-(4-fluorophenyl)-4-[2-(propan-2- | 1H-NMR (400 MHz, d₆-DMSO): δ = 8.38 (bs, 1H), 8.13 (d, 1H), 7.56 (m, | — |
| | ylamino)pyrimidin-4-yl]-1H-pyrazol- | 2H), 7.24 (t, 2H), 6.82 (d, 1H), 6.39 (bs, 1H), 4.48 (t, 2H), 3.75 (bs, 1H), | |
| | 1-yl}propanonitrile | 3.16 (t, 2H), 1.07 (bs, 6H) ppm | |
| 363 | 4-[3-(4-fluorophenyl)-1-(prop-2-yn-1- | 1H-NMR (400 MHz, d₆-DMSO): δ = 8.34 (bs, 1H), 8.12 (d, 1H), 7.55 (m, | — |
| | yl)-1H-pyrazol-4-yl]-N-(propan-2- | 2H), 7.23 (t, 2H), 6.83 (d, 1H), 6.38 (bs, 1H), 5.14 (s, 2H), 3.59 (m, 1H), | |
| | yl)pyrimidin-2-amine | 1.07 (bs, 6H) ppm | |
| 364 | 4-[1-(2,2-difluoroethyl)-3-(4- | 1H-NMR (400 MHz, d₆-DMSO): δ = 8.34 (bs, 1H), 8.12 (d, 1H), 7.55 (m, | — |
| | fluorophenyl)-1H-pyrazol-4-yl]-N- | 2H), 7.24 (t, 2H), 6.84 (d, 1H), 6.46 (tt, 1H), 6.40 (bs, 1H), 4.73 (td, 2H), | |
| | (propan-2-yl)pyrimidin-2-amine | 3.85 (bs, 1H), 1.07 (bs, 6H) ppm | |
| 365 | N-{4-[3-(4-fluorophenyl)-1-(2- | 1H-NMR (400 MHz, d₆-DMSO): δ = 10.30 (s, 1H), 8.45 (d, 1H), 8.34 (s, | — |
| | methylpropyl)-1H-pyrazol-4- | 1H), 7.62 (m, 2H), 7.24 (t, 2H), 6.91 (d, 1H), 4.03 (d, 2H), 2.41 (q, 2H), | |
| | yl]pyrimidin-2-yl}propanamide | 2.28 (m, 1H), 0.99 (t, 3H), 0.91 (d, 6H) ppm | |
| 366 | 4-[3-(4-fluorophenyl)-1-(propan-2-yl)- | 1H-NMR (400 MHz, d₆-DMSO): δ = 8.23 (s, 1H), 8.08 (d, 1H), 7.55 (m, | — |
| | 1H-pyrazol-4-yl]pyrimidin-2-amine | 2H), 7.23 (t, 2H), 6.47 (s, 1H) 6.76 (d, 1H), 6.35 (d, 1H), 4.57 (m, 1H), | |
| | | 1.48 (d, 6H) ppm | |
| 367 | 4-[1-(2,2-difluoroethyl)-3-(4- | 1H-NMR (400 MHz, d₆-DMSO): δ = 8.55 (d, 2H), 8.44 (d, 2H, isomer), | 66/23 |
| | fluorophenyl)-5-methyl-1H-pyrazol-4- | 7.35 (m, 4H), 7.17 (m, 2H), 7.07 (dd, 2H, isomer), 6.47 (tt, 1H), 4.72 (td, | |
| | yl]pyridine | 2H), 4.38 (td, 2H, isomer), 2.31 (s, 3H) ppm | |
| 368 | 4-[3-(4-fluorophenyl)-5-methyl-1-(2- | 1H-NMR (400 MHz, d₆-DMSO): δ = 8.53 (d, 2H), 8.41 (d, 2H, isomer), | 64/28 |
| | methylpropyl)-1H-pyrazol-4- | 7.33 (m, 4H), 7.15 (m, 4H), 7.03 (d, 2H, isomer), 3.96 (d, 2H), 3.74 (d, 2H, | |
| | yl]pyridine | isomer), 2.28 (m, 4H), 0.94 (d, 6H), 0.72 (d, 6H, isomer) ppm | |
| 369 | N-benzyl-4-[3-(4-fluorophenyl)-1- | 1H-NMR (400 MHz, d₆-DMSO): δ = 8.47 (bs, 1H), 8.18 (bs, 1H), 7.57 (m, | — |
| | (propan-2-yl)-1H-pyrazol-4- | H), 7.22 (m, 8H), 6.72 (bs, 1H), 4.58 (m, 1H), 4.32 (bs, 2H), 1.47 (d, 6H) | |
| | yl]pyrimidin-2-amine | ppm | |
| 370 | 4-[1-ethyl-3-(4-fluorophenyl)-1H- | 1H-NMR (400 MHz, d₆-DMSO): δ = 8.29 (bs, 1H), 8.10 (d, 1H), 7.56 (m, | — |
| | pyrazol-4-yl]-N-(propan-2- | 2H), 7.21 (t, 2H), 6.76 (d, 1H), 6.41 (bs, 1H), 4.21 (q, 2H), 3.90 (q, 2H, | |
| | yl)pyrimidin-2-amine | isomer), 3.68 (bs, 1H), 1.44 (t, 3H), 1.24 (t, 3H, isomer), 1.06 (bs, 6H), | |
| | | 0.95 (bs, 6H, isomer) ppm | |
| 371 | 4-[3-(4-fluorophenyl)-1-(propan-2-yl)- | 1H-NMR (400 MHz, d₃-CD3CN): δ = 8.06 (d, 1H), 7.97 (s, 1H), 7.54 (m, | — |
| | 1H-pyrazol-4-yl]-N-(propan-2- | 2H), 7.08 (t, 2H), 6.33 (d, 1H), 4.95 (d, 1H), 4.55 (m, 1H), 4.11 (m, 1H), | |
| | yl)pyrimidin-2-amine | 1.58 (d, 6H), 1.22 (d, 6H), ppm | |
| 372 | N-(1-cyclopropylethyl)-4-[3-(4- | 1H-NMR (400 MHz, d₃-CD3CN): δ = 8.08 (d, 1H), 7.95 (s, 1H), 7.54 (m, | — |
| | fluorophenyl)-1-(2-methylpropyl)-1H- | 2H), 7.11 (t, 2H), 6.36 (d, 1H), 5.25 (bs, 1H), 3.98 (d, 2H), 3.45 (m, 1H), | |
| | pyrazol-4-yl]pyrimidin-2-amine | 2.33 (m, 1H), 127 (d, 3H), 1.02 (d, 6H), 0.92 (m, 1H), 0.5 (m, 2H), 0.37 (m, | |
| | | 1H), 0.26 (m, 1H) ppm | |

¹The isomer ratio is based on a mixture of various pyrazole isomers obtained in some cases (the main isomer is the 1-substituted 3-aryl-1H-pyrazole, the minor isomer is the corresponding 1-substituted 5-aryl-1H-pyrazole). If no isomer ratio was given, only the main isomer is present.

TABLE 3
Mass spectroscopic/logP data for the compounds produced of the type [I-a] and [I-b]

| No. | Name | LogP_{A}¹ | [M + H]_{A}⁺² | LogP_{B}³ | [M + H]_{B}⁺² | Isomer ratio⁴ | Method⁵ |
|---|---|---|---|---|---|---|---|
| 1 | 4-[3-(4-fluorophenyl)-1-(2-methylpropyl)-1H- | 1.63 | 296 | — | — | — | C |
| | pyrazol-4-yl]pyridine | | | | | | |
| 2 | 4-[5-(2,6-difluorophenyl)-1-(2-methylpropyl)- | 1.86 | 314 | — | — | — | C |
| | 1H-pyrazol-4-yl]pyridine | | | | | | |
| 3 | 4-[1-(butan-2-yl)-3-(2,6-difluorophenyl)-1H- | 1.82 | 314 | — | — | — | C |
| | pyrazol-4-yl]pyridine | | | | | | |
| 5 | 4-[3-(3-chlorophenyl)-1-(2-methylpropyl)- | 3.27 | 313 | — | — | — | C |
| | 1H-pyrazol-4-yl]pyrimidine | | | | | | |
| 6 | 4-[5-(2,6-difluorophenyl)-1-(2,2- | 1.79 | 328 | — | — | — | C |
| | dimethylpropyl)-1H-pyrazol-4-yl]pyridine | | | | | | |
| 7 | 4-[3-(2,6-difluorophenyl)-1-(3-methylbut-2- | 1.69 | 326 | — | — | — | C |
| | en-1-yl)-1H-pyrazol-4-yl]pyridine | | | | | | |
| 8 | 4-[1-cyclobutyl-3-(2,6-difluorophenyl)-1H- | 1.56 | 312 | — | — | — | C |
| | pyrazol-4-yl]pyridine | | | | | | |
| 9 | 4-[3-(4-fluorophenyl)-1-(tetrahydro-2H- | 1.29 | 324 | — | — | — | B |
| | pyran-2-yl)-1H-pyrazol-4-yl]pyridine | | | | | | |
| 10 | 4-[5-(2,6-difluorophenyl)-1-(propan-2-yl)-1H- | 1.54 | 300 | — | — | — | C |
| | pyrazol-4-yl]pyridine | | | | | | |
| 11 | 4-[3-(2,6-difluorophenyl)-1-(2,2- | 1.84 | 328 | — | — | — | C |
| | dimethylpropyl)-1H-pyrazol-4-yl]pyridine | | | | | | |
| 12 | 4-[3-(2,6-difluorophenyl)-1-(2,2,2- | 1.39 | 340 | — | — | — | C |
| | trifluoroethyl)-1H-pyrazol-4-yl]pyridine | | | | | | |
| 13 | 4-[3-(2,6-difluorophenyl)-1-(2-methylpropyl)- | 1.62 | 314 | — | — | — | C |
| | 1H-pyrazol-4-yl]pyridine | | | | | | |
| 14 | 4-[3-(2,6-difluorophenyl)-1-(propan-2-yl)-1H- | 1.4 | 300 | — | — | — | C |
| | pyrazol-4-yl]pyridine | | | | | | |
| 15 | 4-[1-cyclobutyl-5-(2,6-difluorophenyl)-1H- | 1.49 | 312 | — | — | — | C |
| | pyrazol-4-yl]pyridine | | | | | | |
| 16 | 4-[1-cyclopentyl-3-(2,6-difluorophenyl)-1H- | 1.79 | 326 | — | — | — | C |
| | pyrazol-4-yl]pyridine | | | | | | |
| 17 | 4-[1-cyclopentyl-5-(2,6-difluorophenyl)-1H- | 1.74 | 326 | — | — | — | C |
| | pyrazol-4-yl]pyridine | | | | | | |
| 19 | N-{4-[3-(4-fluorophenyl)-1-(tetrahydro-2H- | 2.32 | 395 | — | — | — | A |
| | pyran-2-yl)-1H-pyrazol-4-yl]pyridin-2- | | | | | | |
| | yl}propanamide | | | | | | |
| 20 | 4-[3-(4-fluorophenyl)-1-(tetrahydro-2H- | 2.08 | 363 | — | — | — | B |
| | pyran-2-yl)-1H-pyrazol-4-yl]-1H-pyrrolo[2,3- | | | | | | |
| | b]pyridine | | | | | | |
| 21 | N-{4-[3-(4-fluorophenyl)-1-(tetrahydro-2H- | 2.76 | 409 | — | — | — | B |
| | pyran-2-yl)-1H-pyrazol-4-yl[pyridin-2-yl}-2- | | | | | | |
| | methylpropanamide | | | | | | |
| 22 | 4-[5-(2,6-difluorophenyl)-1-(3-methylbut-2- | 1.72 | 326 | — | — | — | C |
| | en-1-yl)-1H-pyrazol-4-yl]pyridine | | | | | | |
| 23 | N-{4-[3-(4-fluorophenyl)-1-(tetrahydro-2H- | 2.64 | 411 | — | — | — | B |
| | pyran-2-yl)-1H-pyrazol-4-yl]pyridin-2-yl}-2- | | | | | | |
| | methoxyacetamide | | | | | | |
| 24 | 4-[5-(4-fluorophenyl)-1-(2-methylpropyl)-1H- | 1.59 | 296 | — | — | — | C |
| | pyrazol-4-yl]pyridine | | | | | | |
| 25 | 4-[5-(4-fluorophenyl)-1-(3-methylbut-2-en-1- | 2.68 | 308 | — | — | — | C |
| | yl)-1H-pyrazol-4-yl]pyridine | | | | | | |
| 26 | 4-[3-(2,6-difluorophenyl)-1-(tetrahydrofuran- | 1.16 | 342 | — | — | — | C |
| | 2-ylmethyl)-1H-pyrazol-4-yl]pyridine | | | | | | |
| 27 | 4-[5-(2,6-difluorophenyl)-1-(tetrahydrofuran- | 1.21 | 342 | — | — | — | C |
| | 2-ylmethyl)-1H-pyrazol-4-yl]pyridine | | | | | | |
| 28 | 4-[3-(3-chlorophenyl)-1-(tetrahydro-2H- | 1.55 | 340 | — | — | — | B |
| | pyran-2-yl)-1H-pyrazol-4-yl]pyridine | | | | | | |
| 29 | 4-[3-(4-fluorophenyl)-1-(tetrahydro-2H- | 2.53 | 339 | — | — | — | B |
| | pyran-2-yl)-1H-pyrazol-4-yl]-2- | | | | | | |
| | methylpyrimidine | | | | | | |
| 30 | 4-[3-(4-fluorophenyl)-1-(3-methylbut-2-en-1- | 2.11 | 308 | — | — | — | C |
| | yl)-1H-pyrazol-4-yl]pyridine | | | | | | |
| 31 | 4-[3-(4-fluorophenyl)-1-(tetrahydro-2H- | 1.38 | 338 | — | — | — | B |
| | pyran-2-yl)-1H-pyrazol-4-yl]-2- | | | | | | |
| | methylpyridine | | | | | | |
| 32 | 4-[3-(3-chlorophenyl)-1-(tetrahydro-2H- | 2.46 | 379 | — | — | — | B |
| | pyran-2-yl)-1H-pyrazol-4-yl]-1H-pyrrolo[2,3- | | | | | | |
| | b]pyridine | | | | | | |
| 33 | 4-[5-(2,2-difluoro-1,3-benzodioxo1-4-yl)-1- | 1.64 | 386 | — | — | — | B |
| | (tetrahydro-2H-pyran-2-yl)-1H-pyrazol-4- | | | | | | |
| | yl]pyridine | | | | | | |
| 34 | 4-[3-(3-chlorophenyl)-1-(3-methylbut-2-en-1- | 3.5 | 325 | — | — | — | C |
| | yl)-1H-pyrazol-4-yl]pyrimidine | | | | | | |
| 36 | 4-(6-{1-(tetrahydro-2H-pyran-2-yl)-3-[3- | 2.1 | 460 | — | — | — | B |
| | (trifluoromethyl)phenyl]-1H-pyrazol-4- | | | | | | |
| | yl}pyrimidin-4-yl)morpholine | | | | | | |
| 37 | 4-[5-(4-fluoro-2-methylphenyl)-1-(tetrahydro- | 1.40 | 338 | — | — | — | B |
| | 2H-pyran-2-yl)-1H-pyrazol-4-yl]pyridine | | | | | | |
| 38 | 4-[1-(butan-2-yl)-5-(2,6-difluorophenyl)-1H- | 1.9 | 314 | — | — | — | C |
| | pyrazol-4-yl]pyridine | | | | | | |
| 39 | 4-[3-(4-fluorophenyl)-1-(tetrahydro-2H- | 1.38 | 339 | — | — | — | B |
| | pyran-2-yl)-1H-pyrazol-4-yl]pyridin-2-amine | | | | | | |
| 40 | N-{4-[3-(4-fluorophenyl)-1-(propan-2-yl)- | 2.59 | 369 | — | — | — | A |
| | 1H-pyrazol-4-yl]pyridin-2-yl}-2- | | | | | | |
| | methoxyacetamide | | | | | | |
| 41 | N-{4-[3-(4-fluorophenyl)-1-(tetrahydro-2H- | 3.19 | 423 | — | — | — | B |
| | pyran-2-yl)-1H-pyrazol-4-yl]pyridin-2-yl}- | | | | | | |
| | 2,2-dimethylpropanamide | | | | | | |
| 42 | 4-{1-(tetrahydro-2H-pyran-2-yl)-3-[3- | 1.84 | 374 | — | — | — | B |
| | (trifluoromethyl)phenyl]-1H-pyrazol-4- | | | | | | |
| | yl}pyridine | | | | | | |
| 43 | 4-[3-(4-methoxyphenyl)-1-(tetrahydro-2H- | 1.24 | 336 | — | — | — | B |
| | pyran-2-yl)-1H-pyrazol-4-yl]pyridine | | | | | | |
| 44 | 4-[3-(4-fluorophenyl)-1-(propan-2-yl)-1H- | 2.24 | 332 | — | — | — | B |
| | pyrazol-4-yl]quinoline | | | | | | |
| 45 | 4-[5-(4-methoxynaphthalen-1-yl)-1- | 1.69 | 386 | — | — | — | B |
| | (tetrahydro-2H-pyran-2-yl)-1H-pyrazol-4- | | | | | | |
| | yl]pyridine | | | | | | |
| 46 | 4-[3-(4-fluorophenyl)-1-(propan-2-yl)-1H- | 1.18 | 297 | — | — | — | A |
| | pyrazol-4-yl]pyridin-2-amine | | | | | | |
| 47 | 4-[3-(4-fluorophenyl)-1-(propan-2-yl)-1H- | 3.63 | 359 | — | — | 80:20 | B |
| | pyrazol-4-yl]-1-(prop-2-yn-1-yl)-1H- | | | | | | |
| | pyrrolo[2,3-b]pyridine | | | | | | |
| 48 | 2,2-dimethyl-N-(4-{1-(tetrahydro-2H-pyran- | 4.1 | 473 | — | — | — | B |
| | 2-yl)-3-[3-(trifluoromethyl)phenyl]-1H- | | | | | | |
| | pyrazol-4-yl}pyridin-2-yl)propanamide | | | | | | |
| 49 | 4-[3-(4-fluorophenyl)-1-(tetrahydro-2H- | 3.47 | 401 | — | — | — | B |
| | pyran-2-yl)-1H-pyrazol-4-yl]-1-(prop-2-yn-1- | | | | | | |
| | yl)-1H-pyrrolo[2,3-b]pyridine | | | | | | |
| 50 | 4-{1-(tetrahydro-2H-pyran-2-yl)-3-[3- | 2.93 | 413 | — | — | — | B |
| | (trifluoromethyl)phenyl]-1H-pyrazol-4-yl}- | | | | | | |
| | 1H-pyrrolo[2,3-b]pyridine | | | | | | |
| 51 | 4-[5-(3-chloro-4-fluorophenyl)-1-(tetrahydro- | 1.57 | 359 | — | — | — | B |
| | 2H-pyran-2-yl)-1H-pyrazol-4-yl]pyridine | | | | | | |
| 52 | 4-{6-[3-(4-fluorophenyl)-1-(tetrahydro-2H- | 1.49 | 410 | — | — | — | B |
| | pyran-2-yl)-1H-pyrazol-4-yl]pyrimidin-4- | | | | | | |
| | yl}morpholine | | | | | | |
| 53 | 4-[3-(4-fluorophenyl)-1-(propan-2-yl)-1H- | 2.74 | 339 | — | — | — | A |
| | pyrazol-4-yl]-N-methylpyridine-2- | | | | | | |
| | carboxamide | | | | | | |
| 54 | 4-[3-(4-fluorophenyl)-1-(tetrahydro-2H- | 2.46 | 374 | — | — | — | B |
| | pyran-2-yl)-1H-pyrazol-4-yl]quinoline | | | | | | |
| 55 | 4-[5-(4-fluoro-3-methylphenyl)-1-(tetrahydro- | 1.47 | 338 | — | — | — | B |
| | 2H-pyran-2-yl)-1H-pyrazol-4-yl]pyridine | | | | | | |
| 56 | 4-[5-(2,4-difluorophenyl)-1-(tetrahydro-2H- | 1.35 | 342 | — | — | — | B |
| | pyran-2-yl)-1H-pyrazol-4-yl]pyridine | | | | | | |
| 57 | 4-[5-(4-tert-butylphenyl)-1-(tetrahydro-2H- | 2.02 | 362 | — | — | — | B |
| | pyran-2-yl)-1H-pyrazol-4-yl]pyridine | | | | | | |
| 58 | 4-[1-tert-butyl-3-(4-fluorophenyl)-1H- | 2.62 | 296 | — | — | — | C |
| | pyrazol-4-yl]pyridine | | | | | | |
| 59 | 4-[5-(4-phenoxyphenyl)-1-(tetrahydro-2H- | 1.88 | 398 | — | — | — | B |
| | pyran-2-yl)-1H-pyrazol-4-yl]pyridine | | | | | | |
| 60 | 3-[4-(pyridin-4-yl)-1-(tetrahydro-2H-pyran-2- | 1.23 | 331 | — | — | — | B |
| | yl)-1H-pyrazol-5-yl]benzonitrile | | | | | | |
| 61 | 2-fluoro-5-[4-(pyridin-4-yl)-1-(tetrahydro-2H- | 1.45 | 349 | — | — | — | B |
| | pyran-2-yl)-1H-pyrazol-5-yl]benzonitrile | | | | | | |
| 62 | 4-[3-(4-methoxyphenyl)-1-(tetrahydro-2H- | 3.32 | 403 | — | — | — | B |
| | pyran-2-yl)-1H-pyrazol-4-yl]-6-(1H-pyrazol- | | | | | | |
| | 1-yl)pyrimidine | | | | | | |
| 63 | N-ethyl-6-[3-(4-fluorophenyl)-1-(tetrahydro- | 1.42 | 368 | — | — | — | B |
| | 2H-pyran-2-yl)-1H-pyrazol-4-yl]pyrimidin-4- | | | | | | |
| | amine | | | | | | |
| 64 | 4-[5-(4-fluoro-2-methoxyphenyl)-1- | 1.26 | 354 | — | — | — | B |
| | (tetrahydro-2H-pyran-2-yl)-1H-pyrazol-4- | | | | | | |
| | yl]pyridine | | | | | | |
| 65 | 4-[3-(4-methoxyphenyl)-1-(tetrahydro-2H- | 1.54 | 376 | — | — | — | B |
| | pyran-2-yl)-1H-pyrazol-4-yl]-7H-pyrrolo[2,3- | | | | | | |
| | d]pyrimidine | | | | | | |
| 66 | 4-[3-(4-fluorophenyl)-1-(tetrahydro-2H- | 1.69 | 364 | — | — | — | B |
| | pyran-2-yl)-1H-pyrazol-4-yl]-7H-pyrrolo[2,3- | | | | | | |
| | d]pyrimidine | | | | | | |
| 67 | 4-[5-(3-phenoxyphenyl)-1-(tetrahydro-2H- | 1.88 | 398 | — | — | — | B |
| | pyran-2-yl)-1H-pyrazol-4-yl]pyridine | | | | | | |
| 68 | 4-{5-[4-(methylsulphonyl)phenyl]-1- | 0.96 | 384 | — | — | — | B |
| | (tetrahydro-2H-pyran-2-yl)-1H-pyrazol-4- | | | | | | |
| | yl}pyridine | | | | | | |
| 69 | 4-[3-(4-methoxyphenyl)-1-(tetrahydro-2H- | 1.31 | 351 | — | — | — | B |
| | pyran-2-yl)-1H-pyrazol-4-yl]pyridin-2-amine | | | | | | |
| 70 | 4-[5-(4-chlorophenyl)-1-(tetrahydro-2H- | 1.52 | 340 | — | — | — | B |
| | pyran-2-yl)-1H-pyrazol-4-yl]pyridine | | | | | | |
| 71 | 4-{1-(tetrahydro-2H-pyran-2-yl)-5[4- | 1.78 | 390 | — | — | — | B |
| | (trifluoromethoxy)phenyl]-1H-pyrazol-4- | | | | | | |
| | yl}pyridine | | | | | | |
| 72 | N-{4-[5-(4-fluorophenyl)-1-(propan-2-yl)- | 2.99 | 381 | — | — | — | B |
| | 1H-pyrazol-4-yl]pyridin-2-yl}-2,2- | | | | | | |
| | dimethylpropanamide | | | | | | |
| 73 | 4-[3-(4-methoxyphenyl)-1-(tetrahydro-2H- | 1.33 | 350 | — | — | — | B |
| | pyran-2-yl)-1H-pyrazol-4-yl]-2- | | | | | | |
| | methylpyridine | | | | | | |
| 74 | 4-[3-(4-fluorophenyl)-1-(tetrahydro-2H- | 3.58 | 371 | — | — | — | B |
| | pyran-2-yl)-1H-pyrazol-4-yl]-2- | | | | | | |
| | (methylsulphanyl)pyrimidine | | | | | | |
| 75 | 4-[3-(4-fluorophenyl)-1-(tetrahydro-2H- | 3.58 | 391 | — | — | — | B |
| | pyran-2-yl)-1H-pyrazol-4-yl]-6-(1H-pyrazol- | | | | | | |
| | 1-yl)pyrimidine | | | | | | |
| 76 | 4-[5-(2,3-dichlorophenyl)-1-(tetrahydro-2H- | 1.66 | 374 | 1.60 | 374 | 40:60 | B |
| | pyran-2-yl)-1H-pyrazol-4-yl]pyridine | | | | | | |
| 77 | 4-[3-(4-methoxyphenyl)-1-(tetrahydro-2H- | 2.27 | 351 | — | — | — | B |
| | pyran-2-yl)-1H-pyrazol-4-yl]-2- | | | | | | |
| | methylpyrimidine | | | | | | |
| 78 | N-ethyl-6-[3-(4-methoxyphenyl)-1- | 1.28 | 380 | — | — | — | B |
| | (tetrahydro-2H-pyran-2-yl)-1H-pyrazol-4- | | | | | | |
| | yl]pyrimidin-4-amine | | | | | | |
| 79 | 2-[3-(4-fluorophenyl)-4-(pyridin-4-yl)-1H- | 2.64 | 385 | — | — | — | B |
| | pyrazol-1-yl]-5-(trifluoromethyl)pyridine | | | | | | |
| 80 | 4-[3-(4-methoxyphenyl)-1-(tetrahydro-2H- | 1.87 | 375 | — | — | — | B |
| | pyran-2-yl)-1H-pyrazol-4-yl]-1H-pyrrolo[2,3- | | | | | | |
| | b]pyridine | | | | | | |
| 81 | 1-{4-[3-(4-fluorophenyl)-1-(tetrahydro-2H- | 3.85 | 405 | — | — | — | B |
| | pyran-2-yl)-1H-pyrazol-4-yl]-1H-pyrrolo[2,3- | | | | | | |
| | b]pyridin-1-yl}ethanone | | | | | | |
| 82 | 2,2-dimethyl-N-(4-{1-(tetrahydro-2H-pyran- | 3.84 | 473 | — | — | — | B |
| | 2-yl)-5-[3-(trifluoromethyl)phenyl]-1H- | | | | | | |
| | pyrazol-4-yl}pyridin-2-yl)propanamide | | | | | | |
| 83 | 4-[3-(3-chlorophenyl)-1-(tetrahydro-2H- | 3.15 | 397 | — | — | — | B |
| | pyran-2-yl)-1H-pyrazol-4-yl]-N- | | | | | | |
| | methylpyridine-2-carboxamide | | | | | | |
| 84 | N-{4-[3-(4-fluorophenyl)-1-(propan-2-yl)- | 2.50 | 367 | — | — | — | A |
| | 1H-pyrazol-4-yl]pyridin-2-yl}-2- | | | | | | |
| | methylpropanamide | | | | | | |
| 85 | N-{4-[3-(4-fluorophenyl)-1-(propan-2-yl)- | 2.15 | 353 | — | — | — | A |
| | 1H-pyrazol-4-yl]pyridin-2-yl}propanamide | | | | | | |
| 86 | N-{4-[3-(4-fluorophenyl)-1-(propan-2-yl)- | 2.07 | 365 | — | — | — | B |
| | 1H-pyrazol-4-yl]pyridin-2- | | | | | | |
| | yl}cyclopropanecarboxamide | | | | | | |
| 87 | N-{4-[3-(4-fluorophenyl)-1-(2-methylpropyl)- | 2.89 | 381 | — | — | — | B |
| | 1H-pyrazol-4-yl]pyridin-2-yl}-2- | | | | | | |
| | methylpropanamide | | | | | | |
| 88 | 4-[3-(4-fluorophenyl)-1-(2-methoxyethyl)- | 1.11 | 298 | 1.05 | 298 | 70:30 | A |
| | 1H-pyrazol-4-yl]pyridine | | | | | | |
| 89 | 4-[3-(4-fluorophenyl)-1-(propan-2-yl)-1H- | 1.16 | 282 | — | — | — | B |
| | pyrazol-4-yl]pyridine | | | | | | |
| 90 | 4-[3-(4-fluorophenyl)-1-(tetrahydro-2H- | 2.72 | 381 | — | — | — | B |
| | pyran-2-yl)-1H-pyrazol-4-yl]-N- | | | | | | |
| | methylpyridine-2-carboxamide | | | | | | |
| 91 | 4-[1-(butan-2-yl)-3-(4-fluorophenyl)-1H- | 1.5 | 296 | — | — | — | A |
| | pyrazol-4-yl]pyridine | | | | | | |
| 92 | 4-[1-cyclopentyl-3-(4-fluorophenyl)-1H- | 1.65 | 308 | 1.65 | 308 | 85:15 | A |
| | pyrazol-4-yl]pyridine | | | | | | |
| 92 | 4-[1-cyclopentyl-3-(4-fluorophenyl)-1H- | 1.65 | 308 | 1.65 | 308 | 80:20 | A |
| | pyrazol-4-yl]pyridine | | | | | | |
| 93 | 4-[3-(4-fluorophenyl)-1-(tetrahydro-2H- | 2.46 | 367 | — | — | — | B |
| | pyran-2-yl)-1H-pyrazol-4-yl]pyridine-2- | | | | | | |
| | carboxamide | | | | | | |
| 94 | 4-[3-(4-fluorophenyl)-1-(tetrahydro-2H- | 4.34 | 443 | — | — | — | B |
| | pyran-2-yl)-1H-pyrazol-4-yl]-N- | | | | | | |
| | phenylpyridine-2-carboxamide | | | | | | |
| 95 | 4-[3-(3-chlorophenyl)-1-(tetrahydro-2H- | 2.93 | 383 | — | — | — | B |
| | pyran-2-yl)-1H-pyrazol-4-yl]pyridine-2- | | | | | | |
| | carboxamide | | | | | | |
| 96 | 4-[3-(3-chlorophenyl)-1-(tetrahydro-2H- | 4.89 | 459 | — | — | — | B |
| | pyran-2-yl)-1H-pyrazol-4-yl]-N- | | | | | | |
| | phenylpyridine-2-carboxamide | | | | | | |
| 97 | 4-[5-(4-fluorophenyl)-1-(propan-2-yl)-1H- | 1.25 | 297 | — | — | — | B |
| | pyrazol-4-yl]pyridin-2-amine | | | | | | |
| 98 | 4-{5-[2-(benzyloxy)phenyl]-1-(tetrahydro- | 1.64 | 412 | — | — | — | B |
| | 2H-pyran-2-yl)-1H-pyrazol-4-yl}pyridine | | | | | | |
| 99 | 8-[4-(pyridin-4-yl)-1-(tetrahydro-2H-pyran-2- | 1.09 | 357 | — | — | — | B |
| | yl)-1H-pyrazol-5-yl]quinoline | | | | | | |
| 100 | 3-{[3-(4-fluorophenyl)-4-(pyridin-4-yl)-1H- | 0.78 | 331 | — | — | — | B |
| | pyrazol-1-yl]methyl}pyridine | | | | | | |
| 101 | 4-{1-[1-(3-chlorophenyl)ethyl]-3-(4- | 2.17 | 378 | 2.1 | 378 | 80:20 | A |
| | fluorophenyl)-1H-pyrazol-4-yl}pyridine | | | | | | |
| 102 | 4-{1-[1-(2-chlorophenyl)ethyl]-3-(4- | 2.16 | 378 | 2.13 | 378 | 91:9 | A |
| | fluorophenyl)-1H-pyrazol-4-yl}pyridine | | | | | | |
| 103 | 4-[3-(4-fluorophenyl)-1-(2,2,2-trifluoroethyl)- | 1.41 | 322 | 1.33 | 322 | 91:9 | A |
| | 1H-pyrazol-4-yl]pyridine | | | | | | |
| 104 | 4-[1-(2,3-difluorobenzyl)-3-(4-fluorophenyl)- | 1.80 | 366 | 1.7 | 366 | 75:25 | A |
| | 1H-pyrazol-4-yl]pyridine | | | | | | |
| 105 | tert-butyl[3-(4-fluorophenyl)-4-(pyridin-4- | 1.67 | 354 | 1.59 | 354 | 78:22 | A |
| | yl)-1H-pyrazol-1-yl]acetate | | | | | | |
| 106 | methyl-2-[3-(4-fluorophenyl)-4-(pyridin-4- | 1.67 | 354 | 1.6 | 354 | 93:7 | A |
| | yl)-1H-pyrazol-1-yl]-3-methylbutanoate | | | | | | |
| 107 | methyl-2-[3-(4-fluorophenyl)-4-(pyridin-4- | 1.20 | 326 | — | — | — | A |
| | yl)-1H-pyrazol-1-yl]propanoate | | | | | | |
| 108 | 4-[1-(2-chloro-6-fluorobenzyl)-3-(4- | 1.90 | 382 | 1.82 | 382 | 87:13 | A |
| | fluorophenyl)-1H-pyrazol-4-yl]pyridine | | | | | | |
| 109 | 4-{1-[4-(difluoromethoxy)benzyl]-3-(4- | 1.88 | 396 | 1.79 | 396 | 74:26 | A |
| | fluorophenyl)-1H-pyrazol-4-yl}pyridine | | | | | | |
| 110 | 4-{1-[(2-chloro-1,3-thiazol-5-yl)methyl]-3-(4- | 1.54 | 371 | — | — | — | A |
| | fluorophenyl)-1H-pyrazol-4-yl}pyridine | | | | | | |
| 111 | 4-{3-(4-fluorophenyl)-1-[5-(trifluoromethyl)- | 2.90 | 392 | — | — | — | A |
| | 1,3,4-thiadiazol-2-yl]-1H-pyrazol-4- | | | | | | |
| | yl}pyridine | | | | | | |
| 112 | 4-[3-(4-fluorophenyl)-4-(pyridin-4-yl)-1H- | 2.73 | 386 | — | — | — | A |
| | pyrazol-1-yl]-6-(trifluoromethyl)pyrimidine | | | | | | |
| 113 | 1-cyclohexyl-2-[3-(4-fluorophenyl)-4- | 1.86 | 364 | 1.76 | 364 | 80:20 | A |
| | (pyridin-4-yl)-1H-pyrazol-1-yl]ethanone | | | | | | |
| 114 | 1-cyclopentyl-2-[3-(4-fluorophenyl)-4- | 1.63 | 350 | 1.54 | 350 | 75:25 | A |
| | (pyridin-4-yl)-1H-pyrazol-1-yl]ethanone | | | | | | |
| 115 | 2-{[3-(4-fluorophenyl)-4-(pyridin-4-yl)-1H- | 0.99 | 331 | — | — | — | A |
| | pyrazol-1-yl]methyl}pyridine | | | | | | |
| 115 | 2-{[3-(4-fluorophenyl)-4-(pyridin-4-yl)-1H- | 1.08 | 331 | 0.97 | 331 | 75:25 | A |
| | pyrazol-1-yl]methyl}pyridine | | | | | | |
| 116 | 4-{1-[(2,2-dichlorocyclopropyl)methyl]-3-(4- | 1.76 | 362 | 1.68 | 362 | 75:25 | A |
| | fluorophenyl)-1H-pyrazol-4-yl}pyridine | | | | | | |
| 117 | 4-{3-(4-fluorophenyl)-1-[2-(methyl- | 1.29 | 314 | 1.25 | 314 | 80:20 | A |
| | sulphanyl)ethyl]-1H-pyrazol-4-yl]pyridine | | | | | | |
| 118 | 4-{1-[2-(3-chloropheny)ethyl]-3-(4- | 2.04 | 378 | 1.9 | 378 | 70:30 | A |
| | fluorophenyl)-1H-pyrazol-4-yl}pyridine | | | | | | |
| 119 | 4-{1-[2-(2-chlorophenyl)ethyl]-3-(4- | 2.04 | 378 | 1.9 | 378 | 72:28 | A |
| | fluorophenyl)-1H-pyrazol-4-yl}pyridine | | | | | | |
| 120 | 4-{1-[1-(4-chlorophenyl)ethyl]-3-(4- | 2.18 | 378 | 2.12 | 378 | 80:20 | A |
| | fluorophenyl)-1H-pyrazol-4-yl}pyridine | | | | | | |
| 121 | 4-[3-(4-fluorophenyl)-1-(pyridin-4-ylmethyl)- | 0.25 | 331 | — | — | — | B |
| | 1H-pyrazol-4-yl]pyridine | | | | | | |
| 122 | 4-{3-(4-fluorophenyl)-1-[2-(trifluoro- | 1.66 | 352 | 1.59 | 352 | 88:12 | A |
| | methoxy)ethyl]-1H-pyrazol-4-yl}pyridine | | | | | | |
| 122 | 4-{3-(4-fluorophenyl)-1-[2-(trifluoro- | 1.66 | 352 | 1.59 | 352 | 66:33 | A |
| | methoxy)ethyl]-1H-pyrazol-4-yl}pyridine | | | | | | |
| 123 | 4-[3-(4-fluorophenyl)-1-(tetrahydrofuran-2- | 1.19 | 324 | 1.16 | 324 | 89:11 | A |
| | ylmethyl)-1H-pyrazol-4-yl]pyridine | | | | | | |
| 124 | 3-[3-(4-fluorophenyl)-4-(pyridin-4-yl)-1H- | 0.17 | 314 | — | — | — | A |
| | pyrazol-1-yl]propan-1,2-diol | | | | | | |
| 125 | 4-[1-(biphenyl-3-ylmethyl)-3-(4- | 2.38 | 406 | 2.25 | 406 | 91:9 | A |
| | fluorophenyl)-1H-pyrazol-4-yl]pyridine | | | | | | |
| 126 | 4-[1-(cyclopropylmethyl)-3-(4-fluorophenyl)- | 1.33 | 294 | — | — | — | A |
| | 1H-pyrazol-4-yl]pyridine | | | | | | |
| 127 | 4-[1-benzyl-3-(4-fluorophenyl)-1H-pyrazol-4- | 1.63 | 330 | 1.57 | 330 | 77:23 | A |
| | yl]pyridine | | | | | | |
| 128 | 4-[1-(2-fluorobenzyl)-3-(4-fluorophenyl)-1H- | 1.84 | 348 | 1.76 | 348 | 80:20 | A |
| | pyrazol-4-yl]pyridine | | | | | | |
| 129 | 4-[1-(3-fluorobenzyl)-3-(4-fluorophenyl)-1H- | 1.71 | 348 | 1.61 | 348 | 75:25 | A |
| | pyrazol-4-yl]pyridine | | | | | | |
| 130 | 4-[1-(4-fluorobenzyl)-3-(4-fluorophenyl)-1H- | 1.69 | 348 | 1.61 | 348 | 75:25 | A |
| | pyrazol-4-yl]pyridine | | | | | | |
| 131 | 4-[1-(biphenyl-4-ylmethyl)-3-(4- | 2.39 | 406 | 2.3 | 406 | 95:5 | A |
| | fluorophenyl)-1H-pyrazol-4-yl]pyridine | | | | | | |
| 132 | ethyl-3-[3-(4-fluorophenyl)-4-(pyridin-4-yl)- | 1.29 | 340 | — | — | — | A |
| | 1H-pyrazol-1-yl]propanoate | | | | | | |
| 133 | 4-[3-(4-fluorophenyl)-1-{2-[2-(2- | 1.11 | 386 | 1.06 | 386 | 70:30 | A |
| | methoxyethoxy)ethoxy]ethyl}-1H-pyrazol-4- | | | | | | |
| | yl]pyridine | | | | | | |
| 134 | 4-{3-(4-fluorophenyl)-1-[2-(2- | 1.04 | 342 | — | — | — | A |
| | methoxyethoxy)ethyl]-1H-pyrazol-4- | | | | | | |
| | yl}pyridine | | | | | | |
| 134 | 4-{3-(4-fluorophenyl)-1-[2-(2- | 1.11 | 342 | — | — | — | A |
| | methoxyethoxy)ethyl]-1H-pyrazol-4- | | | | | | |
| | yl}pyridine | | | | | | |
| 135 | 4-[1-(biphenyl-2-ylmethyl)-3-(4- | 2.34 | 406 | — | — | — | B |
| | fluorophenyl)-1H-pyrazol-4-yl]pyridine | | | | | | |
| 136 | 2-{[3-(4-fluorophenyl)-4-(pyridin-4-yl)-1H- | 1.49 | 355 | — | — | — | A |
| | pyrazol-1-yl]methyl}benzonitrile | | | | | | |
| 137 | 4-[3-(4-fluorophenyl)-1-(naphthalen-1- | 2.12 | 380 | 1.99 | 380 | 84:16 | A |
| | ylmethyl)-1H-pyrazol-4-yl]pyridine | | | | | | |
| 138 | 4-[3-(4-fluorophenyl)-1-(3-phenoxybenzyl)- | 2.36 | 422 | — | — | — | A |
| | 1H-pyrazol-4-yl]pyridine | | | | | | |
| 139 | 4-[1-(4-fluoro-3-phenoxybenzyl)-3-(4- | 2.40 | 440 | — | — | — | A |
| | fluorophenyl)-1H-pyrazol-4-yl]pyridine | | | | | | |
| 140 | 3-{[3-(4-fluorophenyl)-4-(pyridin-4-yl)-1H- | 1.50 | 355 | — | — | — | A |
| | pyrazol-1-yl]methyl}benzonitrile | | | | | | |
| 140 | 3-{[3-(4-fluorophenyl)-4-(pyridin-4-yl)-1H- | 1.51 | 355 | 1.38 | 355 | 60:40 | A |
| | pyrazol-1-yl]methyl}benzonitrile | | | | | | |
| 141 | 4-{[3-(4-fluorophenyl)-4-(pyridin-4-yl)-1H- | 1.48 | 355 | — | — | — | A |
| | pyrazol-1-yl]methyl}benzonitrile | | | | | | |
| 141 | 4-{[3-(4-fluorophenyl)-4-(pyridin-4-yl)-1H- | 1.50 | 355 | 1.38 | 355 | 70:30 | A |
| | pyrazol-1-yl]methyl}benzonitrile | | | | | | |
| 142 | 3-[3-(4-fluorophenyl)-4-(pyridin-4-yl)-1H- | 0.95 | 324 | — | — | — | A |
| | pyrazol-1-yl]dihydrofuran-2(3H)-one | | | | | | |
| 143 | 4-[3-(4-fluorophenyl)-1-(1H-imidazol-2- | 0.41 | 320 | — | — | — | B |
| | ylmethyl)-1H-pyrazol-4-yl]pyridine | | | | | | |
| 144 | 1-(1,5-dimethyl-1H-pyrazol-3-yl)-2-[3-(4- | 1.31 | 376 | 1.18 | 376 | 62:38 | A |
| | fluorophenyl)-4-(pyridin-4-yl)-1H-pyrazol-1- | | | | | | |
| | yl]ethanone | | | | | | |
| 144 | 1-(1,5-dimethyl-1H-pyrazol-3-yl)-2-[3-(4- | 1.30 | 376 | — | — | — | A |
| | fluorophenyl)-4-(pyridin-4-yl)-1H-pyrazol-1- | | | | | | |
| | yl]ethanone | | | | | | |
| 145 | 2-[3-(4-fluorophenyl)-4-(pyridin-4-yl)-1H- | 1.05 | 326 | 0.97 | 326 | 91:9 | A |
| | pyrazol-1-yl]ethyl acetate | | | | | | |
| 146 | 3-[3-(4-fluorophenyl)-4-(pyridin-4-yl)-1H- | 0.81 | 293 | — | — | — | A |
| | pyrazol-1-yl]propanonitrile | | | | | | |
| 147 | 4-[3-(4-fluorophenyl)-1-(2-methylpropyl)-1H- | 2.6 | 351 | — | — | — | C |
| | pyrazol-4-yl]-1,3-dihydro-2H-pyrrolo[2,3- | | | | | | |
| | b]pyridine-2-one | | | | | | |
| 148 | 4-[3-(4-fluorophenyl)-1-(2-methylpropyl)-1H- | 3.68 | 393 | — | — | — | C |
| | pyrazol-4-yl]-1,3,3-trimethyl-1,3-dihydro-2H- | | | | | | |
| | pyrrolo[2,3-b]pyridine-2-one | | | | | | |
| 149 | 4-[5-(4-fluorophenyl)-1-(2-methylpropyl)-1H- | 2.37 | 352 | — | — | — | C |
| | pyrazol-4-yl]-N-(prop-2-en-1-yl)pyrimidin-2- | | | | | | |
| | amine | | | | | | |
| 150 | 4-[3-(4-fluorophenyl)-1-(2-methylpropyl)-1H- | 2.46 | 352 | — | — | — | C |
| | pyrazol-4-yl]-N-(prop-2-en-1-yl)pyrimidin-2- | | | | | | |
| | amine | | | | | | |
| 153 | N-{4-[3-(4-fluorophenyl)-1-(2- | 1.61 | 369 | — | — | — | B |
| | methoxyethyl)-1H-pyrazol-4-yl]pyridin-2- | | | | | | |
| | yl}propanamide | | | | | | |
| 154 | 2-fluoro-5-[1-(2-methylpropyl)-4-(pyridin-4- | 1.65 | 321 | — | — | — | A |
| | yl)-1H-pyrazol-3-yl]benzonitrile | | | | | | |
| 155 | N-{4-[3-(4-fluorophenyl)-1-(2-methylpropyl)- | 2.51 | 367 | — | — | — | A |
| | 1H-pyrazol-4-yl]pyridin-2-yl}propanamide | | | | | | |
| 156 | N-{4-[3-(4-fluorophenyl)-1-(propan-2-yl)- | 2.57 | 379 | — | — | — | A |
| | 1H-pyrazol-4-yl]pyridin-2- | | | | | | |
| | yl}cyclobutancarboxamide | | | | | | |
| 157 | N-{4-[1-(butan-2-yl)-3-(4-fluorophenyl)-1H- | 2.82 | 381 | — | — | — | B |
| | pyrazol-4-yl]pyridin-2-yl}-2- | | | | | | |
| | methylpropanamide | | | | | | |
| 158 | 2-cyclopropyl-N-{4-[3-(4-fluorophenyl)-1-(2- | 3.03 | 393 | — | — | — | B |
| | methylpropyl)-1H-pyrazol-4-yl]pyridin-2- | | | | | | |
| | yl}acetamide | | | | | | |
| 159 | N-{4-[1-(butan-2-yl)-3-(4-fluorophenyl)-1H- | 3 | 393 | — | — | — | B |
| | pyrazol-4-yl]pyridin-2-yl}-2- | | | | | | |
| | cyclopropylacetamide | | | | | | |
| 160 | N-{4-[3-(4-fluorophenyl)-1-(propan-2-yl)- | 2.82 | 381 | — | — | — | A |
| | 1H-pyrazol-4-yl]pyridin-2-yl}-3- | | | | | | |
| | methylbutanamide | | | | | | |
| 161 | N-{4-[3-(4-fluorophenyl)-1-(propan-2-yl)- | 1.85 | 339 | — | — | — | A |
| | 1H-pyrazol-4-yl]pyridin-2-yl}acetamide | | | | | | |
| 162 | N-{4-[1-(butan-2-yl)-3-(4-fluorophcnyl)-1H- | 2.95 | 383 | — | — | — | B |
| | pyrazol-4-yl]pyridin-2-yl}-2- | | | | | | |
| | methoxyacetamide | | | | | | |
| 163 | N-{4-[3-(4-fluorophenyl)-1-(2- | 2.05 | 385 | — | — | — | A |
| | methoxyethyl)-1H-pyrazol-4-yl]pyridin-2- | | | | | | |
| | yl}-2-methoxyacetamide | | | | | | |
| 164 | N-{4-[1-(butan-2-yl)-3-(4-fluorophenyl)-1H- | 2.46 | 367 | — | — | — | B |
| | pyrazol-4-yl]pyridin-2-yl}propanamide | | | | | | |
| 165 | N-{4-[3-(4-fluorophenyl)-1-(2-methylpropyl)- | 2.6 | 379 | — | — | — | B |
| | 1H-pyrazol-4-yl]pyridin-2- | | | | | | |
| | yl}cyclopropanecarboxamide | | | | | | |
| 166 | N-{4-[3-(4-fluorophenyl)-1-(2-methylpropyl)- | 2.96 | 383 | — | — | — | B |
| | 1H-pyrazol-4-yl]pyridin-2-yl}-2- | | | | | | |
| | methoxyacetamide | | | | | | |
| 167 | N-{4-[1-(butan-2-yl)-3-(4-fluorophenyl)-1H- | 2.56 | 379 | — | — | — | B |
| | pyrazol-4-yl]pyridin-2- | | | | | | |
| | yl}cyclopropanecarboxamide | | | | | | |
| 168 | 4-[1-cyclohexyl-3-(4-fluorophenyl)-1H- | 1.91 | 322 | 1.82 | 322 | 69:31 | A |
| | pyrazol-4-yl]pyridine | | | | | | |
| 169 | 4-[1-(2,2-difluoroethyl)-3-(4-fluorophenyl)- | 1 | 304 | — | — | — | A |
| | 1H-pyrazol-4-yl]pyridine | | | | | | |
| 170 | ethyl[3-(4-fluorophenyl)-4-(pyridin-4-yl)-1H- | 1.31 | 326 | — | — | — | B |
| | pyrazol-1-yl]acetate | | | | | | |
| 171 | 4-[3-(4-fluorophenyl)-5-methyl-1-(prop-2-yn- | 2.51 | 292 | 2.33 | 292 | 62:19 | C |
| | 1-yl)-1H-pyrazol-4-yl]pyridinc | | | | | | |
| 172 | 4-[3-(4-fluorophenyl)-1H-pyrazol-4-yl]-N- | 1.37 | 298 | — | — | — | A |
| | (propan-2-yl)pyrimidin-2-amine | | | | | | |
| 173 | 4-[3-(4-fluorophenyl)-1H-pyrazol-4-yl]-N- | 1.44 | 294 | — | — | — | A |
| | (prop-2-yn-1-yl)pyrimidin-2-amine | | | | | | |
| 174 | 4-[3-(4-fluorophenyl)-1-(2-methylpropyl)-1H- | 2.39 | 354 | — | — | — | C |
| | pyrazol-4-yl]-N-(propan-2-yl)pyrimidin-2- | | | | | | |
| | amine | | | | | | |
| 175 | 3-(4-fluorophenyl)-4-(pyridin-4-yl)-1H- | 1.08 | 265 | — | — | — | A |
| | pyrazol-5-carbonitrile | | | | | | |
| 176 | 4-{1-[1-(2-chloro-1,3-thiazol-5-yl)ethyl]-3-(4- | 1.82 | 385 | — | — | — | B |
| | fluorophenyl)-1H-pyrazol-4-yl}pyridine | | | | | | |
| 177 | 4-[1-(1,3-difluoropropan-2-yl)-3-(4- | 1.16 | 318 | — | — | — | B |
| | fluorophenyl)-1H-pyrazol-4-yl]pyridine | | | | | | |
| 178 | 1-[3-(4-fluorophenyl)-4-(pyridin-4-yl)-1H- | 1.37 | 324 | — | — | — | A |
| | pyrazol-1-yl]-3-methylbutan-2-one | | | | | | |
| 179 | 4-[1-(2-ethoxyethyl)-3-(4-fluorophenyl)-1H- | 1.25 | 312 | — | — | — | B |
| | pyrazol-4-yl]pyridine | | | | | | |
| 180 | [3-(4-fluorophenyl)-4-(pyridin-4-yl)-1H- | 0.89 | 279 | — | — | — | A |
| | pyrazol-1-yl]acetonitrile | | | | | | |
| 181 | propan-2-yl {4-[3-(4-fluorophenyl)-1- | 2.99 | 383 | — | — | — | A |
| | (propan-2-yl)-1H-pyrazol-4-yl]pyridin-2- | | | | | | |
| | yl}carbamate | | | | | | |
| 182 | N-benzyl-4-[3-(4-fluorophenyl)-1-(2- | 3.15 | 402 | — | — | — | C |
| | methylpropyl)-1H-pyrazol-4-yl]pyrimidin-2- | | | | | | |
| | amine | | | | | | |
| 183 | 4-[3-(4-fluorophenyl)-1-(2-methylpropyl)-1H- | 2.76 | 350 | — | — | — | C |
| | pyrazol-4-yl]-N-(prop-2-yn-1-yl)pyrimidin-2- | | | | | | |
| | amine | | | | | | |
| 184 | 4-[3-(4-fluorophenyl)-1-(2-methylpropyl)-1H- | 1.81 | 312 | — | — | — | C |
| | pyrazol-4-yl]pyrimidin-2-amine | | | | | | |
| 185 | N-{4-[3-(4-fluorophenyl)-1-(2-methylpropyl)- | 4.25 | 424 | — | — | — | C |
| | 1H-pyrazol-4-yl]pyrimidin-2-yl}-2-methyl-N- | | | | | | |
| | (propan-2-yl)propanamide | | | | | | |
| 186 | N-{4-[3-(4-fluorophenyl)-1-(2-methylpropyl)- | 3.02 | 494 | — | — | — | A |
| | 1H-pyrazol-4-yl]pyrimidin-2-yl}-2,2,4- | | | | | | |
| | trimethyl-3-oxo-N-(propan-2-yl)pentanamide | | | | | | |
| 187 | 1-{4-[3-(4-fluorophenyl)-1-(2-methylpropyl)- | 3.19 | 379 | — | — | — | C |
| | 1H-pyrazol-4-yl]pyridin-2-yl}pyrrolidin-2-one | | | | | | |
| 188 | N-{4-[3-(4-fluorophenyl)-1-(2-methylpropyl)- | 4.74 | 452 | — | — | — | C |
| | 1H-pyrazol-4-yl]pyrimidin-2-yl}-2-methyl-N- | | | | | | |
| | (2-methylpropanoyl)propanamide | | | | | | |
| 189 | N-{4-[3-(4-fluorophenyl)-1-(2-methylpropyl)- | 2.92 | 382 | — | — | — | C |
| | 1H-pyrazol-4-yl]pyrimidin-2-yl}-2- | | | | | | |
| | methylpropanamide | | | | | | |
| 190 | N-{4-[1-ethyl-3-(4-fluorophenyl)-1H-pyrazol- | 2.72 | 401 | — | — | — | B |
| | 4-yl]pyridin-2-yl}-2-phenylacetamide | | | | | | |
| 191 | N-{4-[3-(4-fluorophenyl)-1-(2-methylpropyl)- | 3.54 | 429 | — | — | — | A |
| | 1H-pyrazol-4-yl]pyridin-2-yl}-2- | | | | | | |
| | phenylacetamide | | | | | | |
| 192 | (2S)-N-{4-[3-(4-fluorophenyl)-1-(propan-2- | 2.14 | 369 | — | — | — | B |
| | yl)-1H-pyrazol-4-yl]pyridin-2-yl}-2- | | | | | | |
| | hydroxypropanamide | | | | | | |
| 193 | tert-butyl 4-{3-(4-fluorophenyl)-4-[2- | 3.03 | 494 | — | — | — | B |
| | (propanoylamino)pyridin-4-yl]-1H-pyrazol-1- | | | | | | |
| | yl}piperidin-1-carboxylate | | | | | | |
| 194 | N-{4-[3-(4-cyano-2,5-difluorophenyl)-5- | | 0 | — | — | — | B |
| | (difluoromethoxy)-1-methyl-1H-pyrazol-4- | | | | | | |
| | yl]pyridin-2-yl}propanamide | | | | | | |
| 195 | N-{4-[3-(4-chloro-2-fluorophenyl)-1-methyl- | 2.70 | 405 | — | — | — | A |
| | 5-(methylsulphanyl)-1H-pyrazol-4-yl]pyridin- | | | | | | |
| | 2-yl}propanamide | | | | | | |
| 196 | N-{4-[5-(difluoromethoxy)-3-(4-fluoro-2- | 2.12 | 407 | — | — | — | A |
| | hydroxyphenyl)-1-methyl-1H-pyrazol-4- | | | | | | |
| | yl]pyridin-2-yl}propanamide | | | | | | |
| 197 | N-{4-[3-(4-chloro-2-fluorophenyl)-1-methyl- | 3.32 | 427 | — | — | — | A |
| | 5-(trifluoromethyl)-1H-pyrazol-4-yl]pyridin- | | | | | | |
| | 2-yl}propanamide | | | | | | |
| 198 | N-{4-[1-ethyl-3-(4-fluorophenyl)-1H-pyrazol- | 1.82 | 339 | — | — | — | B |
| | 4-yl]pyridin-2-yl}propanamide | | | | | | |
| 199 | N-{4-[3-(4-cyano-2,5-difluorophenyl)-5- | 2.59 | 450 | — | — | — | B |
| | (difluoromethoxy)-1-methyl-1H-pyrazol-4- | | | | | | |
| | yl]pyridin-2-yl}-2-methoxyacetamide | | | | | | |
| 200 | N-{4-[3-(4-chloro-2-fluorophenyl)-1-methyl- | 3.00 | 421 | — | — | — | B |
| | 5-(methylsulphanyl)-1H-pyrazol-4-yl]pyridin- | | | | | | |
| | 2-yl}-2-methoxyacetamide | | | | | | |
| 201 | N-{4-[5-(difluoromethoxy)-3-(4-fluoro-2- | 2.38 | 423 | — | — | — | B |
| | hydroxyphenyl)-1-methyl-1H-pyrazol-4- | | | | | | |
| | yl]pyridin-2-yl}-2-methoxyacetamide | | | | | | |
| 202 | N-{4-[3-(4-chloro-2-fluorophenyl)-1-methyl- | 3.45 | 443 | — | — | — | B |
| | 5-(trifluoromethyl)-1H-pyrazol-4-yl]pyridin- | | | | | | |
| | 2-yl}-2-methoxyacetamide | | | | | | |
| 203 | N-{4-[1-ethyl-3-(4-fluorophenyl)-1H-pyrazol- | 2.20 | 355 | — | — | — | A |
| | 4-yl]pyridin-2-yl}-2-methoxyacetamide | | | | | | |
| 204 | tert-butyl 4-[3-(4-fluorophenyl)-4-{2- | 3.51 | 510 | — | — | — | A |
| | [(methoxyacetyl)amino]pyridin-4-yl}-1H- | | | | | | |
| | pyrazol-1-yl]piperidin-1-carboxylate | | | | | | |
| 205 | N-{4-[3-(4-fluorophenyl)-1-(propan-2-yl)- | 3.20 | 401 | — | — | — | A |
| | 1H-pyrazol-4-yl]pyridin-2-yl}benzamide | | | | | | |
| 206 | N-{4-[3-(4-fluorophenyl)-1-(propan-2-yl)- | 3.14 | 407 | — | — | — | A |
| | 1H-pyrazol-4-yl]pyridin-2-yl}thiophen-2- | | | | | | |
| | carboxamide | | | | | | |
| 207 | N-{4-[3-(4-fluorophenyl)-1-(propan-2-yl)- | 2.96 | 407 | — | — | — | A |
| | 1H-pyrazol-4-yl]pyridin-2-yl}thiophen-3- | | | | | | |
| | carboxamide | | | | | | |
| 208 | N-{4-[3-(4-fluorophenyl)-1-(propan-2-yl)- | 2.62 | 413 | — | — | — | A |
| | 1H-pyrazol-4-yl]pyridin-2-yl}-2-(2- | | | | | | |
| | methoxyethoxy)acetamide | | | | | | |
| 209 | N-{4-[3-(4-fluorophenyl)-1-(propan-2-yl)- | 2.42 | 383 | — | — | — | A |
| | 1H-pyrazol-4-yl]pyridin-2-yl}-2-hydroxy-2- | | | | | | |
| | methylpropanamide | | | | | | |
| 210 | N-{4-[3-(4-fluorophenyl)-1-(propan-2-yl)- | 1.76 | 385 | — | — | — | A |
| | 1H-pyrazol-4-yl]pyridin-2-yl}-2,3- | | | | | | |
| | dihydroxypropanamide | | | | | | |
| 211 | 1-ethyl-3-{4-[3-(4-fluorophenyl)-1-(propan-2- | 2.02 | 368 | — | — | — | A |
| | yl)-1H-pyrazol-4-yl]pyridin-2-yl}urea | | | | | | |
| 212 | N-{4-[3-(4-fluorophenyl)-1-(2- | 2.6 | 431 | — | — | — | A |
| | methoxyethyl)-1H-pyrazol-4-yl]pyridin-2- | | | | | | |
| | yl}-2-phenylacetamide | | | | | | |
| 213 | N-{4-[1-(2-fluorobenzyl)-3-(4-fluorophenyl)- | 2.69 | 419 | — | — | — | B |
| | 1H-pyrazol-4-yl]pyridin-2-yl}propanamide | | | | | | |
| 214 | N-{4-[1-(cyclopropylmethyl)-3-(4- | 2.20 | 366 | — | — | — | B |
| | fluorophenyl)-1H-pyrazol-4-yl]pyridin-2- | | | | | | |
| | yl}propanamide | | | | | | |
| 215 | N-{4-[1-(2,2-difluoroethyl)-3-(4- | 1.99 | 375 | — | — | — | B |
| | fluorophenyl)-1H-pyrazol-4-yl]pyridin-2- | | | | | | |
| | yl}propanamide | | | | | | |
| 216 | N-{4-[1-(2,2-difluoroethyl)-3-(4- | 2.27 | 391 | — | — | — | B |
| | fluorophenyl)-1H-pyrazol-4-yl]pyridin-2-yl}- | | | | | | |
| | 2-methoxyacetamide | | | | | | |
| 217 | N-{4-[1-(2,2-difluoroethyl)-3-(4- | 2.07 | 388 | — | — | — | B |
| | fluorophenyl)-1H-pyrazol-4-yl]pyridin-2- | | | | | | |
| | yl}cyclopropanecarboxamide | | | | | | |
| 218 | N-{4-[1-(2,2-difluoroethyl)-3-(4- | 2.29 | 389 | — | — | — | B |
| | fluorophenyl)-1H-pyrazol-4-yl]pyridin-2-yl}- | | | | | | |
| | 2-methylpropanamide | | | | | | |
| 219 | N-{4-[1-(2-chloroethyl)-3-(4-fluorophenyl)- | 2.01 | 373 | — | — | — | B |
| | 1H-pyrazol-4-yl]pyridin-2-yl}propanamide | | | | | | |
| 220 | N-{4-[1-(2-chloroethyl)-3-(4-fluorophenyl)- | 2.36 | 389 | — | — | — | B |
| | 1H-pyrazol-4-yl]pyridin-2-yl}-2- | | | | | | |
| | methoxyacetamide | | | | | | |
| 221 | N-{4-[1-(2-chloroethyl)-3-(4-fluorophenyl)- | 2.10 | 385 | — | — | — | B |
| | 1H-pyrazol-4-yl]pyridin-2- | | | | | | |
| | yl}cyclopropanecarboxamide | | | | | | |
| 222 | N-{4-[1-(2-chloroethyl)-3-(4-fluorophenyl)- | 2.33 | 387 | — | — | — | B |
| | 1H-pyrazol-4-yl]pyridin-2-yl}-2- | | | | | | |
| | methylpropanamide | | | | | | |
| 223 | N-{4-[1-(cyclopropylmethyl)-3-(4- | 2.61 | 382 | — | — | — | B |
| | fluorophenyl)-1H-pyrazol-4-yl]pyridin-2-yl}- | | | | | | |
| | 2-methoxyacetamide | | | | | | |
| 224 | N-{4-[1-(cyclopropylmethyl)-3-(4- | 2.29 | 377 | — | — | — | B |
| | fluorophenyl)-1H-pyrazol-4-yl]pyridin-2- | | | | | | |
| | yl}cyclopropanecarboxamide | | | | | | |
| 225 | N-{4-[1-(cyclopropylmethyl)-3-(4- | 2.54 | 380 | — | — | — | B |
| | fluorophenyl)-1H-pyrazol-4-yl]pyridin-2-yl}- | | | | | | |
| | 2-methylpropanamide | | | | | | |
| 226 | N-{4-[3-(4-fluorophenyl)-1-(propan-2-yloxy)- | 2.58 | 369 | — | — | — | A |
| | 1H-pyrazol-4-yl]pyridin-2-yl}propanamide | | | | | | |
| 227 | 4-[3-(4-fluorophenyl)-1-(propan-2-yloxy)-1H- | 1.58 | 298 | — | — | — | A |
| | pyrazol-4-yl]pyridine | | | | | | |
| 228 | 3-(4-fluorophenyl)-4-[3-(4-fluorophenyl)-1- | 2.96 | 390 | — | — | — | B |
| | (2-methylpropyl)-1H-pyrazol-4-yl]pyridine | | | | | | |
| 229 | 3-(3-fluorophenyl)-4-[3-(4-fluorophenyl)-1- | 3.13 | 390 | — | — | — | B |
| | (2-methylpropyl)-1H-pyrazol-4-yl]pyridine | | | | | | |
| 230 | N-{4-[3-(3-cyano-4-fluorophenyl)-1-(2- | 2.67 | 392 | — | — | — | B |
| | methylpropyl)-1H-pyrazol-4-yl]pyridin-2- | | | | | | |
| | yl}propanamide | | | | | | |
| 231 | 1-{4-[1-(2,2-difluoroethyl)-3-(4- | 1.91 | 390 | — | — | — | A |
| | fluorophenyl)-1H-pyrazol-4-yl]pyridin-2-yl]- | | | | | | |
| | 3-ethylurea | | | | | | |
| 232 | N-(4-{3-(4-fluorophenyl)-1-[1-(2- | 3.41 | 447 | — | — | — | B |
| | fluorophenyl)ethyl]-1H-pyrazol-4-yl}pyridin- | | | | | | |
| | 2-yl)-2-methylpropanamide | | | | | | |
| 233 | 3-{4-[3-(4-fluorophenyl)-1-(propan-2-yl)-1H- | 1.60 | 368 | — | — | — | A |
| | pyrazol-4-yl]pyridin-2-yl}-1,1-dimethylurea | | | | | | |
| 234 | N-{4-[1-(2,2-difluoroethyl)-3-(4- | 3.96 | 425 | — | — | — | B |
| | fluorophenyl)-1H-pyrazol-4-yl]pyridin-2- | | | | | | |
| | yl}propan-2-sulphonamide | | | | | | |
| 235 | N-{4-[1-(2,2-difluoroethyl)-3-(4- | 2.69 | 429 | — | — | — | B |
| | fluorophenyl)-1H-pyrazol-4-yl]pyridin-2- | | | | | | |
| | yl}thiophen-3-carboxamide | | | | | | |
| 236 | N-{4-[1-(2,2-difluoroethyl)-3-(4- | 2.82 | 429 | — | — | — | B |
| | fluorophenyl)-1H-pyrazol-4-yl]pyridin-2- | | | | | | |
| | yl}thiophen-2-carboxamide | | | | | | |
| 237 | N-{4-[1-(2,2-difluoroethyl)-3-(4- | 2.90 | 423 | — | — | — | B |
| | fluorophenyl)-1H-pyrazol-4-yl]pyridin-2- | | | | | | |
| | yl}benzamide | | | | | | |
| 238 | N-{4-[1-(2,2-difluoroethyl)-3-(4- | 2.63 | 403 | — | — | — | B |
| | fluorophenyl)-1H-pyrazol-4-yl]pyridin-2-yl}- | | | | | | |
| | 3-methylbutanamide | | | | | | |
| 239 | N-{4-[1-(2,2-difluoroethyl)-3-(4- | 2.62 | 403 | — | — | — | B |
| | fluorophenyl)-1H-pyrazol-4-yl]pyridin-2-yl}- | | | | | | |
| | 2-methylbutanamide | | | | | | |
| 240 | N-{4-[1-(2,2-difluoroethyl)-3-(4- | 2.42 | 401 | — | — | — | B |
| | fluorophenyl)-1H-pyrazol-4-yl]pyridin-2- | | | | | | |
| | yl}cyclobutancarboxamide | | | | | | |
| 241 | ethyl{4-[1-(2,2-difluoroethyl)-3-(4- | 2.45 | 391 | — | — | — | B |
| | fluorophenyl)-1H-pyrazol-4-yl]pyridin-2- | | | | | | |
| | yl}carbamate | | | | | | |
| 242 | N-{4-[1-(2,2-difluoroethyl)-3-(4- | 1.74 | 361 | — | — | — | B |
| | fluorophenyl)-1H-pyrazol-4-yl]pyridin-2- | | | | | | |
| | yl}acetamide | | | | | | |
| 243 | N-{4-[1-(2,2-difluoroethyl)-3-(4- | 2.18 | 405 | — | — | — | B |
| | fluorophenyl)-1H-pyrazol-4-yl]pyridin-2-yl}- | | | | | | |
| | 2-hydroxy-2-methylpropanamide | | | | | | |
| 244 | N-(4-{3-(4-fluorophenyl)-1-[1-(2- | 3.50 | 449 | — | — | — | B |
| | fluorophenyl)ethyl]-1H-pyrazol-4-yl}pyridin- | | | | | | |
| | 2-yl)-2-methoxyacetamide | | | | | | |
| 245 | N-(4-{1-[(2,2-dichlorocyclopropyl)methyl]-3- | 3.22 | 449 | — | — | — | B |
| | (4-fluorophenyl)-1H-pyrazol-4-yl}pyridin-2- | | | | | | |
| | yl)-2-methoxyacetamide | | | | | | |
| 246 | N-{4-[3-(3-cyano-4-fluorophenyl)-1-(2- | 2.98 | 408 | — | — | — | B |
| | methylpropyl)-1H-pyrazol-4-yl]pyridin-2-yl}- | | | | | | |
| | 2-methoxyacetamide | | | | | | |
| 247 | N-{4-[1-(2-fluorobenzyl)-3-(4-fluorophenyl)- | 3.09 | 435 | — | — | — | B |
| | 1H-pyrazol-4-yl]pyridin-2-yl}-2- | | | | | | |
| | methoxyacetamide | | | | | | |
| 248 | N-(4-{3-(4-fluorophenyl)-1-[1-(2- | 3.03 | 433 | — | — | — | B |
| | fluorophenyl)ethyl]-1H-pyrazol-4-yl}pyridin- | | | | | | |
| | 2-yl)propanamide | | | | | | |
| 249 | N-(4-{1-[(2,2-dichlorocyclopropyl)methyl]-3- | 2.80 | 433 | — | — | — | B |
| | (4-fluorophenyl)-1H-pyrazol-4-yl}pyridin-2- | | | | | | |
| | yl)propanamide | | | | | | |
| 250 | 2-cyclopropyl-N-(4-{3-(4-fluorophenyl)-1-[1- | 3.54 | 459 | — | — | — | B |
| | (2-fluorophenyl)ethyl]-1H-pyrazol-4- | | | | | | |
| | yl}pyridin-2-yl)acetamide | | | | | | |
| 251 | 2-cyclopropyl-N-(4-{1-[(2,2- | 3.30 | 459 | — | — | — | B |
| | dichlorocyclopropyl)methyl]-3-(4- | | | | | | |
| | fluorophenyl)-1H-pyrazol-4-yl}pyridin-2- | | | | | | |
| | yl)acetamide | | | | | | |
| 252 | N-{4-[3-(3-cyano-4-fluorophenyl)-1-(2- | 3.14 | 418 | — | — | — | B |
| | methylpropyl)-1H-pyrazol-4-yl]pyridin-2-yl}- | | | | | | |
| | 2-cyclopropylacetamide | | | | | | |
| 253 | 2-cyclopropyl-N-{4-[1-(2-fluorobenzyl)-3-(4- | 3.17 | 445 | — | — | — | B |
| | fluorophenyl)-1H-pyrazol-4-yl]pyridin-2- | | | | | | |
| | yl}acetamide | | | | | | |
| 254 | N-(4-{1-[(2,2-dichlorocyclopropyl)methyl]-3- | 3.17 | 447 | — | — | — | B |
| | (4-fluorophenyl)-1H-pyrazol-4-yl}pyridin-2- | | | | | | |
| | yl)-2-methylpropanamide | | | | | | |
| 255 | N-{4-[3-(3-cyano-4-fluorophenyl)-1-(2- | 3.03 | 406 | — | — | — | B |
| | methylpropyl)-1H-pyrazol-4-yl]pyridin-2-yl}- | | | | | | |
| | 2-methylpropanamide | | | | | | |
| 256 | N-{4-[1-(2-fluorobenzyl)-3-(4-fluorophenyl)- | 3.03 | 433 | — | — | — | B |
| | 1H-pyrazol-4-yl]pyridin-2-yl}-2- | | | | | | |
| | methylpropanamide | | | | | | |
| 257 | N-{4-[5-(difluoromethoxy)-3-(4-fluoro-2- | 2.44 | 421 | — | — | — | B |
| | hydroxyphenyl)-1-methyl-1H-pyrazol-4- | | | | | | |
| | yl]pyridin-2-yl}-2-methylpropanamide | | | | | | |
| 258 | N-{4-[5-(difluoromethoxy)-3-(4-fluoro-2- | 2.22 | 419 | — | — | — | B |
| | hydroxyphenyl)-1-methyl-1H-pyrazol-4- | | | | | | |
| | yl]pyridin-2-yl}cyclopropanecarboxamide | | | | | | |
| 259 | N-(4-{3-(4-fluorophenyl)-1-[1-(2- | 3.13 | 445 | — | — | — | B |
| | fluorophenyl)ethyl]-1H-pyrazol-4-yl}pyridin- | | | | | | |
| | 2-yl)cyclopropanecarboxamide | | | | | | |
| 260 | N-(4-{1-[(2,2-dichlorocyclopropyl)methyl]-3- | 2.67 | 445 | — | — | — | B |
| | (4-fluorophenyl)-1H-pyrazol-4-yl}pyridin-2- | | | | | | |
| | yl)cyclopropanecarboxamide | | | | | | |
| 261 | N-{4-[3-(3-cyano-4-fluorophenyl)-1-(2- | 2.77 | 404 | — | — | — | B |
| | methylpropyl)-1H-pyrazol-4-yl]pyridin-2- | | | | | | |
| | yl}cyclopropanecarboxamide | | | | | | |
| 262 | N-{4-[1-(2-fluorobenzyl)-3-(4-fluorophenyl)- | 2.77 | 431 | — | — | — | B |
| | 1H-pyrazol-4-yl]pyridin-2- | | | | | | |
| | yl}cyclopropanecarboxamide | | | | | | |
| 263 | ethyl(4-{3-(4-fluorophenyl)-1-[1-(2- | 3.65 | 449 | — | — | — | B |
| | fluorophenyl)ethyl]-1H-pyrazol-4-yl}pyridin- | | | | | | |
| | 2-yl)carbamate | | | | | | |
| 264 | ethyl(4-{1-[(2,2-dichlorocyclopropyl)methyl]- | 3.37 | 451 | — | — | — | B |
| | 3-(4-fluorophenyl)-1H-pyrazol-4-yl}pyridin- | | | | | | |
| | 2-yl)carbamate | | | | | | |
| 265 | ethyl{4-[3-(3-cyano-4-fluorophenyl)-1-(2- | 3.19 | 408 | — | — | — | B |
| | methylpropyl)-1H-pyrazol-4-yl]pyridin-2- | | | | | | |
| | yl}carbamate | | | | | | |
| 266 | ethyl{4-[1-(2-fluorobenzyl)-3-(4- | 3.24 | 435 | — | — | — | B |
| | fluorophenyl)-1H-pyrazol-4-yl]pyridin-2- | | | | | | |
| | yl}carbamate | | | | | | |
| 267 | ethyl{4-[3-(4-fluorophenyl)-1-(2- | 3.11 | 383 | — | — | — | B |
| | methylpropyl)-1H-pyrazol-4-yl]pyridin-2- | | | | | | |
| | yl}carbamate | | | | | | |
| 268 | ethyl{4-[1-(butan-2-yl)-3-(4-fluorophenyl)- | 3.08 | 383 | — | — | — | B |
| | 1H-pyrazol-4-yl]pyridin-2-yl}carbamate | | | | | | |
| 269 | N-{4-[1-(cyclopentyloxy)-3-(4-fluorophenyl)- | 3.35 | 407 | — | — | — | A |
| | 1H-pyrazol-4-yl]pyridin-2- | | | | | | |
| | yl}cyclopropanecarboxamide | | | | | | |
| 270 | N-{4-[1-(cyclopentyloxy)-3-(4-fluorophenyl)- | 3.65 | 409 | — | — | — | A |
| | 1H-pyrazol-4-yl]pyridin-2-yl}-2- | | | | | | |
| | methylpropanamide | | | | | | |
| 271 | N-{4-[3-(4-fluorophenyl)-1-(propan-2-yloxy)- | 2.73 | 381 | — | — | — | A |
| | 1H-pyrazol-4-yl]pyridin-2- | | | | | | |
| | yl}cyclopropanecarboxamide | | | | | | |
| 272 | N-{4-[3-(4-fluorophenyl)-1-(propan-2-yloxy)- | 3.01 | 383 | — | — | — | A |
| | 1H-pyrazol-4-yl]pyridin-2-yl}-2- | | | | | | |
| | methylpropanamide | | | | | | |
| 273 | N-(cyclopropylmethyl)-4-[3-(4-fluorophenyl)- | 2.55 | 366 | — | — | — | C |
| | 1-(2-methylpropyl)-1H-pyrazol-4- | | | | | | |
| | yl]pyrimidin-2-amine | | | | | | |
| 274 | methyl-2{3-(4-fluorophenyl)-4-[2- | 2.67 | 425 | — | — | — | A |
| | (propionylamino)pyridin-4-yl]-1H-1-pyrazol-1- | | | | | | |
| | yl}-3-methylbutanoate | | | | | | |
| 275 | 4-[1-(1,3-dioxolan-2-ylmethyl)-3-(4- | 1.02 | 326 | — | — | — | A |
| | fluorophenyl)-1H-pyrazol-4-yl]pyridine | | | | | | |
| 276 | N-{4-[1-(1,3-dioxolan-2-ylmethyl)-3-(4- | 1.99 | 411 | — | — | — | A |
| | fluorophenyl)-1H-pyrazol-4-yl]pyridin-2-yl}- | | | | | | |
| | 2-methylpropanamide | | | | | | |
| 277 | methyl-2-{3-(4-fluorophenyl)-4-[2- | 3.05 | 439 | — | — | — | A |
| | (isobutyrylamino)pyridin-4-yl]-1H-pyrazol-1- | | | | | | |
| | yl}-3-methylbutanoate | | | | | | |
| 278 | N-{4-[1-(1,3-dioxolan-2-ylmethyl)-3-(4- | 1.59 | 397 | — | — | — | B |
| | fluorophenyl)-1H-pyrazol-4-yl]pyridin-2- | | | | | | |
| | yl}propanamide | | | | | | |
| 279 | 4-[3-(4-fluorophenyl)-1-(2-methylpropyl)-1H- | 2.35 | 368 | — | — | — | C |
| | pyrazol-4-yl]-N-(oxetan-3-yl)pyrimidin-2- | | | | | | |
| | amine | | | | | | |
| 280 | 7-[3-(4-fluorophenyl)-1-isopropyl-1H- | 1.82 | 322 | — | — | — | C |
| | pyrazol-4-yl]-3H-imidazo[4,5-b]pyridine | | | | | | |
| 281 | N-cyclopropyl-4-[3-(4-fluorophenyl)-1-(2- | 2.41 | 352 | — | — | — | C |
| | methylpropyl)-1H-pyrazol-4-yl]pyrimidin-2- | | | | | | |
| | amine | | | | | | |
| 282 | 6-[3-(4-fluorophenyl)-1-isopropyl-1H- | 1.79 | 323 | — | — | — | B |
| | pyrazol-4-yl]-9H-purine | | | | | | |
| 283 | 4-[3-(4-fluorophenyl)-1-isopropyl-1H- | 2.05 | 362 | — | — | — | C |
| | pyrazol-4-yl]-2-(4-methyl-1H-imidazol-1- | | | | | | |
| | yl)pyridine | | | | | | |
| 284 | N-tert-butyl-4-[3-(4-fluorophenyl)-1-(2- | 2.94 | 368 | — | — | — | C |
| | methylpropyl)-1H-pyrazol-4-yl]pyrimidin-2- | | | | | | |
| | amine | | | | | | |
| 285 | 4-[3-(4-fluorophenyl)-1-(2-methylpropyl)-1H- | 2.39 | 384 | — | — | — | |
| | pyrazol-4-yl]-N-(1-methoxypropan-2- | | | | | | C |
| | yl)pyrimidin-2-amine | | | | | | |
| 286 | N-(butan-2-yl)-4-[3-(4-fluorophenyl)-1-(2- | 2.68 | 368 | — | — | — | C |
| | methylpropyl)-1H-pyrazol-4-yl]pyrimidin-2- | | | | | | |
| | amine | | | | | | |
| 287 | 4-[3-(4-fluorophenyl)-1-(2-methylpropyl)-1H- | 2.90 | 375 | — | — | — | C |
| | pyrazol-4-yl]-2-(methylsulphonyl)pyrimidine | | | | | | |
| 288 | 4-[3-(4-fluorophenyl)-1-isobutyl-1H-pyrazol- | 3.85 | 367 | — | — | — | B |
| | 4-yl]-7-methylthieno[3,2-d]pyrimidine | | | | | | |
| 289 | 4-[1-(cyclopropylmethyl)-3-(4-fluorophenyl)- | 3.45 | 365 | — | — | — | B |
| | 1H-pyrazol-4-yl]-7-methylthieno[3,2- | | | | | | |
| | d]pyrimidine | | | | | | |
| 290 | N-{4-[1-ethyl-3-(4-fluorophenyl)-1H-pyrazol- | 2.14 | 353 | — | — | — | B |
| | 4-yl]pyridin-2-yl}-2-methylpropanamide | | | | | | |
| 291 | 2-cyclopropyl-N-{4-[1-ethyl-3-(4- | 2.29 | 365 | — | — | — | B |
| | fluorophenyl)-1H-pyrazol-4-yl]pyridin-2- | | | | | | |
| | yl}acetamide | | | | | | |
| 292 | N-{4-[3-(4-fluorophenyl)-1-propyl-1H- | 2.15 | 353 | — | — | — | B |
| | pyrazol-4-yl]pyridin-2-yl}propanamide | | | | | | |
| 293 | N-{4-[3-(4-fluorophenyl)-1-propyl-1H- | 2.58 | 369 | — | — | — | B |
| | pyrazol-4-yl]pyridin-2-yl}-2- | | | | | | |
| | methoxyacetamide | | | | | | |
| 294 | N-{4-[3-(4-fluorophenyl)-1-propyl-1H- | 2.49 | 367 | — | — | — | B |
| | pyrazol-4-yl]pyridin-2-yl}-2- | | | | | | |
| | methylpropanamide | | | | | | |
| 295 | 2-cyclopropyl-N-{4-[3-(4-fluorophenyl)-1- | 2.66 | 379 | — | — | — | B |
| | propyl-1H-pyrazol-4-yl]pyridin-2- | | | | | | |
| | yl}acetamide | | | | | | |
| 296 | N-{4-[3-(4-fluorophenyl)-1-methyl-1H- | 1.56 | 325 | — | — | — | B |
| | pyrazol-4-yl]pyridin-2-yl}propanamide | | | | | | |
| 297 | N-{4-[3-(4-fluorophenyl)-1-methyl-1H- | 1.89 | 341 | — | — | — | B |
| | pyrazol-4-yl]pyridin-2-yl}-2- | | | | | | |
| | methoxyacetamide | | | | | | |
| 298 | N-{4-[3-(4-fluorophenyl)-1-methyl-1H- | 1.83 | 339 | — | — | — | B |
| | pyrazol-4-yl]pyridin-2-yl}-2- | | | | | | |
| | methylpropanamide | | | | | | |
| 299 | 2-cyclopropyl-N-{4-[3-(4-fluorophenyl)-1- | 1.98 | 351 | — | — | — | B |
| | methyl-1H-pyrazol-4-yl]pyridin-2- | | | | | | |
| | yl}acetamide | | | | | | |
| 300 | N-(4-{3-(4-fluorophenyl)-1-[2- | 2.12 | 385 | — | — | — | B |
| | (methylsulphanyl)ethyl]-1H-pyrazol-4- | | | | | | |
| | yl}pyridin-2-yl)propanamide | | | | | | |
| 301 | N-{4-[1-(1-cyclopropylethyl)-3-(4- | 2.54 | 379 | — | — | — | B |
| | fluorophenyl)-1H-pyrazol-4-yl]pyridin-2- | | | | | | |
| | yl}propanamide | | | | | | |
| 302 | N-{4-[1-(3-cyanobenzyl)-3-(4-fluorophenyl)- | 2.38 | 426 | — | — | — | B |
| | 1H-pyrazol-4-yl]pyridin-2-yl}propanamide | | | | | | |
| 303 | N-{4-[1-(2-cyanoethyl)-3-(4-fluorophenyl)- | 1.63 | 364 | — | — | — | B |
| | 1H-pyrazol-4-yl]pyridin-2-yl}propanamide | | | | | | |
| 304 | N-{4-[3-(4-fluorophenyl)-1-isopropyl-5- | 4.07 | 421 | — | — | — | B |
| | (trifluoromethyl)-1H-pyrazol-4-yl]pyridin-2- | | | | | | |
| | yl}propanamide | | | | | | |
| 305 | 4-[1-(1-cyclopropylethyl)-3-(4-fluorophenyl)- | 1.59 | 308 | — | — | — | B |
| | 1H-pyrazol-4-yl]pyridine | | | | | | |
| 306 | N-(4-{3-(4-fluorophenyl)-1-[2- | 2.44 | 399 | — | — | — | B |
| | (methylsulphanyl)ethyl]-1H-pyrazol-4- | | | | | | |
| | yl}pyridin-2-yl)-2-methylpropanamide | | | | | | |
| 307 | N-{4-[1-(1-cyclopropylethyl)-3-(4- | 2.90 | 393 | — | — | — | B |
| | fluorophenyl)-1H-pyrazol-4-yl]pyridin-2-yl}- | | | | | | |
| | 2-methylpropanamide | | | | | | |
| 308 | N-{4-[1-(3-cyanobenzyl)-3-(4-fluorophenyl)- | 2.69 | 440 | — | — | — | B |
| | 1H-pyrazol-4-yl]pyridin-2-yl}-2- | | | | | | |
| | methylpropanamide | | | | | | |
| 309 | N-{4-[1-(2-cyanoethyl)-3-(4-fluorophenyl)- | 1.91 | 378 | — | — | — | B |
| | 1H-pyrazol-4-yl]pyridin-2-yl}-2- | | | | | | |
| | methylpropanamide | | | | | | |
| 310 | 4-[3-(4-fluorophenyl)-1-isopropyl-1H- | 1.61 | 322 | — | — | — | B |
| | pyrazol-4-yl]-7H-pyrrolo[2,3-d]pyrimidine | | | | | | |
| 311 | 4-[3-(4-fluorophenyl)-1-isopropyl-1H- | 1.29 | 338 | — | — | — | B |
| | pyrazol-4-yl]-5,6,7,8-tetrahydropyrido[2,3- | | | | | | |
| | d]pyrimidine | | | | | | |
| 312 | N-{4-[1-(1-cyanopropan-2-yl)-3-(4- | 1.87 | 378 | — | — | — | B |
| | fluorophenyl)-1H-pyrazol-4-yl]pyridin-2- | | | | | | |
| | yl}propanamide | | | | | | |
| 313 | N-{4-[1-(1-cyanopropan-2-yl)-3-(4- | 2.17 | 394 | — | — | — | B |
| | fluorophenyl)-1H-pyrazol-4-yl]pyridin-2-yl}- | | | | | | |
| | 2-methoxyacetamide | | | | | | |
| 314 | N-{4-[1-(1-cyanopropan-2-yl)-3-(4- | 1.29 | 392 | — | — | — | B |
| | fluorophenyl)-1H-pyrazol-4-yl]pyridin-2-yl}- | | | | | | |
| | 2-methylpropanamide | | | | | | |
| 315 | N-{4-[1-(1-cyanopropan-2-yl)-3-(4- | 2.28 | 404 | — | — | — | B |
| | fluorophenyl)-1H-pyrazol-4-yl]pyridin-2-yl}- | | | | | | |
| | 2-cyclopropylacetamide | | | | | | |
| 316 | N-{4-[5-(difluoromethoxy)-3-(4- | 2.33 | 391 | — | — | — | B |
| | fluorophenyl)-1-methyl-1H-pyrazol-4- | | | | | | |
| | yl]pyridin-2-yl}propanamide | | | | | | |
| 317 | 4-[1-(cyclopropylmethyl)-3-(4-fluorophenyl)- | 2.33 | 405 | — | — | — | B |
| | 1H-pyrazol-4-yl]-N-(1,1,1-trifluoropropan-2- | | | | | | |
| | yl)pyridin-2-amine | | | | | | |
| 318 | 4-[3-(4-fluorophenyl)-1-isopropyl-1H- | 2.26 | 393 | — | — | — | B |
| | pyrazol-4-yl]-N-(1,1,1-trifluoropropan-2- | | | | | | |
| | yl)pyridin-2-amine | | | | | | |
| 319 | 4-[3-(4-fluorophenyl)-1-methyl-1H-pyrazol- | 1.73 | 365 | — | — | — | B |
| | 4-yl]-N-(1,1,1-trifluoropropan-2-yl)pyridin-2- | | | | | | |
| | amine | | | | | | |
| 320 | N-{4-[1-cyclopropyl-3-(4-fluorophenyl)-1H- | 1.97 | 351 | — | — | — | B |
| | pyrazol-4-yl]pyridin-2-yl}propanamide | | | | | | |
| 321 | N-{4-[1-cyclopropyl-3-(4-fluorophenyl)-1H- | 2.37 | 367 | — | — | — | B |
| | pyrazol-4-yl]pyridin-2-yl}-2- | | | | | | |
| | methoxyacetamide | | | | | | |
| 322 | N-{4-[1-cyclopropyl-3-(4-fluorophenyl)-1H- | 2.32 | 365 | — | — | — | B |
| | pyrazol-4-yl]pyridin-2-yl}-2- | | | | | | |
| | methylpropanamide | | | | | | |
| 323 | 2-cyclopropyl-N-{4-[1-cyclopropyl-3-(4- | 2.48 | 377 | — | — | — | B |
| | fluorophenyl)-1H-pyrazol-4-yl]pyridin-2- | | | | | | |
| | yl}acetamide | | | | | | |
| 324 | 4-[3-(4-fluorophenyl)-1-isopropyl-1H- | 3.88 | 473 | — | — | — | B |
| | pyrazol-4-yl]-N-[2,2,2-trifluoro-1-(4- | | | | | | |
| | fluorophenyl)ethyl]pyridin-2-amine | | | | | | |
| 325 | 4-[3-(4-fluorophenyl)-1-isopropyl-1H- | 3.66 | 455 | — | — | — | B |
| | pyrazol-4-yl]-N-(2,2,2-trifluoro-1- | | | | | | |
| | phenylethyl)pyridin-2-amine | | | | | | |
| 326 | N-{4-[1-(2-ethoxyethyl)-3-(4-fluorophenyl)- | 2.01 | 383 | — | — | — | B |
| | 1H-pyrazol-4-yl]pyridin-2-yl}propanamide | | | | | | |
| 327 | N-{4-[1-(2-ethoxyethyl)-3-(4-fluorophenyl)- | 2.38 | 399 | — | — | — | B |
| | 1H-pyrazol-4-yl]pyridin-2-yl}-2- | | | | | | |
| | methoxyacetamide | | | | | | |
| 328 | N-{4-[1-(2-ethoxyethyl)-3-(4-fluorophenyl)- | 2.32 | 397 | — | — | — | B |
| | 1H-pyrazol-4-yl]pyridin-2-yl}-2- | | | | | | |
| | methylpropanamide | | | | | | |
| 329 | 2-cyclopropyl-N-{4-[1-(2-ethoxyethyl)-3-(4- | 2.48 | 409 | — | — | — | B |
| | fluorophenyl)-1H-pyrazol-4-yl]pyridin-2- | | | | | | |
| | yl}acetamide | | | | | | |
| 330 | methyl-6-({4-[1-ethyl-3-(4-fluorophenyl)-1H- | 2.16 | 425 | — | — | — | A |
| | pyrazol-4-yl]pyridin-2-yl}amino)-6- | | | | | | |
| | oxohexanoate | | | | | | |
| 331 | methyl-5-({4-[1-ethyl-3-(4-fluorophenyl)-1H- | 2.02 | 411 | — | — | — | A |
| | pyrazol-4-yl]pyridin-2-yl}amino)-5- | | | | | | |
| | oxopentanoate | | | | | | |
| 332 | ethyl-3-ethyl-5-({4-[1-ethyl-3-(4- | 2.90 | 453 | — | — | — | A |
| | fluorophenyl)-1H-pyrazol-4-yl]pyridin-2- | | | | | | |
| | yl}amino)-5-oxopentanoate | | | | | | |
| 333 | 3-(difluoromethyl)-N-{4-[1-ethyl-3-(4- | 2.47 | 441 | — | — | — | A |
| | fluorophenyl)-1H-pyrazol-4-yl]pyridin-2-yl}- | | | | | | |
| | 1-methyl-1H-pyrazol-4-carboxamide | | | | | | |
| 334 | 2-(difluoromethyl)-N-{4-[1-ethyl-3-(4- | 3.27 | 443 | — | — | — | A |
| | fluorophenyl)-1H-pyrazol-4-yl]pyridin-2- | | | | | | |
| | yl}thiophen-3-carboxamide | | | | | | |
| 335 | N-{4-[1-ethyl-3-(4-fluorophenyl)-1H-pyrazol- | 2.72 | 409 | — | — | — | A |
| | 4-yl]pyridin-2-yl}-4-methyl-1,2,3-thiadiazol- | | | | | | |
| | 5-carboxamide | | | | | | |
| 336 | 3-(4-chlorophenyl)-N-{4-[1-ethyl-3-(4- | 3.32 | 449 | — | — | — | A |
| | fluorophenyl)-1H-pyrazol-4-yl]pyridin-2- | | | | | | |
| | yl}propanamide | | | | | | |
| 337 | (1R,2R)-N-{4-[1-ethyl-3-(4-fluorophenyl)- | 3.06 | 427 | — | — | — | A |
| | 1H-pyrazol-4-yl]pyridin-2-yl}-2- | | | | | | |
| | phenylcyclopropanecarboxamide | | | | | | |
| 338 | 2-cyclopentyl-N-{4-[1-ethyl-3-(4- | 3.87 | 393 | — | — | — | A |
| | fluorophenyl)-1H-pyrazol-4-yl]pyridin-2- | | | | | | |
| | yl}acetamide | | | | | | |
| 339 | N{4-[1-ethyl-3-(4-fluorophenyl)-1H-pyrazol- | 3.47 | 367 | — | — | — | A |
| | 4-yl]pyridin-2-yl}-3-methylbutanamide | | | | | | |
| 340 | N-{4-[1-ethyl-3-(4-fluorophenyl)-1H-pyrazol- | 2.42 | 367 | — | — | — | A |
| | 4-yl]pyridin-2-yl}-2-methylbutanamide | | | | | | |
| 341 | N-{4-[1-ethyl-3-(4-fluorophenyl)-1H-pyrazol- | 2.16 | 365 | — | — | — | A |
| | 4-yl]pyridin-2-yl}-2- | | | | | | |
| | methylcyclopropanecarboxamide | | | | | | |
| 342 | N-{4-[1-ethyl-3-(4-fluorophenyl)-1H-pyrazol- | 1.57 | 325 | — | — | — | A |
| | 4-yl]pyridin-2-yl}acetamide | | | | | | |
| 343 | N-{4-[3-(4-fluorophenyl)-1-methyl-1H- | 2.54 | 401 | — | — | — | A |
| | pyrazol-4-yl]pyridin-2-yl}-3- | | | | | | |
| | phenylpropanamide | | | | | | |
| 344 | 4-fluoro-N-{4-[3-(4-fluorophenyl)-1-methyl- | 2.61 | 391 | — | — | — | A |
| | 1H-pyrazol-4-yl]pyridin-2-yl}benzamide | | | | | | |
| 345 | N-{4-[3-(4-fluorophenyl)-1-methyl-1H- | 1.96 | 385 | — | — | — | A |
| | pyrazol-4-yl]pyridin-2-yl}-2-(2- | | | | | | |
| | methoxyethoxy)acetamide | | | | | | |
| 346 | N-{4-[3-(4-fluorophenyl)-1-methyl-1H- | 2.42 | 373 | — | — | — | A |
| | pyrazol-4-yl]pyridin-2-yl}benzamide | | | | | | |
| 347 | N-{4-[3-(4-fluorophenyl)-1-methyl-1H- | 2.12 | 353 | — | — | — | A |
| | pyrazol-4-yl]pyridin-2-yl}-3- | | | | | | |
| | methylbutanamide | | | | | | |
| 348 | N-{4-[3-(4-fluorophenyl)-1-methyl-1H- | 2.14 | 353 | — | — | — | A |
| | pyrazol-4-yl]pyridin-2-yl}-2- | | | | | | |
| | methylbutanamide | | | | | | |
| 349 | N-{4-[3-(4-fluorophenyl)-1-methyl-1H- | 1.93 | 351 | — | — | — | A |
| | pyrazol-4-yl]pyridin-2- | | | | | | |
| | yl}cyclobutancarboxamide | | | | | | |
| 350 | N-{4-[3-(4-fluorophenyl)-1-methyl-1H- | 1.32 | 311 | — | — | — | A |
| | pyrazol-4-yl]pyridin-2-yl}acetamide | | | | | | |
| 351 | N-{4-[1-ethyl-3-(4-fluorophenyl)-1H-pyrazol- | 1.99 | 354 | — | — | — | A |
| | 4-yl]pyridin-2-yl}ethandiamide | | | | | | |
| 352 | (2S)-N-{4-[1-ethyl-3-(4-fluorophenyl)-1H- | 1.83 | 354 | — | — | — | A |
| | pyrazol-4-yl]pyridin-2-yl}-2- | | | | | | |
| | hydroxypropanamide | | | | | | |
| 353 | (2S)-N-{4-[3-(4-fluorophenyl)-1-methyl-1H- | 1.57 | 341 | — | — | — | A |
| | pyrazol-4-yl]pyridin-2-yl}-2- | | | | | | |
| | hydroxypropanamide | | | | | | |
| 354 | N-{4-[3-(4-fluorophenyl)-1-(propan-2-yl)- | 2.37 | 370 | — | — | — | C |
| | 1H-pyrazol-4-yl]pyrimidin-2-yl}-2- | | | | | | |
| | methoxyacetamide | | | | | | |
| 355 | N-{4-[3-(4-fluorophenyl)-1-(propan-2-yl)- | 2.55 | 368 | — | — | — | C |
| | 1H-pyrazol-4-yl]pyrimidin-2-yl}-2- | | | | | | |
| | methylpropanamide | | | | | | |
| 356 | N-{4-[3-(4-fluorophenyl)-1-(propan-2-yl)- | 2.49 | 366 | — | — | — | C |
| | 1H-pyrazol-4-yl]pyrimidin-2- | | | | | | |
| | yl}cyclopropanecarboxamide | | | | | | |
| 357 | N-{4-[3-(4-fluorophenyl)-1-(2-methylpropyl)- | 2.44 | 354 | — | — | — | C |
| | 1H-pyrazol-4-yl]pyrimidin-2-yl}acetamide | | | | | | |
| 358 | 4-[3-(4-fluorophenyl)-1-(2-methoxyethyl)- | 1.79 | 356 | 1.76 | 356 | 76:23 | C |
| | 1H-pyrazol-4-yl]-N-(propan-2-yl)pyrimidin- | | | | | | |
| | 2-amine | | | | | | |
| 359 | 4-[1-(2-chloroethyl)-3-(4-fluorophenyl)-1H- | 2.05 | 360 | 2.01 | 360 | 70:30 | C |
| | pyrazol-4-yl]-N-(propan-2-yl)pyrimidin-2- | | | | | | |
| | amine | | | | | | |
| 360 | 4-[1-(butan-2-yl)-3-(4-fluorophenyl)-1H- | 2.41 | 354 | — | — | — | C |
| | pyrazol-4-yl]-N-(propan-2-yl)pyrimidin-2- | | | | | | |
| | amine | | | | | | |
| 361 | 4-[3-(4-fluorophenyl)-1-methyl-1H-pyrazol- | 1.65 | 312 | — | — | — | C |
| | 4-yl]-N-(propan-2-yl)pyrimidin-2-amine | | | | | | |
| 362 | 3-{3-(4-fluorophenyl)-4-[2-(propan-2- | 1.74 | 351 | — | — | — | C |
| | ylamino)pyrimidin-4-yl]-1H-pyrazol-1- | | | | | | |
| | yl}propanonitrile | | | | | | |
| 363 | 4-[3-(4-fluorophenyl)-1-(prop-2-yn-1-yl)-1H- | 1.99 | 336 | — | — | — | C |
| | pyrazol-4-yl]-N-(propan-2-yl)pyrimidin-2- | | | | | | |
| | amine | | | | | | |
| 364 | 4-[1-(2,2-difluoroethyl)-3-(4-fluorophenyl)- | 2.05 | 362 | — | — | — | C |
| | 1H-pyrazol-4-yl]-N-(propan-2-yl)pyrimidin- | | | | | | |
| | 2-amine | | | | | | |
| 365 | N-{4-[3-(4-fluorophenyl)-1-(2-methylpropyl)- | 2.70 | 368 | — | — | — | C |
| | 1H-pyrazol-4-yl]pyrimidin-2-yl}propanamide | | | | | | |
| 366 | 4-[3-(4-fluorophenyl)-1-(propan-2-yl)-1H- | 1.54 | 298 | — | — | — | C |
| | pyrazol-4-yl]pyrimidin-2-amine | | | | | | |
| 367 | 4-[1-(2,2-difluoroethyl)-3-(4-fluorophenyl)-5- | 1.49 | 318 | 1.43 | 318 | 66:23 | C |
| | methyl-1H-pyrazol-4-yl]pyridine | | | | | | |
| 368 | 4-[3-(4-fluorophenyl)-5-methyl-1-(2 | 2.07 | 310 | 1.98 | 310 | 64:28 | C |
| | methylpropyl)-1H-pyrazol-4-yl]pyridine | | | | | | |
| 369 | N-benzyl-4-[3-(4-fluorophenyl)-1-(propan-2- | 3.31 | 388 | — | — | — | C |
| | yl)-1H-pyrazol-4-yl]pyrimidin-2-amine | | | | | | |
| 370 | 4-[1-ethyl-3-(4-fluorophenyl)-1H-pyrazol-4- | 1.91 | 326 | — | — | — | C |
| | yl]-N-(propan-2-yl)pyrimidin-2-amine | | | | | | |
| 371 | 4-[3-(4-fluorophenyl)-1-(propan-2-yl)-1H- | 2.92 | 340 | — | — | — | C |
| | pyrazol-4-yl]-N-(propan-2-yl)pyrimidin-2- | | | | | | |
| | amine | | | | | | |
| 372 | N-(1-cyclopropylethyl)-4-[3-(4- | 2.73 | 380 | — | — | — | C |
| | fluorophenyl)-1-(2-methylpropyl)-1H- | | | | | | |
| | pyrazol-4-yl]pyrimidin-2-amine | | | | | | |

¹The stated logP value is the value for the stated pure compound or, if a mixture is present, for the main isomer (1-substituted 3-aryl-1H-pyrazole).
²The stated mass is the peak of the isotope pattern of the [M + H]+ ion with the highest intensity; if the [M H]− ion was detected, the mass value is marked with a 6.
³A stated logP value is the value for the minor isomer (1-substituted 5-aryl-1H-pyrazole) present in an isomer mixture (based on the pyrazole substitution).
⁴The isomer ratio is based on a mixture of various pyrazoles obtained in some cases (the main isomer is the 1-substituted 3-aryl-1H-pyrazole, the minor isomer is the corresponding 1-substituted 5-aryl-1H-pyrazole). If no isomer ratio was stated, only the main isomer is present. The isomer ratio is based on the areas in the UV chromatogram (at a wavelength of 210 nm) of the LC-MS spectrum measured with method A, B or C.
⁵In the determination of the logP values, the methods described below were used.
⁶The stated mass is the peak of the isotope pattern of the [M − H]− ion with the highest intensity.

Method A Note on the determination of the log P values and mass detection: The stated log P values were determined in accordance with EEC-Directive 79/831 Annex V.A8 by HPLC (High Performance Liquid Chromatography) on a reverse phase column (C18). Agilent 1100 LC system; 50*4.6 Zorbax Eclipse Plus C18 1.8 micron; eluent A: acetonitrile (0.1% formic acid); eluent B: water (0.09% formic acid); linear gradient from 10% acetonitrile to 95% acetonitrile in 4.25 mins, then 95% acetonitrile for a further 1.25 mins; oven temperature 55° C.; flow rate: 2.0 mL/min. The mass detection was effected with an Agilend MSD system.

Method B Note on the determination of the log P values and mass detection: The stated log P values were determined in accordance with EEC-Directive 79/831 Annex V.A8 by HPLC (High Performance Liquid Chromatography) on a reverse phase column (C18). HP1100; 50*4.6 Zorbax Eclipse Plus C18 1.8 micron; eluent A: acetonitrile (0.1% formic acid); eluent B: water (0.08% formic acid); linear gradient from 5% acetonitrile to 95% acetonitrile in 1.70 min, then 95% acetonitrile for a further 1.00 min; oven temperature 55° C.; flow rate: 2.0 mL/min. The mass detection was effected with the Micronass ZQ2000 mass detector from Waters.

Method C Note on the determination of the log P values and mass detection: The stated log P values were determined in accordance with EEC-Directive 79/831 Annex V.A8 by UPLC (Ultra Performance Liquid Chromatography) on a reverse phase column (C18). HP1100; 50*2.1 Zorbax Eclipse Plus C18 1.8 micron; eluent A: acetonitrile (0.09% formic acid); eluent B: water (0.1% formic acid); linear gradient from 10% A to 95% A in 3.25 min; oven temperature 40° C.; flow rate: 0.8 mL/min. The mass detection was effected with the LCT Premier or SQD mass detector from Waters.

Calibration was performed with unbranched alkan-2-ones (with 3 to 16 carbon atoms), whose log P values are known (determination of the log P values on the basis of the retention times by linear interpolation between two successive alkanones).

The lambda-max values were determined on the basis of the UV spectra from 200 nm to 400 nm in the maxima of the chromatographic signals.

### USE EXAMPLES

### Example A

### In Vivo Test on Peronospora Parasitica (Downy Mildew on White Cabbage)

An aqueous suspension of the active substance was prepared by homogenization of a mixture of acetone/Tween (dispersant)/dimethyl sulphoxide (DMSO) and subsequent dilution with water to the desired concentration. Cabbage plants (variety: Eminence) are sown in cultivation dishes on a peat-pozzolanic earth substrate (50/50) at 18-20° C. and sprayed at the cotyledon stage with the aqueous suspension described above. As a control, plants are sprayed with an aqueous solution with no active substance. After 24 hours, the plants are inoculated by spraying with an aqueous suspension of Peronospora parasitica spores (50,000 spores per ml). The spores are derived from infected plants. The inoculated cabbage plants are incubated for 5 days at ca. 20° C. in a moist atmosphere. After 5 days, they are scored in comparison with the control plants. Under these conditions at a dosage of 500 ppm good (70% activity level) or complete inhibition was observed for the following compounds:

| Bsp. | |
|---|---|
| Verbindung | % |
| Nr. | Wirkungsgrad |
| 1 | 100 |
| 2 | 85 |
| 3 | 97 |
| 6 | 70 |
| 10 | 85 |
| 15 | 75 |
| 19 | 100 |
| 21 | 100 |
| 22 | 97 |
| 23 | 100 |
| 28 | 97 |
| 32 | 79 |
| 33 | 93 |
| 40 | 98 |
| 41 | 72 |
| 44 | 78 |
| 46 | 94 |
| 81 | 89 |
| 84 | 100 |
| 85 | 100 |
| 86 | 100 |
| 88 | 83 |
| 91 | 89 |
| 102 | 70 |
| 103 | 100 |
| 106 | 100 |
| 113 | 73 |
| 116 | 97 |
| 117 | 93 |
| 120 | 85 |
| 122 | 93 |
| 123 | 83 |
| 126 | 89 |
| 127 | 78 |
| 129 | 78 |
| 133 | 89 |
| 134 | 94 |
| 136 | 94 |
| 137 | 72 |
| 140 | 78 |
| 141 | 89 |
| 150 | 100 |
| 171 | 91 |

### Example B

### In Vivo Test on Botrytis Cinerea (Grey Mould on Cucumbers)

An aqueous suspension of the active substance was prepared by homogenization of a mixture of acetone/Tween/dimethyl sulphoxide and subsequent dilution with water to the desired concentration. Cucumber plants (variety: Vert petit de Paris) are sown in cultivation dishes on a peat-pozzolanic earth substrate (50/50) at 18-20° C. and sprayed at the cotyledon stage Z11 with the aqueous suspension described above. As a control, plants are sprayed with an aqueous solution with no active substance. After 24 hours, the plants are inoculated by dropwise application of an aqueous suspension of Botrytis cinerea spores (150,000 spores per ml) onto the leaf surface. The spores are derived from a 15 day-old culture which are suspended in the following nutrient solution:
20 g/l gelatine
50 g/l D-fructose
2 g/l NH₄NO₃
1 g/l KH₂PO₄

The inoculated cucumber plants are kept for 5-7 days in a climatic chamber at 15-11° C. (day/night) and 80% atmospheric humidity. After 5-7 days they are scored in comparison with the control plants. Under these conditions at a dosage of 500 ppm good (70% activity level) or complete inhibition was observed for the following compounds:

| Bsp. | |
|---|---|
| Verbindung Nr. | % Wirkungsgrad |
| 1 | 100 |
| 2 | 74 |
| 8 | 94 |
| 10 | 73 |
| 13 | 100 |
| 14 | 100 |
| 17 | 83 |
| 40 | 100 |
| 44 | 71 |
| 84 | 100 |
| 85 | 100 |
| 86 | 100 |
| 171 | 71 |

### Example C

### In Vivo Test on Alternaria Brassicae (Leaf Spot Disease on Radishes)

An aqueous suspension of the active substance was prepared by homogenization of a mixture of acetone/Tween/dimethyl sulphoxide and subsequent dilution with water to the desired concentration. Radish plants (variety: Pernot) are sown in cultivation dishes on a peat-pozzolanic earth substrate (50/50) at 18-20° C. and sprayed at the cotyledon stage with the aqueous suspension described above. As a control, plants are sprayed with an aqueous solution with no active substance. After 24 hours, the plants are inoculated by spraying with an aqueous suspension of Alternaria brassicae spores (40,000 spores per ml). The spores are derived from a 12 to 13 day-old culture. The inoculated radish plants are incubated for 6-7 days at ca. 18° C. in a humid atmosphere. After 6-7 days they are scored in comparison with the control plants. Under these conditions at a dosage of 500 ppm good (70% activity level) or complete inhibition was observed for the following compounds:

| Ex. Compound No. | % Activity level |
|---|---|
| 1 | 94 |
| 2 | 77 |
| 3 | 88 |
| 10 | 85 |
| 84 | 75 |
| 86 | 75 |

### Example D

### In Vivo Test on Sphaerotheca Fuliginea (Powdery Mildew on Cucumber)

An aqueous suspension of the active substance was prepared by homogenization of a mixture of acetone/Tween/dimethyl sulphoxide and subsequent dilution with water to the desired concentration. Cucumber plants (variety: Vert petit de Paris) are sown in cultivation dishes on a peat-pozzolanic earth substrate (50/50) at 20/23° C. and at the cotyledon stage Z10 sprayed with the aqueous suspension described above. As a control, plants are sprayed with an aqueous solution with no active substance. After 24 hours the plants are inoculated by spraying with an aqueous suspension of Sphaerotheca fuliginea spores (100,000 spores per ml). The spores are derived from a contaminated plant. The inoculated cucumber plants are incubated at ca. 20/25° C. at a relative atmospheric humidity of 60/70%. After 12 days they are scored in comparison with the control plants. Under these conditions at a dosage of 500 ppm good (70% activity level) or complete inhibition was observed for the following compounds:

| Bsp. | |
|---|---|
| Verbindung | % |
| Nr. | Wirkungsgrad |
| 1 | 100 |
| 2 | 93 |
| 3 | 93 |
| 6 | 95 |
| 9 | 75 |
| 10 | 80 |
| 11 | 93 |
| 12 | 95 |
| 13 | 90 |
| 14 | 80 |
| 15 | 87 |
| 17 | 80 |
| 22 | 71 |
| 24 | 88 |
| 26 | 80 |
| 27 | 73 |
| 33 | 71 |
| 37 | 78 |
| 40 | 100 |
| 41 | 98 |
| 44 | 98 |
| 46 | 81 |
| 53 | 98 |
| 81 | 77 |
| 84 | 100 |
| 85 | 100 |
| 86 | 100 |
| 88 | 86 |
| 91 | 88 |
| 92 | 93 |
| 102 | 71 |
| 103 | 100 |
| 105 | 82 |
| 106 | 100 |
| 107 | 91 |
| 108 | 82 |
| 110 | 79 |
| 113 | 82 |
| 114 | 79 |
| 116 | 89 |
| 117 | 93 |
| 122 | 99 |
| 123 | 86 |
| 126 | 85 |
| 127 | 85 |
| 129 | 92 |
| 132 | 82 |
| 133 | 95 |
| 133 | 91 |
| 134 | 85 |
| 136 | 91 |
| 138 | 77 |
| 141 | 77 |
| 150 | 96 |
| 171 | 100 |

### Example E

### In Vivo Test on Pyrenophora Teres (Barley Net Blotch Disease)

An aqueous suspension of the active substance was prepared by homogenization of a mixture of acetone/Tween/dimethyl sulphoxide and subsequent dilution with water to the desired concentration. Barley plants (variety: Plaisant) are sown in cultivation dishes on a peat-pozzolanic earth substrate (50/50) at 12° C. and sprayed at the first-leaf stage (10 cm size) with the aqueous suspension described above. As a control, plants are sprayed with an aqueous solution with no active substance. After 24 hours the plants are inoculated by spraying with an aqueous suspension of Pyrenophora teres spores (12,000 spores per ml). The spores are derived from a 12 day-old culture. The inoculated barley plants are first incubated for 24 hours at ca. 20° C. and 100% relative atmospheric humidity and then for 12 days at 80% relative atmospheric humidity. After 12 days they are scored in comparison with the control plants. Under these conditions at a dosage of 500 ppm good (70% activity level) or complete inhibition was observed for the following compounds:

| Bsp. | |
|---|---|
| Verbindung | % |
| Nr. | Wirkungsgrad |
| 1 | 96 |
| 7 | 73 |
| 19 | 83 |
| 21 | 73 |
| 24 | 92 |
| 85 | 75 |
| 86 | 81 |
| 136 | 71 |
| 140 | 71 |
| 150 | 86 |

### Example F

### In Vivo Test on Puccinia Recondita (Wheat Brown Rust)

An aqueous suspension of the active substance was prepared by homogenization of a mixture of acetone/Tween/dimethyl sulphoxide and subsequent dilution with water to the desired concentration. Wheat plants (variety: Scipion) are sown in cultivation dishes on a peat-pozzolanic earth substrate (50/50) at 12° C. and sprayed at the first-leaf stage (10 cm size) with the aqueous suspension described above. As a control, plants are sprayed with an aqueous solution with no active substance. After 24 hours the plants are inoculated by spraying with an aqueous suspension of Puccinia recondita spores (100,000 spores per ml). The spores are derived from a 10 day-old infected wheat crop and are suspended in water with 2.5 ml/l Tween. The inoculated wheat plants are first incubated for 24 hours at 20° C. and 100% relative atmospheric humidity and then for 10 days at 20° C. and 70% relative atmospheric humidity. After 10 days they are scored in comparison with the control plants. Under these conditions at a dosage of 500 ppm good (70% activity level) or complete inhibition was observed for the following compounds:

| Bsp. | |
|---|---|
| Verbindung | % |
| Nr. | Wirkungsgrad |
| 1 | 89 |
| 40 | 86 |
| 44 | 71 |
| 84 | 78 |
| 91 | 94 |
| 93 | 83 |
| 126 | 75 |
| 171 | 94 |

### Example G

### In Vivo Test on Mycosphaerella Graminicola (Wheat Leaf Blotch Disease)

An aqueous suspension of the active substance was prepared by homogenization of a mixture of acetone/Tween/dimethyl sulphoxide and subsequent dilution with water to the desired concentration. Wheat plants (variety: Scipion) are sown in cultivation dishes on a peat-pozzolanic earth substrate (50/50) at 12° C. and sprayed at the first-leaf stage (10 cm size) with the aqueous suspension described above. As a control, plants are sprayed with an aqueous solution with no active substance. After 24 hours the plants are inoculated by spraying with an aqueous suspension of Mycosphaerella graminicola spores (500,000 spores per ml). The spores are derived from a 7 day-old culture. The inoculated wheat plants are first incubated for 72 hours at 18° C. and 100% relative atmospheric humidity and then for 21 to 28 days at 90% relative atmospheric humidity. After 21 to 28 days they are scored in comparison with the control plants. Under these conditions at a dosage of 500 ppm good (70% activity level) or complete inhibition was observed for the following compounds:

| Bsp. | |
|---|---|
| Verbindung | % |
| Nr. | Wirkungsgrad |
| 2 | 98 |
| 3 | 95 |
| 6 | 85 |
| 8 | 92 |
| 10 | 95 |
| 12 | 77 |
| 13 | 95 |
| 14 | 88 |
| 15 | 97 |
| 16 | 97 |
| 17 | 88 |
| 26 | 88 |
| 27 | 97 |
| 40 | 81 |
| 44 | 81 |
| 47 | 77 |
| 53 | 91 |
| 72 | 83 |
| 81 | 97 |
| 84 | 98 |
| 86 | 94 |
| 92 | 75 |
| 103 | 75 |
| 122 | 88 |
| 132 | 86 |
| 171 | 93 |

### Example H

### In Vivo Test on Pyricularia Grisea (Rice Blast on Rice)

An aqueous suspension of the active substance was prepared by homogenization of a mixture of acetone/Tween/dimethyl sulphoxide and subsequent dilution with water to the desired concentration. Rice plants (variety: Koshihikari) are sown in cultivation dishes on a peat-pozzolanic earth substrate (50/50) at 25° C. and sprayed at the second-leaf stage (13 to 15 cm size) with the aqueous suspension described above. As a control, plants are sprayed with an aqueous acetone/Tween/DMSO solution with no active substance. After 24 hours the plants are inoculated by spraying with an aqueous suspension of Pyricularia grisea spores (30,000 spores per ml). The spores are derived from a 17 day-old culture and are suspended in water which contains 2.5 g/l gelatine. The inoculated rice plants are first incubated for 3 days at ca. 25° C. and 100% relative atmospheric humidity and then 3 days at 25° C. and 80% relative atmospheric humidity during the day and 20% relative atmospheric humidity at night. After 6 days they are scored in comparison with the control plants. Under these conditions at a dosage of 500 ppm good (70% activity level) or complete inhibition was observed for the following compounds:

| Ex. | % |
|---|---|
| Compound | Activity |
| No. | level |
| 1 | 82 |
| 91 | 75 |
| 148 | 71 |

### Example I

### Production of Fumonisin FB1 by Fusarium Proliferatum

The compounds were tested in microtitre plates in a fumonisin-inducing liquid medium (0.5 g malt extract, 1 g yeast extract, 1 g bactopeptone, 20 g fructose, 1 g KH₂PO₄, 0.3 g MgSO₄×7H₂O, 0.3 g KCl, 0.05 g ZnSO₄×7H₂O and 0.01 g CuSO₄×5H₂O per litre) with DMSO (0.5%). The inoculation was effected with a concentrated spore suspension of Fusarium proliferatum with a final concentration of 2000 spores/ml.

The plate was incubated at high atmospheric humidity for 5 days at 20° C.

At the start and after 5 days an OD measurement was made at OD 620 (multiple measurement: 3×3 measurements per well) for calculation of the growth inhibition.

After 5 days a sample of the liquid medium was taken and diluted 1:1000 in 50% acetonitrile. The FB1 concentration of the diluted samples were analyzed by HPLC-MS/MS and the measured values used for calculation of the inhibition of fumonisin FB1 production in comparison to an active substance-free control.

HPLC-MS/MS was performed with the following parameters:

Ionization type: ESI positive

Ion spray voltage: 5500 V

Spray gas temperature: 500° C.

Decluster potential: 114 V

Collision energy: 51 eV

Collision gas: N₂

NMR trace: 722.3>352.3; dwell time 100 ms

HPLC column: Waters Atlantis T3 (trifunctional C18 bonding, sealed)

Particle size: 3 μm

Column dimensions: 50×2 mm

Temperature: 40° C.

Solvent A: water+0.1% HCOOH (v/v)

Solvent B: acetonitrile+0.1% HCOOH (v/v)

Flow rate: 400 μL/minute

Injection volume: 5 μL

Gradient:

| Time [min] | A % | B % |
|---|---|---|
| 0 | 90 | 10 |
| 2 | 5 | 95 |
| 4 | 5 | 95 |
| 4.1 | 90 | 10 |
| 9 | 90 | 10 |

Examples of the Inhibition of Fumonisin FB1 Production

The examples listed below showed >80% inhibition of fumonisin FB1 production at a concentration of 50 μM. The inhibition of the growth of Fusarium proliferatum in the said examples varied from 0 to 99% at 50 μM.

| | % Inhibition der | |
|---|---|---|
| Bsp. Verbindung | FB1 produktion bei | % Inhibition des |
| Nr. | 50 μM | Pilzwachstums bei 50 μM |
| 19 | 100 | 68 |
| 20 | 100 | 65 |
| 21 | 100 | 55 |
| 23 | 100 | 73 |
| 29 | 82 | 34 |
| 33 | 99 | 99 |
| 40 | 100 | 98 |
| 41 | 100 | 56 |
| 44 | 100 | 33 |
| 47 | 98 | 0 |
| 50 | 89 | 8 |
| 53 | 96 | 0 |
| 54 | 100 | 47 |
| 58 | 89 | 42 |
| 59 | 95 | 0 |
| 60 | 100 | 92 |
| 66 | 85 | 40 |
| 70 | 92 | 0 |
| 84 | 100 | 88 |
| 85 | 100 | 95 |
| 86 | 100 | 96 |
| 87 | 100 | 83 |
| 146 | 100 | 41 |
| 153 | 100 | 48 |
| 155 | 100 | 97 |
| 156 | 100 | 93 |
| 163 | 100 | 72 |
| 164 | 100 | 97 |
| 165 | 100 | 90 |
| 166 | 100 | 98 |
| 189 | 90 | 0 |
| 191 | 96 | 1 |
| 193 | 85 | 0 |
| 195 | 100 | 17 |
| 196 | 100 | 25 |
| 198 | 100 | 99 |
| 200 | 99 | 1 |
| 203 | 100 | 99 |
| 213 | 100 | 56 |
| 214 | 100 | 96 |
| 215 | 100 | 94 |
| 216 | 100 | 97 |
| 217 | 100 | 97 |
| 218 | 100 | 79 |
| 219 | 100 | 97 |
| 220 | 100 | 99 |
| 221 | 100 | 99 |
| 222 | 100 | 96 |
| 223 | 100 | 96 |
| 224 | 100 | 99 |
| 225 | 100 | 93 |
| 5 | 100 | 79 |
| 9 | 96 | 40 |

### Example J

### Production of DON/Acetyl-DON by Fusarium Graminearum

The compounds were tested in microtitre plates in a DON-inducing liquid medium (1 g (NH₄)₂HPO₄, 0.2 g MgSO₄×7H₂O, 3 g KH₂PO₄, 10 g glycerine, 5 g NaCl and 40 g saccharose per litre) and DMSO (0.5%). The inoculation was effected with a concentrated spore suspension of Fusarium graminearum with a final concentration of 2000 spores/ml.

The plate was incubated at high atmospheric humidity for 7 days at 28° C.

At the start and after 3 days an OD measurement was made at OD 620 (multiple measurement: 3×3 measurements per well) for calculation of the growth inhibition.

After 7 days, 1 volume of an 84/16 acetonitrile/water mixture was added and a sample of the liquid medium was then taken from each well and diluted 1:100 in 10% acetonitrile. The DON and acetyl-DON contents of the samples were analyzed by HPLC-MS/MS and the measured values were used for the calculation of the inhibition of DON/AcDON in comparison to an active substance-free control.

The HPLC-MS/MS measurements were performed with the following parameters:

Ionization type: ESI negative

Ion spray voltage: −4500 V

Spray gas temperature: 500° C.

Decluster potential: −40 V

Collision energy: −22 eV

Collision gas: N₂

NMR Spur: 355.0>264.9;

HPLC column: Waters Atlantis T3 (trifunctional C18 bonding, sealed)

Particle size: 3 μm

Column dimensions: 50×2 mm

Temperature: 40° C.

Solvent A: water/2.5 mM NH₄OAc+0.05% CH₃COOH (v/v)

Solvent B: methanol/2.5 mM NH₄OAc+0.05% CH₃COOH (v/v)

Flow rate: 400 μL/minute

Injection volume: 11 μL

Gradient:

| Time [min] | A % | B % |
|---|---|---|
| 0 | 100 | 0 |
| 0.75 | 100 | 0 |
| 1.5 | 5 | 95 |
| 4 | 5 | 95 |
| 5 | 100 | 0 |
| 10 | 100 | 0 |

Examples of DON Inhibition

The examples listed below showed >=80% inhibition of DON/AcDON production at 50 μM. The inhibition of the growth of Fusarium graminearum in the stated examples varied from 0 to 100% at 50 μM.

| | | % Inhibition des |
|---|---|---|
| Bsp. Verbindung | % Inhibition der DON/AcDON | Pilzwachstums bei |
| Nr. | produktion bei 50 μM | 50 μM |
| 5 | 100 | 97 |
| 20 | 87 | 21 |
| 37 | 80 | 13 |
| 40 | 100 | 100 |
| 54 | 85 | 31 |
| 84 | 99 | 92 |
| 85 | 99 | 100 |
| 86 | 98 | 94 |
| 87 | 100 | 97 |
| 142 | 89 | 0 |
| 146 | 100 | 97 |
| 150 | 99 | 97 |
| 153 | 100 | 56 |
| 154 | 93 | 0 |
| 155 | 100 | 97 |
| 156 | 81 | 99 |
| 157 | 100 | 100 |
| 159 | 99 | 102 |
| 160 | 100 | 100 |
| 161 | 100 | 104 |
| 162 | 99 | 99 |
| 163 | 100 | 100 |
| 164 | 99 | 100 |
| 165 | 100 | 64 |
| 166 | 100 | 84 |
| 167 | 99 | 100 |
| 169 | 100 | 101 |
| 183 | 99 | 65 |
| 195 | 80 | 0 |
| 198 | 100 | 100 |
| 203 | 100 | 100 |
| 206 | 99 | 75 |
| 207 | 100 | 75 |
| 208 | 100 | 101 |
| 210 | 100 | 85 |
| 211 | 99 | 16 |
| 213 | 100 | 0 |
| 214 | 93 | 100 |
| 219 | 100 | 97 |
| 220 | 99 | 100 |
| 221 | 100 | 100 |
| 222 | 82 | 99 |
| 223 | 97 | 99 |
| 224 | 92 | 100 |
| 225 | 89 | 92 |
| 226 | 100 | 85 |
| 227 | 100 | 100 |
| 229 | 97 | 0 |
| 230 | 97 | 0 |
| 232 | 80 | 0 |
| 233 | 100 | 100 |
| 245 | 99 | 95 |
| 246 | 99 | 88 |
| 247 | 99 | 96 |
| 252 | 99 | 0 |
| 254 | 99 | 16 |
| 255 | 96 | 0 |
| 257 | 94 | 0 |
| 258 | 98 | 79 |
| 260 | 99 | 5 |
| 261 | 99 | 39 |
| 262 | 99 | 35 |
| 264 | 99 | 17 |
| 265 | 98 | 9 |
| 267 | 97 | 59 |
| 268 | 90 | 32 |
| 271 | 99 | 71 |
| 272 | 99 | 33 |
| 326 | 100 | 107 |
| 327 | 100 | 105 |
| 328 | 100 | 101 |
| 329 | 100 | 104 |

### Example K

### Production of Aflatoxins by Aspergillus Parasiticus

The compounds were tested in microtitre plates (black 96-well plates with flat and transparent base) in an aflatoxin-inducing liquid medium (20 g saccharose, 4 g yeast extract, 1 g KH₂PO₄ and 0.5 g MgSO₄×7H₂O per litre) treated with 20 mM Cavasol (hydroxypropyl-beta-cyclodextrin) and 1% DMSO. The inoculation was effected with a concentrated spore suspension of Aspergillus parasiticus with a final concentration of 1000 spores/ml.

The plate was incubated at high atmospheric humidity for 7 days at 20° C.

After 7 days an OD measurement was made at OD 620 (multiple measurement: 4×4 measurements per well) for calculation of the growth inhibition. At the same time, through the base of the plate, a fluorescence measurement Em_{360 nm} and Ex_{426 nm} (multiple measurement: 3×3 measurements per well) was made for calculation of the inhibition of aflatoxin production in comparison to an active substance-free control.

Examples of Inhibition of Aflatoxin Production

The examples listed below showed >80% inhibition of aflatoxin production at 50 μM. The growth inhibition of Aspergillus parasiticus at 50 μM in these examples was also >80%.

| | | % Inhibition des |
|---|---|---|
| | Inhibition der Aflatoxin | Pilzwachstums bei |
| Bsp Verbindung Nr. | produktion bei 50 μM | 50 μM |
| 40 | 99 | 88 |
| 84 | 100 | 98 |
| 85 | 100 | 98 |
| 86 | 100 | 97 |
| 87 | 100 | 97 |
| 146 | 100 | 99 |
| 155 | 100 | 100 |
| 203 | 100 | 100 |

### Example L

### In Vivo Test on Spaerotheca Fuliginea (Powdery Mildew on Cucumber/Protective)

Solvent: 49 parts by weight N,N-dimethylformamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young cucumber plants are sprayed with the active substance preparation at the stated application dosage. One day after the treatment, the plants are inoculated with a spore suspension of Sphaerotheca fuliginea. Next, the plants are placed in a greenhouse at 70% relative atmospheric humidity and a temperature of 23° C.

7 days after the inoculation, the assessment takes place. Here 0% means an activity level which corresponds to that of the control, while an activity level of 100% means that no infection is observed.

In this test, the compounds according to the invention of the following formulae at an active substance concentration of 500 ppm display an activity level of 70% or more.

TABLE
Sphaerotheca test (cucumber)/protective

| Bsp. | | Bsp. | | Bsp. | |
|---|---|---|---|---|---|
| Verbindung | % | Verbindung | % | Verbindung | % |
| Nr. | Wirkungsgrad | Nr. | Wirkungsgrad | Nr. | Wirkungsgrad |
| 5 | 70 | 217 | 100 | 299 | 84 |
| 9 | 90 | 218 | 100 | 298 | 100 |
| 31 | 70 | 219 | 100 | 297 | 90 |
| 29 | 70 | 220 | 97 | 296 | 84 |
| 15 | 95 | 221 | 100 | 295 | 94 |
| 11 | 73 | 222 | 100 | 294 | 98 |
| 85 | 100 | 223 | 91 | 293 | 94 |
| 86 | 100 | 224 | 99 | 292 | 95 |
| 84 | 100 | 225 | 100 | 291 | 95 |
| 89 | 88 | 165 | 86 | 290 | 100 |
| 136 | 73 | 226 | 95 | 371 | 94 |
| 91 | 100 | 228 | 75 | 369 | 88 |
| 126 | 95 | 229 | 94 | 368 | 94 |
| 150 | 93 | 232 | 93 | 367 | 84 |
| 122 | 95 | 233 | 95 | 366 | 94 |
| 92 | 94 | 236 | 86 | 307 | 100 |
| 116 | 93 | 237 | 88 | 306 | 94 |
| 104 | 70 | 238 | 100 | 301 | 95 |
| 103 | 100 | 239 | 95 | 300 | 90 |
| 146 | 95 | 240 | 96 | 309 | 100 |
| 87 | 100 | 242 | 97 | 303 | 95 |
| 171 | 100 | 243 | 95 | 312 | 100 |
| 174 | 100 | 245 | 88 | 313 | 98 |
| 169 | 89 | 162 | 99 | 314 | 100 |
| 178 | 75 | 249 | 92 | 315 | 95 |
| 179 | 74 | 250 | 84 | 316 | 75 |
| 156 | 94 | 251 | 96 | 320 | 94 |
| 181 | 85 | 253 | 92 | 320 | 89 |
| 183 | 94 | 158 | 97 | 320 | 95 |
| 184 | 95 | 159 | 97 | 322 | 95 |
| 192 | 85 | 254 | 92 | 323 | 86 |
| 198 | 99 | 256 | 92 | 326 | 94 |
| 155 | 95 | 157 | 100 | 328 | 100 |
| 203 | 95 | 258 | 80 | 329 | 94 |
| 166 | 97 | 260 | 95 | 331 | 94 |
| 163 | 93 | 167 | 100 | 335 | 74 |
| 161 | 100 | 267 | 98 | 337 | 88 |
| 160 | 100 | 268 | 100 | 338 | 91 |
| 205 | 100 | 271 | 79 | 339 | 95 |
| 206 | 94 | 272 | 94 | 340 | 95 |
| 207 | 100 | 286 | 89 | 341 | 100 |
| 208 | 100 | 285 | 89 | 342 | 94 |
| 209 | 100 | 284 | 91 | 346 | 95 |
| 210 | 100 | 283 | 78 | 347 | 95 |
| 211 | 93 | 281 | 94 | 348 | 95 |
| 212 | 96 | 280 | 93 | 349 | 95 |
| 213 | 74 | 279 | 100 | 350 | 94 |
| 164 | 100 | 278 | 89 | 351 | 80 |
| 214 | 100 | 277 | 94 | 352 | 73 |
| 215 | 100 | 276 | 99 | | |
| 216 | 89 | 274 | 94 | | |

### Example M

### In Vivo Test on Alternaria Solani (Leaf Blotch Disease/Tomato/Protective)

Solvent: 49 parts by weight N,N-dimethylformamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young tomato plants are sprayed with the active substance preparation at the stated application dosage. One day after the treatment, the plants are inoculated with a spore suspension of Alternaria solani and then stand for 24 hrs at 100% rel. humidity and 22° C. Next, the plants stand at 96% rel. atmospheric humidity and a temperature of 20° C.

7 days after the inoculation, the assessment takes place. Here 0% means an activity level which corresponds to that of the control, while an activity level of 100% means that no infection is observed.

In this test, the compounds according to the invention of the following formulae at an active substance concentration of 500 ppm display an activity level of 70% or more.

### EXAMPLE

Alternaria-Test (tomato)/protective

| Bsp. | | Bsp. | | Bsp. | |
|---|---|---|---|---|---|
| Verbindung | % | Verbindung | % | Verbindung | % |
| Nr. | Wirkungsgrad | Nr. | Wirkungsgrad | Nr. | Wirkungsgrad |
| 30 | 90 | 164 | 95 | 298 | 80 |
| 25 | 90 | 214 | 95 | 297 | 100 |
| 5 | 90 | 215 | 94 | 296 | 100 |
| 9 | 89 | 216 | 95 | 295 | 100 |
| 81 | 95 | 218 | 95 | 294 | 100 |
| 29 | 78 | 220 | 95 | 293 | 100 |
| 2 | 100 | 222 | 90 | 292 | 100 |
| 15 | 95 | 223 | 90 | 291 | 100 |
| 11 | 80 | 224 | 70 | 290 | 100 |
| 72 | 95 | 225 | 95 | 371 | 95 |
| 85 | 100 | 165 | 95 | 370 | 95 |
| 84 | 95 | 226 | 95 | 369 | 90 |
| 89 | 89 | 227 | 95 | 310 | 95 |
| 136 | 95 | 228 | 70 | 368 | 95 |
| 135 | 78 | 229 | 95 | 367 | 95 |
| 134 | 100 | 231 | 95 | 366 | 80 |
| 91 | 95 | 232 | 95 | 365 | 80 |
| 126 | 95 | 233 | 90 | 307 | 100 |
| 150 | 95 | 235 | 94 | 306 | 95 |
| 122 | 95 | 236 | 94 | 305 | 100 |
| 92 | 90 | 237 | 100 | 302 | 95 |
| 116 | 95 | 238 | 94 | 301 | 95 |
| 113 | 89 | 239 | 100 | 300 | 80 |
| 108 | 80 | 240 | 88 | 309 | 100 |
| 106 | 95 | 241 | 94 | 364 | 95 |
| 104 | 90 | 242 | 88 | 363 | 90 |
| 103 | 90 | 243 | 100 | 303 | 100 |
| 102 | 95 | 244 | 94 | 362 | 80 |
| 146 | 100 | 245 | 94 | 361 | 70 |
| 144 | 94 | 247 | 94 | 312 | 100 |
| 142 | 94 | 162 | 94 | 313 | 95 |
| 170 | 78 | 248 | 94 | 314 | 100 |
| 87 | 95 | 249 | 94 | 315 | 95 |
| 153 | 90 | 250 | 94 | 316 | 100 |
| 171 | 95 | 251 | 94 | 317 | 70 |
| 173 | 94 | 253 | 94 | 318 | 80 |
| 174 | 100 | 158 | 94 | 319 | 90 |
| 176 | 100 | 159 | 94 | 320 | 95 |
| 178 | 94 | 254 | 94 | 321 | 70 |
| 179 | 100 | 256 | 94 | 322 | 95 |
| 156 | 94 | 157 | 94 | 323 | 95 |
| 181 | 94 | 257 | 94 | 326 | 95 |
| 183 | 100 | 258 | 95 | 327 | 95 |
| 184 | 95 | 259 | 90 | 328 | 95 |
| 189 | 95 | 260 | 90 | 329 | 95 |
| 192 | 100 | 262 | 95 | 332 | 90 |
| 193 | 93 | 267 | 95 | 337 | 95 |
| 195 | 80 | 268 | 80 | 338 | 80 |
| 196 | 100 | 269 | 89 | 339 | 95 |
| 198 | 93 | 271 | 89 | 340 | 95 |
| 155 | 100 | 272 | 94 | 341 | 95 |
| 200 | 93 | 273 | 90 | 342 | 95 |
| 203 | 93 | 286 | 90 | 347 | 95 |
| 166 | 95 | 285 | 95 | 348 | 95 |
| 163 | 93 | 284 | 100 | 349 | 80 |
| 161 | 95 | 283 | 95 | 350 | 95 |
| 160 | 95 | 282 | 70 | 351 | 95 |
| 205 | 95 | 281 | 95 | 352 | 95 |
| 206 | 90 | 280 | 95 | 353 | 80 |
| 207 | 90 | 279 | 70 | 164 | 95 |
| 208 | 80 | 278 | 95 | 214 | 95 |
| 209 | 95 | 277 | 100 | 215 | 94 |
| 210 | 80 | 276 | 95 | 216 | 95 |
| 211 | 70 | 275 | 90 | 218 | 95 |
| 212 | 95 | 274 | 95 | 220 | 95 |
| 213 | 90 | 299 | 100 | 298 | 80 |
| 297 | 100 | 296 | 100 | 295 | 100 |
| 294 | 100 | 293 | 100 | | |

### Example N

### In Vivo Test on Plasmopara Viticola (Downy Mildew, Vine/Protective)

Solvent: 24.5 parts by weight acetone
24.5 parts by weight dimethylacetamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage. After drying of the spray coating, the plants are inoculated with an aqueous spore suspension of Plasmopara viticola and then remain for 1 day in an incubation cabin at ca. 20° C. and 100% relative atmospheric humidity. Next, the plants are placed for 4 days in the greenhouse at ca. 21° C. and ca. 90% atmospheric humidity. The plants are then moistened and placed in an incubation cabin for 1 day.

6 days after the inoculation, the assessment takes place. Here 0% means an activity level which corresponds to that of the control, while an activity level of 100% means that no infection is observed.

In this test, the following compounds according to the invention at an active substance concentration of 100 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 1 | 100 | 169 | 96 | 219 | 95 |
| 40 | 72 | 183 | 86 | 225 | 90 |
| 85 | 94 | 192 | 94 | 236 | 76 |
| 86 | 84 | 198 | 92 | 237 | 84 |
| 84 | 98 | 161 | 95 | 238 | 88 |
| 89 | 73 | 160 | 79 | 239 | 88 |
| 126 | 81 | 207 | 71 | 240 | 90 |
| 92 | 73 | 212 | 83 | 260 | 70 |
| 174 | 70 | 214 | 88 | | |

### Example O

### In Vivo Test on Venturia Inaequalis (Apple Scab/Protective)

Solvent: 24.5 parts by weight acetone
24.5 parts by weight dimethylaminoacetamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage. After drying of the spray coating, the plants are inoculated with an aqueous conidia suspension of the apple scab pathogen Venturia inaequalis and then remain for 1 day at ca. 20° C. and 100% relative atmospheric humidity in an incubation cabin.

The plants are then placed in the greenhouse at ca. 21° C. and a relative atmospheric humidity of ca. 90% .

10 days after the inoculation, the assessment takes place. Here 0% means an activity level which corresponds to that of the control, while an activity level of 100% means that no infection is observed.

In this test the following compounds according to the invention at an active substance concentration of 100 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 1 | 99 | 160 | 94 | 236 | 94 |
| 40 | 97 | 205 | 91 | 237 | 95 |
| 85 | 88 | 206 | 83 | 238 | 98 |
| 86 | 96 | 207 | 86 | 239 | 98 |
| 84 | 100 | 208 | 78 | 240 | 96 |
| 89 | 100 | 209 | 89 | 242 | 89 |
| 126 | 98 | 210 | 78 | 243 | 98 |
| 92 | 93 | 211 | 92 | 162 | 96 |
| 87 | 95 | 212 | 85 | 249 | 96 |
| 174 | 100 | 164 | 97 | 251 | 84 |
| 169 | 99 | 214 | 88 | 159 | 97 |
| 179 | 100 | 215 | 96 | 254 | 73 |
| 180 | 96 | 217 | 98 | 157 | 94 |
| 156 | 91 | 218 | 95 | 260 | 94 |
| 183 | 73 | 219 | 96 | 167 | 89 |
| 184 | 92 | 220 | 96 | 268 | 98 |
| 189 | 84 | 221 | 91 | 271 | 81 |
| 192 | 95 | 222 | 95 | 272 | 89 |
| 198 | 96 | 224 | 71 | | |
| 161 | 95 | 225 | 96 | | |

### Example P

### In Vivo Test on Botrytis Cinerea (Gray Mould on Bean/Protective)

Solvent: 24.5 parts by weight acetone
24.5 parts by weight acetone

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage. After drying of the spray coating, 2 small pieces of agar with Botrytis cinerea growing on them are laid on every leaf. The inoculated plants are set out in a darkened chamber at ca. 20° C. and 100% relative atmospheric humidity.

2 days after the inoculation, the size of the infection blotches on the leaves is assessed. Here 0% means an activity level which corresponds to that of the control, while an activity level of 100% means that no infection is observed.

In this test the following compounds according to the invention at an active substance concentration of 250 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 1 | 99 | 155 | 99 | 224 | 99 |
| 40 | 100 | 161 | 100 | 225 | 96 |
| 85 | 100 | 160 | 100 | 225 | 93 |
| 86 | 100 | 205 | 100 | 236 | 100 |
| 84 | 99 | 206 | 100 | 237 | 100 |
| 89 | 100 | 207 | 100 | 238 | 93 |
| 126 | 100 | 208 | 100 | 240 | 99 |
| 92 | 100 | 209 | 100 | 242 | 99 |
| 87 | 100 | 210 | 98 | 243 | 75 |
| 171 | 84 | 211 | 100 | 162 | 100 |
| 174 | 80 | 212 | 98 | 249 | 100 |
| 169 | 100 | 213 | 91 | 251 | 95 |
| 179 | 100 | 214 | 100 | 159 | 98 |
| 180 | 93 | 215 | 98 | 157 | 78 |
| 156 | 98 | 217 | 95 | 260 | 100 |
| 183 | 93 | 218 | 98 | 167 | 100 |
| 184 | 100 | 219 | 100 | 268 | 100 |
| 189 | 93 | 220 | 100 | 271 | 93 |
| 192 | 100 | 221 | 100 | 272 | 100 |
| 198 | 99 | 222 | 85 | | |

### Example Q

### In Vivo Test on Leptosphaeria Nodorum (Glume Blotch in Wheat/Protective)

Solvent: 49 parts by weight N,N-dimethylacetamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage.

After drying of the spray coating, the plants are sprayed with spores with a spore suspension of Leptosphaeria nodorum. The plants remain for 48 hours at 20° C. and 100% relative atmospheric humidity in an incubation cabin.

The plants are set out in a greenhouse at a temperature of ca. 22° C. and a relative atmospheric humidity of ca. 80%.

8 days after the inoculation, the assessment takes place. Here 0% means an activity level which corresponds to that of the control, while an activity level of 100% means that no infection is observed.

In this test the following compounds according to the invention at an active substance concentration of 1000 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 1 | 100 | 174 | 93 | 164 | 93 |
| 85 | 100 | 169 | 100 | 214 | 71 |
| 86 | 100 | 184 | 88 | 215 | 71 |
| 84 | 100 | 192 | 86 | 218 | 93 |
| 89 | 83 | 198 | 100 | 225 | 86 |
| 87 | 88 | 212 | 71 | 167 | 92 |

### Example R

### In Vivo Test on Septoria Tritici (Leaf Blotch (Black Spot) in Wheat/Protective)

Solvent: 49 parts by weight N,N-dimethylacetamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage. After drying of the spray coating, the plants are sprayed with a spore suspension of Septoria tritici. The plants remain for 48 hours at 20° C. and 100% relative atmospheric humidity in an incubation cabin. After this, the plants are placed for a further 60 hours under a transparent hood at 15° C. and 100% relative atmospheric humidity.

The plants are set out in a greenhouse at a temperature of ca. 15° C. and a relative atmospheric humidity of 80%.

21 days after the inoculation, the assessment takes place. Here 0% means an activity level which corresponds to that of the control, while an activity level of 100% means that no infection is observed.

In this test the following compounds according to the invention at an active substance concentration of 1000 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 1 | 100 | 169 | 100 | 215 | 94 |
| 85 | 100 | 184 | 100 | 218 | 100 |
| 86 | 100 | 192 | 100 | 224 | 100 |
| 84 | 100 | 198 | 100 | 225 | 100 |
| 89 | 100 | 212 | 71 | 240 | 75 |
| 87 | 100 | 164 | 100 | 159 | 100 |
| 174 | 90 | 214 | 100 | 157 | 100 |

### Example S

### In Vivo Test on Rhynchosporium Secalis (Leaf Scald in Barley/Protective)

Solvent: 49 parts by weight N,N-dimethylacetamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage.

After drying of the spray coating, the plants are sprayed with spores with a spore suspension of Rhynchosporium secalis. The plants remain for 48 hours at 20° C. and 100% relative atmospheric humidity in an incubation cabin.

The plants are set out in a greenhouse at a temperature of ca. 20° C. and a relative atmospheric humidity of ca. 80%.

14 days after the inoculation, the assessment takes place. Here 0% means an activity level which corresponds to that of the control, while an activity level of 100% means that no infection is observed.

In this test the following compounds according to the invention at an active substance concentration of 1000 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 86 | 86 | 184 | 71 | 214 | 100 |
| 84 | 90 | 192 | 71 | 215 | 90 |
| 87 | 88 | 198 | 90 | 218 | 100 |
| 169 | 100 | 164 | 100 | 225 | 90 |

### Example T

### In Vivo Test on Fusarium Nivale (Var. Majus) (Head Blight/White-Ear in Wheat/Protective)

Solvent: 49 parts by weight N,N-dimethylacetamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage.

After drying of the spray coating, the plants are sprayed with spores with a spore suspension of Fusarium nivale (var. majus).

The plants are placed in a greenhouse chamber under a transparent incubation hood at 10° C. and 100% relative atmospheric humidity.

5 days after the inoculation, the assessment takes place. Here 0% means an activity level which corresponds to that of the control, while an activity level of 100% means that no infection is observed.

In this test the following compounds according to the invention at an active substance concentration of 1000 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 1 | 92 | 192 | 100 | 225 | 100 |
| 85 | 100 | 198 | 100 | 238 | 75 |
| 86 | 100 | 212 | 75 | 240 | 75 |
| 84 | 100 | 164 | 100 | 159 | 75 |
| 89 | 92 | 214 | 100 | 157 | 88 |
| 87 | 92 | 215 | 100 | 167 | 100 |
| 169 | 71 | 218 | 92 | | |
| 184 | 100 | 224 | 100 | | |

### Example T

### In Vivo Test on Fusarium Graminearum (Head Blight/White-Ear in Barley/Protective)

Solvent: 49 parts by weight N,N-dimethylacetamide

Emulsifier: 1 part by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young plants are sprayed with the active substance preparation at the stated application dosage.

After drying of the spray coating, the plants are sprayed with spores with a spore suspension of Fusarium graminearum.

The plants are placed in a greenhouse chamber under a transparent incubation hood at 22° C. and 100% relative atmospheric humidity.

5 days after the inoculation, the assessment takes place. Here 0% means an activity level which corresponds to that of the control, while an activity level of 100% means that no infection is observed.

In this test the following compounds according to the invention at an active substance concentration of 1000 ppm display an activity level of 70% or more.

| Bsp. | | Bsp. | | Bsp. | |
|---|---|---|---|---|---|
| Verbindung | % | Verbindung | % | Verbindung | % |
| Nr. | Wirkungsgrad | Nr. | Wirkunggrad | Nr. | Wirkungsgrad |
| 1 | 100 | 218 | 100 | 87 | 100 |
| 85 | 100 | 224 | 100 | 169 | 100 |
| 86 | 100 | 225 | 100 | 184 | 100 |
| 84 | 100 | 238 | 92 | 192 | 100 |
| 89 | 100 | 251 | 83 | 198 | 100 |
| 164 | 100 | 159 | 100 | 155 | 88 |
| 214 | 100 | 157 | 100 | | |
| 215 | 100 | 167 | 100 | | |

### Example U

### In Vivo Test on Pythium Ultimum (Root Rot/Damping Off in Cotton/Seed Treatment)

The test was performed under greenhouse conditions.

Cotton seeds, treated with an active compound according to the invention or a combination of active compounds according to the invention were sown in 6*6 cm size vessels, in a mixture of steamed field earth and sand (1:1). The test compound/s were dissolved in N-methyl-2-pyrrolidone and diluted to the desired concentration with water. The plants were grown at 10° C.

Perlite was inoculated with mycelium from Pythium ultimum. 1 mL of the infected perlite was distributed between the treated cotton seeds. The seeds were covered with a covering layer of clay granules and incubated in the greenhouse for 7 days at 20° C. and 80% relative atmospheric humidity.

The assessment was made by counting the emergence. Here 0% means an activity level which corresponds to that of the untreated control, while an activity level of 100% means that all seeds germinated.

In this test the following compounds showed an efficacy of 70% and above at a dose of 50 g/dt of the active compound according to the invention.

| Ex. Compound No. | % Activity level |
|---|---|
| 198 | 88 |

### Example V

### In Vivo Test on Pyricularia Oryzae (Rice Blast/Protective)

Solvent: 28.5 parts by weight acetone

Emulsifier: 1.5 parts by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young rice plants are sprayed with the active substance preparation at the stated application dosage. One day after the treatment, the plants are inoculated with an aqueous spore suspension of Pyricularia oryzae. Next, the plants are set out in a greenhouse at 100% relative atmospheric humidity and 25° C.

5 days after the inoculation, the assessment takes place. Here 0% means an activity level which corresponds to that of the control, while an activity level of 100% means that no infection is observed.

In this test the following compounds according to the invention at an active substance concentration of 250 ppm display an activity level of 80% or more.

| Ex. No. | % Activity level |
|---|---|
| 1 | 80 |
| 4 | 80 |
| 30 | 80 |
| 40 | 95 |
| 84 | 85 |
| 85 | 80 |
| 86 | 93 |
| 102 | 93 |
| 104 | 80 |
| 108 | 85 |
| 113 | 90 |
| 116 | 80 |
| 122 | 85 |
| 136 | 80 |
| 198 | 85 |
| 217 | 85 |
| 221 | 92 |

### Example W

### In Vivo Test on Rhizoctonia Solani (Sheath Blight in Rice/Protective)

Solvent: 28.5 parts by weight acetone

Emulsifier: 1.5 parts by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young rice plants are sprayed with the active substance preparation at the stated application dosage. One day after the treatment, the plants are inoculated with hyphae of Rhizoctonia solani. Next, the plants are set out in a greenhouse at 100% relative atmospheric humidity and 25° C.

4 days after the inoculation, the assessment takes place. Here 0% means an activity level which corresponds to that of the control, while an activity level of 100% means that no infection is observed.

In this test the following compounds according to the invention at an active substance concentration of 250 ppm display an activity level of 80% or more.

| Ex. No. | % Activity level |
|---|---|
| 1 | 100 |
| 4 | 100 |
| 30 | 95 |
| 84 | 100 |
| 85 | 97 |
| 86 | 100 |
| 89 | 92 |
| 101 | 95 |
| 102 | 98 |
| 104 | 95 |
| 108 | 92 |
| 113 | 93 |
| 116 | 97 |
| 120 | 93 |
| 122 | 97 |
| 136 | 97 |
| 169 | 100 |
| 174 | 93 |
| 176 | 97 |
| 198 | 88 |
| 217 | 100 |
| 221 | 100 |

### Example X

### In Vivo Test on Cochliobolus Miyabeanus (Brown Spot Disease, Rice/Protective)

Solvent: 28.5 parts by weight acetone

Emulsifier: 1.5 parts by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young rice plants are sprayed with the active substance preparation at the stated application dosage. One day after the treatment, the plants are inoculated with an aqueous spore suspension of Cochliobolus miyabeanus. Next, the plants are set out in a greenhouse at 100% relative atmospheric humidity and 25° C.

4 days after the inoculation, the assessment takes place. Here 0% means an activity level which corresponds to that of the control, while an activity level of 100% means that no infection is observed.

In this test the following compounds according to the invention at an active substance concentration of 250 ppm displayed an activity level of 80% or more.

| Ex. No. | % Activity level |
|---|---|
| 1 | 85 |
| 40 | 85 |
| 84 | 93 |
| 85 | 90 |
| 86 | 80 |
| 89 | 85 |
| 104 | 80 |
| 108 | 80 |
| 116 | 94 |
| 122 | 90 |
| 198 | 90 |
| 217 | 85 |
| 221 | 80 |

### Example Y

### In Vivo Test on Gibberella Zeae (Head Blight in Rice/Protective)

Solvent: 28.5 parts by weight acetone

Emulsifier: 1.5 parts by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young rice plants are sprayed with the active substance preparation at the stated application dosage. One day after the treatment, the plants are inoculated with an aqueous spore suspension of Gibberella zeae. Next, the plants are set out in a greenhouse at 100% relative atmospheric humidity and 25° C.

5 days after the inoculation, the assessment takes place. Here 0% means an activity level which corresponds to that of the control, while an activity level of 100% means that no infection is observed.

In this test the following compounds according to the invention at an active substance concentration of 250 ppm display an activity level of 80% or more.

| Ex. No. | % Activity level |
|---|---|
| 198 | 80 |

### Example Z

### In Vivo Test on Phakopsora Pachyrhizi (Soya Bean Rust/Protective)

Solvent: 28.5 parts by weight acetone

Emulsifier: 1.5 parts by weight alkylaryl polyglycol ethers

For the preparation of a suitable active substance preparation, 1 part by weight of active substance is mixed with the stated quantities of solvent and emulsifier and the concentrate is diluted to the desired concentration with water.

For the testing for protective activity, young rice plants are sprayed with the active substance preparation at the stated application dosage. One day after the treatment, the plants are inoculated with an aqueous spore suspension of Phakopsora pachyrhizi. Next, the plants are set out in a greenhouse at 80% relative atmospheric humidity and 20° C.

11 days after the inoculation, the assessment takes place. Here 0% means an activity level which corresponds to that of the control, while an activity level of 100% means that no infection is observed.

In this test the following compounds according to the invention at an active substance concentration of 250 ppm displayed an activity level of 80% or more.

| Ex. No. | % Activity level |
|---|---|
| 1 | 91 |
| 84 | 98 |

1. Compounds of the formula [I-a], 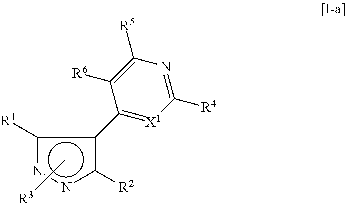
wherein the symbols have the following meanings:
X¹ stands for C—H or N,
R¹ stands for phenyl, naphthalenyl, quinolin-5-yl, quinolin-8-yl, isoquinolin-5-yl, isoquinolin-8-yl, 1-benzothiophen-4-yl, 1-benzothiophen-7-yl, 1-benzo-furan-4-yl, 1-benzofuran-7-yl, 1,3-benzodioxol-4-yl or 1,3-benzodioxol-5-yl, each optionally singly or multiply, identically or differently substituted with R⁷,
R² stands for cyano, nitro, halogen, C₁-C₆ alkyl, C₁-C₆ alkoxy, C₁-C₆ haloalkyl, C₃-C₆ cycloalkyl, C₃-C₆ halocycloalkyl, C₁-C₆ haloalkoxy, C₁-C₆ alkylthio C₂-C₉ heterocyclyl or hydrogen,
R³ stands for C₁-C₆ alkyl, C₂-C₆ alkenyl, C₂-C₆ alkynyl, C₃-C₆ cycloalkyl, C₃-C₆ cycloalkyl-C₁-C₆ alkyl, C₃-C₆ cycloalkyloxy, C₁-C₆ alkoxy, C₁-C₆ alkoxy-C₁-C₆ alkyl, acyloxy-C₁-C₆ alkyl, heteroaryl-C₁-C₆ alkyl, aryl-C₁-C₆ alkyl, C₁-C₆ alkylthio-C₁-C₆ alkyl, C₁-C₄ alkyl-C(O)—C₁-C₆ alkyl, C₃-C₆ cycloalkyl-C(O)—C₁-C₄ alkyl, C₂-C₉ heterocycyl-C(O)—C₁-C₄ alkyl, C₁-C₄ alkyl-C(O)O—C₁-C₆ alkyl, C₁-C₄ alkyl-C(O)O—C₃-C₆ cycloalkyl, C₁-C₄ alkyl-C(O)O heterocyclyl, heterocyclyl-C₁-C₆ alkyl, heterocyclyl, oxoheterocyclyl or heteroaryl, each optionally singly or multiply, identically or differently substituted with halogen, cyano, hydroxy, C₁-C₆ alkyl, C₁-C₆ alkoxy, haloalkoxy, phenyl or phenoxy,
R⁴ stands for hydrogen, halogen, cyano, —C(O)OR¹², —SR¹², —NR¹²R¹³, —C(O)NR¹²R¹³ or —NR¹²R¹⁴, —N═C═NR²², —N═C(H)OR²², —N═C(OR²²)R²³, —N═C(SR²²)R²³, —C(═NR²²)NR²²R²³, —SO(═NR²²)R²³ or —SO₂R²⁰
or for C₁-C₆ alkyl, C₃-C₈ cycloalkyl, C₂-C₆ alkenyl, C₆-C₁₄ aryl, C₂-C₉ heterocyclyl or C₂-C₉ heteroaryl, each optionally singly or multiply, identically or differently substituted with R¹¹,
R⁵ and R⁶ mutually independently stand for hydrogen, fluorine, chlorine, bromine, cyano, nitro, —OH or —SH,
or for C₁-C₆ alkyl, C₃-C₆ cycloalkyl, C₂-C₆ alkenyl, C₂-C₆ alkynyl, C₆-C₁₄ aryl, —O—(C₁-C₄ alkyl), —O—(C₆-C₁₄ aryl), —S—(C₁-C₄ alkyl), —S(O)—(C₁-C₆ alkyl) or —C(O)—(C₁-C₆ alkyl), each optionally singly or multiply, identically or differently substituted with R¹¹
or else together with the carbon atom to which they are bound form a ring with 3 to 8 ring atoms, wherein the ring can contain 1 to 4 hetero atoms from the range oxygen, sulphur or —NR¹⁹, optionally singly or multiply, identically or differently substituted with halogen, oxygen, cyano or C₁-C₄ alkyl,
R⁷ mutually independently stands for one or more of the following groups: fluorine, chlorine, bromine, cyano, nitro, —OH or —SH,
or for C₁-C₆ alkyl, C₃-C₆ cycloalkyl, C₂-C₆ alkenyl, C₂-C₆ alkynyl, tri(C₁-C₄ alkyl)silyl, C₆-C₁₄ aryl, —O—(C₁-C₄ alkyl), —O—(C₆-C₁₄ aryl), —S—(C₁-C₄ alkyl), —S(O)—(C₁-C₆ alkyl) or —S(O)₂—(C₁-C₆ alkyl), each optionally singly or multiply, identically or differently substituted with fluorine, chlorine, bromine, —OH, cyano, C₁-C₆ alkyl or —O—(C₁-C₄ alkyl),
R¹¹ stands for —OH, fluorine, chlorine, bromine, cyano, —NH—C(O)R²⁰, —NR²⁰R²¹, —C(O)R²⁰, —C(O)OR²⁰, —C(O)NR²⁰R²¹ or —SO₂R²⁰
or for C₁-C₆ alkyl, C₂-C₆ alkenyl, C₂-C₆ alkynyl, C₁-C₁₁ heteroalkyl, C₃-C₈ cycloalkyl, —O—(C₁-C₄ alkyl), —S—(C₁-C₄ alkyl), —O—(C₃-C₈ cycloalkyl), —S—(C₃-C₈ cycloalkyl), C₆-C₁₄ aryl, —O—(C₆-C₁₄ aryl), —S—(C₆-C₁₄ aryl), C₂-C₉ heterocyclyl or C₂-C₉ heteroaryl, each optionally singly or multiply, identically or differently substituted with fluorine, chlorine, bromine, —OH, carbonyl, cyano, C₁-C₆ alkyl or —O—(C₁-C₄ alkyl),
R¹² and R¹³ mutually independently stand for one or more of the following groups: H, —C(S)R¹⁵, —C(O)R¹⁵, —SO₂R¹⁵, —C(O)OR¹⁵, —OR¹⁵ or —C(O)NR¹⁵R¹⁶
or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with fluorine, chlorine, bromine, —OH, cyano, C₁-C₆ alkyl, —O—C(O)R¹¹, —O—P(O)(OR¹¹)₂, —O—B(OR¹¹)₂ or —O—(C₁-C₄ alkyl),
R¹⁴ stands for —CH₂—NR²²R²³, piperidin-1-ylmethyl or morpholin-4-ylmethyl,
or for C₁-C₆ alkyl or —O—(C₁-C₄ alkyl), each optionally singly or multiply, identically or differently substituted with fluorine, chlorine, bromine, —OH or cyano,
R¹⁵ and R¹⁶ mutually independently stand for hydrogen or —OH
or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
or together with the nitrogen atom to which they are bound form a 3 to 7-membered ring, which can contain a further hetero atom from the range N or O not adjacent to the nitrogen,
R¹⁷ and R¹⁸ mutually independently stand for one or more of the following groups: H or —C(O)OR¹¹
or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with fluorine, chlorine, bromine, —OH, cyano, C₁-C₆ alkyl, and —O—(C₁-C₄ alkyl),
R¹⁹ stands for H, C₁-C₆ alkyl, C₃-C₆ cycloalkyl, C₂-C₆ alkenyl, C₂-C₆ alkynyl, —C(S)R¹⁵, —C(O)R¹⁵, —SO₂R¹⁵ or —C(O)OR¹⁵,
R²⁰ and R²¹ mutually independently stand for C₁-C₆ alkyl, C₃-C₆ cycloalkyl, C₂-C₆ alkenyl or C₂-C₆ alkynyl each optionally singly or multiply, identically or differently substituted with fluorine, chlorine, bromine, —OH or cyano, or hydrogen, and
R²² and R²³ mutually independently stand for C₁-C₆ alkyl, C₃-C₆ cycloalkyl, C₂-C₆ alkenyl, C₂-C₆ alkynyl or hydrogen,
and agrochemically active salts thereof,
wherein the following compounds are excepted:
a) Compounds wherein,
X¹ stands for N,
R¹ stands for an optionally substituted phenyl,
R³ stands for butyl or propyn-2-yl,
R⁴ stands for —NHR₁₂, and
R¹² stands for optionally substituted phenyl,
and b) Compounds wherein
X¹ stands for N
R², R⁴, R⁵, R⁶ stand for H, and
R³ stands for methyl, ethyl, allyl, 2-methoxyethyl or benzyl, when R¹ stands for 4-chlorophenyl, or
R³ stands for methyl, when R¹ stands for phenyl, 4-methoxyphenyl or 4-fluorophenyl.

2. Compounds of the formula [I-a] according to claim 1,
wherein the symbols have the following meanings,
R¹ stands for phenyl, optionally singly or multiply, identically or differently substituted with R⁷,
R² stands for methyl, ethyl, isopropyl, cyclopropyl or hydrogen,
R³ stands for propan-2-yl, isobutyl, butan-2-yl, 2-methylpropyl, 2,2-dimethyl-propyl, 3-methylbut-2-en-1-yl, but-2-en-1-yl, but-3-en-2-yl, propadienyl, prop-2-yn-1-yl, but-2-yn-1-yl, but-3-yn-2-yl, 2-methylbut-3-yn-2-yl, 2-methylbut-3-yn-2-yl, cyanomethyl, 2-cyanoethyl, 1-cyanopropan-2-yl, cyclopropyl, cyclobutyl, cyclopentyl, cyclohexyl, (2,2-dichlorocyclopropyl)methyl, cyclopropylmethyl, 1-cyclopropylethyl, trichloromethyl, trifluoromethyl, 2-fluoroethyl, 2,2-difluoroethyl, 2,2,2-trifluoroethyl, 2,2,2-trichloroethyl, 2-chloroethyl, 2-bromoethyl, 2-fluoropropyl, 3-fluoropropyl, 2-chloropropyl, 3-chloropropyl, 1,3-difluoropropan-2-yl, 2-fluorobenzyl, 3-fluorobenzyl, 4-fluorobenzyl, 2,3-difluorobenzyl, 2-chloro-6-fluorobenzyl, 1-(2-chlorophenyl)ethyl, 1-(3-chlorophenyl)ethyl, 1-(4-chlorophenyl)ethyl, 3-cyanobenzyl, 4-cyanobenzyl, 4-(difluoromethoxy)benzyl, 2-cyanobenzyl, 2-(3-chlorophenyl)ethyl, 2-(2-chlorophenyl)ethyl, 1-naphthylmethyl, (pyridine-3-ylmethyl, 2-chloro-1,3-thiazol-5-yemethyl, methoxymethyl, 2-methoxyethyl, 2-(methylsulphanyl)ethyl, 2-(trifluoromethoxy)ethyl, 1-methoxypropan-2-yl, 2-[2-(2-methoxyethoxy)ethoxy]ethyl, 2-(2-methoxyethoxy)ethyl, tetrahydrofuran-2-ylmethyl, (3-methyloxetan-3-yl)methyl, 1H-imidazol-2-ylmethyl, tetrahydrofuran-3-yl, 2-oxotetrahydrofuran-3-yl, 2-tert-butoxy-2-oxoethyl, 1-methoxy-3-methyl-1-oxobutan-2-yl, 1-methoxy-1-oxopropan-2-yl or 3-ethoxy-3-oxopropyl, propan-2-yloxy, methyl, ethyl, n-propyl, 2-ethoxyethyl or 2-chloroethyl,
R⁴ stands for hydrogen or —NR¹²R¹³ or —NHR¹³,
R⁵ and R⁶ mutually independently stand for hydrogen, fluorine or cyano
or together with the carbon atoms to which they are bound form a single ring wherein they together stand for —(CH═CH—CH═CH)—, —(CH═CH—N(R¹⁹), —(CH═CH—CH═N)—, —(NH—CH═N)— or —(CH₂—C(O)—N(R¹⁹)—, optionally singly or multiply, identically or differently substituted with R¹¹,
R⁷ mutually independently stands for one or more of the following groups: fluorine, chlorine, cyano or methyl,
R¹¹ stands for —OH, fluorine, chlorine, bromine, cyano, —NH—C(O)R²⁰, —C(O)R²⁰, —C(O)OR²⁰, —C(O)NR²⁰R²¹ or —SO₂R²⁰
or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with fluorine, chlorine, bromine, —OH, cyano, C₁-C₆ alkyl or —O—(C₁-C₄ alkyl),
R¹² stands for hydrogen, —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₂-C₆ alkenyl, C₂-C₆ alkynyl, C₂-C₉ heterocyclyl or C₂-C₉ heteroaryl or hydrogen,
R¹⁵ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with halogen, —OH, cyano or C₁-C₄ alkyl,
or for hydrogen
R¹⁶ stands for hydrogen, methyl, ethyl or propyl,
R¹⁹ stands for H, C₂-C₆ alkynyl, —C(O)R¹⁵, —SO₂R¹⁵ or —C(O)OR¹⁵, and
R²⁰ and R²¹ mutually independently stand for methyl, ethyl, propyl, isopropyl, cyclopropyl or cyclobutyl, each optionally singly or multiply, identically or differently substituted with fluorine, chlorine, bromine, —OH or cyano,
or for hydrogen,
and agrochemically active salts thereof.

3. Compounds of the formula [I-a] according to one or more of claims 1 to 2,
wherein the symbols have the following meanings,
X¹ stands for C—H, and
R⁴ stands for hydrogen
and agrochemically active salts thereof.

4. Compounds of the formula [I-a] according to one or more of claims 1 to 2,
wherein the symbols have the following meanings,
X¹ stands for N,
R⁴ stands for —NHR¹³, and
R¹³ stands for C₁-C₆ alkyl, C₃-C₆ cycloalkyl, C₂-C₆ alkenyl, C₂-C₆ alkynyl, C₂-C₉ heterocyclyl or C₂-C₉ heteroaryl, each optionally singly or multiply, identically or differently substituted with fluorine, chlorine, bromine, —OH or cyano,
and agrochemically active salts thereof.

5. Compounds of the formula [I-b], 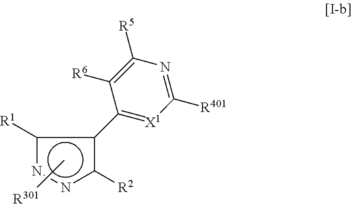
wherein the symbols have the following meanings:
X¹ stands for C—H or N,
R¹ stands for phenyl, naphthalenyl, quinolin-5-yl, quinolin-8-yl, isoquinolin-5-yl, isoquinolin-8-yl, 1-benzothiophen-4-yl, 1-benzothiophen-7-yl, 1-benzo-furan-4-yl, 1-benzofuran-7-yl, 1,3-benzodioxol-4-yl or 1,3-benzodioxol-5-yl, each optionally singly or multiply, identically or differently substituted with R⁷,
R² stands for cyano, nitro, halogen, C₁-C₆ alkyl, C₁-C₆ alkoxy, C₁-C₆ haloalkyl, C₁-C₆ haloalkoxy, C₁-C₆ alkylthio, C₃-C₆ cycloalkyl, C₃-C₆ halocycloalkyl, C₂-C₉ heterocyclyl or hydrogen,
R³⁰¹ stands for —C(O)N(R⁹R¹⁰), —C(O)R⁹, —C(O)OR⁹ or —S(O)₂R⁹ or for C₁-C₆ alkyl, C₂-C₆ alkenyl, C₃-C₆ alkenyl, C₂-C₆ alkynyl, C₃-C₈ cycloalkyl, C₁-C₆ alkoxy, C₂-C₉ heterocyclyl, C₂-C₉ oxoheterocyclyl, or heteroaryl, each optionally singly or multiply, identically or differently substituted with R⁸,
R⁴⁰¹ stands for —NR¹²R¹³, —C(O)NR¹²R¹³ or —N(R¹²)₂
R⁵ and R⁶ mutually independently stand for hydrogen, fluorine, chlorine, bromine, cyano, nitro, —OH or —SH,
or for C₁-C₆ alkyl, C₃-C₆ cycloalkyl, C₂-C₆ alkenyl, C₂-C₆ alkynyl, C₆-C₁₄ aryl, —O—(C₁-C₄ alkyl), —O—(C₆-C₁₄ aryl), —S—(C₁-C₄ alkyl), —S(O)—(C₁-C₆ alkyl) or —C(O)—(C₁-C₆ alkyl), each optionally singly or multiply, identically or differently substituted with R¹¹,
or else together with the carbon atom to which they are bound form a ring with 3 to 8 ring atoms, wherein the ring can contain 1 to 4 hetero atoms from the range oxygen, sulphur or —N—R¹⁹, optionally singly or multiply, identically or differently substituted with halogen, oxygen, cyano or C₁-C₄ alkyl,
R⁷ mutually independently stands for one or more of the following groups: fluorine, chlorine, bromine, cyano, nitro, —OH or —SH,
or for C₁-C₆ alkyl, C₃-C₆ cycloalkyl, C₂-C₆ alkenyl, C₂-C₆ alkynyl, tri(C₁-C₄ alkyl)silyl, C₆-C₁₄ aryl, —O—(C₁-C₄ alkyl), —O—(C₆-C₁₄ aryl), —S—(C₁-C₄ alkyl), —S(O)—(C₁-C₆ alkyl) or —S(O)₂—(C₁-C₆ alkyl), optionally singly or multiply, identically or differently substituted with fluorine, chlorine, bromine, —OH, cyano, C₁-C₆ alkyl or —O—(C₁-C₄ alkyl),
R⁸ stands for —OH, halogen, NO₂, cyano, —NR⁹R¹⁰, —C(O)N(R⁹R¹⁰), —C(O)R⁹, —C(O)OR⁹, —O—C(O)R⁹ or —(CH₂)ₙC(O)R⁹ wherein n=a whole number between 1 and 6
or for C₁-C₆ alkyl, C₂-C₈ alkenyl, C₂-C₈ alkynyl, C₃-C₈ cycloalkyl, C₁-C₆ haloalkyl, C₆-C₁₄ aryl, C₂-C₉ heterocyclyl, C₂-C₉ heteroaryl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with R¹¹,
R⁹ and R¹⁰ mutually independently stand for C₁-C₆ alkyl, C₂-C₈Alkenyl, C₂-C₈ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
or for hydrogen,
R¹¹ stands for —OH, fluorine, chlorine, bromine, cyano, —NH—C(O)R²⁰, —NR²⁰R²¹, —C(O)R²⁰, —C(O)OR²⁰, —C(O)NR²⁰R²¹ or —SO₂R²⁰
or for C₁-C₆ alkyl, C₂-C₆ alkenyl, C₂-C₆ alkynyl, C₁-C₁₁ heteroalkyl, C₃-C₈ cycloalkyl, —O—(C₁-C₄ alkyl), —S—(C₁-C₄ alkyl), —O—(C₃-C₈ cycloalkyl), S—(C₃-C₈ cycloalkyl), C₆-C₁₄ aryl, —O—(C₆-C₁₄ aryl), —S—(C₆-C₁₄ aryl), C₂-C₉ heterocyclyl or C₂-C₉ heteroaryl, optionally singly or multiply identically or differently substituted with fluorine, chlorine, bromine, —OH, carbonyl, cyano, C₁-C₆ alkyl or —O—(C₁-C₄ alkyl),
R¹² stands for —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with fluorine, chlorine, bromine, —OH, cyano, C₁-C₆ alkyl, —O—C(O)—C₁-C₄ alkyl, —O—P(O)(O—C₁-C₄ alkyl)₂, —O—B(O—C₁-C₄ alkyl)₂ or —O—(C₁-C₄ alkyl),
or for hydrogen,
R¹⁵ and R¹⁶ mutually independently stand for hydrogen or —OH,
or for C₁-C₆ alkyl, C₃-C₆ cycloalkyl, C₂-C₆ alkenyl, C₂-C₆ alkynyl, C₃-C₈ cycloalkyl, C₆-C₁₄ aryl, C₂-C₉ heterocyclyl or C₂-C₉ heteroaryl, each optionally singly or multiply, identically or differently substituted with R¹¹,
or together with the nitrogen atom to which they are bound form a 3 to 7-membered ring, which can contain a further hetero atom from the range N or O not adjacent to the nitrogen,
R¹⁹ stands for H, C₁-C₆ alkyl, C₃-C₆ cycloalkyl, C₂-C₆ alkenyl, C₂-C₆ alkynyl, —C(S)R¹⁵, —C(O)R¹⁵, —SO₂R¹⁵ or —C(O)OR¹⁵, and
R²⁰ and R²¹ mutually independently stand for C₁-C₆ alkyl, C₃-C₆ cycloalkyl, C₂-C₆ alkenyl or C₂-C₆ alkynyl, each optionally singly or multiply, identically or differently substituted with fluorine, chlorine, bromine, —OH or cyano or hydrogen,
and agrochemically active salts thereof,
wherein the following compounds are excepted:
a) Compounds wherein,
R³⁰¹ stands for optionally substituted [1,2,4[triazolo[4,3-b]pyridazin-6-yl, 7,8-dihydro[1,2,4[triazolo[4,3-b]pyridazin 6-yl, 6-oxo-1,6-dihydropyridazin-3-yl, 6-oxo-1,4,5,6-tetrahydropyridazin-3-yl or 6-chloropyridazin-3-yl, and
R⁵, R⁶ stand for H, and
b) the compounds 4-{1-[2-(dimethylamino)ethyl]-3-(4-fluorophenyl)-1H-pyrazol-4-yl}-N,N-dimethylpyridin-2-amine and 1-(4-{4-[1-ethyl-3-(4-nitrophenyl)-1H-pyrazol-4-yl]-1H-pyrrolo[2,3-b]pyridin-6-yl}phenyl)-N,N-dimethylmethanamine.

6. Compounds of the formula [I-b] according to claim 5,
wherein the symbols have the following meanings,
R¹ stands for phenyl, optionally singly or multiply, identically or differently substituted with R⁷,
R² stands for methyl, ethyl, isopropyl, cyclopropyl, or hydrogen,
R³⁰¹ stands for C₁-C₆ alkyl, C₂-C₆ alkenyl, C₃-C₆ alkenyl, C₂-C₆ alkynyl, C₃-C₈ cycloalkyl or C₁-C₆ alkoxy, each optionally singly or multiply, identically or differently substituted with R⁸,
R⁴⁰¹ stands for —NR¹²R¹³,
R⁵ and R⁶ mutually independently stand for hydrogen, fluorine or cyano
or together with the carbon atoms to which they are bound form a single ring wherein they together stand for —(CH═CH—CH═CH)—, —(CH═CH—N(R¹⁹)—, —(CH═CH—CH═N)— or —(CH₂—C(O)—N(R¹⁹)—, optionally singly or multiply, identically or differently substituted with R¹¹,
R⁷ stands for fluorine, chlorine, cyano or methyl,
R⁸ stands for fluorine, chlorine or cyano or for C₁-C₆ alkyl, C₂-C₈ alkenyl, C₂-C₈ alkynyl, C₃-C₈ cycloalkyl, C₆-C₁₄ aryl, heterocyclyl, heteroaryl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with R¹¹,
R¹¹ stands for one or more of the following groups: —OH, fluorine, chlorine, bromine, cyano, —NH—C(O)R²⁰, —C(O)R²⁰, —C(O)OR²⁰, —C(O)NR²⁰R²¹ or —SO₂R²⁰
or for C₁-C₆ alkyl, C₃-C₈ cycloalkyl, C₂-C₆ alkenyl, C₂-C₆ alkynyl, —O—(C₁-C₄ alkyl), —S—(C₁-C₄ alkyl), —O—(C₃-C₈ cycloalkyl) or —S—(C₃-C₈ cycloalkyl), each optionally singly or multiply, identically or differently substituted with fluorine, chlorine, bromine, —OH, cyano, C₁-C₆ alkyl or —O—(C₁-C₄ alkyl),
R¹² stands for: —C(S)R¹⁵, —C(O)R¹⁵, —SO₂R¹⁵, —C(O)OR¹⁵, —OR¹⁵ or —C(O)NR¹⁵R¹⁶,
R¹³ stands for C₁-C₆ alkyl, C₂-C₆ alkenyl, C₂-C₆ alkynyl or hydrogen,
R¹⁵ stands for C₁-C₆ alkyl, C₃-C₆ cycloalkyl, C₂-C₆ alkenyl, C₂-C₆ alkynyl, C₃-C₈ cycloalkyl, C₂-C₉ heterocyclyl or C₂-C₉ heteroaryl, each optionally singly or multiply, identically or differently substituted with halogen, —OH, cyano or C₁-C₄ alkyl,
or for hydrogen,
R¹⁶ stands for hydrogen, methyl, ethyl or propyl,
R¹⁹ stands for H, —C₂-C₆ alkynyl, —C(O)R¹⁵, —SO₂R¹⁵ or —C(O)OR¹⁵, and
R²⁰ and R²¹ mutually independently stand for methyl, ethyl, propyl, isopropyl, cyclo-propyl or cyclobutyl, each optionally singly or multiply, identically or differently substituted with fluorine, chlorine, bromine, —OH or cyano,
or for hydrogen,
and agrochemically active salts thereof.

7. Compounds of the formula [I-b] according to one or more of claims 5 to 6,
wherein the symbols have the following meanings,
X¹ stands for N,
R¹ stands for phenyl, 3-methylphenyl, 2-fluorophenyl, 3-fluorophenyl, 4-fluorophenyl, 3-trifluoromethylphenyl, 4-chlorophenyl, 2,5-difluorophenyl, 2,4-difluorophenyl, 2,6-difluorophenyl, 3-methyl-4-fluorophenyl, 3-cyano-4-fluorophenyl or 2,4,6-trifluorophenyl,
R³⁰¹ stands for methyl, ethyl, 1-propyl, propan-2-yl, isobutyl, butan-2-yl, 2-methylpropyl, 2,2-dimethylpropyl, 2-(morpholin-4-yl)ethyl, 2-cyanoethyl, cyanomethyl, 2-cyano-2-methylpropyl, 3-methylbut-2-en-1-yl, but-2-en-1-yl, but-3-en-2-yl, propadienyl, prop-2-en-1-yl, prop-2-yn-1-yl, but-2-yn-1-yl, but-3-yn-2-yl, 2-methylbut-3-yn-2-yl, 2-methylbut-3-yn-2-yl, cyclopropyl, cyclobutyl, cyclopentyl, cyclohexyl, (2,2-dichlorocyclopropyl)methyl, cyclopropylmethyl, 1-cyclopropylethyl, trichloromethyl, trifluoromethyl, 2-fluoroethyl, 2,2-difluoroethyl, 2,2,2-trifluoroethyl, 2,2,2-trichloroethyl, 2-chloroethyl, 2-bromoethyl, 2-fluoropropyl, 3-fluoropropyl, 2-chloropropyl, 3-chloropropyl, 1,3-difluoropropan-2-yl, 1,1,1-trifluoropropan-2-yl, 1,1,1-trifluoro-2-methylpropan-2-yl, 3,3,3-trifluoro-2-hydroxypropyl, 2-fluoro-benzyl, 3-fluorobenzyl, 4-fluorobenzyl, 2,3-difluorobenzyl, 2-chloro-6-fluorobenzyl, 1-(2-chlorophenyl)ethyl, 1-(3-chlorophenyl)ethyl, 1-(4-chloro-phenyl)ethyl, 3-cyanobenzyl, 4-cyanobenzyl, 4-(difluoromethoxy)benzyl, 2-cyanobenzyl, 2-(3-chlorophenyl)ethyl, 2-(2-chlorophenyl)ethyl, 1-naphthylmethyl, (pyridine-3-ylmethyl, 2-chloro-1,3-thiazol-5-yl)methyl, methoxymethyl, 2-methoxyethyl, 2-methoxypropyl, 2-(methylsulphanyl)-ethyl, 2-(trifluoromethoxy)ethyl, 1-methoxypropan-2-yl, 2-[2-(2-methoxyethoxy)ethoxy]ethyl, 2-(2-methoxyethoxy)ethyl, 1,3-dimethoxypropan-2-yl, 2-(cyclopropyloxy)ethyl, tetrahydrofuran-2-ylmethyl, (3-methyloxetan-3-yl)methyl, 1H-imidazol-2-ylmethyl, tetrahydrofuran-3-yl, 2-oxotetrahydro-furan-3-yl, 2-tert-butoxy-2-oxoethyl, 1-methoxy-3-methyl-1-oxobutan-2-yl, 1-methoxy-1-oxopropan-2-yl or 3-ethoxy-3-oxopropyl, 1-cyanopropan-2-yl, propan-2-yloxy or 2-ethoxyethyl,
R⁴⁰¹ stands for —NHR¹²,
R¹¹ stands for one or more of the following groups: —OH, fluorine, chlorine, cyano, methyl, ethyl or cyclopropyl,
R¹² stands for —C(S)R¹⁵, —SO₂R¹⁵, —C(O)OR¹⁵ or —C(O)R¹⁵,
R¹⁵ stands for methyl, ethyl, n-propyl, isopropyl, sec-butyl, isobutyl, cyclopropyl, cyclobutyl, cyclopentyl, cyclohexyl, 2-methoxyethyl, (2-methoxyethoxy)-methyl, cyclopentenyl, cyclohexenyl, oxetanyl, tetrahydrofuran-2-yl, ethinyl, prop-1-yn-1-yl, prop-1-en-1-yl, aminomethyl, aminoethyl, amino-propyl, aminobutyl, aminoisopropyl, aminocyclopropyl, aminocyclobutyl, aminocyclopentyl, dimethylamino, ethyl(methyl)amino, pyrrolidinyl, diethylamino, 2-pyridyl, 3-pyridyl, 4-pyridyl, ethoxycarbonyl, benzyl, phenyl, 2-thienyl or 3-thienyl, each optionally singly or multiply, identically or differently substituted with halogen, OH, cyano or C₁-C₄ alkyl,
or for hydrogen, and
R¹⁹ stands for H, acetyl, ethoxycarbonyl, methoxycarbonyl, prop-2-yn-1-yl or but-2-yn-1-yl,
and agrochemically active salts thereof.

8. Compounds of the formula [I-b] according to one or more of claims 5 to 6,
wherein the symbols have the following meanings,
X¹ stands for C—H,
R¹ stands for 4-fluorophenyl, 3-chlorophenyl, 2,6-difluorophenyl or 3-methylphenyl,
R² stands for cyclopropyl, methyl, H or difluoromethoxy, and
R⁴⁰¹ stands for acetylamino, n-propionylamino, isobutyrylamino, (cyclopropylcarbonyl)amino, (methoxyacetyl)amino, 2-methoxypropanoyl, (2-methylbutanoyl)amino, but-2-enoylamino, prop-2-ynoylamino, 3-(dimethylamino)-prop-2-enoyl]amino, 3,3,3-trifluoropropanoyl)amino, 3,3-difluoro-propanoyl)amino, (cyclopropylacetyl)amino, lactoylamino, (cyclobutyl-carbonyl)amino, (cyclopentylacetyl)amino, 2-methylcyclopropyl)carbonyl]-amino, (3-methylbutanoyl)amino, (phenylacetyl)amino, benzoylamino, (3-thienylcarbonyl)amino, (2-thienylcarbonyl)amino (2-hydroxy-2-methylpropanoyl)amino, [(2-methoxyethoxy)acetyl]amino or 2,3-dihydroxypropanoyl)amino,
and agrochemically active salts thereof.

9. Method for the control of phytopathogenic and mycotoxin-producing fungi, characterized in that phenylpyri(mi)dinylazoles of the formula [I-a] and/or [I-b] according to one or more of claims 1 to 8 are applied onto the fungi and/or their habitat.

10. Agent for the control of phytopathogenic and mycotoxin-producing fungi, characterized by a content of at least one phenylpyri(mi)dinylazole of the formula [I-a] and/or [I-b] according to one or more of claims 1 to 8 as well as thinners and/or surface-active substances.

11. Compounds of the formula [X], 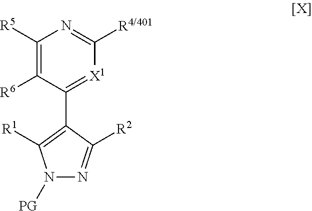
wherein
PG stands for tetrahydro-2H-pyran-2-yl, and
R¹, R², X¹, R⁶, R⁵, R^{4/401} stand for the same residue definitions as defined for formula [I-a] and [I-b] in claims 1 to 8,
and agrochemically active salts thereof.

12. Compounds of the formula [XI] 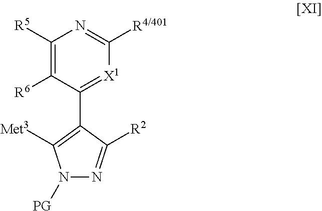
wherein
Met³ stands for tributylstannyl, 4,4,5,5-tetramethyl-1,3,2-dioxaborolan-2-yl,
PG stands for tetrahydro-2H-pyran-2-yl, 2-(trimethylsilyl)ethoxy]methyl, and
R², X¹, R⁶, R⁵, R^{4/401} stand for the same residue definitions as defined for formula [I-a] and [I-b] in claims 1 to 8,
and agrochemically active salts thereof, wherein the compound 1-({4-[1-(2,2-difluoroethyl)-3-(trimethylstannyl)-1H-pyrazol-4-yl]pyrimidin-2-yl}amino)propan-2-ol is excepted.

13. Compounds of the formula [III] 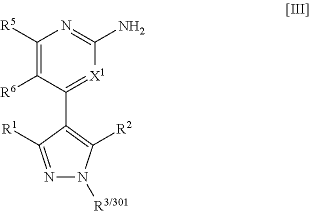
in which
R¹, R², X¹, R⁶, R⁵, R^{3/301} stand for the same residue definitions as defined for formula [I-a] and [I-b] in claims 1 to 8,
and agrochemically active salts thereof, wherein the compounds 4-[3-(4-fluorophenyl)-5-methyl-1H-pyrazol-4-yl]pyridin-2-amine, 4-[3-(4-chlorophenyl)-5-methyl-1H-pyrazol-4-yl]pyridin-2-amine, 4-[3-(4-methoxyphenyl)-5-methyl-1H-pyrazol-4-yl]pyridin-2-amine, 4-[3-(4-fluorophenyl)-1H-pyrazol-4-yl]pyrimidin-2-amine; 4-(5-methyl-3-phenyl-1H-pyrazol-4-yl)pyrimidin-2-amine and [4-(2-aminopyrimidin-4-yl)-3-chloro-5-hydroxy-phenyl)-1H-pyrazol-1-yl]acetonitrile are excepted.

14. Compounds of the formula [V] 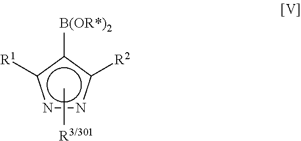
wherein
B(OR*)₂ stands for —B(OiPr)₂ or —B(OH)₂, and
R¹, R², R^{3/301} stand for the same residue definitions as defined for formula [I-a] and [I-b] in claims 1 to 8,
and agrochemically active salts thereof, wherein the compound 1-methyl-3-phenyl-4-(4,4,5,5-tetramethyl-1,3,2-dioxaborolan-2-yl)-1H-pyrazoles is excepted.

15. Compounds of the formula [VI] 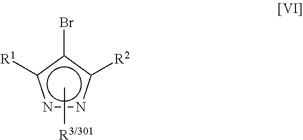
wherein
R¹, R², R^{3/301} stand for the same residue definitions as defined for formula [I-a] and [I-b] in claims 1 to 8,
and agrochemically active salts thereof, wherein compounds in which R^{3/301}═H, CH₃ or C(CH₃)₃ are excepted.
